(19) United States
(12) Patent Application Publication
(10) Pub. No.: US 2022/0119354 A1
(43) Pub. Date: Apr. 21, 2022

(54) DIAMINO-SUBSTITUTED PYRIDINES AND PYRIMIDINES AS HERBICIDES

(71) Applicant: FMC Corporation, Philadelphia, PA (US)

(72) Inventor: DE Saptarshi, Hyderabad (IN)

(21) Appl. No.: 17/426,873

(22) PCT Filed: Jan. 30, 2020
(86) PCT No.: PCT/US2020/015779

§ 371 Date: Jul. 29, 2021

Related U.S. Application Data
(60) Provisional application No. 62/800,418, filed on Feb. 1, 2019.
(60) Provisional application No. 62/940,884, filed on Nov. 26, 2019.

(51) Int. Cl. C07D 239/48; C07D 211/76; C07D 207/277; A01N 43/36; A01N 43/40; A01P 13/00

(52) U.S. Cl. CPC C07D 239/48; C07D 211/76; A01P 13/00; A01N 43/36; A01N 43/40; C07D 207/277

(57) ABSTRACT

Disclosed are compounds of Formula 1, including all stereoisomers, A-oxides, and salts thereof, wherein A is selected from and X, Q¹, Q², Q³, Q⁴, R, R¹, R², R³, R⁴ and n are as defined in the disclosure. Also disclosed are compositions containing the compounds of Formula 1 and methods for controlling undesired vegetation comprising contacting the undesired vegetation or its environment with an effective amount of a compound or a composition of the invention.

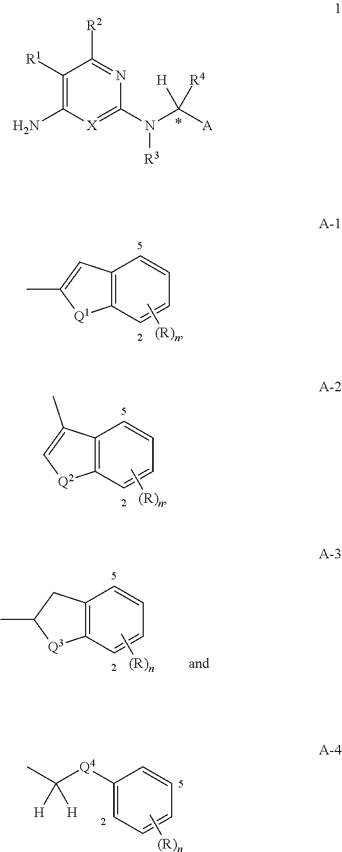

### FIELD OF THE INVENTION

This invention relates to certain amino-substituted pyridines and pyrimidines, their N-oxides, salts and compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, maize, potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds that are more effective, less costly, less toxic, environmentally safer or have different sites of action.

Published patent applications WO 2010/076010, WO 2013/144187 and WO 2017/016914 disclose aminopyrimidine derivatives.

### SUMMARY OF THE INVENTION

This invention is directed to a compound of Formula 1 (including all stereoisomers, (N-oxides, and salts thereof), agricultural compositions containing them and their use as herbicides

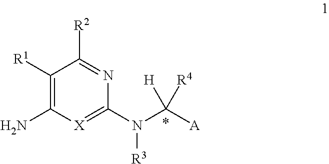

wherein
A is selected from

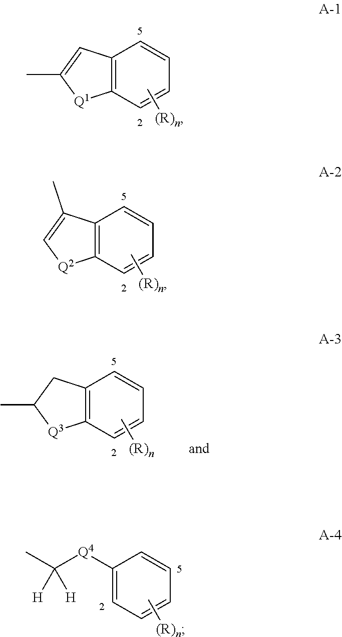

X is N or CR⁵;
R¹ and R² are independently H, halogen, hydroxy, cyano, nitro, amino, SF₅, C(O)OH, C(O)NH₂, C(S)NH₂, C₁-C₆ alkyl, C₁-C₆ haloalkyl, C₂-C₆ alkylcarbonyl, C₂-C₆ haloalkylcarbonyl, C₂-C₆ alkylcarbonyloxy, C₂-C₆ haloalkylcarbonyloxy, C₁-C₆ alkoxy, C₁-C₆ haloalkoxy, C₄-C₁₄ cycloalkylalkyl, C₃-C₈ cycloalkoxy, C₃-C₈ cyclohaloalkoxy, C₄-C₁₂ cycloalkylalkoxy, C₂-C₆ alkoxycarbonyl, C₂-C₆ haloalkoxycarbonyl, C₂-C₆ alkoxycarbonyl-C₁-C₆ haloalkyl, C₂-C₆ alkenyl, C₂-C₆ haloalkenyl, C₃-C₆ alkenylcarbonyl, C₃-C₆ haloalkenylcarbonyl, C₂-C₆ alkenyloxy, C₂-C₆ haloalkenyloxy, C₃-C₆ alkenyloxycarbonyl, C₃-C₆ haloalkenyloxycarbonyl, C₂-C₄ cyanoalkyl, C₂-C₄ cyanoalkoxy, C₁-C₄ nitroalkyl, C₁-C₄ nitroalkoxy, C₂-C₆ alkynyl, C₂-C₆ haloalkynyl, C₃-C₆ alkynylcarbonyl, C₃-C₆ haloalkynylcarbonyl, C₂-C₆ alkynyloxy, C₂-C₆ haloalkynyloxy, C₃-C₆ alkynyloxycarbonyl, C₃-C₆ haloalkynyloxycarbonyl, C₁-C₄ alkylthio, C₁-C₄ haloalkylthio, C₂-C₄ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylsulfonyloxy, C₁-C₄ haloalkylsulfonyloxy C₁-C₆ hydroxyalkyl, C₁-C₆ hydroxyalkoxy, C₂-C₁₂ alkoxyalkyl, C₂-C₁₂ alkylthioalkyl, C₂-C₁₂ haloalkoxyalkyl, C₂-C₁₀ haloalkylthioalkoxy, C₂-C₁₂ alkoxyalkoxy, C₂-C₁₀ alkylthioalkoxy, C₂-C₁₂ haloalkoxyalkoxy, C₂-C₁₀ haloalkylthio, C₁-C₄ aminoalkyl, C₂-C₈ alkylaminoalkyl, C₃-C₁₂ dialkylaminoalkyl, C₁-C₄ aminoalkoxy, C₂-C₈ alkylaminoalkoxy or C₃-C₁₂ dialkylamino; or
R¹ and R² are independently C₃-C₈ cycloalkyl, each cycloalkyl optionally substituted with halogen, hydroxy, cyano, nitro, amino, C(O)OH, C(O)NH₂, C₁-C₆ alkyl, C₁-C₆ haloalkyl, C₁-C₆ haloalkoxy, C₃-C₈ cycloalkoxy, C₃-C₈ cyclohaloalkoxy, C₂-C₆ alkylcarbonyl, C₂-C₆ alkoxycarbonyl, C₂-C₆ alkoxycarbonyloxy, C₂-C₆ haloalkylcarbonyloxy, C₄-C₈ cycloalkylcarbonyl, C₄-C₈ cycloalkoxycarbonyl, C₂-C₆ haloalkoxycarbonyl, C₄-C₁₀ cycloalkylcarbonyloxy, C₃-C₈ cycloalkoxycarbonyloxy, C₂-C₆ haloalkoxycarbonyloxy;
R³ is H, C₁-C₄ alkyl, C₁-C₆ alkylcarbonyl, C₁-C₆ haloalkylcarbonyl, C₂-C₆ alkoxycarbonyl or C₂-C₆ haloalkoxycarbonyl;
R⁴ is C₁-C₆ alkyl, C₁-C₆ haloalkyl, C₂-C₆ alkenyl, C₂-C₆ alkynyl, C₃-C₇ cycloalkyl or C₃-C₇ cyclohaloalkyl;
R⁵ is H, halogen, cyano, C₁-C₆ alkyl or C₁-C₆ haloalkyl;
each R is independently halogen, hydroxy, cyano, amino, nitro, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₁-C₄ hydroxyalkyl, C₃-C₇ cycloalkyl, C₃-C₇ cyclohaloalkyl, C₄-C₈ cycloalkylalkyl, C₁-C₄ alkoxy, C₁-C₄ haloalkoxy, C₃-C₇ cycloalkoxy, C₃-C₇ cyclohaloalkoxy, C₄-C₈ cycloalkylalkoxy, C₂-C₄ alkenyloxy, C₂-C₄ alkynyloxy, C₂-C₄ alkoxyalkyl, C₂-C₄ alkoxyhaloalkyl, C₂-C₆ alkylcarbonyloxy, C₁-C₄ alkylthio, C₁-C₄ haloalkylthio, C₁-C₄ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylsulfonyloxy, C₂-C₄ cyanoalkyl, C₂-C₄ cyanoalkoxy, C₁-C₄ nitroalkyl, C₁-C₄ alkylamino, C₂-C₈ dialkylamino, C₃-C₆ cycloalkylamino, C₂-C₄ alkylcarbonyl, C₂-C₆ alkoxycarbonyl, C₂-C₆ alkylaminocarbonyl, C₃-C₈ dialkylaminocarbonyl, CONH₂ or CO₂H; or
each R is independently phenyl, phenylW¹, a 5- or 6-membered heterocyclic ring, a 5- or 6-membered heterocyclic ringW², naphthalenyl, or naphthalenylW², each optionally substituted with up to five substituents independently selected from the group consisting of H, halogen, hydroxy, cyano, amino, nitro, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₁-C₄ hydroxyalkyl, C₃-C₇ cycloalkyl, C₃-C₇ cyclohaloalkyl, C₄-C₈ cycloalkylalkyl, C₁-C₄ alkoxy, C₁-C₄ haloalkoxy, C₃-C₇ cycloalkoxy, C₃-C₇ cyclohaloalkoxy, C₄-C₈ cycloalkylalkoxy, C₂-C₄ alkenyloxy, C₂-C₄ alkynyloxy, C₂-C₄ alkoxyalkyl, C₂-C₄ alkoxyhaloalkyl, C₂-C₆ alkylcarbonyloxy, C₁-C₄ alkylthio, C₁-C₄ haloalkylthio, C₂-C₄ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylsulfonyloxy, C₂-C₄ cyanoalkyl, C₂-C₄ cyanoalkoxy, C₁-C₄ nitroalkyl, C₁-C₄ alkylamino, C₂-C₈ dialkylamino, C₃-C₆ cycloalkylamino, C₂-C₄ alkylcarbonyl, C₂-C₆ alkoxycarbonyl, C₂-C₆ alkylaminocarbonyl, C₃-C₈ dialkylaminocarbonyl, C(O)OH, C(O)NH₂ and C(S)NH₂;
each W¹ is independently C₁-C₆ alkanediyl or C₂-C₆ alkenediyl;
each W² is independently C₁-C₆ alkanediyl;
n is 0, 1, 2, 3 or 4;
Q¹ is O, S, carbonyl, sulfonyl, sulfinyl, CR^{6a}R^{6b}, —C(R⁶)═C(R⁷)—, —C(R^{6a})(R^{6b})—C(R^{7a})C(R^{7b})— or NR⁸;
Q² is O, S, carbonyl, sulfonyl, sulfinyl, CR^{6a}R^{6b}, —C(R⁶)═C(R⁷)—, —C(R^{6a})(R^{6b})—C(R^{7a})C(R^{7b})— or NR⁸;
Q³ is O, S, carbonyl, sulfonyl, sulfinyl, CR^{6a}R^{6b}, —C(R⁶)═C(R⁷)—, —C(R^{6a})(R^{6b})—C(R^{7a})C(R^{7b})— or NR⁸;
Q⁴ is O, S, carbonyl, sulfonyl, sulfinyl, CR^{6a}R^{6b}, —C(R⁶)═C(R⁷)—, —C(R^{6a})(R^{6b})—C(R^{7a})C(R^{7b})— or NR⁸;
wherein the bond projecting to the right of the —C(R⁶)═C(R⁷)— or —C(R^{6a})(R^{6b})—C(R^{7a})C(R^{7b})— moieties of Q¹, Q², Q³ or Q⁴ is attached to the benzene moiety of A-1, A-2, A-3 or A-4, respectively; and
each R⁶, R^{6a}, R^{6b}, R⁷, R^{7a}, R^{7b} and R⁸ is independently H, C₁-C₆ alkyl or C₁-C₆ haloalkyl.

More particularly, this invention pertains to a compound of Formula 1 (including all stereoisomers), an N-oxide or a salt thereof. This invention also relates to a herbicidal composition comprising a compound of the invention (i.e. in a herbicidally effective amount) and at least one component selected from the group consisting of surfactants, solid diluents and liquid diluents. This invention further relates to a method for controlling the growth of undesired vegetation comprising contacting the vegetation or its environment with a herbicidally effective amount of a compound of the invention (e.g., as a composition described herein). This invention also includes a herbicidal mixture comprising (a) a compound selected from Formula 1, N-oxides, and salts thereof, and (b) at least one additional active ingredient selected from (b1) through (b16); and salts of compounds of (b1) through (b16), as described below.

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method. The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, mixture, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling”, used either alone or in a combination of words means a young plant developing from the embryo of a seed.

As referred to herein, the term “broadleaf” used either alone or in words such as “broadleaf weed” means dicot or dicotyledon, a term used to describe a group of angiosperms characterized by embryos having two cotyledons.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

The term “alkanediyl” denotes a straight-chain or branched divalent hydrocarbon radical. Examples include CH₂, CH₂CH₂, CH(CH₃), CH₂CH₂CH₂, CH₂CH(CH₃), and the different butylene, pentylene or hexylene isomers. “Alkenediyl” denotes a straight-chain or branched divalent hydrocarbon radical containing one olefinic bond. Examples include CH═CH, CH₂CH═CH and CH═C(CH₃).

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkenyloxy” includes straight-chain or branched alkenyloxy moieties. Examples of “alkenyloxy” include H₂C═CHCH₂O, (CH₃)₂C═CHCH₂O, (CH₃)CH═CHCH₂O, (CH₃)CH═C(CH₃)CH₂O and CH₂═CHCH₂CH₂O. “Alkynyloxy” includes straight-chain or branched alkynyloxy moieties. Examples of “alkynyloxy” include HC≡CCH₂O, CH₃C≡CCH₂O and CH₃C≡CCH₂CH₂O. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. “Cyanoalkyl” denotes an alkyl group substituted with one cyano group. Examples of “cyanoalkyl” include NCCH₂, NCCH₂CH₂ and CH₃CH(CN)CH₂. “Alkylamino”, “dialkylamino” and the like, are defined analogously to the above examples.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “cycloalkoxy” denotes cycloalkyl linked through an oxygen atom such as cyclopentyloxy and cyclohexyloxy. “Cycloalkylalkoxy” denotes cycloalkylalkyl linked through an oxygen atom attached to the alkyl chain. Examples of “cycloalkylalkoxy” include cyclopropylmethoxy, cyclopentylethoxy, and other cycloalkyl moieties bonded to straight-chain or branched alkoxy groups.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkoxy”, “haloalkylthio”, “haloalkenyl”, “haloalkynyl”, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—. Examples of “haloalkenyl” include (Cl)₂C═CHCH₂— and CF₃CH₂CH═CHCH₂—. Examples of “haloalkynyl” include HC≡CCHCl—, CF₃C≡C—, CCl₃C≡C— and FCH₂C≡CCH₂—. Examples of “haloalkoxyalkoxy” include CF₃OCH₂O—, C₁CH₂CH₂OCH₂CH₂O—, Cl₃CCH₂OCH₂O— as well as branched alkyl derivatives.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. Examples of “alkoxycarbonyl” include CH₃OC(═O)—, CH₃CH₂OC(═O)—, CH₃CH₂CH₂OC(═O)—, (CH₃)₂CHOC(═O)— and the different butoxy- or pentoxycarbonyl isomers. The term “phenylW¹” means that phenyl is bonded through W¹ to the remainder of Formula 1. The term “5- or 6-membered heterocyclic ringW²” means that the 5- or 6-membered heterocyclic ring is bonded through W² to the remainder of Formula 1. The term naphthalenylW² means that naphthalene is bonded through W² to the remainder of Formula 1.

The total number of carbon atoms in a substituent group is indicated by the “Cᵢ-Cⱼ” prefix where i and j are numbers from 1 to 10. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a compound is substituted with a substituent bearing a subscript that indicates the number of said substituents can exceed 1, said substituents (when they exceed 1) are independently selected from the group of defined substituents, (e.g., (R)ₙ, n is 1, 2, 3 or 4). When a group contains a substituent which can be hydrogen, for example (R¹ or R²), then when this substituent is taken as hydrogen, it is recognized that this is equivalent to said group being unsubstituted at the position indicated for the substituent. When a variable group is shown to be optionally attached to a position, for example (R)ₙ wherein n may be 0, then hydrogen may be at the position even if not recited in the variable group definition. When one or more positions on a group are said to be “not substituted” or “unsubstituted”, then hydrogen atoms are attached to take up any free valency.

Unless otherwise indicated, a “ring” or “ring system” as a component of Formula 1 (e.g., substituent R) is carbocyclic or heterocyclic. The term “ring system” denotes two or more fused rings. The term “ring member” refers to an atom or other moiety (e.g., C(═O), C(═S), S(O) or S(O)₂) forming the backbone of a ring or ring system.

The terms “carbocyclic ring”, “carbocycle” or “carbocyclic ring system” denote a ring or ring system wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

The terms “heterocyclic ring”, “heterocycle” or “heterocyclic ring system” denote a ring or ring system in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings and ring systems can be attached through any available carbon or nitrogen by replacement of a hydrogen on said carbon or nitrogen.

“Aromatic” indicates that each of the ring atoms is essentially in the same plane and has a p-orbital perpendicular to the ring plane, and that (4n+2) π electrons, where n is a positive integer, are associated with the ring to comply with Hückel's rule. The term “nonaromatic ring” denotes a carbocyclic or heterocyclic ring that may be fully saturated, as well as partially or fully unsaturated, provided that at least one of the ring atoms in the ring does not have a p-orbital perpendicular to the ring plane.

The term “optionally substituted” in connection with the heterocyclic rings refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “unsubstituted or substituted” or with the term “(un)substituted”. Unless otherwise indicated, an optionally substituted group may have a substituent at each substitutable position of the group, and each substitution is independent of the other.

As noted above, R can be (among others) phenyl optionally substituted with one or more substituents selected from a group of substituents as defined in the Summary of the Invention. An example of phenyl optionally substituted with one to five substituents is the ring illustrated as U-1 in Exhibit 1, wherein R^{v} is one of the substituents on phenyl as defined in the Summary of the Invention for R and r is an integer (from 0 to 5).

As noted above, R can be (among others) a 5- or 6-membered heterocyclic ring, which may be saturated or unsaturated, optionally substituted with one or more substituents selected from a group of substituents as defined in the Summary of the Invention. When R is a 5- or 6-membered nitrogen-containing heterocyclic ring, it may be attached to the remainder of Formula 1 though any available carbon or nitrogen ring atom, unless otherwise described. Examples of a 5- or 6-membered unsaturated aromatic heterocyclic ring optionally substituted with from one or more substituents include the rings U-2 through U-61 illustrated in Exhibit 1 wherein R^{v} is any substituent as defined in the Summary of the Invention for R and r is an integer from 0 to 4, limited by the number of available positions on each U group. As U-29, U-30, U-36, U-37, U-38, U-39, U-40, U-41, U-42 and U-43 have only one available position, for these U groups r is limited to the integers 0 or 1, and r being 0 means that the U group is unsubstituted and a hydrogen is present at the position indicated by (R^{v})ᵣ.

### Exhibit 1

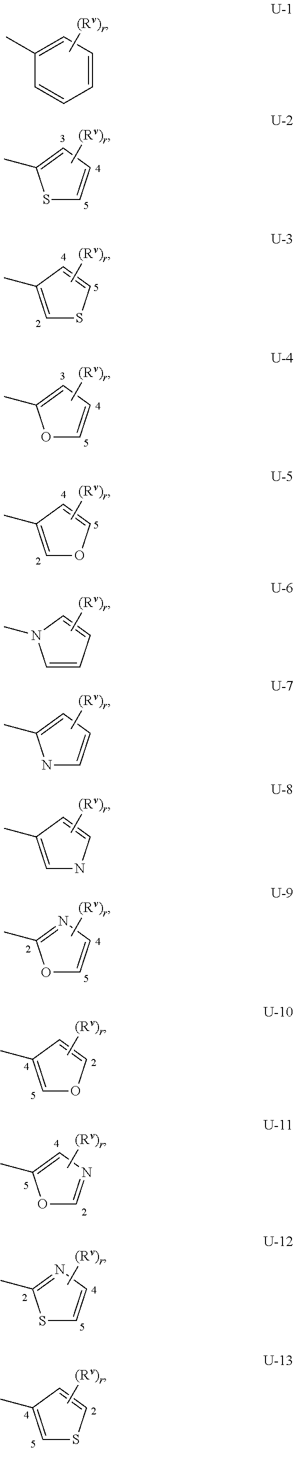

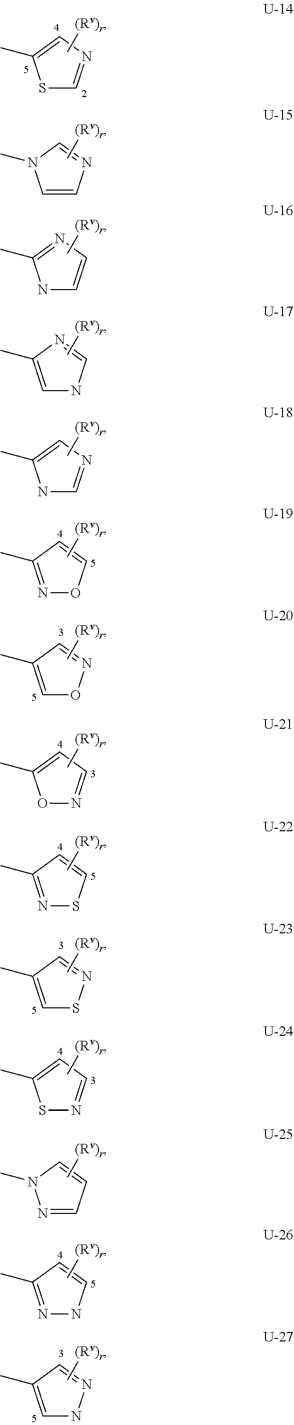

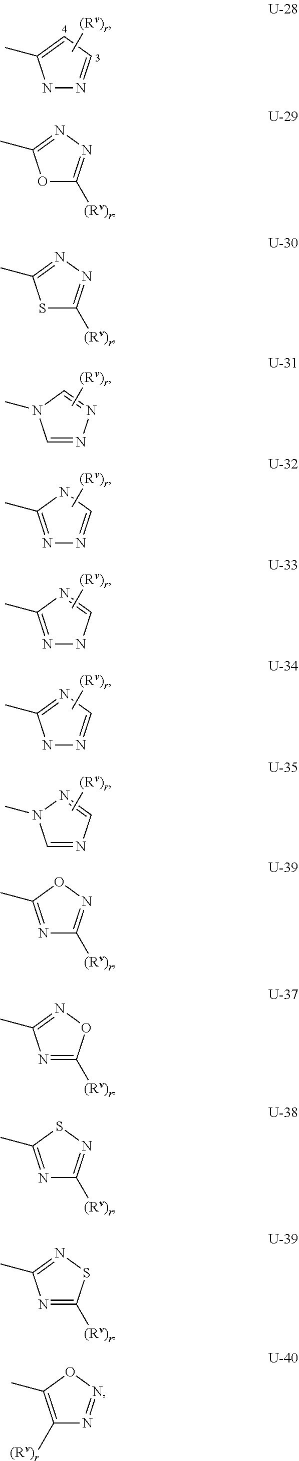

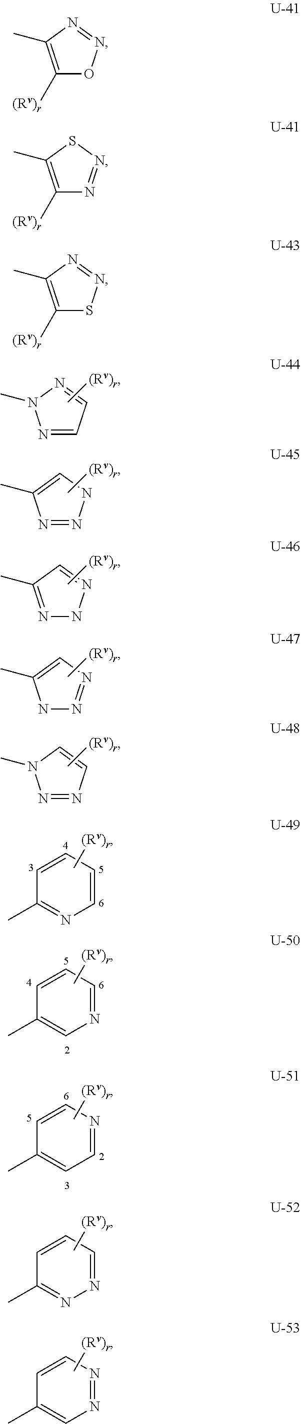

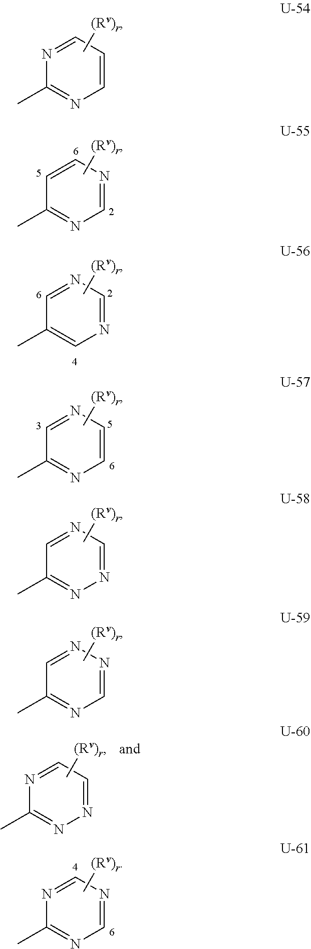

Note that when R is a 5- or 6-membered saturated or unsaturated nonaromatic heterocyclic ring optionally substituted with one or more substituents selected from the group of substituents as defined in the Summary of the Invention for R, one or two carbon ring members of the heterocycle can optionally be in the oxidized form of a carbonyl moiety.

Examples of a 5- or 6-membered saturated or nonaromatic unsaturated heterocyclic ring containing ring members selected from up to two O atoms and up to two S atoms, and optionally substituted on carbon atom ring members with up to five halogen atoms includes the rings G-1 through G-35 as illustrated in Exhibit 2. Note that when the attachment point on the G group is illustrated as floating, the G group can be attached to the remainder of Formula 1 through any available carbon or nitrogen of the G group by replacement of a hydrogen atom. The optional substituents corresponding to R^{v} can be attached to any available carbon or nitrogen by replacing a hydrogen atom. For these G rings, r is typically an integer from 0 to 4, limited by the number of available positions on each G group.

Note that when R comprises a ring selected from G-28 through G-35, G² is selected from O, S or N. Note that when G² is N, the nitrogen atom can complete its valence by substitution with either H or the substituents corresponding to R^{v} as defined in the Summary of the Invention for R.

### Exhibit 2

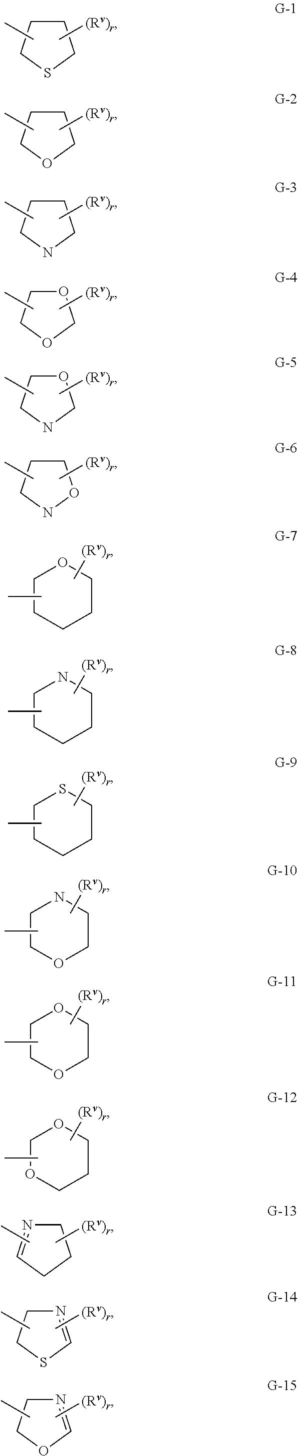

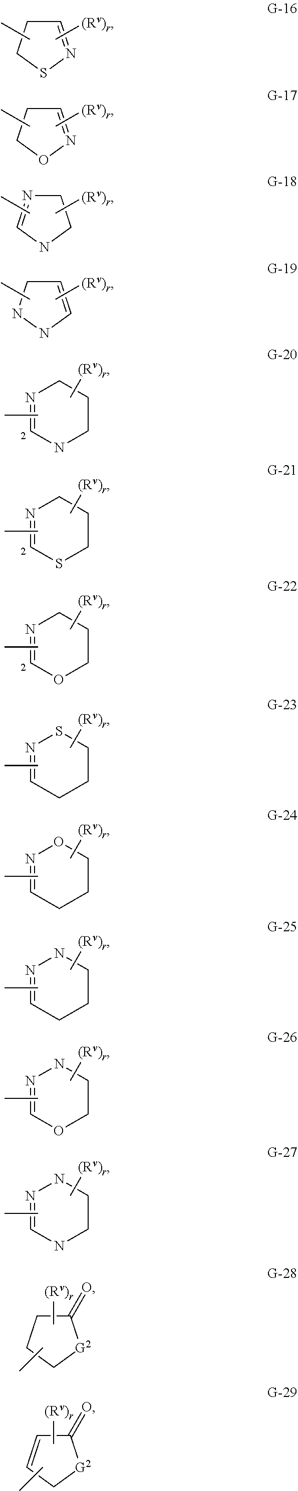

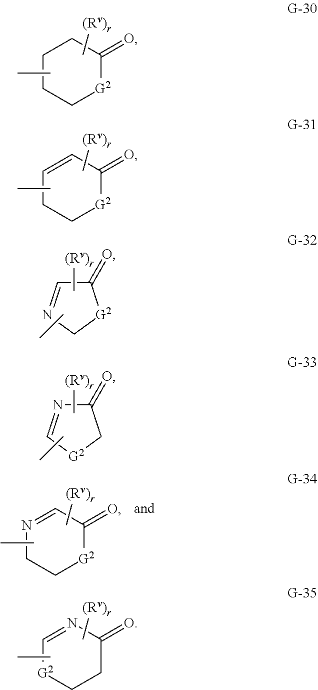

As noted above, R can be (among others) a naphthalenyl ring system optionally substituted with one or more substituents selected from a group of substituents as defined in the Summary of the Invention for R. Examples of a naphthalenyl ring system optionally substituted with from one or more substituents include the ring system U-62 illustrated in Exhibit 3 wherein R^{v} is any naphthalenyl substituent as defined in the Summary of the Invention for R, and r is typically an integer from 0 to 4.

### Exhibit 3

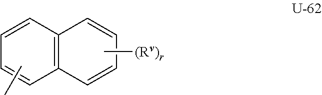

Although R^{v} groups are shown in the structure U-62, it is noted that they do not need to be present since they are optional substituents. Note that when R^{v} is H when attached to an atom, this is the same as if said atom is unsubstituted. Note that the attachment point between (R^{v})ᵣ and the U group is illustrated as floating, so (R^{v})ᵣ can be attached to any available carbon atom of the U group. Note that the attachment point on the U group is illustrated as floating, so the U group can be attached to the remainder of Formula 1 through any available carbon of the U group by replacement of a hydrogen atom.

A wide variety of synthetic methods are known in the art to enable preparation of aromatic and nonaromatic heterocyclic rings and ring systems; for extensive reviews see the eight-volume set of Comprehensive Heterocyclic Chemistry, A. R. Katritzky and C. W. Rees editors-in-chief, Pergamon Press, Oxford, 1984 and the twelve-volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

For example, Formula 1 possesses a chiral center at the carbon atom to which R⁴ is bonded. The two enantiomers are depicted as Formula 1′ and Formula 1″ with the chiral center identified with an asterisk (*). For a comprehensive discussion of all aspects of stereoisomerism, see Ernest L. Eliel and Samuel H. Wilen, Stereochemistry of Organic Compounds, John Wiley & Sons, 1994.

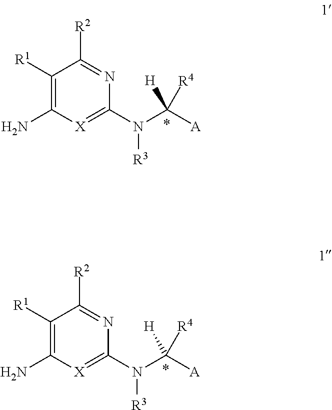

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the narrow end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified. As used herein, a wavy line attached to an asymmetric center represents a condition wherein the configuration at that center can be either R— or S—.

The more herbicidally-active enantiomer is believed to be Formula 1′. When R⁴ is CH₃, Formula 1′ has the R configuration at the carbon atom to which R⁴ is bonded.

This invention comprises racemic mixtures, for example, equal amounts of the enantiomers of Formulae 1′ and 1″. In addition, this invention includes compounds that are enriched in one enantiomer of Formula 1 compared to the racemic mixture. Also included are enantiomers of compounds of Formula 1′ that are substantially free of the enantiomers of Formula 1″. Also included are the essentially pure enantiomers of compounds of Formula 1, for example, Formula 1′ and Formula 1″, preferably Formula 1′.

When enantiomerically enriched, one enantiomer is present in greater amounts than the other, and the extent of enrichment can be defined by an expression of enantiomeric excess (“ee”), which is defined as (Fₘₐⱼ−Fₘᵢₙ)·100%, where Fₘₐⱼ is the mole fraction of the dominant enantiomer in the mixture and Fₘᵢₙ is the mole fraction of the lesser enantiomer in the mixture (e.g., an ee of 20% corresponds to a 60:40 ratio of enantiomers).

As used herein, the term “predominantly in the R-configuration” refers to a sterocenter wherein at least 60% of the molecules have the stereocenter in the R-configuration. For example, a compound with a single stereocenter, such as indicated by a *, would have an enatiomeric excess of 20%. Preferably the compositions of this invention have at least a 50% enantiomeric excess; at least a 60% enantiomeric excess; more preferably at least a 75% enantiomeric excess; still more preferably at least a 90% enantiomeric excess; more preferably at least a 94% enantiomeric excess; more preferably at least a 95% enantiomeric excess; more preferably at least a 98% enantiomeric excess; more preferably at least a 99% enantiomeric excess; of the more active isomer.

As used herein, the term “substantially free of the enantiomer of Formula 1″” refers to an enantiomer of Formula 1′ having at least a 90% enantiomeric excess; more preferably at least a 94% enantiomeric excess; more preferably at least a 95% enantiomeric excess; more preferably at least a 98% enantiomeric excess; most preferably at least a 99% enantiomeric excess. Of note are enantiomerically pure embodiments of the more active isomer.

Compounds of Formula 1 can comprise chiral centers in addition to the chiral center indicated by a *. For example, substituents and other molecular constituents such as R, R¹ and R² may themselves contain chiral centers. This invention comprises racemic mixtures as well as enriched and essentially pure stereoconfigurations at these additional chiral centers. Preferably compounds of Formula 1 comprising additional chiral centers are enriched or essentially pure at the carbon atom to which R⁴ is bonded, such that when R⁴ is CH₃, Formula 1′ has the R configuration at the carbon atom to which R⁴ is bonded.

Compounds of this invention can exist as one or more conformational isomers due to restricted rotation about an amide bond (e.g., when R³ is C₁-C₆ alkylcarbonyl) in Formula 1. This invention comprises mixtures of conformational isomers. In addition, this invention includes compounds that are enriched in one conformer relative to others.

Compounds of Formula 1 typically exist in more than one form, and Formula 1 thus includes all crystalline and non-crystalline forms of the compounds it represents. Non-crystalline forms include embodiments that are solids such as waxes and gums as well as embodiments that are liquids such as solutions and melts. Crystalline forms include embodiments that represent essentially a single crystal type and embodiments that represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of Formula 1 can exhibit beneficial effects (e.g., suitability for preparation of useful formulations, improved biological performance) relative to another polymorph or a mixture of polymorphs of the same compound of Formula 1. Preparation and isolation of a particular polymorph of a compound of Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus, a wide variety of salts of a compound of Formula 1 are useful for control of undesired vegetation (i.e. are agriculturally suitable). The salts of a compound of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Embodiments of the present invention as described in the Summary of the Invention include those wherein a compound of Formula 1 is as described in any of the following Embodiments:
Embodiment 1. A compound of Formula 1, including all stereoisomers, N-oxides, and salts thereof, agricultural compositions containing them and their use as herbicides as described in the Summary of the Invention.
Embodiment 2. A compound of Embodiment 1 wherein X in N.
Embodiment 3. A compound of Embodiment 1 wherein X is CR⁵.
Embodiment 4. A compound of any one of Embodiments 1 through 3 wherein A is selected from the group consisting of A-1, A-2 and A-3.
Embodiment 5. A compound of Embodiment 4 wherein A is selected from the group consisting of A-2 and A-3.
Embodiment 6. A compound of Embodiment 5 wherein A is selected from the group consisting of A-1 and A-2.
Embodiment 7. A compound of Embodiment 6 wherein A is A-1.
Embodiment 8. A compound of Embodiment 6 wherein A is A-2.
Embodiment 9. A compound of any one of Embodiments 1 through 3 wherein A is A-4.
Embodiment 10. A compound of any one of Embodiments 1 through 9 wherein R¹ is H, halogen, cyano, nitro, C₁-C₆ alkyl, C₁-C₆ haloalkyl, C₁-C₆ haloalkoxy, C₂-C₆ alkoxycarbonyl or C₂-C₆ haloalkoxycarbonyl.
Embodiment 11. A compound of Embodiment 10 wherein R¹ is H, halogen, cyano, nitro, C₁-C₆ haloalkyl or C₁-C₆ haloalkoxy.
Embodiment 12. A compound of Embodiment 11 wherein R¹ is halogen, cyano, nitro, C₁-C₂ haloalkyl or C₁-C₂ haloalkoxy.
Embodiment 13. A compound of Embodiment 12 wherein R¹ is halogen, cyano, nitro or C₁-C₂ haloalkyl.
Embodiment 14. A compound of Embodiment 13 wherein R¹ is halogen, cyano or C₁-C₂ haloalkyl.
Embodiment 15. A compound of Embodiment 14 wherein R¹ is cyano or C₁-C₂ haloalkyl.
Embodiment 16. A compound of Embodiment 15 wherein R¹ is C₁-C₂ haloalkyl.
Embodiment 17. A compound of Embodiment 16 wherein R¹ is CF₃.
Embodiment 18. A compound of Embodiment 15 wherein R¹ is cyano.
Embodiment 19. A compound of Embodiment 14 wherein R¹ is Cl, Br or I.
Embodiment 20. A compound of any one of Embodiments 1 through 11 wherein R¹ is other than H.
Embodiment 21. A compound of any one of Embodiments 1 through 20 wherein R² is H, halogen, C₁-C₆ alkyl, C₁-C₆ haloalkyl, C₁-C₆ alkylcarbonyl, C₁-C₆ haloalkylcarbonyl, C₁-C₆ alkoxy, C₁-C₆ haloalkoxy, C₂-C₆ alkoxycarbonyl or C₂-C₆ haloalkoxycarbonyl.
Embodiment 22. A compound of Embodiment 21 wherein R² is H, halogen, C₁-C₆ alkyl or C₁-C₆ haloalkyl.
Embodiment 23. A compound of Embodiment 22 wherein R² is H, C₁-C₆ alkyl or C₁-C₆ haloalkyl.
Embodiment 24. A compound of Embodiment 23 wherein R² is H or C₁-C₆ alkyl.
Embodiment 25. A compound of Embodiment 24 wherein R² is H or methyl.
Embodiment 26. A compound of any one of Embodiments 1 through 25 wherein R² is other than H.
Embodiment 27. A compound of any one of Embodiments 1 through 9 wherein when R² is H, R¹ is C₁-C₂ haloalkyl.
Embodiment 28. A compound Embodiment 27 wherein R¹ is CF₃.
Embodiment 29. A compound of any one of Embodiments 1 through 9 wherein when R² is H, R¹ is nitro.
Embodiment 30. A compound of any one of Embodiments 1 through 9 wherein when R² is Me, R¹ is halogen, cyano, nitro, C₁-C₆ haloalkyl, C₁-C₆ haloalkoxy, C₂-C₆ alkoxycarbonyl or C₂-C₆ haloalkoxycarbonyl.
Embodiment 31. A compound of Embodiment 30 wherein R¹ is Cl, Br or I.
Embodiment 32. A compound of Embodiment 30 wherein R¹ is cyano.
Embodiment 33. A compound of Embodiment 30 wherein R¹ is nitro.
Embodiment 34. A compound of any one of Embodiments 1 through 33 wherein R³ is H, C₁-C₄ alkyl or C₂-C₆ alkylcarbonyl.
Embodiment 35. A compound of Embodiment 34 wherein R³ is H or C₁-C₄ alkyl.
Embodiment 36. A compound of Embodiment 35 wherein R³ is H or CH₃.
Embodiment 37. A compound of Embodiment 36 wherein R³ is H.
Embodiment 38. A compound of any one of Embodiments 1 through 37 wherein R⁴ is C₁-C₆ alkyl or C₃-C₇ cycloalkyl.
Embodiment 39. A compound of Embodiment 38 wherein R⁴ is C₁-C₆ alkyl.
Embodiment 40. A compound of Embodiment 39 wherein R⁴ is CH₃ or CH₂CH₃.
Embodiment 41. A compound of Embodiment 40 wherein R⁴ is CH₃.
Embodiment 42. A compound of Embodiment 41 wherein R⁴ is C₃-C₇ cycloalkyl.
Embodiment 43. A compound of Embodiment 42 wherein R⁴ is cyclopropyl.
Embodiment 44. A compound of any one of Embodiments 1 through 43 wherein each R is independently halogen, cyano, C₁-C₄ alkyl, C₁-C₄ haloalkyl, C₃-C₇ cycloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl, C₂-C₄ cyanoalkyl, C₂-C₄ alkylcarbonyl or C₂-C₆ alkoxycarbonyl.
Embodiment 45. A compound of Embodiment 44 wherein each R is independently halogen, cyano, C₁-C₄ alkyl, C₁-C₄ haloalkyl, C₁-C₄ alkoxy, C₁-C₄ haloalkoxy, C₂-C₄ cyanoalkyl or C₂-C₆ alkoxycarbonyl.
Embodiment 46. A compound of Embodiment 45 wherein each R is independently halogen, C₁-C₄ alkyl or C₁-C₄ haloalkyl.
Embodiment 47. A compound of Embodiment 46 wherein each R is independently halogen, CH₃ or CF₃.
Embodiment 48. A compound of any one of Embodiments 1 through 47 wherein n is 0, 1, 2 or 3.
Embodiment 49. A compound of Embodiment 48 wherein n is 0, 1 or 2.
Embodiment 50. A compound of Embodiment 49 wherein n is 1.
Embodiment 51. A compound of Embodiment 49 wherein n is 0.
Embodiment 52. A compound of any one of Embodiments 1 through 48 wherein n is 1, 2 or 3.
Embodiment 53. A compound of any one of Embodiments 1 through 52 wherein A is A-1.
Embodiment 54. A compound of Embodiment 53 wherein Q¹ is O, S or —C(R⁶)═C(R⁷)—.
Embodiment 55. A compound of Embodiment 54 wherein Q¹ is O.
Embodiment 56. A compound of Embodiment 54 wherein Q¹ is S.
Embodiment 57. A compound of Embodiment 54 wherein Q¹ is —C(R⁶)═C(R⁷)—.
Embodiment 58. A compound of Embodiment 57 wherein R⁶ and R⁷ are both H.
Embodiment 59. A compound of any one of Embodiments 1 through 54 wherein Q¹ is O or S and A-1 is substituted with R at the 5- or 6-position; or substituted with R at both the 5- and 6-position of the bicyclic ring.
Embodiment 60. A compound of Embodiment 59 wherein A-1 is further substituted at the 3-position.
Embodiment 61. A compound of any one of Embodiments 1 through 52 wherein A is A-2.
Embodiment 62. A compound of Embodiment 61 wherein Q² is O, S or —C(R⁶)═C(R⁷)—.
Embodiment 63. A compound of Embodiment 62 wherein Q² is O.
Embodiment 64. A compound of Embodiment 62 wherein Q² is S.
Embodiment 65. A compound of Embodiment 62 wherein Q² is —C(R⁶)═C(R⁷)—.
Embodiment 66. A compound of Embodiment 65 wherein R⁶ and R⁷ are both H.
Embodiment 67. A compound of any of Embodiments 1 through 52 and 61 and 62 wherein Q² is O or S and A-2 is substituted with R at the 5- or 6-position; or substituted with R at both the 5- and 6-position of the bicyclic ring.
Embodiment 68. A compound of Embodiment 67 wherein A-2 is further substituted at the 2-position.
Embodiment 69. A compound of any one of Embodiments 1 through 52 wherein A is A-3.
Embodiment 70. A compound of Embodiment 69 wherein Q³ is O, S or —C(R⁶)═C(R⁷)—.
Embodiment 71. A compound of Embodiment 70 wherein Q³ is O.
Embodiment 72. A compound of Embodiment 70 wherein Q³ is S.
Embodiment 73. A compound of Embodiment 70 wherein Q³ is —C(R⁶)═C(R⁷)—.
Embodiment 74. A compound of Embodiment 73 wherein R⁶ and R⁷ are both H.
Embodiment 75. A compound of any of Embodiments 1 through 52 and 69 and 70 wherein Q³ is O or S and A-3 is substituted with R at the 5- or 6-position; or substituted with R at both the 5- and 6-position of the bicyclic ring.
Embodiment 76. A compound of Embodiment 75 wherein A-3 is further substituted at the 3-position.
Embodiment 77. A compound of any one of Embodiments 1 through 52 wherein A is A-4.
Embodiment 78. A compound of Embodiment 77 wherein Q⁴ is O, S or CR⁶R⁷.
Embodiment 79. A compound of Embodiment 78 wherein Q⁴ is O.
Embodiment 80. A compound of Embodiment 78 wherein Q⁴ is CR⁶R⁷.
Embodiment 81. A compound of Embodiment 79 wherein R⁶ and R⁷ are both H.
Embodiment 82. A compound of any one of Embodiments 1 through 52 and 77 and 78 wherein A-4 substituted with R at the 3-, 4- or 5-position, or any combination thereof.
Embodiment 83. A compound of Embodiment 82 wherein A-4 is further substituted at the 2-position.
Embodiment 84. A compound of any one of Embodiments 1 through 52 and 77 and 78 wherein A-4 is substituted with R at the 4- or 5-position.
Embodiment 85. A compound of any of Embodiments 1 to 84 wherein the stereocenter indicated by the * is predominantly in the R-configuration.

Embodiments of this invention, including Embodiments 1-85 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-85 above as well as any other embodiments described herein, and any combination thereof, pertain to the compositions and methods of the present invention.

Embodiment A. A compound of Formula 1 wherein
X is N;
R¹ is H, halogen, cyano, nitro, C₁-C₆ alkyl, C₁-C₆ haloalkyl, C₁-C₆ haloalkoxy, C₂-C₆ alkoxycarbonyl or C₂-C₆ haloalkoxycarbonyl;
R² is H, halogen, C₁-C₆ alkyl, C₁-C₆ haloalkyl, C₂-C₆ alkylcarbonyl, C₂-C₆ haloalkylcarbonyl, C₁-C₆ alkoxy, C₁-C₆ haloalkoxy, C₂-C₆ alkoxycarbonyl or C₂-C₆ haloalkoxycarbonyl;
R³ is H, C₁-C₄ alkyl or C₂-C₆ alkylcarbonyl; and
R⁴ is C₁-C₆ alkyl or C₃-C₇ cycloalkyl.

Embodiment B. A compound of Embodiment A wherein
R¹ is H, halogen, cyano, nitro, C₁-C₆ haloalkyl or C₁-C₆ haloalkoxy;
R² is H, halogen, C₁-C₆ alkyl or C₁-C₆ haloalkyl;
R³ is H or C₁-C₄ alkyl; and
R⁴ is C₁-C₆ alkyl.

Embodiment C. A compound of Embodiment B wherein
R¹ is C₁-C₂ haloalkyl;
R² is H or C₁-C₆ alkyl;
R³ is H or CH₃; and
R⁴ is CH₃ or CH₂CH₃.

Embodiment D. A compound of Embodiment C wherein
R¹ is CF₃;
R² is H;
R³ is H; and
R⁴ is CH₃.

Embodiment E. A compound of any one of Embodiments A through D wherein
A is A-1; and
Q¹ is O.

Embodiment F. A compound of one of Embodiments A through D wherein
A is A-4; and
Q⁴ is O.

Embodiment G. A compound of any one of Embodiments A through D wherein
A is A-4; and
Q⁴ is CH₂—

Embodiment H. A compound of any of Embodiments A through G wherein
each R is independently halogen, C₁-C₄ alkyl or C₁-C₄ haloalkyl; and
n is 0, 1, 2 or 3.

Embodiment I. A compound of any one of Embodiments A through H wherein the
stereocenter indicated by the * is predominantly in the R-configuration.

Specific Embodiments of the Invention are the following compounds of the Summary of the Invention selected from the group consisting of:
N2-[(1R)-1-(6-fluoro-2-benzofuranyl)ethyl]-5-(trifluoromethyl)-2,4-pyrimidinediamine (Compound 8);
N2-[(1R)-1-(4-fluoro-2-benzofuranyl)ethyl]-5-(trifluoromethyl)-2,4-pyrimidinediamine (Compound 73);
N2-[(1R)-1-(7-fluoro-2-benzofuranyl)ethyl]-5-(trifluoromethyl)-2,4-pyrimidinediamine (Compound 71);
N2-[(1R)-1-benzo[b]thien-2-ylethyl]-5-(trifluoromethyl)-2,4-pyrimidinediamine (Compound 4)
N2-[(1R)-1-(4-fluorobenzo[b]thien-2-yl)ethyl]-5-(trifluoromethyl)-2,4-pyrimidinediamine; (Compound 61),
N2-[(1R)-1-(7-fluorobenzo[b]thien-2-yl)ethyl]-5-(trifluoromethyl)-2,4-pyrimidinediamine (Compound 75);
N2-[(1R)-1-(3-benzofuranyl)ethyl]-5-(trifluoromethyl)-2,4-pyrimidinediamine (Compound 52);
N2-[(R)-3-benzofuranylcyclopropylmethyl]-5-(trifluoromethyl)-2,4-pyrimidinediamine (Compound 79); and
N2-[(1R)-1-(2,3-dihydro-1H-inden-2-yl)ethyl]-5-(trifluoromethyl)-2,4-pyrimidinediamine (Compound 13).

Specific Embodiments of the Invention are the following compounds of the Summary of the Invention selected from the group consisting of
N2-[(1R)-1-(2-benzofuranyl)ethyl]-5-(trifluoromethyl)-2,4-pyrimidinediamine (i.e. Compound No. 22); and
N2-[(1R)-2-(3,5-dimethylphenoxy)-1-methylethyl]-5-(trifluoromethyl)-2,4-pyrimidinediamine (i.e. Compound No. 25).

Specific Embodiments of the Invention are the following compounds of the Summary of the Invention selected from the group consisting of:
a compound of Formula 1 wherein X is N; R¹ is CF₃; R² is H; R³ is H; R⁴ is Me; A is A-1, Q¹ is S; and n is 0 (i.e. Compound No. 4); and
a compound of Formula 1 wherein X is N; R¹ is CF₃; R² is H; R³ is H; R⁴ is Me; A is A-1, Q¹ is 0; and (R)ₙ is 3-F (i.e. Compound No. 8).

This invention also relates to a method for controlling undesired vegetation comprising applying to the locus of the vegetation herbicidally effective amounts of the compounds of the invention (e.g., as a composition described herein). Of note as embodiments relating to methods of use are those involving the compounds of embodiments described above. Compounds of the invention are particularly useful for selective control of weeds in crops such as wheat, barley, maize, soybean, sunflower, cotton, oilseed rape and rice, and specialty crops such as sugarcane, citrus, fruit and nut crops, notably wheat, corn and rice.

Also noteworthy as embodiments are herbicidal compositions of the present invention comprising the compounds of any of the embodiments described above.

This invention also includes a herbicidal mixture comprising (a) a compound selected from Formula 1, N-oxides, and salts thereof, and (b) at least one additional active ingredient selected from the group consisting of (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solenesyltransererase (HST) inhibitors, (b14) cellulose biosynthesis inhibitors, (b15) other herbicides including mitotic disruptors, organic arsenicals, asulam, bromobutide, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb (b16) herbicide safeners, and salts of compounds of (b1) through (b16).

“Photosystem II inhibitors” (b1) are chemical compounds that bind to the D-1 protein at the Q_{B}-binding niche and thus block electron transport from Q_{A} to Q_{B} in the chloroplast thylakoid membranes. The electrons blocked from passing through photosystem II are transferred through a series of reactions to form toxic compounds that disrupt cell membranes and cause chloroplast swelling, membrane leakage, and ultimately cellular destruction. The Q_{B}-binding niche has three different binding sites: binding site A binds the triazines such as atrazine, triazinones such as hexazinone, and uracils such as bromacil, binding site B binds the phenylureas such as diuron, and binding site C binds benzothiadiazoles such as bentazon, nitriles such as bromoxynil and phenyl-pyridazines such as pyridate. Examples of photosystem II inhibitors include ametryn, amicarbazone, atrazine, bentazon, bromacil, bromofenoxim, bromoxynil, chlorbromuron, chloridazon, chlorotoluron, chloroxuron, cumyluron, cyanazine, daimuron, desmedipham, desmetryn, dimefuron, dimethametryn, diuron, ethidimuron, fenuron, fluometuron, hexazinone, ioxynil, isoproturon, isouron, lenacil, linuron, metamitron, methabenzthiazuron, metobromuron, metoxuron, metribuzin, monolinuron, neburon, pentanochlor, phenmedipham, prometon, prometryn, propanil, propazine, pyridafol, pyridate, siduron, simazine, simetryn, tebuthiuron, terbacil, terbumeton, terbuthylazine, terbutryn and trietazine.

“AHAS inhibitors” (b2) are chemical compounds that inhibit acetohydroxy acid synthase (AHAS), also known as acetolactate synthase (ALS), and thus kill plants by inhibiting the production of the branched-chain aliphatic amino acids such as valine, leucine and isoleucine, which are required for protein synthesis and cell growth. Examples of AHAS inhibitors include amidosulfuron, azimsulfuron, bensulfuron-methyl, bispyribac-sodium, cloransulam-methyl, chlorimuron-ethyl, chlorsulfuron, cinosulfuron, cyclosulfamuron, diclosulam, ethametsulfuron-methyl, ethoxysulfuron, flazasulfuron, florasulam, flucarbazone-sodium, flumetsulam, flupyrsulfuron-methyl, flupyrsulfuron-sodium, foramsulfuron, halosulfuron-methyl, imazamethabenz-methyl, imazamox, imazapic, imazapyr, imazaquin, imazethapyr, imazosulfuron, iodosulfuron-methyl (including sodium salt), iofensulfuron (2-iodo-N-[[(4-methoxy-6-methyl-1,3,5-triazin-2-yl)amino]carbonyl]benzenesulfonamide), mesosulfuron-methyl, metazosulfuron (3-chloro-4-(5,6-dihydro-5-methyl-1,4,2-dioxazin-3-yl)-N-[[(4,6-dimethoxy-2-pyrimidinyl)amino]carbonyl]-1-methyl-1H-pyrazole-5-sulfonamide), metosulam, metsulfuron-methyl, nicosulfuron, oxasulfuron, penoxsulam, primisulfuron-methyl, propoxycarbazone-sodium, propyrisulfuron (2-chloro-N-[[(4,6-dimethoxy-2-pyrimidinyl)amino]carbonyl]-6-propylimidazo[1,2-b]pyridazine-3-sulfonamide), prosulfuron, pyrazosulfuron-ethyl, pyribenzoxim, pyriftalid, pyriminobac-methyl, pyrithiobac-sodium, rimsulfuron, rinskor, sulfometuron-methyl, sulfosulfuron, thiencarbazone, thifensulfuron-methyl, triafamone (N-[2-[(4,6-dimethoxy-1,3,5-triazin-2-yl)carbonyl]-6-fluorophenyl]-1,1-difluoro-N-methylmethanesulfonamide), triasulfuron, tribenuron-methyl, trifloxysulfuron (including sodium salt), triflusulfuron-methyl and tritosulfuron.

“ACCase inhibitors” (b3) are chemical compounds that inhibit the acetyl-CoA carboxylase enzyme, which is responsible for catalyzing an early step in lipid and fatty acid synthesis in plants. Lipids are essential components of cell membranes, and without them, new cells cannot be produced. The inhibition of acetyl CoA carboxylase and the subsequent lack of lipid production leads to losses in cell membrane integrity, especially in regions of active growth such as meristems. Eventually shoot and rhizome growth ceases, and shoot meristems and rhizome buds begin to die back. Examples of ACCase inhibitors include alloxydim, butroxydim, clethodim, clodinafop, cycloxydim, cyhalofop, diclofop, fenoxaprop, fluazifop, haloxyfop, pinoxaden, profoxydim, propaquizafop, quizalofop, sethoxydim, tepraloxydim and tralkoxydim, including resolved forms such as fenoxaprop-P, fluazifop-P, haloxyfop-P and quizalofop-P and ester forms such as clodinafop-propargyl, cyhalofop-butyl, diclofop-methyl and fenoxaprop-P-ethyl.

Auxin is a plant hormone that regulates growth in many plant tissues. “Auxin mimics” (b4) are chemical compounds mimicking the plant growth hormone auxin, thus causing uncontrolled and disorganized growth leading to plant death in susceptible species. Examples of auxin mimics include aminocyclopyrachlor (6-amino-5-chloro-2-cyclopropyl-4-pyrimidinecarboxylic acid) and its methyl and ethyl esters and its sodium and potassium salts, aminopyralid, benazolin-ethyl, chloramben, clacyfos, clomeprop, clopyralid, dicamba, 2,4-D, 2,4-DB, dichlorprop, fluroxypyr, halauxifen (4-amino-3-chloro-6-(4-chloro-2-fluoro-3-methoxyphenyl)-2-pyridinecarboxylic acid), halauxifen-methyl (methyl 4-amino-3-chloro-6-(4-chloro-2-fluoro-3-methoxyphenyl)-2-pyridinecarboxylate), MCPA, MCPB, mecoprop, picloram, quinclorac, quinmerac, 2,3,6-TBA, triclopyr, and methyl 4-amino-3-chloro-6-(4-chloro-2-fluoro-3-methoxyphenyl)-5-fluoro-2-pyridinecarboxylate.

“EPSP synthase inhibitors” (b5) are chemical compounds that inhibit the enzyme, 5-enol-pyruvylshikimate-3-phosphate synthase, which is involved in the synthesis of aromatic amino acids such as tyrosine, tryptophan and phenylalanine. EPSP inhibitor herbicides are readily absorbed through plant foliage and translocated in the phloem to the growing points. Glyphosate is a relatively nonselective postemergence herbicide that belongs to this group. Glyphosate includes esters and salts such as ammonium, isopropylammonium, potassium, sodium (including sesquisodium) and trimesium (alternatively named sulfosate).

“Photosystem I electron diverters” (b6) are chemical compounds that accept electrons from Photosystem I, and after several cycles, generate hydroxyl radicals. These radicals are extremely reactive and readily destroy unsaturated lipids, including membrane fatty acids and chlorophyll. This destroys cell membrane integrity, so that cells and organelles “leak”, leading to rapid leaf wilting and desiccation, and eventually to plant death. Examples of this second type of photosynthesis inhibitor include diquat and paraquat.

“PPO inhibitors” (b7) are chemical compounds that inhibit the enzyme protoporphyrinogen oxidase, quickly resulting in formation of highly reactive compounds in plants that rupture cell membranes, causing cell fluids to leak out. Examples of PPO inhibitors include acifluorfen-sodium, azafenidin, benzfendizone, bifenox, butafenacil, carfentrazone, carfentrazone-ethyl, chlomethoxyfen, cinidon-ethyl, fluazolate, flufenpyr-ethyl, flumiclorac-pentyl, flumioxazin, fluoroglycofen-ethyl, fluthiacet-methyl, fomesafen, halosafen, lactofen, oxadiargyl, oxadiazon, oxyfluorfen, pentoxazone, profluazol, pyraclonil, pyraflufen-ethyl, saflufenacil, sulfentrazone, thidiazimin, trifludimoxazin (dihydro-1,5-dimehyl-6-thioxo-3-[2,2,7-trifluoro-3,4-dihydro-3-oxo-4-(2-propyn-1-yl)-2H-1,4-benzoxazin-6-yl]-1,3,5-triazine-2,4(1H,3H)-dione) and tiafenacil (methyl N-[2-[[2-chloro-5-[3,6-dihydro-3-methyl-2,6-dioxo-4-(trifluoromethyl)-1(2H)-pyrimidinyl]-4-fluorophenyl]thio]-1-oxopropyl]-β-alaninate).

“GS inhibitors” (b8) are chemical compounds that inhibit the activity of the glutamine synthetase enzyme, which plants use to convert ammonia into glutamine. Consequently, ammonia accumulates and glutamine levels decrease. Plant damage probably occurs due to the combined effects of ammonia toxicity and deficiency of amino acids required for other metabolic processes. The GS inhibitors include glufosinate and its esters and salts such as glufosinate-ammonium and other phosphinothricin derivatives, glufosinate-P ((2S)-2-amino-4-(hydroxymethylphosphinyl)butanoic acid) and bilanaphos.

“VLCFA elongase inhibitors” (b9) are herbicides having a wide variety of chemical structures, which inhibit the elongase. Elongase is one of the enzymes located in or near chloroplasts which are involved in biosynthesis of VLCFAs. In plants, very-long-chain fatty acids are the main constituents of hydrophobic polymers that prevent desiccation at the leaf surface and provide stability to pollen grains. Such herbicides include acetochlor, alachlor, anilofos, butachlor, cafenstrole, dimethachlor, dimethenamid, diphenamid, fenoxasulfone (3-[[(2,5-dichloro-4-ethoxyphenyl)methyl]sulfonyl]-4,5-dihydro-5,5-dimethylisoxazole), fentrazamide, flufenacet, indanofan, mefenacet, metazachlor, metolachlor, naproanilide, napropamide, napropamide-M ((2R)—N,N-diethyl-2-(1-naphthalenyloxy)propanamide), pethoxamid, piperophos, pretilachlor, propachlor, propisochlor, pyroxasulfone, and thenylchlor, including resolved forms such as S-metolachlor and chloroacetamides and oxyacetamides.

“Auxin transport inhibitors” (b10) are chemical substances that inhibit auxin transport in plants, such as by binding with an auxin-carrier protein. Examples of auxin transport inhibitors include diflufenzopyr, naptalam (also known as N-(1-naphthyl)phthalamic acid and 2-[(1-naphthalenylamino)carbonyl]benzoic acid).

“PDS inhibitors” (b11) are chemical compounds that inhibit carotenoid biosynthesis pathway at the phytoene desaturase step. Examples of PDS inhibitors include beflubutamid, S-beflubutamid, diflufenican, fluridone, flurochloridone, flurtamone norflurzon and picolinafen.

“HPPD inhibitors” (b12) are chemical substances that inhibit the biosynthesis of synthesis of 4-hydroxyphenyl-pyruvate dioxygenase. Examples of HPPD inhibitors include benzobicyclon, benzofenap, bicyclopyrone (4-hydroxy-3-[[2-[(2-methoxyethoxy)methyl]-6-(trifluoromethyl)-3-pyridinyl]carbonyl]bicyclo[3.2.1]oct-3-en-2-one), fenquinotrione (2-[[8-chloro-3,4-dihydro-4-(4-methoxyphenyl)-3-oxo-2-quinoxalinyl]carbonyl]-1,3-cyclohexanedione), isoxachlortole, isoxaflutole, mesotrione, pyrasulfotole, pyrazolynate, pyrazoxyfen, sulcotrione, tefuryltrione, tembotrione, tolpyralate (1-[[1-ethyl-4-[3-(2-methoxyethoxy)-2-methyl-4-(methylsulfonyl)benzoyl]-1H-pyrazol-5-yl]oxy]ethyl methyl carbonate), topramezone, 5-chloro-3-[(2-hydroxy-6-oxo-1-cyclohexen-1-yl)carbonyl]-1-(4-methoxyphenyl)-2(1H)-quinoxalinone, 4-(2,6-diethyl-4-methylphenyl)-5-hydroxy-2,6-dimethyl-3(2H)-pyridazinone, 4-(4-fluorophenyl)-6-[(2-hydroxy-6-oxo-1-cyclohexen-1-yl)carbonyl]-2-methyl-1,2,4-triazine-3,5(2H,4H)-dione, 5-[(2-hydroxy-6-oxo-1-cyclohexen-1-yl)carbonyl]-2-(3-methoxyphenyl)-3-(3-methoxypropyl)-4(3H)-pyrimidinone, 2-methyl-N-(4-methyl-1,2,5-oxadiazol-3-yl)-3-(methylsulfinyl)-4-(trifluoromethyl)benzamide and 2-methyl-3-(methylsulfonyl)-N-(1-methyl-1H-tetrazol-5-yl)-4-(trifluoromethyl)benzamide.

“HST inhibitors” (b13) disrupt a plant's ability to convert homogentisate to 2-methyl-6-solanyl-1,4-benzoquinone, thereby disrupting carotenoid biosynthesis. Examples of HST inhibitors include haloxydine, pyriclor, 3-(2-chloro-3,6-difluorophenyl)-4-hydroxy-1-methyl-1,5-naphthyridin-2(1H)-one, 7-(3,5-dichloro-4-pyridinyl)-5-(2,2-difluoroethyl)-8-hydroxypyrido[2,3-b]pyrazin-6(5H)-one and 4-(2,6-diethyl-4-methylphenyl)-5-hydroxy-2,6-dimethyl-3(2H)-pyridazinone.

HST inhibitors also include compounds of Formulae A and B.

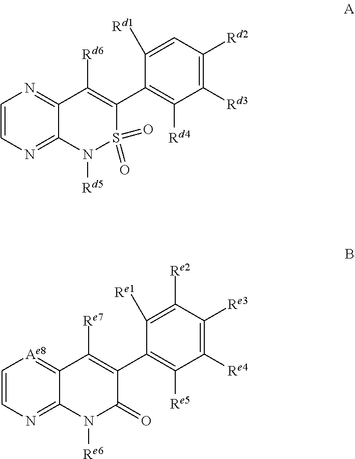

wherein R^{d1} is H, Cl or CF₃; R^{d2} is H, Cl or Br; R^{d3} is H or Cl; R_{d4} is H, Cl or CF₃; R^{d5} is CH₃, CH₂CH₃ or CH₂CHF₂; and R^{d6} is OH, or —OC(═O)-i-Pr; and R^{e1} is H, F, Cl, CH₃ or CH₂CH₃; R^{e2} is H or CF₃; R^{e3} is H, CH₃ or CH₂CH₃; R^{e4} is H, F or Br; R^{e5} is Cl, CH₃, CF₃, OCF₃ or CH₂CH₃; R^{e6} is H, CH₃, CH₂CHF₂ or C≡CH; R^{e7} is OH, —OC(═O)Et, —OC(═O)-i-Pr or —OC(═O)-t-Bu; and A^{e8} is N or CH.

“Cellulose biosynthesis inhibitors” (b14) inhibit the biosynthesis of cellulose in certain plants. They are most effective when applied preemergence or early postemergence on young or rapidly growing plants. Examples of cellulose biosynthesis inhibitors include chlorthiamid, dichlobenil, flupoxam, indaziflam (N²-[(1R,2S)-2,3-dihydro-2,6-dimethyl-1H-inden-1-yl]-6-(1-fluoroethyl)-1,3,5-triazine-2,4-diamine), isoxaben and triaziflam.

“Other herbicides” (b15) include herbicides that act through a variety of different modes of action such as mitotic disruptors (e.g., flamprop-M-methyl and flamprop-M-isopropyl), organic arsenicals (e.g., DSMA, and MSMA), 7,8-dihydropteroate synthase inhibitors, chloroplast isoprenoid synthesis inhibitors and cell-wall biosynthesis inhibitors. Other herbicides include those herbicides having unknown modes of action or do not fall into a specific category listed in (b1) through (b14) or act through a combination of modes of action listed above. Examples of other herbicides include aclonifen, asulam, amitrole, bromobutide, cinmethylin, clomazone, cumyluron, cyclopyrimorate (6-chloro-3-(2-cyclopropyl-6-methylphenoxy)-4-pyridazinyl 4-morpholinecarboxylate), daimuron, difenzoquat, etobenzanid, fluometuron, flurenol, fosamine, fosamine-ammonium, dazomet, dymron, 2-[(2,4-dichlorophenyl)methyl]-4,4-dimethyl-3-isoxazolidinone (CA No. 81777-95-9), 2-[(2,5-dichlorophenyl)methyl]-4,4-dimethyl-3-isoxazolidinone (CA No. 81778-66-7), ipfencarbazone (1-(2,4-dichlorophenyl)-N-(2,4-difluorophenyl)-1,5-dihydro-N-(1-methylethyl)-5-oxo-4H-1,2,4-triazole-4-carboxamide), metam, methyldymron, oleic acid, oxaziclomefone, pelargonic acid, pyributicarb and 5-[[(2,6-difluorophenyl)methoxy]methyl]-4,5-dihydro-5-methyl-3-(3-methyl-2-thienyl)isoxazole. “Other herbicides” (b15) also include a compound of Formula (b15A)

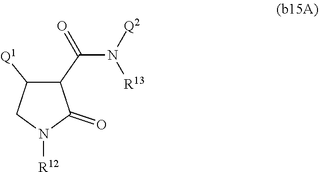

wherein
R¹² is H, C₁-C₆ alkyl, C₁-C₆ haloalkyl or C₄-C₈ cycloalkyl;
R¹³ is H, C₁-C₆ alkyl or C₁-C₆ alkoxy;
Q¹ is an optionally substituted ring system selected from the group consisting of phenyl, thienyl, pyridinyl, benzodioxolyl, naphthalenyl, benzofuranyl, furanyl, benzothiophenyl and pyrazolyl, wherein when substituted said ring system is substituted with 1 to 3 R¹⁴;
Q² is and optionally substituted ring system selected from the group consisting of phenyl, pyridinyl, benzodioxolyl, pyridinonyl, thiadiazolyl, thiazolyl, and oxazolyl, wherein when substituted said ring system is substituted with 1 to 3 R¹⁵;
each R¹⁴ is independently halogen, C₁-C₆ alkyl, C₁-C₆ haloalkyl, C₁-C₆ alkoxy, C₁-C₆ haloalkoxy, C₃-C₈ cyaloalkyl, cyano, C₁-C₆ alkylthio, C₁-C₆ alkylsulfinyl, C₁-C₆ alkylsulfonyl, SF₅, NHR¹⁷; or phenyl optionally substituted by 1 to 3 R¹⁶; or pyrazolyl optionally substituted by 1 to 3 R¹⁶;
each R¹⁵ is independently halogen, C₁-C₆ alkyl, C₁-C₆ haloalkyl, C₁-C₆ alkoxy, C₁-C₆ haloalkoxy, cyano, nitro, C₁-C₆ alkylthio, C₁-C₆ alkylsulfinyl, C₁-C₆ alkylsulfonyl;
each R¹⁶ is independently halogen, C₁-C₆ alkyl or C₁-C₆ haloalkyl; and
R¹⁷ is C₁-C₄ alkoxycarbonyl.

In one Embodiment wherein “other herbicides” (b15) also include a compound of Formula (b15A), it is preferred that R¹² is H or C₁-C₆ alkyl; more preferably R¹² is H or methyl. Preferrably R¹³ is H. Preferably Q¹ is either a phenyl ring or a pyridinyl ring, each ring substituted by 1 to 3 R¹⁴; more preferably Q¹ is a phenyl ring substituted by 1 to 2 R¹⁴. Preferably Q² is a phenyl ring substituted with 1 to 3 R¹⁵; more preferably Q² is a phenyl ring substituted by 1 to 2 R¹⁵. Preferably each R¹⁴ is independently halogen, C₁-C₄ alkyl, C₁-C₃ haloalkyl, C₁-C₃ alkoxy or C₁-C₃ haloalkoxy; more preferably each R¹⁴ is independently chloro, fluoro, bromo, C₁-C₂ haloalkyl, C₁-C₂ haloalkoxy or C₁-C₂ alkoxy. Preferrably each R¹⁵ is independently halogen, C₁-C₄ alkyl, C₁-C₃ haloalkoxy; more preferably each R¹⁵ is independently chloro, fluoro, bromo, C₁-C₂ haloalkyl, C₁-C₂ haloalkoxy or C₁-C₂ alkoxy. Specifically preferred as “other herbicides” (b15) include any one of the following (b15A-1) through (b15A-15):

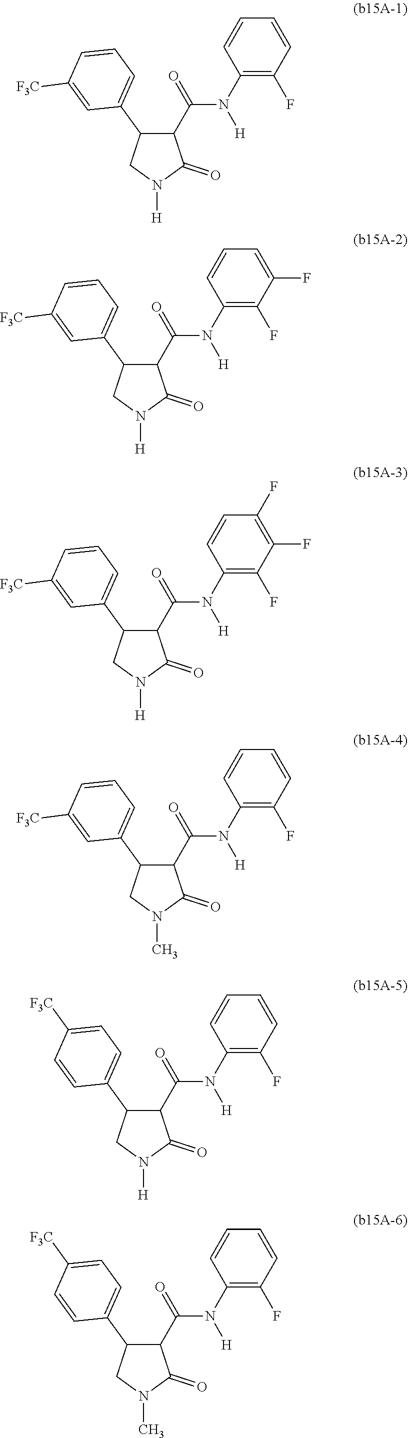 [SMILES: O=C1NCC(c2cccc(C(F)(F)F)c2)C1C(=O)Nc1ccccc1F ; O=C1NCC(c2cccc(C(F)(F)F)c2)C1C(=O)Nc1cccc(F)c1F ; O=C1NCC(c2cccc(C(F)(F)F)c2)C1C(=O)Nc1ccc(F)c(F)c1F ; CN1CC(c2cccc(C(F)(F)F)c2)C(C(=O)Nc2ccccc2F)C1=O ; O=C1NCC(c2ccc(C(F)(F)F)cc2)C1C(=O)Nc1ccccc1F ; CN1CC(c2ccc(C(F)(F)F)cc2)C(C(=O)Nc2ccccc2F)C1=O]

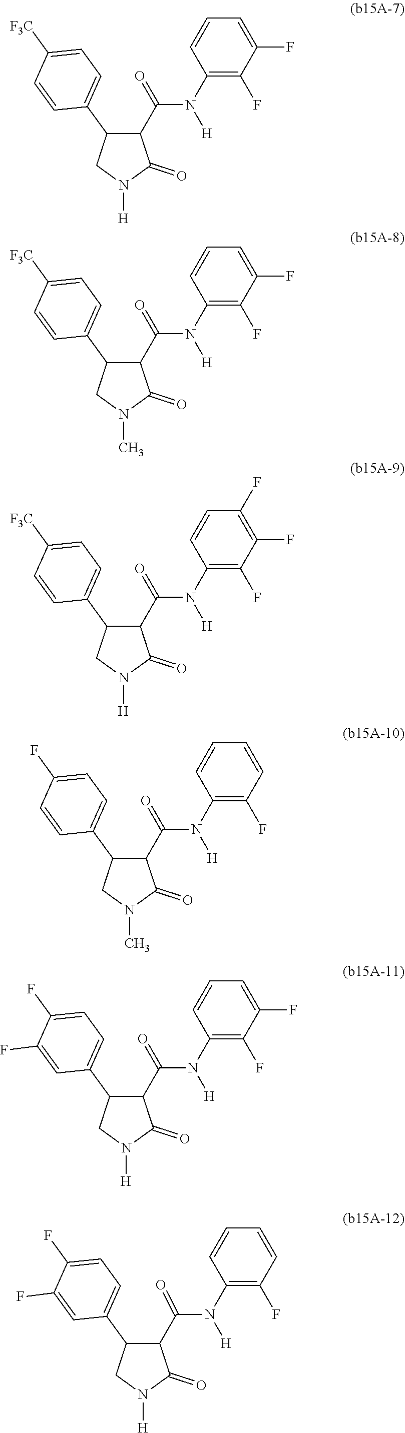 [SMILES: O=C1NCC(c2ccc(C(F)(F)F)cc2)C1C(=O)Nc1cccc(F)c1F ; CN1CC(c2ccc(C(F)(F)F)cc2)C(C(=O)Nc2cccc(F)c2F)C1=O ; O=C1NCC(c2ccc(C(F)(F)F)cc2)C1C(=O)Nc1ccc(F)c(F)c1F ; CN1CC(c2ccc(F)cc2)C(C(=O)Nc2ccccc2F)C1=O ; O=C1NCC(c2ccc(F)c(F)c2)C1C(=O)Nc1cccc(F)c1F ; O=C1NCC(c2ccc(F)c(F)c2)C1C(=O)Nc1ccccc1F]

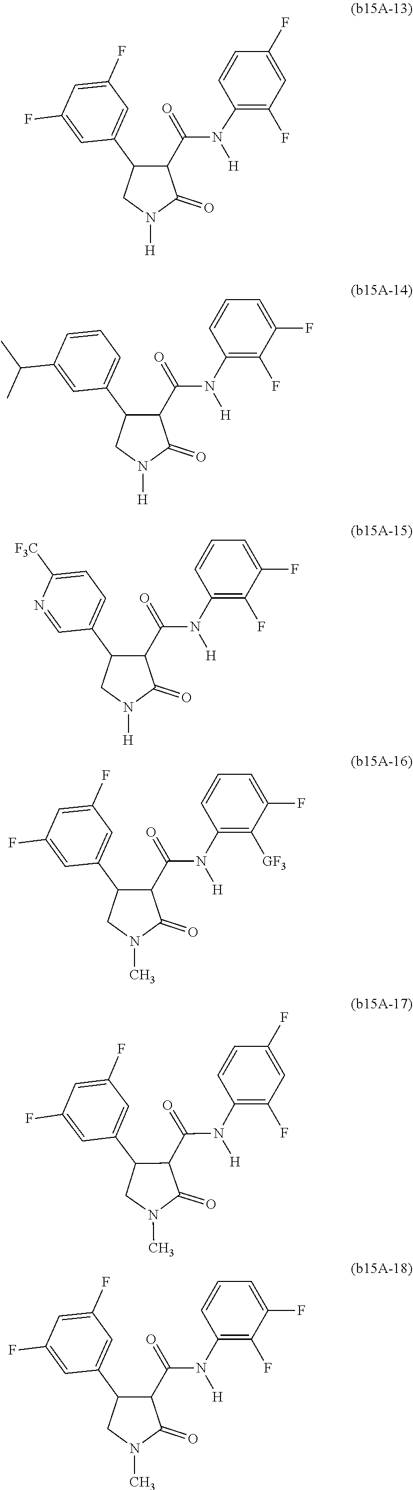

“Other herbicides” (b15) also include a compound of Formula (b15B)

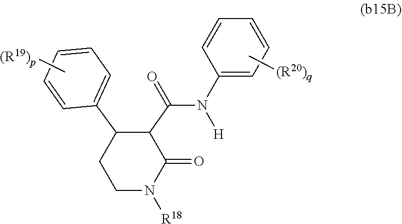

wherein
R¹⁸ is H, C₁-C₆ alkyl, C₁-C₆ haloalkyl or C₄-C₈ cycloalkyl;
each R¹⁹ is independently halogen, C₁-C₆ haloalkyl or C₁-C₆ haloalkoxy;
p is an integer of 0, 1, 2 or 3;
each R²⁰ is independently halogen, C₁-C₆ haloalkyl or C₁-C₆ haloalkoxy; and q is an integer of 0, 1, 2 or 3.

In one Embodiment wherein “other herbicides” (b15) also include a compound of Formula (b15B), it is preferred that R¹⁸ is H, methyl, ethyl or propyl; more preferably R¹⁸ is H or methyl; most preferably R¹⁸ is H. Preferrably each R¹⁹ is independently chloro, fluoro, C₁-C₃ haloalkyl or C₁-C₃ haloalkoxy; more preferably each R¹⁹ is independently chloro, fluoro, C₁ fluoroalkyl (i.e. fluoromethyl, difluoromethyl or trifluoromethyl) or C₁ fluoroalkoxy (i.e. trifluoromethoxy, difluoromethoxy or fluoromethoxy). Preferably each R²⁰ is independently chloro, fluoro, C₁ haloalkyl or C₁ haloalkoxy; more preferably each R²⁰ is independently chloro, fluoro, C₁ fluoroalkyl (i.e. fluoromethyl, difluorormethyl or trifluromethyl) or C₁ fluoroalkoxy (i.e. trifluoromethoxy, difluoromethoxy or fluoromethoxy). Specifically preferred as “other herbicides” (b15) include any one of the following (b15B-1) through (b15B-19):

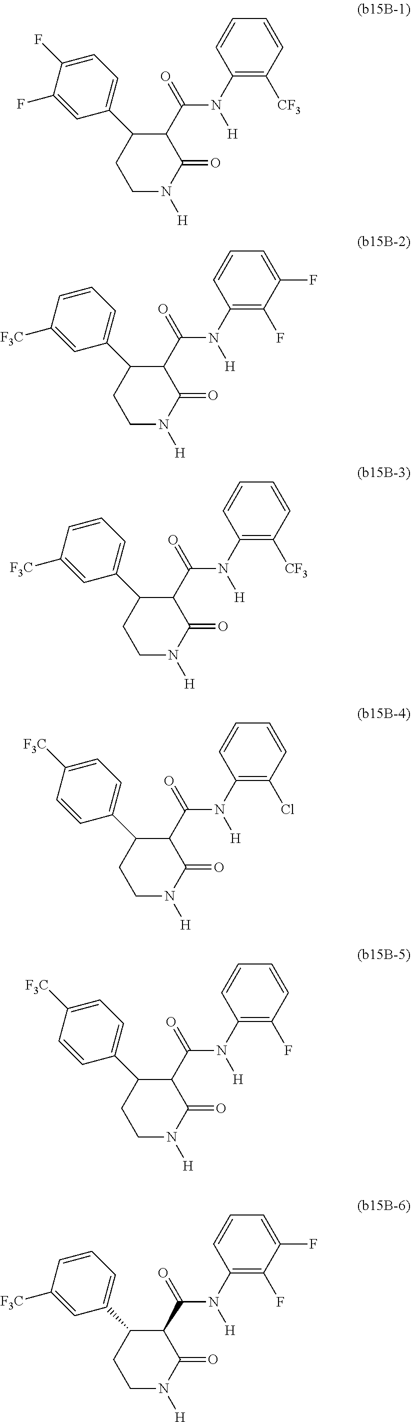 [SMILES: O=C1NCCC(c2ccc(F)c(F)c2)C1C(=O)Nc1ccccc1C(F)(F)F ; O=C1NCCC(c2cccc(C(F)(F)F)c2)C1C(=O)Nc1cccc(F)c1F ; O=C1NCCC(c2cccc(C(F)(F)F)c2)C1C(=O)Nc1ccccc1C(F)(F)F ; O=C1NCCC(c2ccc(C(F)(F)F)cc2)C1C(=O)Nc1ccccc1Cl ; O=C1NCCC(c2ccc(C(F)(F)F)cc2)C1C(=O)Nc1ccccc1F ; O=C1NCC[C@H](c2cccc(C(F)(F)F)c2)[C@H]1C(=O)Nc1cccc(F)c1F]

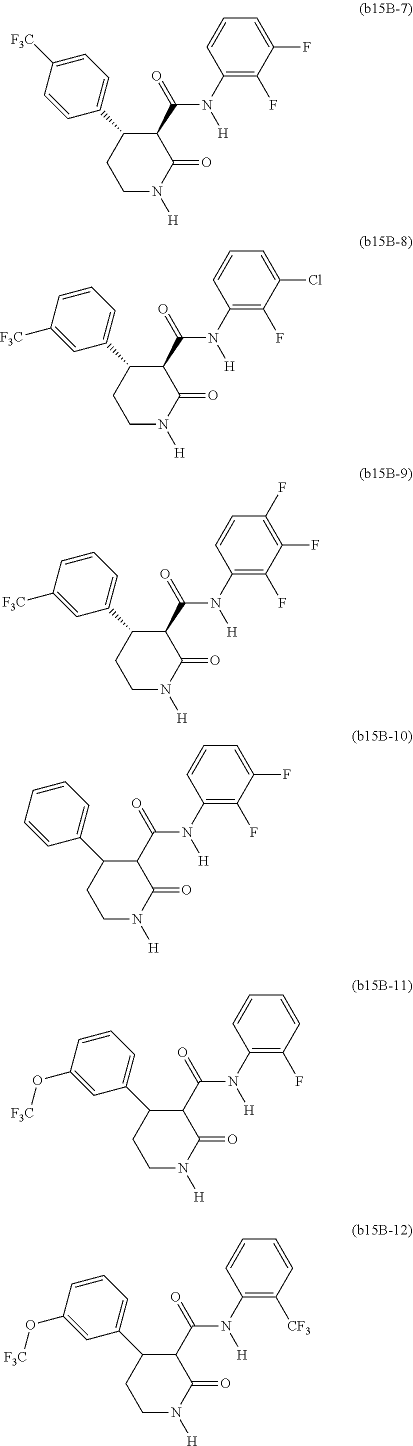

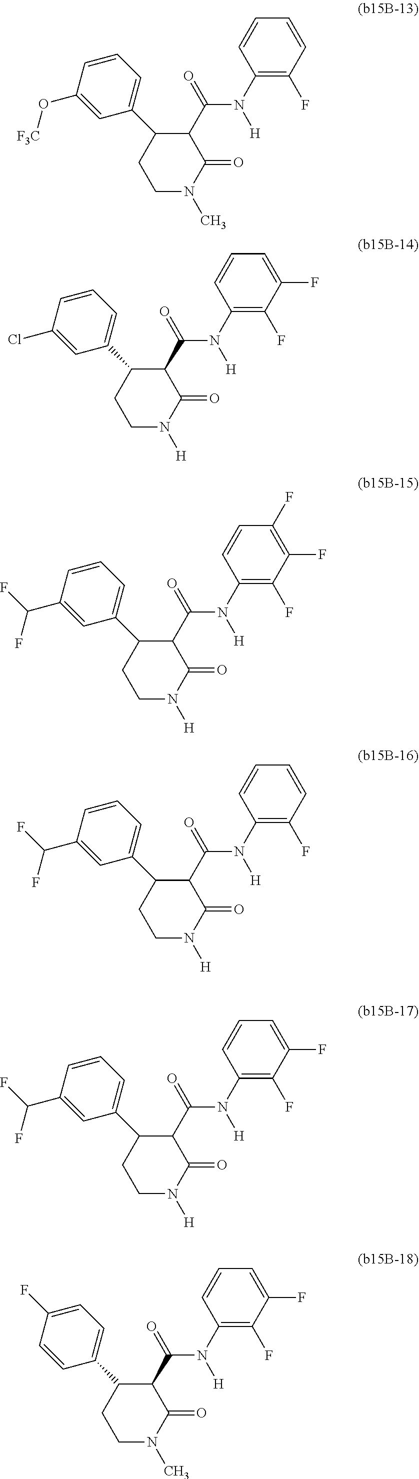

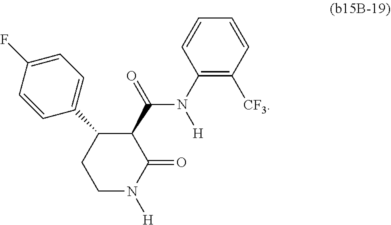 [SMILES: O=C1NCC[C@H](c2ccc(F)cc2)[C@H]1C(=O)Nc1ccccc1C(F)(F)F]

Another Embodiment wherein “other herbicides” (b15) also include a compound of Formula (b15C),

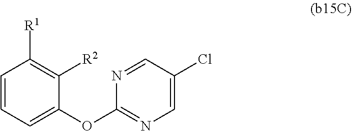

wherein R¹ is Cl, Br or CN; and R² is C(═O)CH₂CH₂CF₃, CH₂CH₂CH₂CH₂CF₃ or 3-CHF₂-isoxazol-5-yl.

“Herbicide safeners” (b16) are substances added to a herbicide formulation to eliminate or reduce phytotoxic effects of the herbicide to certain crops. These compounds protect crops from injury by herbicides but typically do not prevent the herbicide from controlling undesired vegetation. Examples of herbicide safeners include but are not limited to benoxacor, cloquintocet-mexyl, cumyluron, cyometrinil, cyprosulfamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone, naphthalic anhydride, oxabetrinil, N-(aminocarbonyl)-2-methylbenzenesulfonamide and N-(aminocarbonyl)-2-fluorobenzenesulfonamide, 1-bromo-4-[(chloromethyl)sulfonyl]benzene, 2-(dichloromethyl)-2-methyl-1,3-dioxolane (MG 191), 4-(dichloroacetyl)-1-oxa-4-azospiro[4.5]decane (MON 4660), 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide.

The compounds of Formula 1 can be prepared by general methods known in the art of synthetic organic chemistry. One or more of the following methods and variations as described in Schemes 1 through 8 can be used to prepare compounds of Formula 1. The definitions of groups A-1, A-2, A-3, A-4, R¹ to R⁸, W¹, W² and Q¹ to Q⁴ in the compounds of Formulae 1 through 13 are as defined above in the Summary of the Invention unless otherwise noted. Compounds of Formulae 2A, 2B, 3A, 3B, 3C, 3D, 3E, 7A and 11A are various subsets of the compounds of Formulae 2, 3, 7 and 11 and all substituents for Formulae 2A, 2B, 3A, 3B, 3C, 3D, 3E, 7A and 11A are as defined above for Formula 1 unless otherwise noted.

As shown in Scheme 1, a compound of Formula 1 can be prepared by nucleophilic substitution by heating a compound of Formula 2 (for example where LG is halogen) in a suitable solvent, such as acetonitrile, tetrahydrofuran or N,N-dimethylformamide in the presence of a base such as potassium or cesium carbonate, at temperatures ranging from 50 to 110° C., with the respective amine compound of Formula 3 or acid addition salt thereof. The corresponding enantiomers can be separated using a chiral HPLC column. The desired “A” variable in the compound of Formula 1 corresponds to the “A” variable (i.e. selected from 3-a, 3-b, 3-c and 3-d) in the compound of Formula 3 as shown in Scheme 1. The transformation in Scheme 1 may be conducted similarly with compounds of Formula 2 comprising other leaving groups such as wherein LG is C₁-C₄ haloalkylsulfonyl, C₁-C₄ alkylsulfonyloxy or C₁-C₄ haloalkylsulfonyloxy.

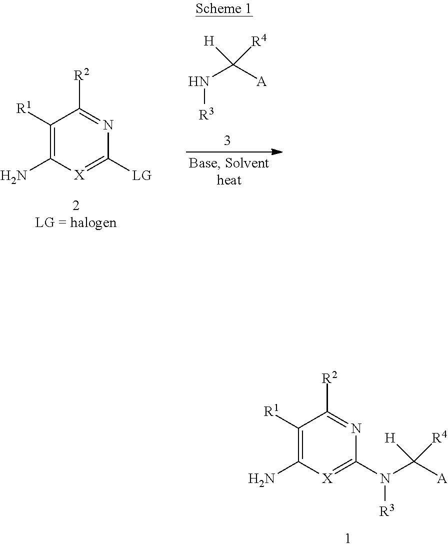

A is selected from

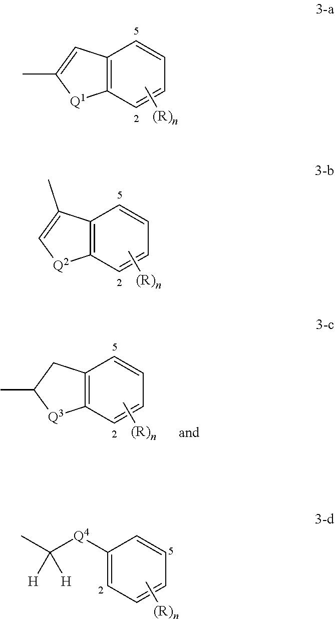

Aminopyridines (X is CR⁵) and aminopyrimidines (X is N) of Formula 2A (wherein LG is Cl) can be purchased commercially or can be prepared as shown in Scheme 2 by reacting a dichloropyridine or dichloropyrimidine of Formula 4 with ammonia in a suitable solvent such as methanol or ethanol, at temperatures typically ranging from 0° C. to the reflux temperature of the solvent. The resulting mixture of regioisomers of Formulae 2A and 5 can be separated by chromatography. The dichloropyridine or dichloropyrimidine compounds of Formula 4 are commercially available or can be prepared according the methods described in WO2008/077885.

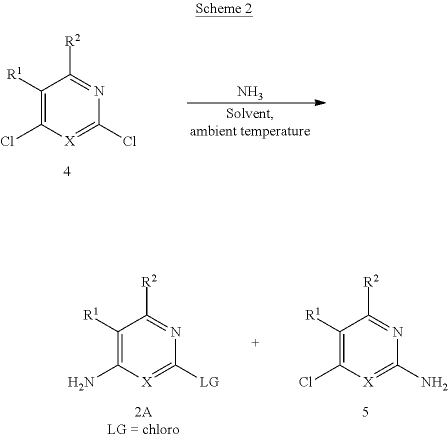

Aminopyrimidines of Formula 2B can be prepared in a single regioisomeric step by CF₃ insertion reactions as shown in Scheme 3. The CF₃ insertion can be achieved by reacting commercially available 2-chloropyrimidin-4-amine of Formula 6 with iodotrifluoromethane (CF₃I) in the presence of ferrous sulfate (FeSO₄.7 H₂O), hydrogen peroxide (H₂O₂) and hydrochloric acid (HCl) or sulfuric acid (H₂SO₄) at a temperature from 0° C. to ambient temperature. Specific examples of similar reactions can be found in WO 2007/055170. Alternatively, a similar CF₃ insertion can also be achieved by reacting the compound of formula 6 with sodium trifluromethanesufinate (CF₃SO₂Na) and manganese(III) acetate, using acetic acid as solvent at room temperature. Representative procedures are reported in Chem. Comm. 2014, 50, 3359-3362

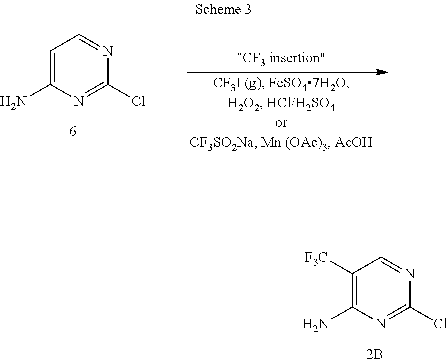 [SMILES: Nc1ccnc(Cl)n1 ; Nc1nc(Cl)ncc1C(F)(F)F]

Amines of Formula 3 or the acid addition salts thereof are commercially available or can be made as shown in Scheme 4. Racemic amines of Formulae 3A (i.e. R³ is H) can be prepared by reductive amination of corresponding keto compound of Formula 7 as shown in Scheme 4, in the presence of catalytic amount of acid (e.g., acetic acid), at a temperature from 0° C. to ambient temperature. Ammonia sources used for the reaction can be ammonia, ammonium hydroxide or ammonium acetate. Suitable reducing agents for the reaction include sodium cyanoborohydride, sodium borohydride or sodium tri-acetoxyborohydride in methanol or ethanol as solvent. Molecular sieves can be used for better efficiency of the reaction by removal of water. The desired “A” variable in the compound of Formula 3A correspond to the “A” variable (i.e. selected from 7-a, 7-b, 7-c and 7-d) in the compound of Formula 7 as shown in Scheme 4. Ketones of Formula 7 are available commercially or readily made by literature methods.

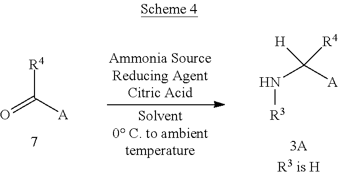

A is selected from

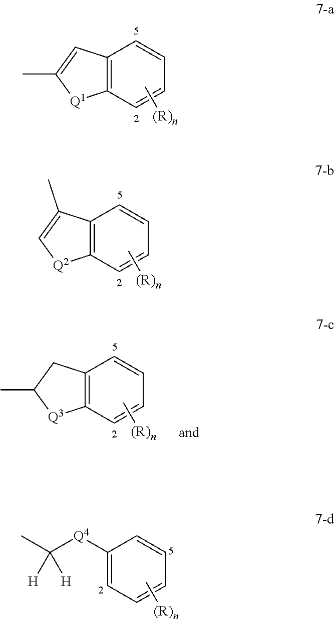

As shown in Scheme 5, the chiral amines of the Formula 3B or acid addition salts thereof, (i.e. A is A-4 and Q⁴ is O) can be prepared by a Mitsunobu substitution of an appropriately substituted phenol of Formula 8 and N-Boc-(D or L)-alaninol of Formula 9, in the presence of triphenylphosphine at a temperature from 0° C. to ambient temperature. Activating reagents used for the reaction include di(C₁-C₄ alkyl) azodicarboxylate such as diethyl azodicarboxylate (DEAD), diisopropyl azodicarboxylate (DIAD) or di-t-butyl azodicarboxylate (DTAD). Anhydrous solvents used for this reaction include tetrahydrofuran, diethyl ether, dioxane, toluene dimethoxyethane or dichloromethane. These methods are detailed in a review of Mitsunobu reactions in Chem. Rev. 2009, 109, 2551-2651 and references therein. The BOC protecting group can then be subsequently removed by treatment with acid to give the desired chiral amine of Formula 3B in the corresponding salt form. Acids used in this reaction include trifluoracetic acid or any other inorganic acids. Specific examples of this reaction are described in WO 2005/082859.

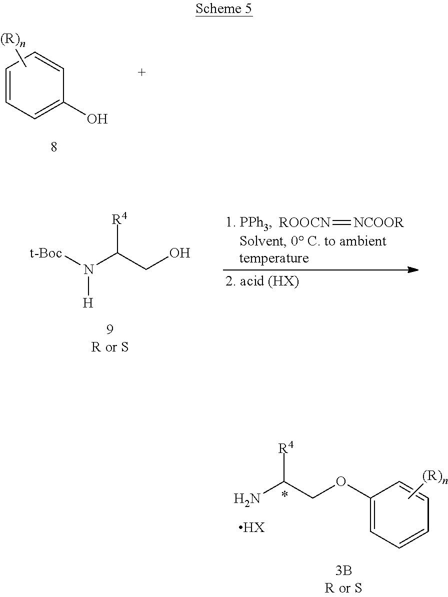

As shown in the Scheme 6, amines of Formula 3D (A is A-3; Q³=O, S or NR⁸ can be prepared by hydrogenation of amines of Formula 3C (A is A-1; Q¹=O, S or NR⁸) using palladium on charcoal in acetic acid in the presence of hydrogen gas. The synthesis can be achieved using methods reported in WO 2000/076990.

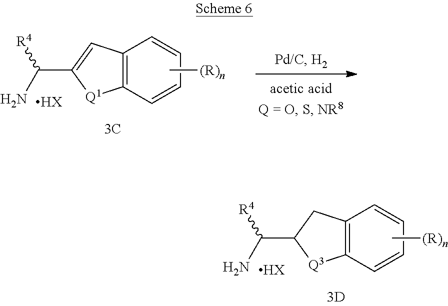

As shown in Scheme 7, chiral amines of the Formula 3C (i.e. Q¹=O, S or NR⁸) or acid addition salts thereof are commercially available or can be prepared in one pot by a Sonogashira coupling followed by cyclization, using a suitable chiral BOC-protected alkyne amine of Formula 10 and a properly substituted iodophenol, iodothiophenol or iodoaniline of Formula 11 in a dry solvent such as acetonitrile, 1,4-dioxane, tetrahydrofuran, dimethylsulfoxide or N,N-dimethylformamide. Sonogashira couplings typically are conducted in the presence of palladium(0) or a palladium(II) salt, a ligand, a copper(I) salt (e.g., copper(I) iodide) and a base (e.g., piperidine). Temperatures typically range from ambient temperature to the reflux temperature of the solvent. For conditions and reagents employed in Sonogashira couplings see Chemical Reviews 2007, 107(3), 874-922 and references cited therein. Specific examples can be found in Synthesis 1986, 9, 749-751. BOC removal from the protected amine can be easily achieved by treatment with a suitable acid to give the acid salt of the desired amine. The alkynes of Formula 10 are commercially available or can be synthesized from commercially available enantiomers of N-Boc-(D or L)-alaninol (Formula 9 in Scheme 5) as described in published literature procedures in WO 2008/130464, WO 2014/141104 or J. Org. Chem. 2014, 79(3), 1254-1264.

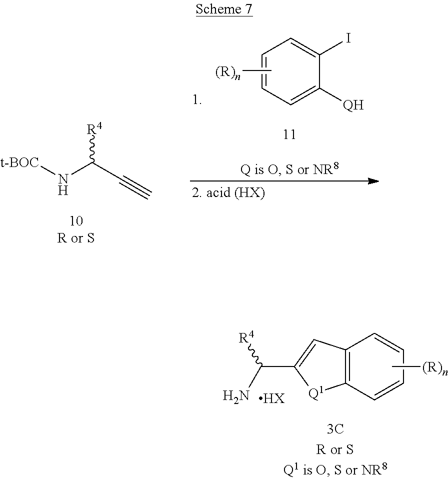

Ketones of Formula 7 can be prepared as shown in Scheme 8 from the corresponding commercially available aldehydes of Formula 12, by reaction with an appropriate Grignard reagent of Formula 13, followed by oxidation of the resulting alcohol. The desired “A” variable in the compound of Formula 7 correspond to the “A” variable (i.e. selected from 12-a, 12-b, 12-c and 12-d) in the compound of Formula 12 as shown in Scheme 8. Grignard reagents of Formula 13 can be purchased commercially. Oxidation methods that can be used for this reaction sequence include the Swern oxidation, Dess-Martin oxidation, PCC/PDC oxidation and TEMPO oxidation. Specific oxidation examples can be found in Eur. J. Med. Chem. 2016, 124, 17-35.

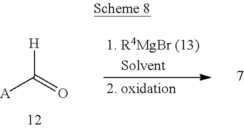

A is selected from

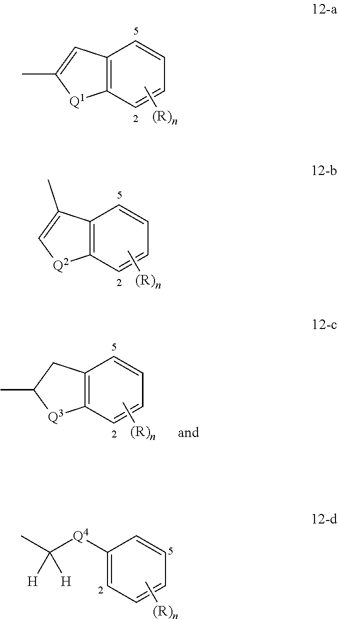

As shown in the Scheme 9, ketones of Formula 7A (i.e. wherein Q³=CH₂) can be prepared by treatment of compounds of Formula 14 (e.g. wherein LG is halogen) with 2,4-diketo compounds of Formula 15, with a suitable base in an appropriate solvent under heating conditions. For example, bases such as sodium or potassium hydroxide in a solvent such as toluene in the presence of phase transfer catalysts such as tetrabutylammonium bromide (TBAB) at temperatures from 60 to 120° C. are notable, as reported in Org. Lett. 2011, 13(16), 4304-4307.

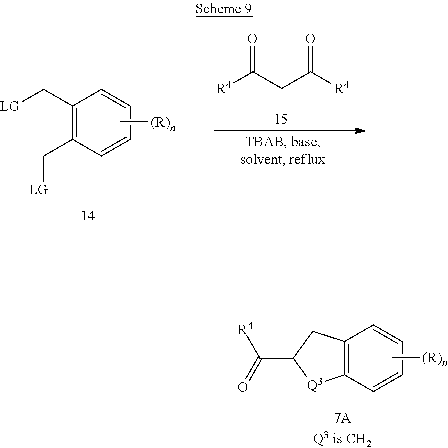

Chiral amines or acid addition salts thereof of Formula 3E can be alternatively prepared using an Ellman auxiliary with very good enantioselectivity. As shown in Scheme 10, (S)-chiral sufinylimines of Formula 14 with a high degree of stereoselectivity can synthesized from the condensation reactions of the aldehydes of Formula 12 with commercially available (S)-(−)-2-methyl-2-propanesulfinamide (Formula 16) in presence of Lewis acids like titanium tetraethoxide, copper sulfate or magnesium sulfate. Anhydrous solvents used for this reaction include tetrahydrofuran, diethyl ether, 1,4-dioxane or dichloromethane. For detailed condition and reagents for the Ellman procedure see Chemical Reviews 2010, 110(6), 3600-3740 and references cited therein. Chiral amines having the desired R-stereochemistry can be obtained from the addition of appropriate Grignard reagents (R₄MgBr) to (S)-sufinyl imines of Formula (?) at temperature from 0° C. to ambient temperature in dichloromethane solvent. Grignard reagents of Formula 13 can be purchased commercially. The N-tert-butanesulfinyl group can be easily cleaved by treatment with strong acids like hydrochloric acid in either methanol or 1,4-dioxan as solvent.

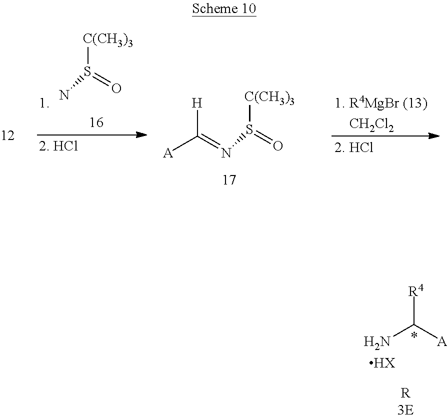

wherein A is selected from

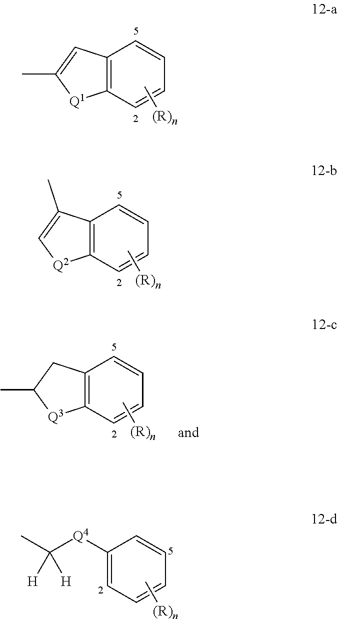

It is recognized by one skilled in the art that various functional groups can be converted into others to provide different compounds of Formula 1. For a valuable resource that illustrates the interconversion of functional groups in a simple and straightforward fashion, see Larock, R. C., Comprehensive Organic Transformations: A Guide to Functional Group Preparations, 2nd Ed., Wiley-VCH, New York, 1999. For example, intermediates for the preparation of compounds of Formula 1 may contain aromatic nitro groups, which can be reduced to amino groups, and then be converted via reactions well known in the art such as the Sandmeyer reaction, to various halides, providing compounds of Formula 1. The above reactions can also in many cases be performed in alternate order.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular order presented to prepare the compounds of Formula 1.

For example, derivatives of the Formula 1, wherein R¹, R² or R is halogen, in particular iodine or bromine, can be reacted with an alkene, acetylene, benzene, or 5- or 6-membered heteroaryl ring, with transition metal catalysis, e.g. palladium(0) or a palladium(II) catalyst, in an appropriate solvent in presence of suitable base at temperatures between 20 and 150° C. to give compounds of the Formula 1 wherein R¹, R² or R are substituted or unsubstituted alkene, alkyne, phenyl, or 5- or 6-membered heteroaryl etc. Compounds of Formula 1, wherein R¹, R² or R is CN, can be hydrolyzed under acidic or basic conditions to give carboxylic acids that can be subsequently transformed into acid chlorides and, in turn, these can be converted into amides, by simple organic transformations. Derivatives of Formula 1 wherein R¹, R² or R is halogen can also be converted to the corresponding alkoxyalkyl, aminoalkyl or diaminoalkyl substituted compounds through treatment with a suitable alcohol or amine in an appropriate solvent in presence of a suitable base at temperatures between 0 and 150° C. One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following non-limiting Examples are illustrative of the invention. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra (CDCl₃, 500 MHz unless indicated otherwise) are reported in ppm downfield from tetramethylsilane; “s” means singlet, “d” means doublet, “t” means triplet, “q” means quartet, “m” means multiplet, and “br s” means broad singlet. The abbreviation “LCMS” stands for liquid chromatographic mass spectroscopy.

### Synthesis Example 1

### Preparation of N2-[(1R)-1-(2-benzofuranyl)ethyl]-5-(trifluoromethyl)-2,4-pyrimidinediamine (i.e. Compound 22)

### Step A: Preparation of 2-chloro-5-(trifluoromethyl)-4-pyrimidinamine

To 2,4-dichloro-5-(trifluoromethyl)pyrimidine (5 g, 23 mmol) was slowly added 7 N ammonia in methanol (15 mL) at −10° C. and stirred at ambient temperature for 3 h, during which time an off-white precipitate formed in the reaction mixture. The reaction mixture was concentrated under reduced pressure to afford crude material. The crude material was purified by column chromatography on silica gel and eluted with ethyl acetate/petroleum ether (1:10) to provide the title product as a white solid (1.0 g, 22% yield). The undesired regioisomer (i.e. 4-chloro-5-(trifluoromethyl)-2-pyrimidinamine) (1.2 g) was also obtained as a white solid.

¹H NMR (CD₃OD, 400 MHz) δ 8.30 (s, 1H).

### Step A2: Alternative Preparation of 2-chloro-5-(trifluoromethyl)-4-pyrimidinamine

In a round-bottom flask, trifluoroiodomethane (CF₃I) gas (113.95 g, 581.39 mmol was sparged into dimethylsulfoxide (150 mL) at 10° C. for 2 h). The resulting solution was added dropwise at 6° C. for 10 min to a stirred solution of 4-amino-2-chloropyrimidine (25.0 g, 193.8 mmol) in dimethylsulfoxide (120 mL). Ferrous sulfate (FeSO₄.7 H₂O) (16.0 g, 58.1 mmol) in water (75 mL) was added to this mixture dropwise at 0° C. and then 30% hydrogen peroxide solution (13.17 g, 44 mL, 387.6 mmol) was added very slowly (dropwise) at 0° C. for 1 h. The resulting mixture was stirred at room temperature for 2 h. Concentrated hydrochloric acid (50 mL) was added dropwise to the reaction mixture at 0° C. for 30 min and the reaction mixture was stirred at 0° C. for 30 min. Progress of the reaction was monitored by thin layer chromatography. The reaction mixture was poured into ice water, and the resultant precipitated solid was collected by filtration and dried. The crude solid material was purified by column chromatography on silica gel and eluted with ethyl acetate/petroleum ether (1:10) to isolate the title compound as an off-white solid (12.0 g, 31% yield), the identity of which was confirmed by ¹H NMR and LCMS (94%).

### Step A3: Alternative Preparation of 2-chloro-5-(trifluoromethyl)-4-pyrimidinamine

To a stirred solution of 4-amino-2-chloropyrimidine (1.0 g, 7.8 mmol) in acetic acid (10 mL) was added sodium trifluromethanesufinate (2.13 g, 23.3 mmol) at 10° C. To this mixture was added portionwise manganese(I) acetate (8.31 g, 31.0 mmol) at the same temperature. The resulting mixture was stirred at room temperature for 24 h. The mixture was poured into ice water and extracted with ethyl acetate (2×50 mL). The combined organic layer was washed with water and brine solution, dried over anhydrous sodium sulfate and concentrated under reduced pressure. The crude material was purified by silica gel column chromatography eluting with ethyl acetate and petroleum ether (1:10) to give the title compound as an off-white solid (0.30 g, 19% yield), the identity of which was confirmed by ¹H NMR and LCMS (94%).

### Step B: Preparation of 1,1-dimethylethyl N-[(1R)-1-(2-benzofuranyl)ethyl]carbamate

A stirred solution of 2-iodophenol (2.0 g, 9.1 mmol), N-[(1R)-1-methyl-2-propyny-1-yl] carbamic acid 1,1-dimethylethyl ester (1.53 g, 9.1 mmol) and piperidine (0.77 g, 9.1 mmol) in N,N-dimethylformamide (25 mL) was purged with nitrogen gas for 10 to 15 min, then bis(triphenylphosphine)palladium(II) diacetate (0.136 g, 0.18 mmol) and copper(I) iodide (0.069 g, 0.36 mmol) were added. The reaction mixture was purged with nitrogen gas for a further 10 to 15 min and stirred for 4 d at ambient temperature. After complete consumption of starting material, the reaction mixture was diluted with ethyl acetate (50 mL) and washed with water and brine solution. The organic layer was dried over anhydrous sodium sulfate. The solvent was removed under reduced pressure to afford a crude material, which was purified by column chromatography on silica gel, eluting in ethyl acetate/petroleum ether (1:20) to provide the title compound (1.5 g) as a light brown liquid which was used directly in the next step.

### Step C: Preparation of ((αR)-α-methyl-2-benzofuranmethanamine

To a stirred solution of 1,1-dimethylethyl N-[(1R)-1-(2-benzofuranyl)ethyl]carbamate, (i.e. the product of Step B, 1.0 g, 3.6 mmol), in dichloromethane (10 mL) was added trifluoracetic acid (4.14 g, 36.3 mmol) at 0° C., and the reaction mixture was stirred at ambient temperature for 2 h. Upon complete consumption of starting material, the reaction mixture was distilled under reduced pressure to give a crude material. The crude material was made basic with saturated aqueous sodium bicarbonate solution, then extracted with dichloromethane (2×15 mL). The combined organic layers were dried over anhydrous Na₂SO₄, filtered, and concentrated under reduced pressure. The material was triturated with n-pentane to yield the title compound (0.35 g) as a light brown semisolid.

¹H NMR (DMSO-d₆, 500 MHz) δ 7.55 (d, 1H), 7.49 (d, 1H), 7.24-7.18 (m, 2H), 6.65 (s, 1H), 4.08 (q, 1H), 2.21 (br s, 2H), 1.38 (d, 3H).

### Step D: Preparation of N2-[(1R)-1-(2-benzofuranyl)ethyl]-5-(trifluoromethyl)-2,4-pyrimidineamine

To a stirred solution of 2-chloro-5-(trifluoromethyl)-4-pyrimidinamine, i.e. the product of Step A, (0.20 g, 1.0 mmol) and (αR)-α-methyl-2-benzofuranmethanamine (i.e. the product of Step C, 0.163 g, 1.0 mmol) in anhydrous N,N-dimethylformamide (5.0 mL) was added anhydrous potassium carbonate (0.420 g, 3.0 mmol) at ambient temperature, then the mixture was heated to 120° C. for 4 h. Upon complete consumption of the starting material, the reaction mixture was allowed to cool to ambient temperature, diluted with ethyl acetate (10 mL), then filtered through Celite® diatomaceous earth filter aid. The collected filtrate was distilled under reduced pressure to afford a crude material, which was purified by column chromatography on silica gel, eluting with ethyl acetate/petroleum ether (1:10) to provide the title compound (0.052 g) as an off-white solid.

¹H NMR δ 8.15 (br s, 1H), 7.50 (d, 1H), 7.43 (d, 1H), 7.24-7.18 (m, 2H), 6.56 (s, 1H), 5.83 (br s, 1H), 5.43 (br s, 1H), 5.13 (br s, 2H), 1.63 (t, 3H).

### Synthesis Example 2

### Preparation of N2-[(1R)-2-(3,5-dimethylphenoxy)-1-methylethyl]-5-(trifluoromethyl)-2,4-pyrimidinediamine (i.e. Compound 25)

### Step A: Preparation of 1,1-dimethylethyl N-[(1R)-2-(3,5-dimethylphenoxy)-1-methylethyl]carbamate

To a solution of (R)-(+)-2-(tert-butoxycarbonylamino)-1-propanol (1 g, 5.6 mmol) and 3,5-dimethylphenol (0.7 g, 5.6 mmol) in anhydrous tetrahydrofuran (10 mL), was added triphenylphosphine (2.2 g, 8.6 mmol) at 0° C. Diisopropyl azodicarboxylate (2 g, 8.6 mmol) in tetrahydrofuran (10 mL) was added dropwise to the solution above, which was then stirred at ambient temperature for 18 h. The mixture was poured into water (300 mL) and brought to pH 10 with 5 N aqueous sodium hydroxide. The mixture was extracted with diethyl ether (3×100 mL). The organic phase was washed with brine, dried over Na₂SO₄, filtered and concentrated. The crude material was purified by chromatography on silica gel, eluting with ethyl acetate/petroleum ether (1:10) to give the title compound as an off-white solid (160 mg).

¹H NMR δ 6.6 (s, 1H), 6.53 (s, 2H), 4.91-4.69 (bs, 1H), 4.13-3.97 (br s, 1H), 3.96-3.83 (m, 2H), 2.28 (s, 6H), 1.45 (s, 11H), 1.28-1.27 (d, 3H).

### Step B: Preparation of (2R)-1-(3,5-dimethylphenoxy)-2-propanamine trifluoracetic acid salt (1:1)

To a stirred solution of 1,1-dimethylethyl N-[(1R)-2-(3,5-dimethylphenoxy)-1-methylethyl]carbamate (i.e. the product obtained in Step A, 500 mg) in dichloromethane (10 mL) was added trifluoracetic acid (5 mL) at 0° C. and the mixture was stirred at ambient temperature for 2 h. Upon complete consumption of the starting material, the reaction mixture was distilled under reduced pressure to give a crude material. The crude material was used directly in the next step.

### Step C: Preparation of N2-[(1R)-2-(3,5-dimethylphenoxy)-1-methylethyl]-5-(trifluoromethyl)-2,4-pyrimidinediamine

To a stirred solution of 2-chloro-5-(trifluoromethyl)-4-pyrimidinamine (i.e. the product of Synthesis Example 1, Step A, 0.40 g, 2.0 mmol) and crude (2R)-1-(3,5-dimethylphenoxy)-2-propanamine trifluoracetic acid salt (1:1), (i.e. the product of Step B, 0.356 g, 2.0 mmol) in acetonitrile (10.0 mL) was added anhydrous potassium carbonate (0.8 g, 5.8 mmol) at ambient temperature, then the mixture was heated at the reflux temperature for 8 h. Upon complete consumption of starting material, the reaction mixture was cooled to ambient temperature, diluted with ethyl acetate (10 mL), then filtered through Celite® diatomaceous earth filter aid. The filtrate was collected, then distilled under reduced pressure to afford a crude material. The crude material was purified by column chromatography on silica gel, eluting with ethyl acetate/petroleum ether (1:10) to provide the title compound (0.25 g) as an off-white solid.

¹H NMR δ 8.2-8.1 (br s, 1H), 6.7-6.6 (m, 1H), 6.6-6.5 (m, 2H), 5.6-5.4 (br s, 1H), 5.2-5.0 (m, 2H), 4.5-4.3 (m, 1H1), 4.1-4.0 (m, 1H1), 4.0-3.9 (m, 1H1), 2.3-2.2 (s, 6H), 1.4-1.3 (d, 3H).

### Synthesis Example 3

### Preparation of N2-[(1R)-1-benzo[b]thien-2-ylethyl]-5-(trifluoromethyl)-2,4-pyrimidinediamine (i.e. Compound 4)

### Step A: Preparation of (S)—N-(benzothiophen-2-ylmethylene)-2-methyl-propane-2-sulfinamide

To a solution of benzothiophene-2-carbaldehyde (7 g, 43 mmol) in tetrahydrofuran (150 mL) at room temperature, (S)-(−)-2-methyl-2-propanesulfinamide (5.23 g, 43.2 mmol) and titanium tetraethoxide (19.67 g, 86.31 mmol) were added sequentially and the reaction mixture was stirred for 64 h. The reaction mixture was quenched with water and filtered though a short pad of Celite® diatomaceous earth and washed with ethyl acetate. The filtrate was extracted with ethyl acetate (2×150 mL). The combined organic layer was dried over anhydrous sodium sulfate, filtered, and concentrated under reduced pressure. The crude material was purified by column chromatography on silica gel eluting with ethyl acetate and petroleum ether (1:5) to provide the title compound as a white solid (8.7 g, 76% yield).

¹H NMR δ ppm 8.81 (s, 1H), 7.86-7.87 (m, 2H), 7.77 (s, 1H), 7.48-7.44 (m, 2H), 1.28 (s, 9H).

### Step B: Synthesis of (S_{S},R)—N-[1-(benzothiophen-2-yl)ethyl]-2-methyl-propane-2-sulfinamide

To a solution of (S)—N-(benzothiophen-2-ylmethylene)-2-methyl-propane-2-sulfinamide (i.e. the title compound of Step A, Synthesis Example 3, 4.39 g, 16.2 mmol) in dichloromethane (60 mL) at −40° C., methyl magnesium bromide (3 M solution in diethyl ether, 16.2 mL, 48.7 mmol) solution was added dropwise. The reaction mixture brought to room temperature and was stirred for additional 16 h. The reaction mixture was then quenched with slow addition of saturated aqueous solution of ammonium chloride at 0° C. The reaction mixture was then extracted with dichloromethane (2×100 mL). The combined organic layer was dried over anhydrous sodium sulfate, filtered, and concentrated under reduced pressure. The crude material was purified by column chromatography on silica gel and eluted with ethyl acetate/petroleum ether (1:5) to give the title compound as an off-white solid (5.59 g, 60% yield).

¹H NMR δ ppm 7.78-7.79 (d, 1H), 7.70-7.71 (d, 1H), 7.32-7.35 (m, 2H), 7.2 (s, 1H), 4.90-4.91 (q, 1H), 1.71-1.73 (d, 3H) 1.26 (s, 9H).

### Step C: Synthesis of (1R)-1-(benzothiophen-2-yl)ethanamine

To a solution of (S_{S},R)—N-[1-(benzothiophen-2-yl)ethyl]-2-methyl-propane-2-sulfinamide (i.e. the title compound of Step B, Synthesis Example 3 5.5 g, 19.6 mmol), in methanol (125 mL), was added dropwise 4 M HCl in 1,4-dioxane solution (50 mL) at room temperature. The reaction mixture was stirred for 90 min. After completion of the reaction, the solvent was evaporated, and the solid residue was washed with diethyl ether and dried. The obtained acid salt was dissolved in 50 mL water and the pH of the solution was adjusted to 12 with 15% aqueous sodium hydroxide solution. The aqeuous layer was extracted with dichloromethane (3×150 mL). The combined organic layer was washed with brine and concentrated to provide the title compound as an oil (3.49 g, 98% yield).

¹H NMR δ ppm 7.77-7.79 (d, 1H), 7.67-7.68 (d, 1H), 7.32-7.35 (m, 2H), 7.2 (s, 1H), 4.47-4.50 (q, 1H), 1.58-1.59 (d, 3H).

### Step D: Synthesis of N2-[(1R)-1-benzo[b]thien-2-ylethyl]-5-(trifluoromethyl)-2,4-pyrimidinediamine

To a stirred solution of 2-chloro-5-(trifluoromethyl)-4-pyrimidinamine (2.49 g, 12.7 mmol) and (1R)-1-(benzothiophen-2-yl)ethanamine (i.e. the title compound of Step C, Synthesis Example 3 (2.89 g, 15.8 mmol), in anhydrous acetonitrile (50 mL) was added anhydrous potassium carbonate (6.5 g, 47.5 mmol) at ambient temperature, then heated to reflux for 20 h. Progress of the reaction was monitored by thin layer chromatography analysis. Upon complete consumption of starting material, the reaction mixture was brought to room temperature and diluted with ethyl acetate (10 mL), then filtered through Celite® diatomaceous earth filter aid. The collected filtrate was distilled under reduced pressure to afford the crude material. The crude material was purified by column chromatography on silica gel and eluted with ethyl acetate/petroleum ether (1:3) to provide the title compound (1.5 g, 28% yield) as an off-white solid.

¹H NMR δ ppm 8.13 (bs, 1H), 7.75-7.77 (d, 1H), 7.68-7.69 (d, 1H), 7.26-7.33 (m, 2H), 7.2 (s, 1H), 5.54 (bs, 1H, 5.27 (bs, 2H), 1.69-1.70 (d, 3H).

By the procedures described herein together with methods known in the art, the following compounds of Tables 1 to 1218 can be prepared. The following abbreviations are used in the Tables which follow: t means tertiary, s means secondary, n means normal, i means iso, c means cyclo, Me means methyl, Et means ethyl, Pr means propyl, Bu means butyl, i-Pr means isopropyl, t-Bu means tert-butyl, c-Pr means cyclopropyl, 1-F-c-Pr means 1-fluorocyclopropyl, 2,2-di-F-c-Pr means 2,2-difluorocyclopropyl, c-Bu means cyclobutyl, c-Pn means cyclopentyl, c-Hx means cyclohexyl, Ph means phenyl, CN means cyano, NO₂ means nitro, S(O)CH₃ means methylsulfinyl and S(O)₂CH₃ means methylsulfonyl.

In the following Tables, A-1, A-2 and A-3 are defined as shown:

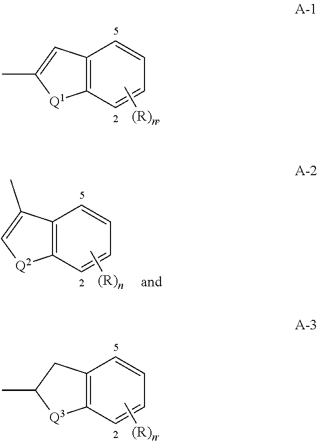

TABLE 1

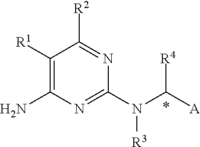

wherein A is
A is A-1, Q¹ is O, R¹ is CF₃, R² is H, R³ is H, R⁴ is CH₃ and (R)ₙ
H (i.e. n ═ 0)
3-F
3-Cl
3-Br
3-I
3-CH₃
3-OCH₃
3-CF₃
3-OCF₃
3-CHF₂
3-OCHF₂
3-COCH₃
3-CN
3-C≡CH
3-C≡CCH₃
3-CH═CH₂
3-Et
5-CN
2-F
2-Cl
2-Br
2-I
2-CH₃
2-OCH₃
2-CF₃
2-OCF₃
2-CN
3,4-di-F
2,4-di-F
4,5-di-F
3,5-di-F
2,5-di-F
3,4-di-Cl
2,4-di-Cl
4-F,3-CH₃
4-F,2-CH₃
3-F,2-CH₃
5-Cl,4-CH₃
5-Cl,3-CH₃
5-Cl,2-CH₃
4-Cl,3-CH₃
4-Cl,2-CH₃
3-Cl,2-CH₃
5-F,4-Cl
5-F,3-Cl
5-F,2-Cl
4-F,3-Cl
4-F,2-Cl
3-F,2-Cl
5-F,4-CF₃
5-F,3-CF₃
4-F
4-Cl
4-Br
4-I
4-CH₃
4-OCH₃
4-CF₃
4-OCF₃
4-CN
5-F
5-Cl
5-Br
5-I
5-CH₃
5-OCH₃
5-CF₃
5-OCF₃
4,5-di-Cl
3,5-di-Cl
2,5-di-Cl
3,4-di-CH₃
2,4-di-CH₃
4,5-di-CH₃
3,5-di-CH₃
2,5-di-CH₃
3,4-di-CF₃
2,4-di-CF₃
4,5-di-CF₃
3,5-di-CF₃
2,5-di-CF₃
5-F,4-CH₃
5-F,3-CH₃
5-F,2-CH₃
5-F,2-CF₃
4-F,3-CF₃
4-F,2-CF₃
3-F,2-CF₃
3,4,5-tri-F
2,3,4-tri-F
2,3,5-tri-F
3,4,5-tri-Cl
2,3,4-tri-Cl
2,3,5-tri-Cl
3,4,5-tri-CH₃
2,3,4-tri-CH₃
2,3,5-tri-CH₃
2,3,4,5-tetra-F
2,3,4,5-tetra-Cl
2,3,4,5-tetra-CH₃

The present disclosure also includes Tables 2 through 918, each of which is constructed the same as Table 1 above, except that the Header Row in Table 1 (i.e. A is A-1, Q¹ is O, R¹ is CF₃, R² is H, R³ is H, and R⁴ is CH₃) is replaced with the respective Header Row shown below in Tables 2 through 918. For example, the first entry in Table 2 is a compound of Formula 1 wherein A is A-1, Q¹ is O, R¹ is CF₃, R² is H, R³ is H, R⁴ is Et and (R)ₙ is H (i.e. n=0). Tables 3 through 918 are constructed similarly.

| Table | Header Row | | | | | |
|---|---|---|---|---|---|---|
| 2 | A is A-1, | Q¹ is O, | R¹ is CF₃, | R² is H, | R³ is H, | R⁴ is Et and |
| 3 | A is A-1, | Q¹ is O, | R¹ is CF₃, | R² is H, | R³ is H, | R⁴ is c-Pr and |
| 4 | A is A-1, | Q¹ is O, | R¹ is CF₃, | R² is H, | R³ is H, | R⁴ is Pr and |
| 5 | A is A-1, | Q¹ is O, | R¹ is CF₃, | R² is H, | R³ is H, | R⁴ is i-Pr and |
| 6 | A is A-1, | Q¹ is O, | R¹ is CF₃, | R² is H, | R³ is H, | R⁴ is Bu and |
| 7 | A is A-1, | Q¹ is O, | R¹ is CF₃, | R² is H, | R³ is H, | R⁴ is CF₃ and |
| 8 | A is A-1, | Q¹ is O, | R¹ is CF₃, | R² is H, | R³ is H, | R⁴ is CHF₂ and |
| 9 | A is A-1, | Q¹ is O, | R¹ is CF₃, | R² is CH₃, | R³ is H, | R⁴ is CH₃ and |
| 10 | A is A-1, | Q¹ is O, | R¹ is CF₃, | R² is CH₃, | R³ is H, | R⁴ is Et and |
| 11 | A is A-1, | Q¹ is O, | R¹ is CF₃, | R² is CH₃, | R³ is H, | R⁴ is c-Pr and |
| 12 | A is A-1, | Q¹ is O, | R¹ is CF₃, | R² is CH₃, | R³ is H, | R⁴ is Pr and |
| 13 | A is A-1, | Q¹ is O, | R¹ is CF₃, | R² is CH₃, | R³ is H, | R⁴ is i-Pr and |
| 14 | A is A-1, | Q¹ is O, | R¹ is CF₃, | R² is CH₃, | R³ is H, | R⁴ is Bu and |
| 15 | A is A-1, | Q¹ is O, | R¹ is CF₃, | R² is CH₃, | R³ is H, | R⁴ is CF₃ and |
| 16 | A is A-1, | Q¹ is O, | R¹ is CF₃, | R² is CH₃, | R³ is H, | R⁴ is CHF₂ and |
| 17 | A is A-1, | Q¹ is O, | R¹ is CF₃, | R² is Cl, | R³ is H, | R⁴ is CH₃ and |
| 18 | A is A-1, | Q¹ is O, | R¹ is CF₃, | R² is Cl, | R³ is H, | R⁴ is Et and |
| 19 | A is A-1, | Q¹ is O, | R¹ is CF₃, | R² is Cl, | R³ is H, | R⁴ is c-Pr and |
| 20 | A is A-1, | Q¹ is O, | R¹ is CF₃, | R² is Cl, | R³ is H, | R⁴ is Pr and |
| 21 | A is A-1, | Q¹ is O, | R¹ is CF₃, | R² is Cl, | R³ is H, | R⁴ is i-Pr and |
| 22 | A is A-1, | Q¹ is O, | R¹ is CF₃, | R² is Cl, | R³ is H, | R⁴ is Bu and |
| 23 | A is A-1, | Q¹ is O, | R¹ is CF₃, | R² is Cl, | R³ is H, | R⁴ is CF₃ and |
| 24 | A is A-1, | Q¹ is O, | R¹ is CF₃, | R² is Cl, | R³ is H, | R⁴ is CHF₂ and |
| 25 | A is A-1, | Q¹ is O, | R¹ is CN, | R² is H, | R³ is H, | R⁴ is CH₃ and |
| 26 | A is A-1, | Q¹ is O, | R¹ is CN, | R² is H, | R³ is H, | R⁴ is Et and |
| 27 | A is A-1, | Q¹ is O, | R¹ is CN, | R² is H, | R³ is H, | R⁴ is c-Pr and |
| 28 | A is A-1, | Q¹ is O, | R¹ is CN, | R² is H, | R³ is H, | R⁴ is Pr and |
| 29 | A is A-1, | Q¹ is O, | R¹ is CN, | R² is H, | R³ is H, | R⁴ is i-Pr and |
| 30 | A is A-1, | Q¹ is O, | R¹ is CN, | R² is H, | R³ is H, | R⁴ is Bu and |
| 31 | A is A-1, | Q¹ is O, | R¹ is CN, | R² is H, | R³ is H, | R⁴ is CF₃ and |
| 32 | A is A-1, | Q¹ is O, | R¹ is CN, | R² is H, | R³ is H, | R⁴ is CHF₂ and |
| 33 | A is A-1, | Q¹ is O, | R¹ is CN, | R² is CH₃, | R³ is H, | R⁴ is CH₃ and |
| 34 | A is A-1, | Q¹ is O, | R¹ is CN, | R² is CH₃, | R³ is H, | R⁴ is Et and |
| 35 | A is A-1, | Q¹ is O, | R¹ is CN, | R² is CH₃, | R³ is H, | R⁴ is c-Pr and |
| 36 | A is A-1, | Q¹ is O, | R¹ is CN, | R² is CH₃, | R³ is H, | R⁴ is Pr and |
| 37 | A is A-1, | Q¹ is O, | R¹ is CN, | R² is CH₃, | R³ is H, | R⁴ is i-Pr and |
| 38 | A is A-1, | Q¹ is O, | R¹ is CN, | R² is CH₃, | R³ is H, | R⁴ is Bu and |
| 39 | A is A-1, | Q¹ is O, | R¹ is CN, | R² is CH₃, | R³ is H, | R⁴ is CF₃ and |
| 40 | A is A-1, | Q¹ is O, | R¹ is CN, | R² is CH₃, | R³ is H, | R⁴ is CHF₂ and |
| 41 | A is A-1, | Q¹ is O, | R¹ is CN, | R² is Cl, | R³ is H, | R⁴ is CH₃ and |
| 42 | A is A-1, | Q¹ is O, | R¹ is CN, | R² is Cl, | R³ is H, | R⁴ is Et and |
| 43 | A is A-1, | Q¹ is O, | R¹ is CN, | R² is Cl, | R³ is H, | R⁴ is c-Pr and |
| 44 | A is A-1, | Q¹ is O, | R¹ is CN, | R² is Cl, | R³ is H, | R⁴ is Pr and |
| 45 | A is A-1, | Q¹ is O, | R¹ is CN, | R² is Cl, | R³ is H, | R⁴ is i-Pr and |
| 46 | A is A-1, | Q¹ is O, | R¹ is CN, | R² is Cl, | R³ is H, | R⁴ is Bu and |
| 47 | A is A-1, | Q¹ is O, | R¹ is CN, | R² is Cl, | R³ is H, | R⁴ is CF₃ and |
| 48 | A is A-1, | Q¹ is O, | R¹ is CN, | R² is Cl, | R³ is H, | R⁴ is CHF₂ and |
| 49 | A is A-1, | Q¹ is O, | R¹ is CHF₂, | R² is H, | R³ is H, | R⁴ is CH₃ and |
| 50 | A is A-1, | Q¹ is O, | R¹ is CHF₂, | R² is H, | R³ is H, | R⁴ is Et and |
| 51 | A is A-1, | Q¹ is O, | R¹ is CHF₂, | R² is H, | R³ is H, | R⁴ is c-Pr and |
| 52 | A is A-1, | Q¹ is O, | R¹ is CHF₂, | R² is H, | R³ is H, | R⁴ is Pr and |
| 53 | A is A-1, | Q¹ is O, | R¹ is CHF₂, | R² is H, | R³ is H, | R⁴ is i-Pr and |
| 54 | A is A-1, | Q¹ is O, | R¹ is CHF₂, | R² is H, | R³ is H, | R⁴ is Bu and |
| 55 | A is A-1, | Q¹ is O, | R¹ is CHF₂, | R² is H, | R³ is H, | R⁴ is CF₃ and |
| 56 | A is A-1, | Q¹ is O, | R¹ is CHF₂, | R² is H, | R³ is H, | R⁴ is CHF₂ and |
| 57 | A is A-1, | Q¹ is O, | R¹ is CHF₂, | R² is CH₃, | R³ is H, | R⁴ is CH₃ and |
| 58 | A is A-1, | Q¹ is O, | R¹ is CHF₂, | R² is CH₃, | R³ is H, | R⁴ is Et and |
| 59 | A is A-1, | Q¹ is O, | R¹ is CHF₂, | R² is CH₃, | R³ is H, | R⁴ is c-Pr and |
| 60 | A is A-1, | Q¹ is O, | R¹ is CHF₂, | R² is CH₃, | R³ is H, | R⁴ is Pr and |
| 61 | A is A-1, | Q¹ is O, | R¹ is CHF₂, | R² is CH₃, | R³ is H, | R⁴ is i-Pr and |
| 62 | A is A-1, | Q¹ is O, | R¹ is CHF₂, | R² is CH₃, | R³ is H, | R⁴ is Bu and |
| 63 | A is A-1, | Q¹ is O, | R¹ is CHF₂, | R² is CH₃, | R³ is H, | R⁴ is CF₃ and |
| 64 | A is A-1, | Q¹ is O, | R¹ is CHF₂, | R² is CH₃, | R³ is H, | R⁴ is CHF₂ and |
| 65 | A is A-1, | Q¹ is O, | R¹ is CHF₂, | R² is Cl, | R³ is H, | R⁴ is CH₃ and |
| 66 | A is A-1, | Q¹ is O, | R¹ is CHF₂, | R² is Cl, | R³ is H, | R⁴ is Et and |
| 67 | A is A-1, | Q¹ is O, | R¹ is CHF₂, | R² is Cl, | R³ is H, | R⁴ is c-Pr and |
| 68 | A is A-1, | Q¹ is O, | R¹ is CHF₂, | R² is Cl, | R³ is H, | R⁴ is Pr and |
| 69 | A is A-1, | Q¹ is O, | R¹ is CHF₂, | R² is Cl, | R³ is H, | R⁴ is i-Pr and |
| 70 | A is A-1, | Q¹ is O, | R¹ is CHF₂, | R² is Cl, | R³ is H, | R⁴ is Bu and |
| 71 | A is A-1, | Q¹ is O, | R¹ is CHF₂, | R² is Cl, | R³ is H, | R⁴ is CF₃ and |
| 72 | A is A-1, | Q¹ is O, | R¹ is CHF₂, | R² is Cl, | R³ is H, | R⁴ is CHF₂ and |
| 73 | A is A-1, | Q¹ is CH═CH, | R¹ is CF₃, | R² is H, | R³ is H, | R⁴ is CH₃ and |
| 74 | A is A-1, | Q¹ is CH═CH, | R¹ is CF₃, | R² is H, | R³ is H, | R⁴ is Et and |
| 75 | A is A-1, | Q¹ is CH═CH, | R¹ is CF₃, | R² is H, | R³ is H, | R⁴ is c-Pr and |
| 76 | A is A-1, | Q¹ is CH═CH, | R¹ is CF₃, | R² is H, | R³ is H, | R⁴ is Pr and |
| 77 | A is A-1, | Q¹ is CH═CH, | R¹ is CF₃, | R² is H, | R³ is H, | R⁴ is i-Pr and |
| 78 | A is A-1, | Q¹ is CH═CH, | R¹ is CF₃, | R² is H, | R³ is H, | R⁴ is Bu and |
| 79 | A is A-1, | Q¹ is CH═CH, | R¹ is CF₃, | R² is H, | R³ is H, | R⁴ is CF₃ and |
| 80 | A is A-1, | Q¹ is CH═CH, | R¹ is CF₃, | R² is H, | R³ is H, | R⁴ is CHF₂ and |
| 81 | A is A-1, | Q¹ is CH═CH, | R¹ is CF₃, | R² is CH₃, | R³ is H, | R⁴ is CH₃ and |
| 82 | A is A-1, | Q¹ is CH═CH, | R¹ is CF₃, | R² is CH₃, | R³ is H, | R⁴ is Et and |
| 83 | A is A-1, | Q¹ is CH═CH, | R¹ is CF₃, | R² is CH₃, | R³ is H, | R⁴ is c-Pr and |
| 84 | A is A-1, | Q¹ is CH═CH, | R¹ is CF₃, | R² is CH₃, | R³ is H, | R⁴ is Pr and |
| 85 | A is A-1, | Q¹ is CH═CH, | R¹ is CF₃, | R² is CH₃, | R³ is H, | R⁴ is i-Pr and |
| 86 | A is A-1, | Q¹ is CH═CH, | R¹ is CF₃, | R² is CH₃, | R³ is H, | R⁴ is Bu and |
| 87 | A is A-1, | Q¹ is CH═CH, | R¹ is CF₃, | R² is CH₃, | R³ is H, | R⁴ is CF₃ and |
| 88 | A is A-1, | Q¹ is CH═CH, | R¹ is CF₃, | R² is CH₃, | R³ is H, | R⁴ is CHF₂ and |
| 89 | A is GA-1, | Q¹ is CH═CH, | R¹ is CF₃, | R² is Cl, | R³ is H, | R⁴ is CH₃ and |
| 90 | A is A-1, | Q¹ is CH═CH, | R¹ is CF₃, | R² is Cl, | R³ is H, | R⁴ is Et and |
| 91 | A is A-1, | Q¹ is CH═CH, | R¹ is CF₃, | R² is Cl, | R³ is H, | R⁴ is c-Pr and |
| 92 | A is A-1, | Q¹ is CH═CH, | R¹ is CF₃, | R² is Cl, | R³ is H, | R⁴ is Pr and |
| 93 | A is A-1, | Q¹ is CH═CH, | R¹ is CF₃, | R² is Cl, | R³ is H, | R⁴ is i-Pr and |
| 94 | A is A-1, | Q¹ is CH═CH, | R¹ is CF₃, | R² is Cl, | R³ is H, | R⁴ is Bu and |
| 95 | A is A-1, | Q¹ is CH═CH, | R¹ is CF₃, | R² is Cl, | R³ is H, | R⁴ is CF₃ and |
| 96 | A is A-1, | Q¹ is CH═CH, | R¹ is CF₃, | R² is Cl, | R³ is H, | R⁴ is CHF₂ and |
| 97 | A is A-1, | Q¹ is S, | R¹ is CF₃, | R² is H, | R³ is H, | R⁴ is CH₃ and |
| 98 | A is A-1, | Q¹ is S, | R¹ is CF₃, | R² is H, | R³ is H, | R⁴ is Et and |
| 99 | A is A-1, | Q¹ is S, | R¹ is CF₃, | R² is H, | R³ is H, | R⁴ is c-Pr and |
| 100 | A is A-1, | Q¹ is S, | R¹ is CF₃, | R² is H, | R³ is H, | R⁴ is Pr and |
| 101 | A is A-1, | Q¹ is S, | R¹ is CF₃, | R² is H, | R³ is H, | R⁴ is i-Pr and |
| 102 | A is A-1, | Q¹ is S, | R¹ is CF₃, | R² is H, | R³ is H, | R⁴ is Bu and |
| 103 | A is A-1, | Q¹ is S, | R¹ is CF₃, | R² is H, | R³ is H, | R⁴ is CF₃ and |
| 104 | A is A-1, | Q¹ is S, | R¹ is CF₃, | R² is H, | R³ is H, | R⁴ is CHF₂ and |
| 105 | A is A-1, | Q¹ is S, | R¹ is CF₃, | R² is CH₃, | R³ is H, | R⁴ is CH₃ and |
| 106 | A is A-1, | Q¹ is S, | R¹ is CF₃, | R² is CH₃, | R³ is H, | R⁴ is Et and |
| 107 | A is A-1, | Q¹ is S, | R¹ is CF₃, | R² is CH₃, | R³ is H, | R⁴ is c-Pr and |
| 108 | A is A-1, | Q¹ is S, | R¹ is CF₃, | R² is CH₃, | R³ is H, | R⁴ is Pr and |
| 109 | A is A-1, | Q¹ is S, | R¹ is CF₃, | R² is CH₃, | R³ is H, | R⁴ is i-Pr and |
| 110 | A is A-1, | Q¹ is S, | R¹ is CF₃, | R² is CH₃, | R³ is H, | R⁴ is Bu and |
| 111 | A is A-1, | Q¹ is S, | R¹ is CF₃, | R² is CH₃, | R³ is H, | R⁴ is CF₃ and |
| 112 | A is A-1, | Q¹ is S, | R¹ is CF₃, | R² is CH₃, | R³ is H, | R⁴ is CHF₂ and |
| 113 | A is A-1, | Q¹ is S, | R¹ is CF₃, | R² is Cl, | R³ is H, | R⁴ is CH₃ and |
| 114 | A is A-1, | Q¹ is S, | R¹ is CF₃, | R² is Cl, | R³ is H, | R⁴ is Et and |
| 115 | A is A-1, | Q¹ is S, | R¹ is CF₃, | R² is Cl, | R³ is H, | R⁴ is c-Pr and |
| 116 | A is A-1, | Q¹ is S, | R¹ is CF₃, | R² is Cl, | R³ is H, | R⁴ is Pr and |
| 117 | A is A-1, | Q¹ is S, | R¹ is CF₃, | R² is Cl, | R³ is H, | R⁴ is i-Pr and |
| 118 | A is A-1, | Q¹ is S, | R¹ is CF₃, | R² is Cl, | R³ is H, | R⁴ is Bu and |
| 119 | A is A-1, | Q¹ is S, | R¹ is CF₃, | R² is Cl, | R³ is H, | R⁴ is CF₃ and |
| 120 | A is A-1, | Q¹ is S, | R¹ is CF₃, | R² is Cl, | R³ is H, | R⁴ is CHF₂ and |
| 121 | A is A-1, | Q¹ is SO₂, | R¹ is CF₃, | R² is H, | R³ is H, | R⁴ is CH₃ and |
| 122 | A is A-1, | Q¹ is SO₂, | R¹ is CF₃, | R² is H, | R³ is H, | R⁴ is Et and |
| 123 | A is A-1, | Q¹ is SO₂, | R¹ is CF₃, | R² is H, | R³ is H, | R⁴ is c-Pr and |
| 124 | A is A-1, | Q¹ is SO₂, | R¹ is CF₃, | R² is H, | R³ is H, | R⁴ is Pr and |
| 125 | A is A-1, | Q¹ is SO₂, | R¹ is CF₃, | R² is H, | R³ is H, | R⁴ is i-Pr and |
| 126 | A is A-1, | Q¹ is SO₂, | R¹ is CF₃, | R² is H, | R³ is H, | R⁴ is Bu and |
| 127 | A is A-1, | Q¹ is SO₂, | R¹ is CF₃, | R² is H, | R³ is H, | R⁴ is CF₃ and |
| 128 | A is A-1, | Q¹ is SO₂, | R¹ is CF₃, | R² is H, | R³ is H, | R⁴ is CHF₂ and |
| 129 | A is A-1, | Q¹ is SO₂, | R¹ is CF₃, | R² is CH₃, | R³ is H, | R⁴ is CH₃ and |
| 130 | A is A-1, | Q¹ is SO₂, | R¹ is CF₃, | R² is CH₃, | R³ is H, | R⁴ is Et and |
| 131 | A is A-1, | Q¹ is SO₂, | R¹ is CF₃, | R² is CH₃, | R³ is H, | R⁴ is c-Pr and |
| 132 | A is A-1, | Q¹ is SO₂, | R¹ is CF₃, | R² is CH₃, | R³ is H, | R⁴ is Pr and |
| 133 | A is A-1, | Q¹ is SO₂, | R¹ is CF₃, | R² is CH₃, | R³ is H, | R⁴ is i-Pr and |
| 134 | A is A-1, | Q¹ is SO₂, | R¹ is CF₃, | R² is CH₃, | R³ is H, | R⁴ is Bu and |
| 135 | A is A-1, | Q¹ is SO₂, | R¹ is CF₃, | R² is CH₃, | R³ is H, | R⁴ is CF₃ and |
| 136 | A is A-1, | Q¹ is SO₂, | R¹ is CF₃, | R² is CH₃, | R³ is H, | R⁴ is CHF₂ and |
| 137 | A is A-1, | Q¹ is SO₂, | R¹ is CF₃, | R² is Cl, | R³ is H, | R⁴ is CH₃ and |
| 138 | A is A-1, | Q¹ is SO₂, | R¹ is CF₃, | R² is Cl, | R³ is H, | R⁴ is Et and |
| 139 | A is A-1, | Q¹ is SO₂, | R¹ is CF₃, | R² is Cl, | R³ is H, | R⁴ is c-Pr and |
| 140 | A is A-1, | Q¹ is SO₂, | R¹ is CF₃, | R² is Cl, | R³ is H, | R⁴ is Pr and |
| 141 | A is A-1, | Q¹ is SO₂, | R¹ is CF₃, | R² is Cl, | R³ is H, | R⁴ is i-Pr and |
| 142 | A is A-1, | Q¹ is SO₂, | R¹ is CF₃, | R² is Cl, | R³ is H, | R⁴ is Bu and |
| 143 | A is A-1, | Q¹ is SO₂, | R¹ is CF₃, | R² is Cl, | R³ is H, | R⁴ is CF₃ and |
| 144 | A is A-1, | Q¹ is SO₂, | R¹ is CF₃, | R² is Cl, | R³ is H, | R⁴ is CHF₂ and |
| 145 | A is A-1, | Q¹ is NCH₃, | R¹ is CF₃, | R² is H, | R³ is H, | R⁴ is CH₃ and |
| 146 | A is A-1, | Q¹ is NCH₃, | R¹ is CF₃, | R² is H, | R³ is H, | R⁴ is Et and |
| 147 | A is A-1, | Q¹ is NCH₃, | R¹ is CF₃, | R² is H, | R³ is H, | R⁴ is c-Pr and |
| 148 | A is A-1, | Q¹ is NCH₃, | R¹ is CF₃, | R² is H, | R³ is H, | R⁴ is Pr and |
| 149 | A is A-1, | Q¹ is NCH₃, | R¹ is CF₃, | R² is H, | R³ is H, | R⁴ is i-Pr and |
| 150 | A is A-1, | Q¹ is NCH₃, | R¹ is CF₃, | R² is H, | R³ is H, | R⁴ is Bu and |
| 151 | A is A-1, | Q¹ is NCH₃, | R¹ is CF₃, | R² is H, | R³ is H, | R⁴ is CF₃ and |
| 152 | A is A-1, | Q¹ is NCH₃, | R¹ is CF₃, | R² is H, | R³ is H, | R⁴ is CHF₂ and |
| 153 | A is A-1, | Q¹ is NCH₃, | R¹ is CF₃, | R² is CH₃, | R³ is H, | R⁴ is CH₃ and |
| 154 | A is A-1, | Q¹ is NCH₃, | R¹ is CF₃, | R² is CH₃, | R³ is H, | R⁴ is Et and |
| 155 | A is A-1, | Q¹ is NCH₃, | R¹ is CF₃, | R² is CH₃, | R³ is H, | R⁴ is c-Pr and |
| 156 | A is A-1, | Q¹ is NCH₃, | R¹ is CF₃, | R² is CH₃, | R³ is H, | R⁴ is Pr and |
| 157 | A is A-1, | Q¹ is NCH₃, | R¹ is CF₃, | R² is CH₃, | R³ is H, | R⁴ is i-Pr and |
| 158 | A is A-1, | Q¹ is NCH₃, | R¹ is CF₃, | R² is CH₃, | R³ is H, | R⁴ is Bu and |
| 159 | A is A-1, | Q¹ is NCH₃, | R¹ is CF₃, | R² is CH₃, | R³ is H, | R⁴ is CF₃ and |
| 160 | A is A-1, | Q¹ is NCH₃, | R¹ is CF₃, | R² is CH₃, | R³ is H, | R⁴ is CHF₂ and |
| 161 | A is A-1, | Q¹ is NCH₃, | R¹ is CF₃, | R² is Cl, | R³ is H, | R⁴ is CH₃ and |
| 162 | A is A-1, | Q¹ is NCH₃, | R¹ is CF₃, | R² is Cl, | R³ is H, | R⁴ is Et and |
| 163 | A is A-1, | Q¹ is NCH₃, | R¹ is CF₃, | R² is Cl, | R³ is H, | R⁴ is c-Pr and |
| 164 | A is A-1, | Q¹ is NCH₃, | R¹ is CF₃, | R² is Cl, | R³ is H, | R⁴ is Pr and |
| 165 | A is A-1, | Q¹ is NCH₃, | R¹ is CF₃, | R² is Cl, | R³ is H, | R⁴ is i-Pr and |
| 166 | A is A-1, | Q¹ is NCH₃, | R¹ is CF₃, | R² is Cl, | R³ is H, | R⁴ is Bu and |
| 167 | A is A-1, | Q¹ is NCH₃, | R¹ is CF₃, | R² is Cl, | R³ is H, | R⁴ is CF₃ and |
| 168 | A is A-1, | Q¹ is NCH₃, | R¹ is CF₃, | R² is Cl, | R³ is H, | R⁴ is CHF₂ and |
| 169 | A is A-1, | Q¹ is NCH₃, | R¹ is CF₃, | R² is H, | R³ is H, | R⁴ is CH₃ and |
| 170 | A is A-1, | Q¹ is NCH₃, | R¹ is CF₃, | R² is H, | R³ is H, | R⁴ is Et and |
| 171 | A is A-1, | Q¹ is NCH₃, | R¹ is CF₃, | R² is H, | R³ is H, | R⁴ is c-Pr and |
| 172 | A is A-1, | Q¹ is NCH₃, | R¹ is CF₃, | R² is H, | R³ is H, | R⁴ is Pr and |
| 173 | A is A-1, | Q¹ is NCH₃, | R¹ is CF₃, | R² is H, | R³ is H, | R⁴ is i-Pr and |
| 174 | A is A-1, | Q¹ is NCH₃, | R¹ is CF₃, | R² is H, | R³ is H, | R⁴ is Bu and |
| 175 | A is A-1, | Q¹ is NCH₃, | R¹ is CF₃, | R² is H, | R³ is H, | R⁴ is CF₃ and |
| 176 | A is A-1, | Q¹ is NCH₃, | R¹ is CF₃, | R² is H, | R³ is H, | R⁴ is CHF₂ and |
| 177 | A is A-1, | Q¹ is NCH₃, | R¹ is CF₃, | R² is CH₃, | R³ is H, | R⁴ is CH₃ and |
| 178 | A is A-1, | Q¹ is NCH₃, | R¹ is CF₃, | R² is CH₃, | R³ is H, | R⁴ is Et and |
| 179 | A is A-1, | Q¹ is NCH₃, | R¹ is CF₃, | R² is CH₃, | R³ is H, | R⁴ is c-Pr and |
| 180 | A is A-1, | Q¹ is NCH₃, | R¹ is CF₃, | R² is CH₃, | R³ is H, | R⁴ is Pr and |
| 181 | A is A-1, | Q¹ is NCH₃, | R¹ is CF₃, | R² is CH₃, | R³ is H, | R⁴ is i-Pr and |
| 182 | A is A-1, | Q¹ is NCH₃, | R¹ is CF₃, | R² is CH₃, | R³ is H, | R⁴ is Bu and |
| 183 | A is A-1, | Q¹ is NCH₃, | R¹ is CF₃, | R² is CH₃, | R³ is H, | R⁴ is CF₃ and |
| 184 | A is A-1, | Q¹ is NCH₃, | R¹ is CF₃, | R² is CH₃, | R³ is H, | R⁴ is CHF₂ and |
| 185 | A is A-1, | Q¹ is NCH₃, | R¹ is CF₃, | R² is Cl, | R³ is H, | R⁴ is CH₃ and |
| 186 | A is A-1, | Q¹ is NCH₃, | R¹ is CF₃, | R² is Cl, | R³ is H, | R⁴ is Et and |
| 187 | A is A-1, | Q¹ is NCH₃, | R¹ is CF₃, | R² is Cl, | R³ is H, | R⁴ is c-Pr and |
| 188 | A is A-1, | Q¹ is NCH₃, | R¹ is CF₃, | R² is Cl, | R³ is H, | R⁴ is Pr and |
| 189 | A is A-1, | Q¹ is NCH₃, | R¹ is CF₃, | R² is Cl, | R³ is H, | R⁴ is i-Pr and |
| 190 | A is A-1, | Q¹ is NCH₃, | R¹ is CF₃, | R² is Cl, | R³ is H, | R⁴ is Bu and |
| 191 | A is A-1, | Q¹ is NCH₃, | R¹ is CF₃, | R² is Cl, | R³ is H, | R⁴ is CF₃ and |
| 192 | A is A-1, | Q¹ is NCH₃, | R¹ is CF₃, | R² is Cl, | R³ is H, | R⁴ is CHF₂ and |
| 193 | A is A-1, | Q¹ is CO, | R¹ is CF₃, | R² is H, | R³ is H, | R⁴ is CH₃ and |
| 194 | A is A-1, | Q¹ is CO, | R¹ is CF₃, | R² is H, | R³ is H, | R⁴ is Et and |
| 195 | A is A-1, | Q¹ is CO, | R¹ is CF₃, | R² is H, | R³ is H, | R⁴ is c-Pr and |
| 196 | A is A-1, | Q¹ is CO, | R¹ is CF₃, | R² is H, | R³ is H, | R⁴ is Pr and |
| 197 | A is A-1, | Q¹ is CO, | R¹ is CF₃, | R² is H, | R³ is H, | R⁴ is i-Pr and |
| 198 | A is A-1, | Q¹ is CO, | R¹ is CF₃, | R² is H, | R³ is H, | R⁴ is Bu and |
| 199 | A is A-1, | Q¹ is CO, | R¹ is CF₃, | R² is H, | R³ is H, | R⁴ is CF₃ and |
| 200 | A is A-1, | Q¹ is CO, | R¹ is CF₃, | R² is H, | R³ is H, | R⁴ is CHF₂ and |
| 201 | A is A-1, | Q¹ is CO, | R¹ is CF₃, | R² is CH₃, | R³ is H, | R⁴ is CH₃ and |
| 202 | A is A-1, | Q¹ is CO, | R¹ is CF₃, | R² is CH₃, | R³ is H, | R⁴ is Et and |
| 203 | A is A-1, | Q¹ is CO, | R¹ is CF₃, | R² is CH₃, | R³ is H, | R⁴ is c-Pr and |
| 204 | A is A-1, | Q¹ is CO, | R¹ is CF₃, | R² is CH₃, | R³ is H, | R⁴ is Pr and |
| 205 | A is A-1, | Q¹ is CO, | R¹ is CF₃, | R² is CH₃, | R³ is H, | R⁴ is i-Pr and |
| 206 | A is A-1, | Q¹ is CO, | R¹ is CF₃, | R² is CH₃, | R³ is H, | R⁴ is Bu and |
| 207 | A is A-1, | Q¹ is CO, | R¹ is CF₃, | R² is CH₃, | R³ is H, | R⁴ is CF₃ and |
| 208 | A is A-1, | Q¹ is CO, | R¹ is CF₃, | R² is CH₃, | R³ is H, | R⁴ is CHF₂ and |
| 209 | A is A-1, | Q¹ is CO, | R¹ is CF₃, | R² is Cl, | R³ is H, | R⁴ is CH₃ and |
| 210 | A is A-1, | Q¹ is CO, | R¹ is CF₃, | R² is Cl, | R³ is H, | R⁴ is Et and |
| 211 | A is A-1, | Q¹ is CO, | R¹ is CF₃, | R² is Cl, | R³ is H, | R⁴ is c-Pr and |
| 212 | A is A-1, | Q¹ is CO, | R¹ is CF₃, | R² is Cl, | R³ is H, | R⁴ is Pr and |
| 213 | A is A-1, | Q¹ is CO, | R¹ is CF₃, | R² is Cl, | R³ is H, | R⁴ is i-Pr and |
| 214 | A is A-1, | Q¹ is CO, | R¹ is CF₃, | R² is Cl, | R³ is H, | R⁴ is Bu and |
| 215 | A is A-1, | Q¹ is CO, | R¹ is CF₃, | R² is Cl, | R³ is H, | R⁴ is CF₃ and |
| 216 | A is A-1, | Q¹ is CO, | R¹ is CF₃, | R² is Cl, | R³ is H, | R⁴ is CHF₂ and |
| 217 | A is A-1, | Q¹ is CH═CH, | R¹ is CF₃, | R² is H, | R³ is CH₃, | R⁴ is CH₃ and |
| 218 | A is A-1, | Q¹ is CH═CH, | R¹ is CF₃, | R² is H, | R³ is COCH₃, | R⁴ is CH₃ and |
| 219 | A is A-1, | Q¹ is CH═CH, | R¹ is CF₃, | R² is H, | R³ is CO₂CH₃, | R⁴ is CH₃ and |
| 220 | A is A-1, | Q¹ is CH═CH, | R¹ is CF₃, | R² is H, | R³ is COCF₃, | R⁴ is CH₃ and |
| 221 | A is A-1, | Q¹ is CH═CH, | R¹ is CF₃, | R² is CHF₂, | R³ is H, | R⁴ is CH₃ and |
| 222 | A is A-1, | Q¹ is CH═CH, | R¹ is CF₃, | R² is F, | R³ is H, | R⁴ is CH₃ and |
| 223 | A is A-1, | Q¹ is CH═CH, | R¹ is CF₃, | R² is Et, | R³ is H, | R⁴ is CH₃ and |
| 224 | A is A-1, | Q¹ is CH═CH, | R¹ is CF₃, | R² is CF₃, | R³ is H, | R⁴ is CH₃ and |
| 225 | A is A-1, | Q¹ is CH═CH, | R¹ is CF₃, | R² is CCH, | R³ is H, | R⁴ is CH₃ and |
| 226 | A is A-1, | Q¹ is O, | R¹ is CF₃, | R² is H, | R³ is CH₃, | R⁴ is CH₃ and |
| 227 | A is A-1, | Q¹ is O, | R¹ is CF₃, | R² is H, | R³ is COCH₃, | R⁴ is CH₃ and |
| 228 | A is A-1, | Q¹ is O, | R¹ is CF₃, | R² is H, | R³ is CO₂CH₃, | R⁴ is CH₃ and |
| 229 | A is A-1, | Q¹ is O, | R¹ is CF₃, | R² is CHF₂ | R³ is COCF₃, | R⁴ is CH₃ and |
| 230 | A is A-1, | Q¹ is O, | R¹ is CF₃, | R² is F, | R³ is H, | R⁴ is CH₃ and |
| 231 | A is A-1, | Q¹ is O, | R¹ is CF₃, | R² is F, | R³ is H, | R⁴ is CH₃ and |
| 232 | A is A-1, | Q¹ is O, | R¹ is CF₃, | R² is Et, | R³ is H, | R⁴ is CH₃ and |
| 233 | A is A-1, | Q¹ is O, | R¹ is CF₃, | R² is CF₃, | R³ is H, | R⁴ is CH₃ and |
| 234 | A is A-1, | Q¹ is O, | R¹ is CF₃, | R² is CCH, | R³ is H, | R⁴ is CH₃ and |
| 235 | A is A-2, | Q² is O, | R¹ is CF₃, | R² is H, | R³ is H, | R⁴ is CH₃ and |
| 236 | A is A-2, | Q² is O, | R¹ is CF₃, | R² is H, | R³ is H, | R⁴ is Et and |
| 237 | A is A-2, | Q² is O, | R¹ is CF₃, | R² is H, | R³ is H, | R⁴ is c-Pr and |
| 238 | A is A-2, | Q² is O, | R¹ is CF₃, | R² is H, | R³ is H, | R⁴ is Pr and |
| 239 | A is A-2, | Q² is O, | R¹ is CF₃, | R² is H, | R³ is H, | R⁴ is i-Pr and |
| 240 | A is A-2, | Q² is O, | R¹ is CF₃, | R² is H, | R³ is H, | R⁴ is Bu and |
| 241 | A is A-2, | Q² is O, | R¹ is CF₃, | R² is H, | R³ is H, | R⁴ is CF₃ and |
| 242 | A is A-2, | Q² is O, | R¹ is CF₃, | R² is H, | R³ is H, | R⁴ is CHF₂ and |
| 243 | A is A-2, | Q² is O, | R¹ is CF₃, | R² is CH₃, | R³ is H, | R⁴ is CH₃ and |
| 244 | A is A-2, | Q² is O, | R¹ is CF₃, | R² is CH₃, | R³ is H, | R⁴ is Et and |
| 245 | A is A-2, | Q² is O, | R¹ is CF₃, | R² is CH₃, | R³ is H, | R⁴ is c-Pr and |
| 246 | A is A-2, | Q² is O, | R¹ is CF₃, | R² is CH₃, | R³ is H, | R⁴ is Pr and |
| 247 | A is A-2, | Q² is O, | R¹ is CF₃, | R² is CH₃, | R³ is H, | R⁴ is i-Pr and |
| 248 | A is A-2, | Q² is O, | R¹ is CF₃, | R² is CH₃, | R³ is H, | R⁴ is Bu and |
| 249 | A is A-2, | Q² is O, | R¹ is CF₃, | R² is CH₃, | R³ is H, | R⁴ is CF₃ and |
| 250 | A is A-2, | Q² is O, | R¹ is CF₃, | R² is CH₃, | R³ is H, | R⁴ is CHF₂ and |
| 251 | A is A-2, | Q² is O, | R¹ is CF₃, | R² is Cl, | R³ is H, | R⁴ is CH₃ and |
| 252 | A is A-2, | Q² is O, | R¹ is CF₃, | R² is Cl, | R³ is H, | R⁴ is Et and |
| 253 | A is A-2, | Q² is O, | R¹ is CF₃, | R² is Cl, | R³ is H, | R⁴ is c-Pr and |
| 254 | A is A-2, | Q² is O, | R¹ is CF₃, | R² is Cl, | R³ is H, | R⁴ is Pr and |
| 255 | A is A-2, | Q² is O, | R¹ is CF₃, | R² is Cl, | R³ is H, | R⁴ is i-Pr and |
| 256 | A is A-2, | Q² is O, | R¹ is CF₃, | R² is Cl, | R³ is H, | R⁴ is Bu and |
| 257 | A is A-2, | Q² is O, | R¹ is CF₃, | R² is Cl, | R³ is H, | R⁴ is CF₃ and |
| 258 | A is A-2, | Q² is O, | R¹ is CF₃, | R² is Cl, | R³ is H, | R⁴ is CHF₂ and |
| 259 | A is A-2, | Q² is O, | R¹ is CN, | R² is H, | R³ is H, | R⁴ is CH₃ and |
| 260 | A is A-2, | Q² is O, | R¹ is CN, | R² is H, | R³ is H, | R⁴ is Et and |
| 261 | A is A-2, | Q² is O, | R¹ is CN, | R² is H, | R³ is H, | R⁴ is c-Pr and |
| 262 | A is A-2, | Q² is O, | R¹ is CN, | R² is H, | R³ is H, | R⁴ is Pr and |
| 263 | A is A-2, | Q² is O, | R¹ is CN, | R² is H, | R³ is H, | R⁴ is i-Pr and |
| 264 | A is A-2, | Q² is O, | R¹ is CN, | R² is H, | R³ is H, | R⁴ is Bu and |
| 265 | A is A-2, | Q² is O, | R¹ is CN, | R² is H, | R³ is H, | R⁴ is CF₃ and |
| 266 | A is A-2, | Q² is O, | R¹ is CN, | R² is H, | R³ is H, | R⁴ is CHF₂ and |
| 267 | A is A-2, | Q² is O, | R¹ is CN, | R² is CH₃, | R³ is H, | R⁴ is CH₃ and |
| 268 | A is A-2, | Q² is O, | R¹ is CN, | R² is CH₃, | R³ is H, | R⁴ is Et and |
| 269 | A is A-2, | Q² is O, | R¹ is CN, | R² is CH₃, | R³ is H, | R⁴ is c-Pr and |
| 270 | A is A-2, | Q² is O, | R¹ is CN, | R² is CH₃, | R³ is H, | R⁴ is Pr and |
| 271 | A is A-2, | Q² is O, | R¹ is CN, | R² is CH₃, | R³ is H, | R⁴ is i-Pr and |
| 272 | A is A-2, | Q² is O, | R¹ is CN, | R² is CH₃, | R³ is H, | R⁴ is Bu and |
| 273 | A is A-2, | Q² is O, | R¹ is CN, | R² is CH₃, | R³ is H, | R⁴ is CF₃ and |
| 274 | A is A-2, | Q² is O, | R¹ is CN, | R² is CH₃, | R³ is H, | R⁴ is CHF₂ and |
| 275 | A is A-2, | Q² is O, | R¹ is CN, | R² is Cl, | R³ is H, | R⁴ is CH₃ and |
| 276 | A is A-2, | Q² is O, | R¹ is CN, | R² is Cl, | R³ is H, | R⁴ is Et and |
| 277 | A is A-2, | Q² is O, | R¹ is CN, | R² is Cl, | R³ is H, | R⁴ is c-Pr and |
| 278 | A is A-2, | Q² is O, | R¹ is CN, | R² is Cl, | R³ is H, | R⁴ is Pr and |
| 279 | A is A-2, | Q² is O, | R¹ is CN, | R² is Cl, | R³ is H, | R⁴ is i-Pr and |
| 280 | A is A-2, | Q² is O, | R¹ is CN, | R² is Cl, | R³ is H, | R⁴ is Bu and |
| 281 | A is A-2, | Q² is O, | R¹ is CN, | R² is Cl, | R³ is H, | R⁴ is CF₃ and |
| 282 | A is A-2, | Q² is O, | R¹ is CN, | R² is Cl, | R³ is H, | R⁴ is CHF₂ and |
| 283 | A is A-2, | Q² is O, | R¹ is CHF₂, | R² is H, | R³ is H, | R⁴ is CH₃ and |
| 284 | A is A-2, | Q² is O, | R¹ is CHF₂, | R² is H, | R³ is H, | R⁴ is Et and |
| 285 | A is A-2, | Q² is O, | R¹ is CHF₂, | R² is H, | R³ is H, | R⁴ is c-Pr and |
| 286 | A is A-2, | Q² is O, | R¹ is CHF₂, | R² is H, | R³ is H, | R⁴ is Pr and |
| 287 | A is A-2, | Q² is O, | R¹ is CHF₂, | R² is H, | R³ is H, | R⁴ is i-Pr and |
| 288 | A is A-2, | Q² is O, | R¹ is CHF₂, | R² is H, | R³ is H, | R⁴ is Bu and |
| 289 | A is A-2, | Q² is O, | R¹ is CHF₂, | R² is H, | R³ is H, | R⁴ is CF₃ and |
| 290 | A is A-2, | Q² is O, | R¹ is CHF₂, | R² is H, | R³ is H, | R⁴ is CHF₂ and |
| 291 | A is A-2, | Q² is O, | R¹ is CHF₂, | R² is CH₃, | R³ is H, | R⁴ is CH₃ and |
| 292 | A is A-2, | Q² is O, | R¹ is CHF₂, | R² is CH₃, | R³ is H, | R⁴ is Et and |
| 293 | A is A-2, | Q² is O, | R¹ is CHF₂, | R² is CH₃, | R³ is H, | R⁴ is c-Pr and |
| 294 | A is A-2, | Q² is O, | R¹ is CHF₂, | R² is CH₃, | R³ is H, | R⁴ is Pr and |
| 295 | A is A-2, | Q² is O, | R¹ is CHF₂, | R² is CH₃, | R³ is H, | R⁴ is i-Pr and |
| 296 | A is A-2, | Q² is O, | R¹ is CHF₂, | R² is CH₃, | R³ is H, | R⁴ is Bu and |
| 297 | A is A-2, | Q² is O, | R¹ is CHF₂, | R² is CH₃, | R³ is H, | R⁴ is CF₃ and |
| 298 | A is A-2, | Q² is O, | R¹ is CHF₂, | R² is CH₃, | R³ is H, | R⁴ is CHF₂ and |
| 299 | A is A-2, | Q² is O, | R¹ is CHF₂, | R² is Cl, | R³ is H, | R⁴ is CH₃ and |
| 300 | A is A-2, | Q² is O, | R¹ is CHF₂, | R² is Cl, | R³ is H, | R⁴ is Et and |
| 301 | A is A-2, | Q² is O, | R¹ is CHF₂, | R² is Cl, | R³ is H, | R⁴ is c-Pr and |
| 302 | A is A-2, | Q² is O, | R¹ is CHF₂, | R² is Cl, | R³ is H, | R⁴ is Pr and |
| 303 | A is A-2, | Q² is O, | R¹ is CHF₂, | R² is Cl, | R³ is H, | R⁴ is i-Pr and |
| 304 | A is A-2, | Q² is O, | R¹ is CHF₂, | R² is Cl, | R³ is H, | R⁴ is Bu and |
| 305 | A is A-2, | Q² is O, | R¹ is CHF₂, | R² is Cl, | R³ is H, | R⁴ is CF₃ and |
| 306 | A is A-2, | Q² is O, | R¹ is CHF₂, | R² is Cl, | R³ is H, | R⁴ is CHF₂ and |
| 307 | A is A-2, | Q² is CH═CH, | R¹ is CF₃, | R² is H, | R³ is H, | R⁴ is CH₃ and |
| 308 | A is A-2, | Q² is CH═CH, | R¹ is CF₃, | R² is H, | R³ is H, | R⁴ is Et and |
| 309 | A is A-2, | Q² is CH═CH, | R¹ is CF₃, | R² is H, | R³ is H, | R⁴ is c-Pr and |
| 310 | A is A-2, | Q² is CH═CH, | R¹ is CF₃, | R² is H, | R³ is H, | R⁴ is Pr and |
| 311 | A is A-2, | Q² is CH═CH, | R¹ is CF₃, | R² is H, | R³ is H, | R⁴ is i-Pr and |
| 312 | A is A-2, | Q² is CH═CH, | R¹ is CF₃, | R² is H, | R³ is H, | R⁴ is Bu and |
| 313 | A is A-2, | Q² is CH═CH, | R¹ is CF₃, | R² is H, | R³ is H, | R⁴ is CF₃ and |
| 314 | A is A-2, | Q² is CH═CH, | R¹ is CF₃, | R² is H, | R³ is H, | R⁴ is CHF₂ and |
| 315 | A is A-2, | Q² is CH═CH, | R¹ is CF₃, | R² is CH₃, | R³ is H, | R⁴ is CH₃ and |
| 316 | A is A-2, | Q² is CH═CH, | R¹ is CF₃, | R² is CH₃, | R³ is H, | R⁴ is Et and |
| 317 | A is A-2, | Q² is CH═CH, | R¹ is CF₃, | R² is CH₃, | R³ is H, | R⁴ is c-Pr and |
| 318 | A is A-2, | Q² is CH═CH, | R¹ is CF₃, | R² is CH₃, | R³ is H, | R⁴ is Pr and |
| 319 | A is A-2, | Q² is CH═CH, | R¹ is CF₃, | R² is CH₃, | R³ is H, | R⁴ is i-Pr and |
| 320 | A is A-2, | Q² is CH═CH, | R¹ is CF₃, | R² is CH₃, | R³ is H, | R⁴ is Bu and |
| 321 | A is A-2, | Q² is CH═CH, | R¹ is CF₃, | R² is CH₃, | R³ is H, | R⁴ is CF₃ and |
| 322 | A is A-2, | Q² is CH═CH, | R¹ is CF₃, | R² is CH₃, | R³ is H, | R⁴ is CHF₂ and |
| 323 | A is A-2, | Q² is CH═CH, | R¹ is CF₃, | R² is Cl, | R³ is H, | R⁴ is CH₃ and |
| 324 | A is A-2, | Q² is CH═CH, | R¹ is CF₃, | R² is Cl, | R³ is H, | R⁴ is Et and |
| 325 | A is A-2, | Q² is CH═CH, | R¹ is CF₃, | R² is Cl, | R³ is H, | R⁴ is c-Pr and |
| 326 | A is A-2, | Q² is CH═CH, | R¹ is CF₃, | R² is Cl, | R³ is H, | R⁴ is Pr and |
| 327 | A is A-2, | Q² is CH═CH, | R¹ is CF₃, | R² is Cl, | R³ is H, | R⁴ is i-Pr and |
| 328 | A is A-2, | Q² is CH═CH, | R¹ is CF₃, | R² is Cl, | R³ is H, | R⁴ is Bu and |
| 329 | A is A-2, | Q² is CH═CH, | R¹ is CF₃, | R² is Cl, | R³ is H, | R⁴ is CF₃ and |
| 330 | A is A-2, | Q² is CH═CH, | R¹ is CF₃, | R² is Cl, | R³ is H, | R⁴ is CHF₂ and |
| 331 | A is A-2, | Q² is S, | R¹ is CF₃, | R² is H, | R³ is H, | R⁴ is CH₃ and |
| 332 | A is A-2, | Q² is S, | R¹ is CF₃, | R² is H, | R³ is H, | R⁴ is Et and |
| 333 | A is A-2, | Q² is S, | R¹ is CF₃, | R² is H, | R³ is H, | R⁴ is c-Pr and |
| 334 | A is A-2, | Q² is S, | R¹ is CF₃, | R² is H, | R³ is H, | R⁴ is Pr and |
| 335 | A is A-2, | Q² is S, | R¹ is CF₃, | R² is H, | R³ is H, | R⁴ is i-Pr and |
| 336 | A is A-2, | Q² is S, | R¹ is CF₃, | R² is H, | R³ is H, | R⁴ is Bu and |
| 337 | A is A-2, | Q² is S, | R¹ is CF₃, | R² is H, | R³ is H, | R⁴ is CF₃ and |
| 338 | A is A-2, | Q² is S, | R¹ is CF₃, | R² is H, | R³ is H, | R⁴ is CHF₂ and |
| 339 | A is A-2, | Q² is S, | R¹ is CF₃, | R² is CH₃, | R³ is H, | R⁴ is CH₃ and |
| 340 | A is A-2, | Q² is S, | R¹ is CF₃, | R² is CH₃, | R³ is H, | R⁴ is Et and |
| 341 | A is A-2, | Q² is S, | R¹ is CF₃, | R² is CH₃, | R³ is H, | R⁴ is c-Pr and |
| 342 | A is A-2, | Q² is S, | R¹ is CF₃, | R² is CH₃, | R³ is H, | R⁴ is Pr and |
| 343 | A is A-2, | Q² is S, | R¹ is CF₃, | R² is CH₃, | R³ is H, | R⁴ is i-Pr and |
| 344 | A is A-2, | Q² is S, | R¹ is CF₃, | R² is CH₃, | R³ is H, | R⁴ is Bu and |
| 345 | A is A-2, | Q² is S, | R¹ is CF₃, | R² is CH₃, | R³ is H, | R⁴ is CF₃ and |
| 346 | A is A-2, | Q² is S, | R¹ is CF₃, | R² is CH₃, | R³ is H, | R⁴ is CHF₂ and |
| 347 | A is A-2, | Q² is S, | R¹ is CF₃, | R² is Cl, | R³ is H, | R⁴ is CH₃ and |
| 348 | A is A-2, | Q² is S, | R¹ is CF₃, | R² is Cl, | R³ is H, | R⁴ is Et and |
| 349 | A is A-2, | Q² is S, | R¹ is CF₃, | R² is Cl, | R³ is H, | R⁴ is c-Pr and |
| 350 | A is A-2, | Q² is S, | R¹ is CF₃, | R² is Cl, | R³ is H, | R⁴ is Pr and |
| 351 | A is A-2, | Q² is S, | R¹ is CF₃, | R² is Cl, | R³ is H, | R⁴ is i-Pr and |
| 352 | A is A-2, | Q² is S, | R¹ is CF₃, | R² is Cl, | R³ is H, | R⁴ is Bu and |
| 353 | A is A-2, | Q² is S, | R¹ is CF₃, | R² is Cl, | R³ is H, | R⁴ is CF₃ and |
| 354 | A is A-2, | Q² is S, | R¹ is CF₃, | R² is Cl, | R³ is H, | R⁴ is CHF₂ and |
| 355 | A is A-2, | Q² is SO₂, | R¹ is CF₃, | R² is H, | R³ is H, | R⁴ is CH₃ and |
| 356 | A is A-2, | Q² is SO₂, | R¹ is CF₃, | R² is H, | R³ is H, | R⁴ is Et and |
| 357 | A is A-2, | Q² is SO₂, | R¹ is CF₃, | R² is H, | R³ is H, | R⁴ is c-Pr and |
| 358 | A is A-2, | Q² is SO₂, | R¹ is CF₃, | R² is H, | R³ is H, | R⁴ is Pr and |
| 359 | A is A-2, | Q² is SO₂, | R¹ is CF₃, | R² is H, | R³ is H, | R⁴ is i-Pr and |
| 360 | A is A-2, | Q² is SO₂, | R¹ is CF₃, | R² is H, | R³ is H, | R⁴ is Bu and |
| 361 | A is A-2, | Q² is SO₂, | R¹ is CF₃, | R² is H, | R³ is H, | R⁴ is CF₃ and |
| 362 | A is A-2, | Q² is SO₂, | R¹ is CF₃, | R² is H, | R³ is H, | R⁴ is CHF₂ and |
| 363 | A is A-2, | Q² is SO₂, | R¹ is CF₃, | R² is CH₃, | R³ is H, | R⁴ is CH₃ and |
| 364 | A is A-2, | Q² is SO₂, | R¹ is CF₃, | R² is CH₃, | R³ is H, | R⁴ is Et and |
| 365 | A is A-2, | Q² is SO₂, | R¹ is CF₃, | R² is CH₃, | R³ is H, | R⁴ is c-Pr and |
| 366 | A is A-2, | Q² is SO₂, | R¹ is CF₃, | R² is CH₃, | R³ is H, | R⁴ is Pr and |
| 367 | A is A-2, | Q² is SO₂, | R¹ is CF₃, | R² is CH₃, | R³ is H, | R⁴ is i-Pr and |
| 368 | A is A-2, | Q² is SO₂, | R¹ is CF₃, | R² is CH₃, | R³ is H, | R⁴ is Bu and |
| 369 | A is A-2, | Q² is SO₂, | R¹ is CF₃, | R² is CH₃, | R³ is H, | R⁴ is CF₃ and |
| 370 | A is A-2, | Q² is SO₂, | R¹ is CF₃, | R² is CH₃, | R³ is H, | R⁴ is CHF₂ and |
| 371 | A is A-2, | Q² is SO₂, | R¹ is CF₃, | R² is Cl, | R³ is H, | R⁴ is CH₃ and |
| 372 | A is A-2, | Q² is SO₂, | R¹ is CF₃, | R² is Cl, | R³ is H, | R⁴ is Et and |
| 373 | A is A-2, | Q² is SO₂, | R¹ is CF₃, | R² is Cl, | R³ is H, | R⁴ is c-Pr and |
| 374 | A is A-2, | Q² is SO₂, | R¹ is CF₃, | R² is Cl, | R³ is H, | R⁴ is Pr and |
| 375 | A is A-2, | Q² is SO₂, | R¹ is CF₃, | R² is Cl, | R³ is H, | R⁴ is i-Pr and |
| 376 | A is A-2, | Q² is SO₂, | R¹ is CF₃, | R² is Cl, | R³ is H, | R⁴ is Bu and |
| 377 | A is A-2, | Q² is SO₂, | R¹ is CF₃, | R² is Cl, | R³ is H, | R⁴ is CF₃ and |
| 378 | A is A-2, | Q² is SO₂, | R¹ is CF₃, | R² is Cl, | R³ is H, | R⁴ is CHF₂ and |
| 379 | A is A-2, | Q² is NCH₃, | R¹ is CF₃, | R² is H, | R³ is H, | R⁴ is CH₃ and |
| 380 | A is A-2, | Q² is NCH₃, | R¹ is CF₃, | R² is H, | R³ is H, | R⁴ is Et and |
| 381 | A is A-2, | Q² is NCH₃, | R¹ is CF₃, | R² is H, | R³ is H, | R⁴ is c-Pr and |
| 382 | A is A-2, | Q² is NCH₃, | R¹ is CF₃, | R² is H, | R³ is H, | R⁴ is Pr and |
| 383 | A is A-2, | Q² is NCH₃, | R¹ is CF₃, | R² is H, | R³ is H, | R⁴ is i-Pr and |
| 384 | A is A-2, | Q² is NCH₃, | R¹ is CF₃, | R² is H, | R³ is H, | R⁴ is Bu and |
| 385 | A is A-2, | Q² is NCH₃, | R¹ is CF₃, | R² is H, | R³ is H, | R⁴ is CF₃ and |
| 386 | A is A-2, | Q² is NCH₃, | R¹ is CF₃, | R² is H, | R³ is H, | R⁴ is CHF₂ and |
| 387 | A is A-2, | Q² is NCH₃, | R¹ is CF₃, | R² is CH₃, | R³ is H, | R⁴ is CH₃ and |
| 388 | A is A-2, | Q² is NCH₃, | R¹ is CF₃, | R² is CH₃, | R³ is H, | R⁴ is Et and |
| 389 | A is A-2, | Q² is NCH₃, | R¹ is CF₃, | R² is CH₃, | R³ is H, | R⁴ is c-Pr and |
| 390 | A is A-2, | Q² is NCH₃, | R¹ is CF₃, | R² is CH₃, | R³ is H, | R⁴ is Pr and |
| 391 | A is A-2, | Q² is NCH₃, | R¹ is CF₃, | R² is CH₃, | R³ is H, | R⁴ is i-Pr and |
| 392 | A is A-2, | Q² is NCH₃, | R¹ is CF₃, | R² is CH₃, | R³ is H, | R⁴ is Bu and |
| 393 | A is A-2, | Q² is NCH₃, | R¹ is CF₃, | R² is CH₃, | R³ is H, | R⁴ is CF₃ and |
| 394 | A is A-2, | Q² is NCH₃, | R¹ is CF₃, | R² is CH₃, | R³ is H, | R⁴ is CHF₂ and |
| 395 | A is A-2, | Q² is NCH₃, | R¹ is CF₃, | R² is Cl, | R³ is H, | R⁴ is CH₃ and |
| 396 | A is A-2, | Q² is NCH₃, | R¹ is CF₃, | R² is Cl, | R³ is H, | R⁴ is Et and |
| 397 | A is A-2, | Q² is NCH₃, | R¹ is CF₃, | R² is Cl, | R³ is H, | R⁴ is c-Pr and |
| 398 | A is A-2, | Q² is NCH₃, | R¹ is CF₃, | R² is Cl, | R³ is H, | R⁴ is Pr and |
| 399 | A is A-2, | Q² is NCH₃, | R¹ is CF₃, | R² is Cl, | R³ is H, | R⁴ is i-Pr and |
| 400 | A is A-2, | Q² is NCH₃, | R¹ is CF₃, | R² is Cl, | R³ is H, | R⁴ is Bu and |
| 401 | A is A-2, | Q² is NCH₃, | R¹ is CF₃, | R² is Cl, | R³ is H, | R⁴ is CF₃ and |
| 402 | A is A-2, | Q² is NCH₃, | R¹ is CF₃, | R² is Cl, | R³ is H, | R⁴ is CHF₂ and |
| 403 | A is A-2, | Q² is NCH₃, | R¹ is CF₃, | R² is H, | R³ is H, | R⁴ is CH₃ and |
| 404 | A is A-2, | Q² is NCH₃, | R¹ is CF₃, | R² is H, | R³ is H, | R⁴ is Et and |
| 405 | A is A-2, | Q² is NCH₃, | R¹ is CF₃, | R² is H, | R³ is H, | R⁴ is c-Pr and |
| 406 | A is A-2, | Q² is NCH₃, | R¹ is CF₃, | R² is H, | R³ is H, | R⁴ is Pr and |
| 407 | A is A-2, | Q² is NCH₃, | R¹ is CF₃, | R² is H, | R³ is H, | R⁴ is i-Pr and |
| 408 | A is A-2, | Q² is NCH₃, | R¹ is CF₃, | R² is H, | R³ is H, | R⁴ is Bu and |
| 409 | A is A-2, | Q² is NCH₃, | R¹ is CF₃, | R² is H, | R³ is H, | R⁴ is CF₃ and |
| 410 | A is A-2, | Q² is NCH₃, | R¹ is CF₃, | R² is H, | R³ is H, | R⁴ is CHF₂ and |
| 411 | A is A-2, | Q² is NCH₃, | R¹ is CF₃, | R² is CH₃, | R³ is H, | R⁴ is CH₃ and |
| 412 | A is A-2, | Q² is NCH₃, | R¹ is CF₃, | R² is CH₃, | R³ is H, | R⁴ is Et and |
| 413 | A is A-2, | Q² is NCH₃, | R¹ is CF₃, | R² is CH₃, | R³ is H, | R⁴ is c-Pr and |
| 414 | A is A-2, | Q² is NCH₃, | R¹ is CF₃, | R² is CH₃, | R³ is H, | R⁴ is Pr and |
| 415 | A is A-2, | Q² is NCH₃, | R¹ is CF₃, | R² is CH₃, | R³ is H, | R⁴ is i-Pr and |
| 416 | A is A-2, | Q² is NCH₃, | R¹ is CF₃, | R² is CH₃, | R³ is H, | R⁴ is Bu and |
| 417 | A is A-2, | Q² is NCH₃, | R¹ is CF₃, | R² is CH₃, | R³ is H, | R⁴ is CF₃ and |
| 418 | A is A-2, | Q² is NCH₃, | R¹ is CF₃, | R² is CH₃, | R³ is H, | R⁴ is CHF₂ and |
| 419 | A is A-2, | Q² is NCH₃, | R¹ is CF₃, | R² is Cl, | R³ is H, | R⁴ is CH₃ and |
| 420 | A is A-2, | Q² is NCH₃, | R¹ is CF₃, | R² is Cl, | R³ is H, | R⁴ is Et and |
| 421 | A is A-2, | Q² is NCH₃, | R¹ is CF₃, | R² is Cl, | R³ is H, | R⁴ is c-Pr and |
| 422 | A is A-2, | Q² is NCH₃, | R¹ is CF₃, | R² is Cl, | R³ is H, | R⁴ is Pr and |
| 423 | A is A-2, | Q² is NCH₃, | R¹ is CF₃, | R² is Cl, | R³ is H, | R⁴ is i-Pr and |
| 424 | A is A-2, | Q² is NCH₃, | R¹ is CF₃, | R² is Cl, | R³ is H, | R⁴ is Bu and |
| 425 | A is A-2, | Q² is NCH₃, | R¹ is CF₃, | R² is Cl, | R³ is H, | R⁴ is CF₃ and |
| 426 | A is A-2, | Q² is NCH₃, | R¹ is CF₃, | R² is Cl, | R³ is H, | R⁴ is CHF₂ and |
| 427 | A is A-2, | Q² is CO, | R¹ is CF₃, | R² is H, | R³ is H, | R⁴ is CH₃ and |
| 428 | A is A-2, | Q² is CO, | R¹ is CF₃, | R² is H, | R³ is H, | R⁴ is Et and |
| 429 | A is A-2, | Q² is CO, | R¹ is CF₃, | R² is H, | R³ is H, | R⁴ is c-Pr and |
| 430 | A is A-2, | Q² is CO, | R¹ is CF₃, | R² is H, | R³ is H, | R⁴ is Pr and |
| 431 | A is A-2, | Q² is CO, | R¹ is CF₃, | R² is H, | R³ is H, | R⁴ is i-Pr and |
| 432 | A is A-2, | Q² is CO, | R¹ is CF₃, | R² is H, | R³ is H, | R⁴ is Bu and |
| 433 | A is A-2, | Q² is CO, | R¹ is CF₃, | R² is H, | R³ is H, | R⁴ is CF₃ and |
| 434 | A is A-2, | Q² is CO, | R¹ is CF₃, | R² is H, | R³ is H, | R⁴ is CHF₂ and |
| 435 | A is A-2, | Q² is CO, | R¹ is CF₃, | R² is CH₃, | R³ is H, | R⁴ is CH₃ and |
| 436 | A is A-2, | Q² is CO, | R¹ is CF₃, | R² is CH₃, | R³ is H, | R⁴ is Et and |
| 437 | A is A-2, | Q² is CO, | R¹ is CF₃, | R² is CH₃, | R³ is H, | R⁴ is c-Pr and |
| 438 | A is A-2, | Q² is CO, | R¹ is CF₃, | R² is CH₃, | R³ is H, | R⁴ is Pr and |
| 439 | A is A-2, | Q² is CO, | R¹ is CF₃, | R² is CH₃, | R³ is H, | R⁴ is i-Pr and |
| 440 | A is A-2, | Q² is CO, | R¹ is CF₃, | R² is CH₃, | R³ is H, | R⁴ is Bu and |
| 441 | A is A-2, | Q² is CO, | R¹ is CF₃, | R² is CH₃, | R³ is H, | R⁴ is CF₃ and |
| 442 | A is A-2, | Q² is CO, | R¹ is CF₃, | R² is CH₃, | R³ is H, | R⁴ is CHF₂ and |
| 443 | A is A-2, | Q² is CO, | R¹ is CF₃, | R² is Cl, | R³ is H, | R⁴ is CH₃ and |
| 444 | A is A-2, | Q² is CO, | R¹ is CF₃, | R² is Cl, | R³ is H, | R⁴ is Et and |
| 445 | A is A-2, | Q² is CO, | R¹ is CF₃, | R² is Cl, | R³ is H, | R⁴ is c-Pr and |
| 446 | A is A-2, | Q² is CO, | R¹ is CF₃, | R² is Cl, | R³ is H, | R⁴ is Pr and |
| 447 | A is A-2, | Q² is CO, | R¹ is CF₃, | R² is Cl, | R³ is H, | R⁴ is i-Pr and |
| 448 | A is A-2, | Q² is CO, | R¹ is CF₃, | R² is Cl, | R³ is H, | R⁴ is Bu and |
| 449 | A is A-2, | Q² is CO, | R¹ is CF₃, | R² is Cl, | R³ is H, | R⁴ is CF₃ and |
| 450 | A is A-2, | Q² is CO, | R¹ is CF₃, | R² is Cl, | R³ is H, | R⁴ is CHF₂ and |
| 451 | A is A-2, | Q² is CH═CH, | R¹ is CF₃, | R² is H, | R³ is H, | R⁴ is CH₃ and |
| 452 | A is A-2, | Q² is CH═CH, | R¹ is CF₃, | R² is H, | R³ is H, | R⁴ is CH₃ and |
| 453 | A is A-2, | Q² is CH═CH, | R¹ is CF₃, | R² is H, | R³ is H, | R⁴ is CH₃ and |
| 454 | A is A-2, | Q² is CH═CH, | R¹ is CF₃, | R² is H, | R³ is H, | R⁴ is CH₃ and |
| 455 | A is A-2, | Q² is CH═CH, | R¹ is CF₃, | R² is CHF₂, | R³ is H, | R⁴ is CH₃ and |
| 456 | A is A-2, | Q² is CH═CH, | R¹ is CF₃, | R² is F, | R³ is H, | R⁴ is CH₃ and |
| 457 | A is A-2, | Q² is CH═CH, | R¹ is CF₃, | R² is Et, | R³ is H, | R⁴ is CH₃ and |
| 458 | A is A-2, | Q² is CH═CH, | R¹ is CF₃, | R² is CF₃, | R³ is H, | R⁴ is CH₃ and |
| 459 | A is A-2, | Q² is CH═CH, | R¹ is CF₃, | R² is CCH, | R³ is H, | R⁴ is CH₃ and |
| 460 | A is A-2, | Q² is O, | R¹ is CF₃, | R² is H, | R³ is CH₃, | R⁴ is CH₃ and |
| 461 | A is A-2, | Q² is O, | R¹ is CF₃, | R² is H, | R³ is COCH₃, | R⁴ is CH₃ and |
| 462 | A is A-2, | Q² is O, | R¹ is CF₃, | R² is H, | R³ is CO₂CH₃, | R⁴ is CH₃ and |
| 463 | A is A-2, | Q² is O, | R¹ is CF₃, | R² is CHF₂ | R³ is COCF₃, | R⁴ is CH₃ and |
| 464 | A is A-2, | Q² is O, | R¹ is CF₃, | R² is F, | R³ is H, | R⁴ is CH₃ and |
| 465 | A is A-2, | Q² is O, | R¹ is CF₃, | R² is F, | R³ is H, | R⁴ is CH₃ and |
| 466 | A is A-2, | Q² is O, | R¹ is CF₃, | R² is Et, | R³ is H, | R⁴ is CH₃ and |
| 467 | A is A-2, | Q² is O, | R¹ is CF₃, | R² is CF₃, | R³ is H, | R⁴ is CH₃ and |
| 468 | A is A-2, | Q² is O, | R¹ is CF₃, | R² is CCH, | R³ is H, | R⁴ is CH₃ and |
| 469 | A is A-3, | Q³ is O, | R¹ is CF₃, | R² is H, | R³ is H, | R⁴ is CH₃ and |
| 470 | A is A-3, | Q³ is O, | R¹ is CF₃, | R² is H, | R³ is H, | R⁴ is Et and |
| 471 | A is A-3, | Q³ is O, | R¹ is CF₃, | R² is H, | R³ is H, | R⁴ is c-Pr and |
| 472 | A is A-3, | Q³ is O, | R¹ is CF₃, | R² is H, | R³ is H, | R⁴ is Pr and |
| 473 | A is A-3, | Q³ is O, | R¹ is CF₃, | R² is H, | R³ is H, | R⁴ is i-Pr and |
| 474 | A is A-3, | Q³ is O, | R¹ is CF₃, | R² is H, | R³ is H, | R⁴ is Bu and |
| 475 | A is A-3, | Q³ is O, | R¹ is CF₃, | R² is H, | R³ is H, | R⁴ is CF₃ and |
| 476 | A is A-3, | Q³ is O, | R¹ is CF₃, | R² is H, | R³ is H, | R⁴ is CHF₂ and |
| 477 | A is A-3, | Q³ is O, | R¹ is CF₃, | R² is CH₃, | R³ is H, | R⁴ is CH₃ and |
| 478 | A is A-3, | Q³ is O, | R¹ is CF₃, | R² is CH₃, | R³ is H, | R⁴ is Et and |
| 479 | A is A-3, | Q³ is O, | R¹ is CF₃, | R² is CH₃, | R³ is H, | R⁴ is c-Pr and |
| 480 | A is A-3, | Q³ is O, | R¹ is CF₃, | R² is CH₃, | R³ is H, | R⁴ is Pr and |
| 481 | A is A-3, | Q³ is O, | R¹ is CF₃, | R² is CH₃, | R³ is H, | R⁴ is i-Pr and |
| 482 | A is A-3, | Q³ is O, | R¹ is CF₃, | R² is CH₃, | R³ is H, | R⁴ is Bu and |
| 483 | A is A-3, | Q³ is O, | R¹ is CF₃, | R² is CH₃, | R³ is H, | R⁴ is CF₃ and |
| 484 | A is A-3, | Q³ is O, | R¹ is CF₃, | R² is CH₃, | R³ is H, | R⁴ is CHF₂ and |
| 485 | A is A-3, | Q³ is O, | R¹ is CF₃, | R² is Cl, | R³ is H, | R⁴ is CH₃ and |
| 486 | A is A-3, | Q³ is O, | R¹ is CF₃, | R² is Cl, | R³ is H, | R⁴ is Et and |
| 487 | A is A-3, | Q³ is O, | R¹ is CF₃, | R² is Cl, | R³ is H, | R⁴ is c-Pr and |
| 488 | A is A-3, | Q³ is O, | R¹ is CF₃, | R² is Cl, | R³ is H, | R⁴ is Pr and |
| 489 | A is A-3, | Q³ is O, | R¹ is CF₃, | R² is Cl, | R³ is H, | R⁴ is i-Pr and |
| 490 | A is A-3, | Q³ is O, | R¹ is CF₃, | R² is Cl, | R³ is H, | R⁴ is Bu and |
| 491 | A is A-3, | Q³ is O, | R¹ is CF₃, | R² is Cl, | R³ is H, | R⁴ is CF₃ and |
| 492 | A is A-3, | Q³ is O, | R¹ is CF₃, | R² is Cl, | R³ is H, | R⁴ is CHF₂ and |
| 493 | A is A-3, | Q³ is O, | R¹ is CN, | R² is H, | R³ is H, | R⁴ is CH₃ and |
| 494 | A is A-3, | Q³ is O, | R¹ is CN, | R² is H, | R³ is H, | R⁴ is Et and |
| 495 | A is A-3, | Q³ is O, | R¹ is CN, | R² is H, | R³ is H, | R⁴ is c-Pr and |
| 496 | A is A-3, | Q³ is O, | R¹ is CN, | R² is H, | R³ is H, | R⁴ is Pr and |
| 497 | A is A-3, | Q³ is O, | R¹ is CN, | R² is H, | R³ is H, | R⁴ is i-Pr and |
| 498 | A is A-3, | Q³ is O, | R¹ is CN, | R² is H, | R³ is H, | R⁴ is Bu and |
| 499 | A is A-3, | Q³ is O, | R¹ is CN, | R² is H, | R³ is H, | R⁴ is CF₃ and |
| 500 | A is A-3, | Q³ is O, | R¹ is CN, | R² is H, | R³ is H, | R⁴ is CHF₂ and |
| 501 | A is A-3, | Q³ is O, | R¹ is CN, | R² is CH₃, | R³ is H, | R⁴ is CH₃ and |
| 502 | A is A-3, | Q³ is O, | R¹ is CN, | R² is CH₃, | R³ is H, | R⁴ is Et and |
| 503 | A is A-3, | Q³ is O, | R¹ is CN, | R² is CH₃, | R³ is H, | R⁴ is c-Pr and |
| 504 | A is A-3, | Q³ is O, | R¹ is CN, | R² is CH₃, | R³ is H, | R⁴ is Pr and |
| 505 | A is A-3, | Q³ is O, | R¹ is CN, | R² is CH₃, | R³ is H, | R⁴ is i-Pr and |
| 506 | A is A-3, | Q³ is O, | R¹ is CN, | R² is CH₃, | R³ is H, | R⁴ is Bu and |
| 507 | A is A-3, | Q³ is O, | R¹ is CN, | R² is CH₃, | R³ is H, | R⁴ is CF₃ and |
| 508 | A is A-3, | Q³ is O, | R¹ is CN, | R² is CH₃, | R³ is H, | R⁴ is CHF₂ and |
| 509 | A is A-3, | Q³ is O, | R¹ is CN, | R² is Cl, | R³ is H, | R⁴ is CH₃ and |
| 510 | A is A-3, | Q³ is O, | R¹ is CN, | R² is Cl, | R³ is H, | R⁴ is Et and |
| 511 | A is A-3, | Q³ is O, | R¹ is CN, | R² is Cl, | R³ is H, | R⁴ is c-Pr and |
| 512 | A is A-3, | Q³ is O, | R¹ is CN, | R² is Cl, | R³ is H, | R⁴ is Pr and |
| 513 | A is A-3, | Q³ is O, | R¹ is CN, | R² is Cl, | R³ is H, | R⁴ is i-Pr and |
| 514 | A is A-3, | Q³ is O, | R¹ is CN, | R² is Cl, | R³ is H, | R⁴ is Bu and |
| 515 | A is A-3, | Q³ is O, | R¹ is CN, | R² is Cl, | R³ is H, | R⁴ is CF₃ and |
| 516 | A is A-3, | Q³ is O, | R¹ is CN, | R² is Cl, | R³ is H, | R⁴ is CHF₂ and |
| 517 | A is A-3, | Q³ is O, | R¹ is CHF₂, | R² is H, | R³ is H, | R⁴ is CH₃ and |
| 518 | A is A-3, | Q³ is O, | R¹ is CHF₂, | R² is H, | R³ is H, | R⁴ is Et and |
| 519 | A is A-3, | Q³ is O, | R¹ is CHF₂, | R² is H, | R³ is H, | R⁴ is c-Pr and |
| 520 | A is A-3, | Q³ is O, | R¹ is CHF₂, | R² is H, | R³ is H, | R⁴ is Pr and |
| 521 | A is A-3, | Q³ is O, | R¹ is CHF₂, | R² is H, | R³ is H, | R⁴ is i-Pr and |
| 522 | A is A-3, | Q³ is O, | R¹ is CHF₂, | R² is H, | R³ is H, | R⁴ is Bu and |
| 523 | A is A-3, | Q³ is O, | R¹ is CHF₂, | R² is H, | R³ is H, | R⁴ is CF₃ and |
| 524 | A is A-3, | Q³ is O, | R¹ is CHF₂, | R² is H, | R³ is H, | R⁴ is CHF₂ and |
| 525 | A is A-3, | Q³ is O, | R¹ is CHF₂, | R² is CH₃, | R³ is H, | R⁴ is CH₃ and |
| 526 | A is A-3, | Q³ is O, | R¹ is CHF₂, | R² is CH₃, | R³ is H, | R⁴ is Et and |
| 527 | A is A-3, | Q³ is O, | R¹ is CHF₂, | R² is CH₃, | R³ is H, | R⁴ is c-Pr and |
| 528 | A is A-3, | Q³ is O, | R¹ is CHF₂, | R² is CH₃, | R³ is H, | R⁴ is Pr and |
| 529 | A is A-3, | Q³ is O, | R¹ is CHF₂, | R² is CH₃, | R³ is H, | R⁴ is i-Pr and |
| 530 | A is A-3, | Q³ is O, | R¹ is CHF₂, | R² is CH₃, | R³ is H, | R⁴ is Bu and |
| 531 | A is A-3, | Q³ is O, | R¹ is CHF₂, | R² is CH₃, | R³ is H, | R⁴ is CF₃ and |
| 532 | A is A-3, | Q³ is O, | R¹ is CHF₂, | R² is CH₃, | R³ is H, | R⁴ is CHF₂ and |
| 533 | A is A-3, | Q³ is O, | R¹ is CHF₂, | R² is Cl, | R³ is H, | R⁴ is CH₃ and |
| 534 | A is A-3, | Q³ is O, | R¹ is CHF₂, | R² is Cl, | R³ is H, | R⁴ is Et and |
| 535 | A is A-3, | Q³ is O, | R¹ is CHF₂, | R² is Cl, | R³ is H, | R⁴ is c-Pr and |
| 536 | A is A-3, | Q³ is O, | R¹ is CHF₂, | R² is Cl, | R³ is H, | R⁴ is Pr and |
| 537 | A is A-3, | Q³ is O, | R¹ is CHF₂, | R² is Cl, | R³ is H, | R⁴ is i-Pr and |
| 538 | A is A-3, | Q³ is O, | R¹ is CHF₂, | R² is Cl, | R³ is H, | R⁴ is Bu and |
| 539 | A is A-3, | Q³ is O, | R¹ is CHF₂, | R² is Cl, | R³ is H, | R⁴ is CF₃ and |
| 540 | A is A-3, | Q³ is O, | R¹ is CHF₂, | R² is Cl, | R³ is H, | R⁴ is CHF₂ and |
| 541 | A is A-3, | Q³ is CH═CH, | R¹ is CF₃, | R² is H, | R³ is H, | R⁴ is CH₃ and |
| 542 | A is A-3, | Q³ is CH═CH, | R¹ is CF₃, | R² is H, | R³ is H, | R⁴ is Et and |
| 543 | A is A-3, | Q³ is CH═CH, | R¹ is CF₃, | R² is H, | R³ is H, | R⁴ is c-Pr and |
| 544 | A is A-3, | Q³ is CH═CH, | R¹ is CF₃, | R² is H, | R³ is H, | R⁴ is Pr and |
| 545 | A is A-3, | Q³ is CH═CH, | R¹ is CF₃, | R² is H, | R³ is H, | R⁴ is i-Pr and |
| 546 | A is A-3, | Q³ is CH═CH, | R¹ is CF₃, | R² is H, | R³ is H, | R⁴ is Bu and |
| 547 | A is A-3, | Q³ is CH═CH, | R¹ is CF₃, | R² is H, | R³ is H, | R⁴ is CF₃ and |
| 548 | A is A-3, | Q³ is CH═CH, | R¹ is CF₃, | R² is H, | R³ is H, | R⁴ is CHF₂ and |
| 549 | A is A-3, | Q³ is CH═CH, | R¹ is CF₃, | R² is CH₃, | R³ is H, | R⁴ is CH₃ and |
| 550 | A is A-3, | Q³ is CH═CH, | R¹ is CF₃, | R² is CH₃, | R³ is H, | R⁴ is Et and |
| 551 | A is A-3, | Q³ is CH═CH, | R¹ is CF₃, | R² is CH₃, | R³ is H, | R⁴ is c-Pr and |
| 552 | A is A-3, | Q³ is CH═CH, | R¹ is CF₃, | R² is CH₃, | R³ is H, | R⁴ is Pr and |
| 553 | A is A-3, | Q³ is CH═CH, | R¹ is CF₃, | R² is CH₃, | R³ is H, | R⁴ is i-Pr and |
| 554 | A is A-3, | Q³ is CH═CH, | R¹ is CF₃, | R² is CH₃, | R³ is H, | R⁴ is Bu and |
| 555 | A is A-3, | Q³ is CH═CH, | R¹ is CF₃, | R² is CH₃, | R³ is H, | R⁴ is CF₃ and |
| 556 | A is A-3, | Q³ is CH═CH, | R¹ is CF₃, | R² is CH₃, | R³ is H, | R⁴ is CHF₂ and |
| 557 | A is A-3, | Q³ is CH═CH, | R¹ is CF₃, | R² is Cl, | R³ is H, | R⁴ is CH₃ and |
| 558 | A is A-3, | Q³ is CH═CH, | R¹ is CF₃, | R² is Cl, | R³ is H, | R⁴ is Et and |
| 559 | A is A-3, | Q³ is CH═CH, | R¹ is CF₃, | R² is Cl, | R³ is H, | R⁴ is c-Pr and |
| 560 | A is A-3, | Q³ is CH═CH, | R¹ is CF₃, | R² is Cl, | R³ is H, | R⁴ is Pr and |
| 561 | A is A-3, | Q³ is CH═CH, | R¹ is CF₃, | R² is Cl, | R³ is H, | R⁴ is i-Pr and |
| 562 | A is A-3, | Q³ is CH═CH, | R¹ is CF₃, | R² is Cl, | R³ is H, | R⁴ is Bu and |
| 563 | A is A-3, | Q³ is CH═CH, | R¹ is CF₃, | R² is Cl, | R³ is H, | R⁴ is CF₃ and |
| 564 | A is A-3, | Q³ is CH═CH, | R¹ is CF₃, | R² is Cl, | R³ is H, | R⁴ is CHF₂ and |
| 565 | A is A-3, | Q³ is S, | R¹ is CF₃, | R² is H, | R³ is H, | R⁴ is CH₃ and |
| 566 | A is A-3, | Q³ is S, | R¹ is CF₃, | R² is H, | R³ is H, | R⁴ is Et and |
| 567 | A is A-3, | Q³ is S, | R¹ is CF₃, | R² is H, | R³ is H, | R⁴ is c-Pr and |
| 568 | A is A-3, | Q³ is S, | R¹ is CF₃, | R² is H, | R³ is H, | R⁴ is Pr and |
| 569 | A is A-3, | Q³ is S, | R¹ is CF₃, | R² is H, | R³ is H, | R⁴ is i-Pr and |
| 570 | A is A-3, | Q³ is S, | R¹ is CF₃, | R² is H, | R³ is H, | R⁴ is Bu and |
| 571 | A is A-3, | Q³ is S, | R¹ is CF₃, | R² is H, | R³ is H, | R⁴ is CF₃ and |
| 572 | A is A-3, | Q³ is S, | R¹ is CF₃, | R² is H, | R³ is H, | R⁴ is CHF₂ and |
| 573 | A is A-3, | Q³ is S, | R¹ is CF₃, | R² is CH₃, | R³ is H, | R⁴ is CH₃ and |
| 574 | A is A-3, | Q³ is S, | R¹ is CF₃, | R² is CH₃, | R³ is H, | R⁴ is Et and |
| 575 | A is A-3, | Q³ is S, | R¹ is CF₃, | R² is CH₃, | R³ is H, | R⁴ is c-Pr and |
| 576 | A is A-3, | Q³ is S, | R¹ is CF₃, | R² is CH₃, | R³ is H, | R⁴ is Pr and |
| 577 | A is A-3, | Q³ is S, | R¹ is CF₃, | R² is CH₃, | R³ is H, | R⁴ is i-Pr and |
| 578 | A is A-3, | Q³ is S, | R¹ is CF₃, | R² is CH₃, | R³ is H, | R⁴ is Bu and |
| 579 | A is A-3, | Q³ is S, | R¹ is CF₃, | R² is CH₃, | R³ is H, | R⁴ is CF₃ and |
| 580 | A is A-3, | Q³ is S, | R¹ is CF₃, | R² is CH₃, | R³ is H, | R⁴ is CHF₂ and |
| 581 | A is A-3, | Q³ is S, | R¹ is CF₃, | R² is Cl, | R³ is H, | R⁴ is CH₃ and |
| 582 | A is A-3, | Q³ is S, | R¹ is CF₃, | R² is Cl, | R³ is H, | R⁴ is Et and |
| 583 | A is A-3, | Q³ is S, | R¹ is CF₃, | R² is Cl, | R³ is H, | R⁴ is c-Pr and |
| 584 | A is A-3, | Q³ is S, | R¹ is CF₃, | R² is Cl, | R³ is H, | R⁴ is Pr and |
| 585 | A is A-3, | Q³ is S, | R¹ is CF₃, | R² is Cl, | R³ is H, | R⁴ is i-Pr and |
| 586 | A is A-3, | Q³ is S, | R¹ is CF₃, | R² is Cl, | R³ is H, | R⁴ is Bu and |
| 587 | A is A-3, | Q³ is S, | R¹ is CF₃, | R² is Cl, | R³ is H, | R⁴ is CF₃ and |
| 588 | A is A-3, | Q³ is S, | R¹ is CF₃, | R² is Cl, | R³ is H, | R⁴ is CHF₂ and |
| 589 | A is A-3, | Q³ is SO₂, | R¹ is CF₃, | R² is H, | R³ is H, | R⁴ is CH₃ and |
| 590 | A is A-3, | Q³ is SO₂, | R¹ is CF₃, | R² is H, | R³ is H, | R⁴ is Et and |
| 591 | A is A-3, | Q³ is SO₂, | R¹ is CF₃, | R² is H, | R³ is H, | R⁴ is c-Pr and |
| 592 | A is A-3, | Q³ is SO₂, | R¹ is CF₃, | R² is H, | R³ is H, | R⁴ is Pr and |
| 593 | A is A-3, | Q³ is SO₂, | R¹ is CF₃, | R² is H, | R³ is H, | R⁴ is i-Pr and |
| 594 | A is A-3, | Q³ is SO₂, | R¹ is CF₃, | R² is H, | R³ is H, | R⁴ is Bu and |
| 595 | A is A-3, | Q³ is SO₂, | R¹ is CF₃, | R² is H, | R³ is H, | R⁴ is CF₃ and |
| 596 | A is A-3, | Q³ is SO₂, | R¹ is CF₃, | R² is H, | R³ is H, | R⁴ is CHF₂ and |
| 597 | A is A-3, | Q³ is SO₂, | R¹ is CF₃, | R² is CH₃, | R³ is H, | R⁴ is CH₃ and |
| 598 | A is A-3, | Q³ is SO₂, | R¹ is CF₃, | R² is CH₃, | R³ is H, | R⁴ is Et and |
| 599 | A is A-3, | Q³ is SO₂, | R¹ is CF₃, | R² is CH₃, | R³ is H, | R⁴ is c-Pr and |
| 600 | A is A-3, | Q³ is SO₂, | R¹ is CF₃, | R² is CH₃, | R³ is H, | R⁴ is Pr and |
| 601 | A is A-3, | Q³ is SO₂, | R¹ is CF₃, | R² is CH₃, | R³ is H, | R⁴ is i-Pr and |
| 602 | A is A-3, | Q³ is SO₂, | R¹ is CF₃, | R² is CH₃, | R³ is H, | R⁴ is Bu and |
| 603 | A is A-3, | Q³ is SO₂, | R¹ is CF₃, | R² is CH₃, | R³ is H, | R⁴ is CF₃ and |
| 604 | A is A-3, | Q³ is SO₂, | R¹ is CF₃, | R² is CH₃, | R³ is H, | R⁴ is CHF₂ and |
| 605 | A is A-3, | Q³ is SO₂, | R¹ is CF₃, | R² is Cl, | R³ is H, | R⁴ is CH₃ and |
| 606 | A is A-3, | Q³ is SO₂, | R¹ is CF₃, | R² is Cl, | R³ is H, | R⁴ is Et and |
| 607 | A is A-3, | Q³ is SO₂, | R¹ is CF₃, | R² is Cl, | R³ is H, | R⁴ is c-Pr and |
| 608 | A is A-3, | Q³ is SO₂, | R¹ is CF₃, | R² is Cl, | R³ is H, | R⁴ is Pr and |
| 609 | A is A-3, | Q³ is SO₂, | R¹ is CF₃, | R² is Cl, | R³ is H, | R⁴ is i-Pr and |
| 610 | A is A-3, | Q³ is SO₂, | R¹ is CF₃, | R² is Cl, | R³ is H, | R⁴ is Bu and |
| 611 | A is A-3, | Q³ is SO₂, | R¹ is CF₃, | R² is Cl, | R³ is H, | R⁴ is CF₃ and |
| 612 | A is A-3, | Q³ is SO₂, | R¹ is CF₃, | R² is Cl, | R³ is H, | R⁴ is CHF₂ and |
| 613 | A is A-3, | Q³ is NCH₃, | R¹ is CF₃, | R² is H, | R³ is H, | R⁴ is CH₃ and |
| 614 | A is A-3, | Q³ is NCH₃, | R¹ is CF₃, | R² is H, | R³ is H, | R⁴ is Et and |
| 615 | A is A-3, | Q³ is NCH₃, | R¹ is CF₃, | R² is H, | R³ is H, | R⁴ is c-Pr and |
| 616 | A is A-3, | Q³ is NCH₃, | R¹ is CF₃, | R² is H, | R³ is H, | R⁴ is Pr and |
| 617 | A is A-3, | Q³ is NCH₃, | R¹ is CF₃, | R² is H, | R³ is H, | R⁴ is i-Pr and |
| 618 | A is A-3, | Q³ is NCH₃, | R¹ is CF₃, | R² is H, | R³ is H, | R⁴ is Bu and |
| 619 | A is A-3, | Q³ is NCH₃, | R¹ is CF₃, | R² is H, | R³ is H, | R⁴ is CF₃ and |
| 620 | A is A-3, | Q³ is NCH₃, | R¹ is CF₃, | R² is H, | R³ is H, | R⁴ is CHF₂ and |
| 621 | A is A-3, | Q³ is NCH₃, | R¹ is CF₃, | R² is CH₃, | R³ is H, | R⁴ is CH₃ and |
| 622 | A is A-3, | Q³ is NCH₃, | R¹ is CF₃, | R² is CH₃, | R³ is H, | R⁴ is Et and |
| 623 | A is A-3, | Q³ is NCH₃, | R¹ is CF₃, | R² is CH₃, | R³ is H, | R⁴ is c-Pr and |
| 624 | A is A-3, | Q³ is NCH₃, | R¹ is CF₃, | R² is CH₃, | R³ is H, | R⁴ is Pr and |
| 625 | A is A-3, | Q³ is NCH₃, | R¹ is CF₃, | R² is CH₃, | R³ is H, | R⁴ is i-Pr and |
| 626 | A is A-3, | Q³ is NCH₃, | R¹ is CF₃, | R² is CH₃, | R³ is H, | R⁴ is Bu and |
| 627 | A is A-3, | Q³ is NCH₃, | R¹ is CF₃, | R² is CH₃, | R³ is H, | R⁴ is CF₃ and |
| 628 | A is A-3, | Q³ is NCH₃, | R¹ is CF₃, | R² is CH₃, | R³ is H, | R⁴ is CHF₂ and |
| 629 | A is A-3, | Q³ is NCH₃, | R¹ is CF₃, | R² is Cl, | R³ is H, | R⁴ is CH₃ and |
| 630 | A is A-3, | Q³ is NCH₃, | R¹ is CF₃, | R² is Cl, | R³ is H, | R⁴ is Et and |
| 631 | A is A-3, | Q³ is NCH₃, | R¹ is CF₃, | R² is Cl, | R³ is H, | R⁴ is c-Pr and |
| 632 | A is A-3, | Q³ is NCH₃, | R¹ is CF₃, | R² is Cl, | R³ is H, | R⁴ is Pr and |
| 633 | A is A-3, | Q³ is NCH₃, | R¹ is CF₃, | R² is Cl, | R³ is H, | R⁴ is i-Pr and |
| 634 | A is A-3, | Q³ is NCH₃, | R¹ is CF₃, | R² is Cl, | R³ is H, | R⁴ is Bu and |
| 635 | A is A-3, | Q³ is NCH₃, | R¹ is CF₃, | R² is Cl, | R³ is H, | R⁴ is CF₃ and |
| 636 | A is A-3, | Q³ is NCH₃, | R¹ is CF₃, | R² is Cl, | R³ is H, | R⁴ is CHF₂ and |
| 637 | A is A-3, | Q³ is NCH₃, | R¹ is CF₃, | R² is H, | R³ is H, | R⁴ is CH₃ and |
| 638 | A is A-3, | Q³ is NCH₃, | R¹ is CF₃, | R² is H, | R³ is H, | R⁴ is Et and |
| 639 | A is A-3, | Q³ is NCH₃, | R¹ is CF₃, | R² is H, | R³ is H, | R⁴ is c-Pr and |
| 640 | A is A-3, | Q³ is NCH₃, | R¹ is CF₃, | R² is H, | R³ is H, | R⁴ is Pr and |
| 641 | A is A-3, | Q³ is NCH₃, | R¹ is CF₃, | R² is H, | R³ is H, | R⁴ is i-Pr and |
| 642 | A is A-3, | Q³ is NCH₃, | R¹ is CF₃, | R² is H, | R³ is H, | R⁴ is Bu and |
| 643 | A is A-3, | Q³ is NCH₃, | R¹ is CF₃, | R² is H, | R³ is H, | R⁴ is CF₃ and |
| 644 | A is A-3, | Q³ is NCH₃, | R¹ is CF₃, | R² is H, | R³ is H, | R⁴ is CHF₂ and |
| 645 | A is A-3, | Q³ is NCH₃, | R¹ is CF₃, | R² is CH₃, | R³ is H, | R⁴ is CH₃ and |
| 646 | A is A-3, | Q³ is NCH₃, | R¹ is CF₃, | R² is CH₃, | R³ is H, | R⁴ is Et and |
| 647 | A is A-3, | Q³ is NCH₃, | R¹ is CF₃, | R² is CH₃, | R³ is H, | R⁴ is c-Pr and |
| 648 | A is A-3, | Q³ is NCH₃, | R¹ is CF₃, | R² is CH₃, | R³ is H, | R⁴ is Pr and |
| 649 | A is A-3, | Q³ is NCH₃, | R¹ is CF₃, | R² is CH₃, | R³ is H, | R⁴ is i-Pr and |
| 650 | A is A-3, | Q³ is NCH₃, | R¹ is CF₃, | R² is CH₃, | R³ is H, | R⁴ is Bu and |
| 651 | A is A-3, | Q³ is NCH₃, | R¹ is CF₃, | R² is CH₃, | R³ is H, | R⁴ is CF₃ and |
| 652 | A is A-3, | Q³ is NCH₃, | R¹ is CF₃, | R² is CH₃, | R³ is H, | R⁴ is CHF₂ and |
| 653 | A is A-3, | Q³ is NCH₃, | R¹ is CF₃, | R² is Cl, | R³ is H, | R⁴ is CH₃ and |
| 654 | A is A-3, | Q³ is NCH₃, | R¹ is CF₃, | R² is Cl, | R³ is H, | R⁴ is Et and |
| 655 | A is A-3, | Q³ is NCH₃, | R¹ is CF₃, | R² is Cl, | R³ is H, | R⁴ is c-Pr and |
| 656 | A is A-3, | Q³ is NCH₃, | R¹ is CF₃, | R² is Cl, | R³ is H, | R⁴ is Pr and |
| 657 | A is A-3, | Q³ is NCH₃, | R¹ is CF₃, | R² is Cl, | R³ is H, | R⁴ is i-Pr and |
| 658 | A is A-3, | Q³ is NCH₃, | R¹ is CF₃, | R² is Cl, | R³ is H, | R⁴ is Bu and |
| 659 | A is A-3, | Q³ is NCH₃, | R¹ is CF₃, | R² is Cl, | R³ is H, | R⁴ is CF₃ and |
| 660 | A is A-3, | Q³ is NCH₃, | R¹ is CF₃, | R² is Cl, | R³ is H, | R⁴ is CHF₂ and |
| 661 | A is A-3, | Q³ is CO, | R¹ is CF₃, | R² is H, | R³ is H, | R⁴ is CH₃ and |
| 662 | A is A-3, | Q³ is CO, | R¹ is CF₃, | R² is H, | R³ is H, | R⁴ is Et and |
| 663 | A is A-3, | Q³ is CO, | R¹ is CF₃, | R² is H, | R³ is H, | R⁴ is c-Pr and |
| 664 | A is A-3, | Q³ is CO, | R¹ is CF₃, | R² is H, | R³ is H, | R⁴ is Pr and |
| 665 | A is A-3, | Q³ is CO, | R¹ is CF₃, | R² is H, | R³ is H, | R⁴ is i-Pr and |
| 666 | A is A-3, | Q³ is CO, | R¹ is CF₃, | R² is H, | R³ is H, | R⁴ is Bu and |
| 667 | A is A-3, | Q³ is CO, | R¹ is CF₃, | R² is H, | R³ is H, | R⁴ is CF₃ and |
| 668 | A is A-3, | Q³ is CO, | R¹ is CF₃, | R² is H, | R³ is H, | R⁴ is CHF₂ and |
| 669 | A is A-3, | Q³ is CO, | R¹ is CF₃, | R² is CH₃, | R³ is H, | R⁴ is CH₃ and |
| 670 | A is A-3, | Q³ is CO, | R¹ is CF₃, | R² is CH₃, | R³ is H, | R⁴ is Et and |
| 671 | A is A-3, | Q³ is CO, | R¹ is CF₃, | R² is CH₃, | R³ is H, | R⁴ is c-Pr and |
| 672 | A is A-3, | Q³ is CO, | R¹ is CF₃, | R² is CH₃, | R³ is H, | R⁴ is Pr and |
| 673 | A is A-3, | Q³ is CO, | R¹ is CF₃, | R² is CH₃, | R³ is H, | R⁴ is i-Pr and |
| 674 | A is A-3, | Q³ is CO, | R¹ is CF₃, | R² is CH₃, | R³ is H, | R⁴ is Bu and |
| 675 | A is A-3, | Q³ is CO, | R¹ is CF₃, | R² is CH₃, | R³ is H, | R⁴ is CF₃ and |
| 676 | A is A-3, | Q³ is CO, | R¹ is CF₃, | R² is CH₃, | R³ is H, | R⁴ is CHF₂ and |
| 677 | A is A-3, | Q³ is CO, | R¹ is CF₃, | R² is Cl, | R³ is H, | R⁴ is CH₃ and |
| 678 | A is A-3, | Q³ is CO, | R¹ is CF₃, | R² is Cl, | R³ is H, | R⁴ is Et and |
| 679 | A is A-3, | Q³ is CO, | R¹ is CF₃, | R² is Cl, | R³ is H, | R⁴ is c-Pr and |
| 680 | A is A-3, | Q³ is CO, | R¹ is CF₃, | R² is Cl, | R³ is H, | R⁴ is Pr and |
| 681 | A is A-3, | Q³ is CO, | R¹ is CF₃, | R² is Cl, | R³ is H, | R⁴ is i-Pr and |
| 682 | A is A-3, | Q³ is CO, | R¹ is CF₃, | R² is Cl, | R³ is H, | R⁴ is Bu and |
| 683 | A is A-3, | Q³ is CO, | R¹ is CF₃, | R² is Cl, | R³ is H, | R⁴ is CF₃ and |
| 684 | A is A-3, | Q³ is CO, | R¹ is CF₃, | R² is Cl, | R³ is H, | R⁴ is CHF₂ and |
| 685 | A is A-3, | Q³ is CH═CH, | R¹ is CF₃, | R² is H, | R³ is CH₃, | R⁴ is CH₃ and |
| 686 | A is A-3, | Q³ is CH═CH, | R¹ is CF₃, | R² is H, | R³ is COCH₃, | R⁴ is CH₃ and |
| 687 | A is A-3, | Q³ is CH═CH, | R¹ is CF₃, | R² is H, | R³ is CO₂CH₃, | R⁴ is CH₃ and |
| 688 | A is A-3, | Q³ is CH═CH, | R¹ is CF₃, | R² is H, | R³ is COCF₃, | R⁴ is CH₃ and |
| 689 | A is A-3, | Q³ is CH═CH, | R¹ is CF₃, | R² is CHF₂, | R³ is H, | R⁴ is CH₃ and |
| 690 | A is A-3, | Q³ is CH═CH, | R¹ is CF₃, | R² is F, | R³ is H, | R⁴ is CH₃ and |
| 691 | A is A-3, | Q³ is CH═CH, | R¹ is CF₃, | R² is Et, | R³ is H, | R⁴ is CH₃ and |
| 692 | A is A-3, | Q³ is CH═CH, | R¹ is CF₃, | R² is CF₃, | R³ is H, | R⁴ is CH₃ and |
| 693 | A is A-3, | Q³ is CH═CH, | R¹ is CF₃, | R² is CCH, | R³ is H, | R⁴ is CH₃ and |
| 694 | A is A-3, | Q³ is O, | R¹ is CF₃, | R² is H, | R³ is CH₃, | R⁴ is CH₃ and |
| 695 | A is A-3, | Q³ is O, | R¹ is CF₃, | R² is H, | R³ is COCH₃, | R⁴ is CH₃ and |
| 696 | A is A-3, | Q³ is O, | R¹ is CF₃, | R² is H, | R³ is CO₂CH₃, | R⁴ is CH₃ and |
| 697 | A is A-3, | Q³ is O, | R¹ is CF₃, | R² is CHF₂ | R³ is COCF₃, | R⁴ is CH₃ and |
| 698 | A is A-3, | Q³ is O, | R¹ is CF₃, | R² is F, | R³ is H, | R⁴ is CH₃ and |
| 699 | A is A-3, | Q³ is O, | R¹ is CF₃, | R² is F, | R³ is H, | R⁴ is CH₃ and |
| 700 | A is A-3, | Q³ is O, | R¹ is CF₃, | R² is Et, | R³ is H, | R⁴ is CH₃ and |
| 701 | A is A-3, | Q³ is O, | R¹ is CF₃, | R² is CF₃, | R³ is H, | R⁴ is CH₃ and |
| 702 | A is A-3, | Q³ is O, | R¹ is CF₃, | R² is CCH, | R³ is H, | R⁴ is CH₃ and |
| 703 | A is A-1, | Q¹ is CH₂, | R¹ is CF₃, | R² is H, | R³ is H, | R⁴ is CH₃ and |
| 704 | A is A-1, | Q¹ is CH₂, | R¹ is CF₃, | R² is H, | R³ is H, | R⁴ is Et and |
| 705 | A is A-1, | Q¹ is CH₂, | R¹ is CF₃, | R² is H, | R³ is H, | R⁴ is c-Pr and |
| 706 | A is A-1, | Q¹ is CH₂, | R¹ is CF₃, | R² is H, | R³ is H, | R⁴ is Pr and |
| 707 | A is A-1, | Q¹ is CH₂, | R¹ is CF₃, | R² is H, | R³ is H, | R⁴ is i-Pr and |
| 708 | A is A-1, | Q¹ is CH₂, | R¹ is CF₃, | R² is H, | R³ is H, | R⁴ is Bu and |
| 709 | A is A-1, | Q¹ is CH₂, | R¹ is CF₃, | R² is H, | R³ is H, | R⁴ is CF₃ and |
| 710 | A is A-1, | Q¹ is CH₂, | R¹ is CF₃, | R² is H, | R³ is H, | R⁴ is CHF₂ and |
| 711 | A is A-1, | Q¹ is CH₂, | R¹ is CF₃, | R² is CH₃, | R³ is H, | R⁴ is CH₃ and |
| 712 | A is A-1, | Q¹ is CH₂, | R¹ is CF₃, | R² is CH₃, | R³ is H, | R⁴ is Et and |
| 713 | A is A-1, | Q¹ is CH₂, | R¹ is CF₃, | R² is CH₃, | R³ is H, | R⁴ is c-Pr and |
| 714 | A is A-1, | Q¹ is CH₂, | R¹ is CF₃, | R² is CH₃, | R³ is H, | R⁴ is Pr and |
| 715 | A is A-1, | Q¹ is CH₂, | R¹ is CF₃, | R² is CH₃, | R³ is H, | R⁴ is i-Pr and |
| 716 | A is A-1, | Q¹ is CH₂, | R¹ is CF₃, | R² is CH₃, | R³ is H, | R⁴ is Bu and |
| 717 | A is A-1, | Q¹ is CH₂, | R¹ is CF₃, | R² is CH₃, | R³ is H, | R⁴ is CF₃ and |
| 718 | A is A-1, | Q¹ is CH₂, | R¹ is CF₃, | R² is CH₃, | R³ is H, | R⁴ is CHF₂ and |
| 719 | A is A-1, | Q¹ is CH₂, | R¹ is CF₃, | R² is Cl, | R³ is H, | R⁴ is CH₃ and |
| 720 | A is A-1, | Q¹ is CH₂, | R¹ is CF₃, | R² is Cl, | R³ is H, | R⁴ is Et and |
| 721 | A is A-1, | Q¹ is CH₂, | R¹ is CF₃, | R² is Cl, | R³ is H, | R⁴ is c-Pr and |
| 722 | A is A-1, | Q¹ is CH₂, | R¹ is CF₃, | R² is Cl, | R³ is H, | R⁴ is Pr and |
| 723 | A is A-1, | Q¹ is CH₂, | R¹ is CF₃, | R² is Cl, | R³ is H, | R⁴ is i-Pr and |
| 724 | A is A-1, | Q¹ is CH₂, | R¹ is CF₃, | R² is Cl, | R³ is H, | R⁴ is Bu and |
| 725 | A is A-1, | Q¹ is CH₂, | R¹ is CF₃, | R² is Cl, | R³ is H, | R⁴ is CF₃ and |
| 726 | A is A-1, | Q¹ is CH₂, | R¹ is CF₃, | R² is Cl, | R³ is H, | R⁴ is CHF₂ and |
| 727 | A is A-1, | Q¹ is CH₂, | R¹ is CN, | R² is H, | R³ is H, | R⁴ is CH₃ and |
| 728 | A is A-1, | Q¹ is CH₂, | R¹ is CN, | R² is H, | R³ is H, | R⁴ is Et and |
| 729 | A is A-1, | Q¹ is CH₂, | R¹ is CN, | R² is H, | R³ is H, | R⁴ is c-Pr and |
| 730 | A is A-1, | Q¹ is CH₂, | R¹ is CN, | R² is H, | R³ is H, | R⁴ is Pr and |
| 731 | A is A-1, | Q¹ is CH₂, | R¹ is CN, | R² is H, | R³ is H, | R⁴ is i-Pr and |
| 732 | A is A-1, | Q¹ is CH₂, | R¹ is CN, | R² is H, | R³ is H, | R⁴ is Bu and |
| 733 | A is A-1, | Q¹ is CH₂, | R¹ is CN, | R² is H, | R³ is H, | R⁴ is CF₃ and |
| 734 | A is A-1, | Q¹ is CH₂, | R¹ is CN, | R² is H, | R³ is H, | R⁴ is CHF₂ and |
| 735 | A is A-1, | Q¹ is CH₂, | R¹ is CN, | R² is CH₃, | R³ is H, | R⁴ is CH₃ and |
| 736 | A is A-1, | Q¹ is CH₂, | R¹ is CN, | R² is CH₃, | R³ is H, | R⁴ is Et and |
| 737 | A is A-1, | Q¹ is CH₂, | R¹ is CN, | R² is CH₃, | R³ is H, | R⁴ is c-Pr and |
| 738 | A is A-1, | Q¹ is CH₂, | R¹ is CN, | R² is CH₃, | R³ is H, | R⁴ is Pr and |
| 739 | A is A-1, | Q¹ is CH₂, | R¹ is CN, | R² is CH₃, | R³ is H, | R⁴ is i-Pr and |
| 740 | A is A-1, | Q¹ is CH₂, | R¹ is CN, | R² is CH₃, | R³ is H, | R⁴ is Bu and |
| 741 | A is A-1, | Q¹ is CH₂, | R¹ is CN, | R² is CH₃, | R³ is H, | R⁴ is CF₃ and |
| 742 | A is A-1, | Q¹ is CH₂, | R¹ is CN, | R² is CH₃, | R³ is H, | R⁴ is CHF₂ and |
| 743 | A is A-1, | Q¹ is CH₂, | R¹ is CN, | R² is Cl, | R³ is H, | R⁴ is CH₃ and |
| 744 | A is A-1, | Q¹ is CH₂, | R¹ is CN, | R² is Cl, | R³ is H, | R⁴ is Et and |
| 745 | A is A-1, | Q¹ is CH₂, | R¹ is CN, | R² is Cl, | R³ is H, | R⁴ is c-Pr and |
| 746 | A is A-1, | Q¹ is CH₂, | R¹ is CN, | R² is Cl, | R³ is H, | R⁴ is Pr and |
| 747 | A is A-1, | Q¹ is CH₂, | R¹ is CN, | R² is Cl, | R³ is H, | R⁴ is i-Pr and |
| 748 | A is A-1, | Q¹ is CH₂, | R¹ is CN, | R² is Cl, | R³ is H, | R⁴ is Bu and |
| 749 | A is A-1, | Q¹ is CH₂, | R¹ is CN, | R² is Cl, | R³ is H, | R⁴ is CF₃ and |
| 750 | A is A-1, | Q¹ is CH₂, | R¹ is CN, | R² is Cl, | R³ is H, | R⁴ is CHF₂ and |
| 751 | A is A-1, | Q¹ is CH₂, | R¹ is CHF₂, | R² is H, | R³ is H, | R⁴ is CH₃ and |
| 752 | A is A-1, | Q¹ is CH₂, | R¹ is CHF₂, | R² is H, | R³ is H, | R⁴ is Et and |
| 753 | A is A-1, | Q¹ is CH₂, | R¹ is CHF₂, | R² is H, | R³ is H, | R⁴ is c-Pr and |
| 754 | A is A-1, | Q¹ is CH₂, | R¹ is CHF₂, | R² is H, | R³ is H, | R⁴ is Pr and |
| 755 | A is A-1, | Q¹ is CH₂, | R¹ is CHF₂, | R² is H, | R³ is H, | R⁴ is i-Pr and |
| 756 | A is A-1, | Q¹ is CH₂, | R¹ is CHF₂, | R² is H, | R³ is H, | R⁴ is Bu and |
| 757 | A is A-1, | Q¹ is CH₂, | R¹ is CHF₂, | R² is H, | R³ is H, | R⁴ is CF₃ and |
| 758 | A is A-1, | Q¹ is CH₂, | R¹ is CHF₂, | R² is H, | R³ is H, | R⁴ is CHF₂ and |
| 759 | A is A-1, | Q¹ is CH₂, | R¹ is CHF₂, | R² is CH₃, | R³ is H, | R⁴ is CH₃ and |
| 760 | A is A-1, | Q¹ is CH₂, | R¹ is CHF₂, | R² is CH₃, | R³ is H, | R⁴ is Et and |
| 761 | A is A-1, | Q¹ is CH₂, | R¹ is CHF₂, | R² is CH₃, | R³ is H, | R⁴ is c-Pr and |
| 762 | A is A-1, | Q¹ is CH₂, | R¹ is CHF₂, | R² is CH₃, | R³ is H, | R⁴ is Pr and |
| 763 | A is A-1, | Q¹ is CH₂, | R¹ is CHF₂, | R² is CH₃, | R³ is H, | R⁴ is i-Pr and |
| 764 | A is A-1, | Q¹ is CH₂, | R¹ is CHF₂, | R² is CH₃, | R³ is H, | R⁴ is Bu and |
| 765 | A is A-1, | Q¹ is CH₂, | R¹ is CHF₂, | R² is CH₃, | R³ is H, | R⁴ is CF₃ and |
| 766 | A is A-1, | Q¹ is CH₂, | R¹ is CHF₂, | R² is CH₃, | R³ is H, | R⁴ is CHF₂ and |
| 767 | A is A-1, | Q¹ is CH₂, | R¹ is CHF₂, | R² is Cl, | R³ is H, | R⁴ is CH₃ and |
| 768 | A is A-1, | Q¹ is CH₂, | R¹ is CHF₂, | R² is Cl, | R³ is H, | R⁴ is Et and |
| 769 | A is A-1, | Q¹ is CH₂, | R¹ is CHF₂, | R² is Cl, | R³ is H, | R⁴ is c-Pr and |
| 770 | A is A-1, | Q¹ is CH₂, | R¹ is CHF₂, | R² is Cl, | R³ is H, | R⁴ is Pr and |
| 771 | A is A-1, | Q¹ is CH₂, | R¹ is CHF₂, | R² is Cl, | R³ is H, | R⁴ is i-Pr and |
| 772 | A is A-1, | Q¹ is CH₂, | R¹ is CHF₂, | R² is Cl, | R³ is H, | R⁴ is Bu and |
| 773 | A is A-1, | Q¹ is CH₂, | R¹ is CHF₂, | R² is Cl, | R³ is H, | R⁴ is CF₃ and |
| 774 | A is A-1, | Q¹ is CH₂, | R¹ is CHF₂, | R² is Cl, | R³ is H, | R⁴ is CHF₂ and |
| 775 | A is A-2, | Q² is CH₂, | R¹ is CF₃, | R² is H, | R³ is H, | R⁴ is CH₃ and |
| 776 | A is A-2, | Q² is CH₂, | R¹ is CF₃, | R² is H, | R³ is H, | R⁴ is Et and |
| 777 | A is A-2, | Q² is CH₂, | R¹ is CF₃, | R² is H, | R³ is H, | R⁴ is c-Pr and |
| 778 | A is A-2, | Q² is CH₂, | R¹ is CF₃, | R² is H, | R³ is H, | R⁴ is Pr and |
| 779 | A is A-2, | Q² is CH₂, | R¹ is CF₃, | R² is H, | R³ is H, | R⁴ is i-Pr and |
| 780 | A is A-2, | Q² is CH₂, | R¹ is CF₃, | R² is H, | R³ is H, | R⁴ is Bu and |
| 781 | A is A-2, | Q² is CH₂, | R¹ is CF₃, | R² is H, | R³ is H, | R⁴ is CF₃ and |
| 782 | A is A-2, | Q² is CH₂, | R¹ is CF₃, | R² is H, | R³ is H, | R⁴ is CHF₂ and |
| 783 | A is A-2, | Q² is CH₂, | R¹ is CF₃, | R² is CH₃, | R³ is H, | R⁴ is CH₃ and |
| 784 | A is A-2, | Q² is CH₂, | R¹ is CF₃, | R² is CH₃, | R³ is H, | R⁴ is Et and |
| 785 | A is A-2, | Q² is CH₂, | R¹ is CF₃, | R² is CH₃, | R³ is H, | R⁴ is c-Pr and |
| 786 | A is A-2, | Q² is CH₂, | R¹ is CF₃, | R² is CH₃, | R³ is H, | R⁴ is Pr and |
| 787 | A is A-2, | Q² is CH₂, | R¹ is CF₃, | R² is CH₃, | R³ is H, | R⁴ is i-Pr and |
| 788 | A is A-2, | Q² is CH₂, | R¹ is CF₃, | R² is CH₃, | R³ is H, | R⁴ is Bu and |
| 789 | A is A-2, | Q² is CH₂, | R¹ is CF₃, | R² is CH₃, | R³ is H, | R⁴ is CF₃ and |
| 790 | A is A-2, | Q² is CH₂, | R¹ is CF₃, | R² is CH₃, | R³ is H, | R⁴ is CHF₂ and |
| 791 | A is A-2, | Q² is CH₂, | R¹ is CF₃, | R² is Cl, | R³ is H, | R⁴ is CH₃ and |
| 792 | A is A-2, | Q² is CH₂, | R¹ is CF₃, | R² is Cl, | R³ is H, | R⁴ is Et and |
| 793 | A is A-2, | Q² is CH₂, | R¹ is CF₃, | R² is Cl, | R³ is H, | R⁴ is c-Pr and |
| 794 | A is A-2, | Q² is CH₂, | R¹ is CF₃, | R² is Cl, | R³ is H, | R⁴ is Pr and |
| 795 | A is A-2, | Q² is CH₂, | R¹ is CF₃, | R² is Cl, | R³ is H, | R⁴ is i-Pr and |
| 796 | A is A-2, | Q² is CH₂, | R¹ is CF₃, | R² is Cl, | R³ is H, | R⁴ is Bu and |
| 797 | A is A-2, | Q² is CH₂, | R¹ is CF₃, | R² is Cl, | R³ is H, | R⁴ is CF₃ and |
| 798 | A is A-2, | Q² is CH₂, | R¹ is CF₃, | R² is Cl, | R³ is H, | R⁴ is CHF₂ and |
| 799 | A is A-2, | Q² is CH₂, | R¹ is CN, | R² is H, | R³ is H, | R⁴ is CH₃ and |
| 800 | A is A-2, | Q² is CH₂, | R¹ is CN, | R² is H, | R³ is H, | R⁴ is Et and |
| 801 | A is A-2, | Q² is CH₂, | R¹ is CN, | R² is H, | R³ is H, | R⁴ is c-Pr and |
| 802 | A is A-2, | Q² is CH₂, | R¹ is CN, | R² is H, | R³ is H, | R⁴ is Pr and |
| 803 | A is A-2, | Q² is CH₂, | R¹ is CN, | R² is H, | R³ is H, | R⁴ is i-Pr and |
| 804 | A is A-2, | Q² is CH₂, | R¹ is CN, | R² is H, | R³ is H, | R⁴ is Bu and |
| 805 | A is A-2, | Q² is CH₂, | R¹ is CN, | R² is H, | R³ is H, | R⁴ is CF₃ and |
| 806 | A is A-2, | Q² is CH₂, | R¹ is CN, | R² is H, | R³ is H, | R⁴ is CHF₂ and |
| 807 | A is A-2, | Q² is CH₂, | R¹ is CN, | R² is CH₃, | R³ is H, | R⁴ is CH₃ and |
| 808 | A is A-2, | Q² is CH₂, | R¹ is CN, | R² is CH₃, | R³ is H, | R⁴ is Et and |
| 809 | A is A-2, | Q² is CH₂, | R¹ is CN, | R² is CH₃, | R³ is H, | R⁴ is c-Pr and |
| 810 | A is A-2, | Q² is CH₂, | R¹ is CN, | R² is CH₃, | R³ is H, | R⁴ is Pr and |
| 811 | A is A-2, | Q² is CH₂, | R¹ is CN, | R² is CH₃, | R³ is H, | R⁴ is i-Pr and |
| 812 | A is A-2, | Q² is CH₂, | R¹ is CN, | R² is CH₃, | R³ is H, | R⁴ is Bu and |
| 813 | A is A-2, | Q² is CH₂, | R¹ is CN, | R² is CH₃, | R³ is H, | R⁴ is CF₃ and |
| 814 | A is A-2, | Q² is CH₂, | R¹ is CN, | R² is CH₃, | R³ is H, | R⁴ is CHF₂ and |
| 815 | A is A-2, | Q² is CH₂, | R¹ is CN, | R² is Cl, | R³ is H, | R⁴ is CH₃ and |
| 816 | A is A-2, | Q² is CH₂, | R¹ is CN, | R² is Cl, | R³ is H, | R⁴ is Et and |
| 817 | A is A-2, | Q² is CH₂, | R¹ is CN, | R² is Cl, | R³ is H, | R⁴ is c-Pr and |
| 818 | A is A-2, | Q² is CH₂, | R¹ is CN, | R² is Cl, | R³ is H, | R⁴ is Pr and |
| 819 | A is A-2, | Q² is CH₂, | R¹ is CN, | R² is Cl, | R³ is H, | R⁴ is i-Pr and |
| 820 | A is A-2, | Q² is CH₂, | R¹ is CN, | R² is Cl, | R³ is H, | R⁴ is Bu and |
| 821 | A is A-2, | Q² is CH₂, | R¹ is CN, | R² is Cl, | R³ is H, | R⁴ is CF₃ and |
| 822 | A is A-2, | Q² is CH₂, | R¹ is CN, | R² is Cl, | R³ is H, | R⁴ is CHF₂ and |
| 823 | A is A-2, | Q² is CH₂, | R¹ is CHF₂, | R² is H, | R³ is H, | R⁴ is CH₃ and |
| 824 | A is A-2, | Q² is CH₂, | R¹ is CHF₂, | R² is H, | R³ is H, | R⁴ is Et and |
| 825 | A is A-2, | Q² is CH₂, | R¹ is CHF₂, | R² is H, | R³ is H, | R⁴ is c-Pr and |
| 826 | A is A-2, | Q² is CH₂, | R¹ is CHF₂, | R² is H, | R³ is H, | R⁴ is Pr and |
| 827 | A is A-2, | Q² is CH₂, | R¹ is CHF₂, | R² is H, | R³ is H, | R⁴ is i-Pr and |
| 828 | A is A-2, | Q² is CH₂, | R¹ is CHF₂, | R² is H, | R³ is H, | R⁴ is Bu and |
| 829 | A is A-2, | Q² is CH₂, | R¹ is CHF₂, | R² is H, | R³ is H, | R⁴ is CF₃ and |
| 830 | A is A-2, | Q² is CH₂, | R¹ is CHF₂, | R² is H, | R³ is H, | R⁴ is CHF₂ and |
| 831 | A is A-2, | Q² is CH₂, | R¹ is CHF₂, | R² is CH₃, | R³ is H, | R⁴ is CH₃ and |
| 832 | A is A-2, | Q² is CH₂, | R¹ is CHF₂, | R² is CH₃, | R³ is H, | R⁴ is Et and |
| 833 | A is A-2, | Q² is CH₂, | R¹ is CHF₂, | R² is CH₃, | R³ is H, | R⁴ is c-Pr and |
| 834 | A is A-2, | Q² is CH₂, | R¹ is CHF₂, | R² is CH₃, | R³ is H, | R⁴ is Pr and |
| 835 | A is A-2, | Q² is CH₂, | R¹ is CHF₂, | R² is CH₃, | R³ is H, | R⁴ is i-Pr and |
| 836 | A is A-2, | Q² is CH₂, | R¹ is CHF₂, | R² is CH₃, | R³ is H, | R⁴ is Bu and |
| 837 | A is A-2, | Q² is CH₂, | R¹ is CHF₂, | R² is CH₃, | R³ is H, | R⁴ is CF₃ and |
| 838 | A is A-2, | Q² is CH₂, | R¹ is CHF₂, | R² is CH₃, | R³ is H, | R⁴ is CHF₂ and |
| 839 | A is A-2, | Q² is CH₂, | R¹ is CHF₂, | R² is Cl, | R³ is H, | R⁴ is CH₃ and |
| 840 | A is A-2, | Q² is CH₂, | R¹ is CHF₂, | R² is Cl, | R³ is H, | R⁴ is Et and |
| 841 | A is A-2, | Q² is CH₂, | R¹ is CHF₂, | R² is Cl, | R³ is H, | R⁴ is c-Pr and |
| 842 | A is A-2, | Q² is CH₂, | R¹ is CHF₂, | R² is Cl, | R³ is H, | R⁴ is Pr and |
| 843 | A is A-2, | Q² is CH₂, | R¹ is CHF₂, | R² is Cl, | R³ is H, | R⁴ is i-Pr and |
| 844 | A is A-2, | Q² is CH₂, | R¹ is CHF₂, | R² is Cl, | R³ is H, | R⁴ is Bu and |
| 845 | A is A-2, | Q² is CH₂, | R¹ is CHF₂, | R² is Cl, | R³ is H, | R⁴ is CF₃ and |
| 846 | A is A-2, | Q² is CH₂, | R¹ is CHF₂, | R² is Cl, | R³ is H, | R⁴ is CHF₂ and |
| 847 | A is A-3, | Q³ is CH₂, | R¹ is CF₃, | R² is H, | R³ is H, | R⁴ is CH₃ and |
| 848 | A is A-3, | Q³ is CH₂, | R¹ is CF₃, | R² is H, | R³ is H, | R⁴ is Et and |
| 849 | A is A-3, | Q³ is CH₂, | R¹ is CF₃, | R² is H, | R³ is H, | R⁴ is c-Pr and |
| 850 | A is A-3, | Q³ is CH₂, | R¹ is CF₃, | R² is H, | R³ is H, | R⁴ is Pr and |
| 851 | A is A-3, | Q³ is CH₂, | R¹ is CF₃, | R² is H, | R³ is H, | R⁴ is i-Pr and |
| 852 | A is A-3, | Q³ is CH₂, | R¹ is CF₃, | R² is H, | R³ is H, | R⁴ is Bu and |
| 853 | A is A-3, | Q³ is CH₂, | R¹ is CF₃, | R² is H, | R³ is H, | R⁴ is CF₃ and |
| 854 | A is A-3, | Q³ is CH₂, | R¹ is CF₃, | R² is H, | R³ is H, | R⁴ is CHF₂ and |
| 855 | A is A-3, | Q³ is CH₂, | R¹ is CF₃, | R² is CH₃, | R³ is H, | R⁴ is CH₃ and |
| 856 | A is A-3, | Q³ is CH₂, | R¹ is CF₃, | R² is CH₃, | R³ is H, | R⁴ is Et and |
| 857 | A is A-3, | Q³ is CH₂, | R¹ is CF₃, | R² is CH₃, | R³ is H, | R⁴ is c-Pr and |
| 858 | A is A-3, | Q³ is CH₂, | R¹ is CF₃, | R² is CH₃, | R³ is H, | R⁴ is Pr and |
| 859 | A is A-3, | Q³ is CH₂, | R¹ is CF₃, | R² is CH₃, | R³ is H, | R⁴ is i-Pr and |
| 860 | A is A-3, | Q³ is CH₂, | R¹ is CF₃, | R² is CH₃, | R³ is H, | R⁴ is Bu and |
| 861 | A is A-3, | Q³ is CH₂, | R¹ is CF₃, | R² is CH₃, | R³ is H, | R⁴ is CF₃ and |
| 862 | A is A-3, | Q³ is CH₂, | R¹ is CF₃, | R² is CH₃, | R³ is H, | R⁴ is CHF₂ and |
| 863 | A is A-3, | Q³ is CH₂, | R¹ is CF₃, | R² is Cl, | R³ is H, | R⁴ is CH₃ and |
| 864 | A is A-3, | Q³ is CH₂, | R¹ is CF₃, | R² is Cl, | R³ is H, | R⁴ is Et and |
| 865 | A is A-3, | Q³ is CH₂, | R¹ is CF₃, | R² is Cl, | R³ is H, | R⁴ is c-Pr and |
| 866 | A is A-3, | Q³ is CH₂, | R¹ is CF₃, | R² is Cl, | R³ is H, | R⁴ is Pr and |
| 867 | A is A-3, | Q³ is CH₂, | R¹ is CF₃, | R² is Cl, | R³ is H, | R⁴ is i-Pr and |
| 868 | A is A-3, | Q³ is CH₂, | R¹ is CF₃, | R² is Cl, | R³ is H, | R⁴ is Bu and |
| 869 | A is A-3, | Q³ is CH₂, | R¹ is CF₃, | R² is Cl, | R³ is H, | R⁴ is CF₃ and |
| 870 | A is A-3, | Q³ is CH₂, | R¹ is CF₃, | R² is Cl, | R³ is H, | R⁴ is CHF₂ and |
| 871 | A is A-3, | Q³ is CH₂, | R¹ is CN, | R² is H, | R³ is H, | R⁴ is CH₃ and |
| 872 | A is A-3, | Q³ is CH₂, | R¹ is CN, | R² is H, | R³ is H, | R⁴ is Et and |
| 873 | A is A-3, | Q³ is CH₂, | R¹ is CN, | R² is H, | R³ is H, | R⁴ is c-Pr and |
| 874 | A is A-3, | Q³ is CH₂, | R¹ is CN, | R² is H, | R³ is H, | R⁴ is Pr and |
| 875 | A is A-3, | Q³ is CH₂, | R¹ is CN, | R² is H, | R³ is H, | R⁴ is i-Pr and |
| 876 | A is A-3, | Q³ is CH₂, | R¹ is CN, | R² is H, | R³ is H, | R⁴ is Bu and |
| 877 | A is A-3, | Q³ is CH₂, | R¹ is CN, | R² is H, | R³ is H, | R⁴ is CF₃ and |
| 878 | A is A-3, | Q³ is CH₂, | R¹ is CN, | R² is H, | R³ is H, | R⁴ is CHF₂ and |
| 879 | A is A-3, | Q³ is CH₂, | R¹ is CN, | R² is CH₃, | R³ is H, | R⁴ is CH₃ and |
| 880 | A is A-3, | Q³ is CH₂, | R¹ is CN, | R² is CH₃, | R³ is H, | R⁴ is Et and |
| 881 | A is A-3, | Q³ is CH₂, | R¹ is CN, | R² is CH₃, | R³ is H, | R⁴ is c-Pr and |
| 882 | A is A-3, | Q³ is CH₂, | R¹ is CN, | R² is CH₃, | R³ is H, | R⁴ is Pr and |
| 883 | A is A-3, | Q³ is CH₂, | R¹ is CN, | R² is CH₃, | R³ is H, | R⁴ is i-Pr and |
| 884 | A is A-3, | Q³ is CH₂, | R¹ is CN, | R² is CH₃, | R³ is H, | R⁴ is Bu and |
| 885 | A is A-3, | Q³ is CH₂, | R¹ is CN, | R² is CH₃, | R³ is H, | R⁴ is CF₃ and |
| 886 | A is A-3, | Q³ is CH₂, | R¹ is CN, | R² is CH₃, | R³ is H, | R⁴ is CHF₂ and |
| 887 | A is A-3, | Q³ is CH₂, | R¹ is CN, | R² is Cl, | R³ is H, | R⁴ is CH₃ and |
| 888 | A is A-3, | Q³ is CH₂, | R¹ is CN, | R² is Cl, | R³ is H, | R⁴ is Et and |
| 889 | A is A-3, | Q³ is CH₂, | R¹ is CN, | R² is Cl, | R³ is H, | R⁴ is c-Pr and |
| 890 | A is A-3, | Q³ is CH₂, | R¹ is CN, | R² is Cl, | R³ is H, | R⁴ is Pr and |
| 891 | A is A-3, | Q³ is CH₂, | R¹ is CN, | R² is Cl, | R³ is H, | R⁴ is i-Pr and |
| 892 | A is A-3, | Q³ is CH₂, | R¹ is CN, | R² is Cl, | R³ is H, | R⁴ is Bu and |
| 893 | A is A-3, | Q³ is CH₂, | R¹ is CN, | R² is Cl, | R³ is H, | R⁴ is CF₃ and |
| 894 | A is A-3, | Q³ is CH₂, | R¹ is CN, | R² is Cl, | R³ is H, | R⁴ is CHF₂ and |
| 895 | A is A-3, | Q³ is CH₂, | R¹ is CHF₂, | R² is H, | R³ is H, | R⁴ is CH₃ and |
| 896 | A is A-3, | Q³ is CH₂, | R¹ is CHF₂, | R² is H, | R³ is H, | R⁴ is Et and |
| 897 | A is A-3, | Q³ is CH₂, | R¹ is CHF₂, | R² is H, | R³ is H, | R⁴ is c-Pr and |
| 898 | A is A-3, | Q³ is CH₂, | R¹ is CHF₂, | R² is H, | R³ is H, | R⁴ is Pr and |
| 899 | A is A-3, | Q³ is CH₂, | R¹ is CHF₂, | R² is H, | R³ is H, | R⁴ is i-Pr and |
| 900 | A is A-3, | Q³ is CH₂, | R¹ is CHF₂, | R² is H, | R³ is H, | R⁴ is Bu and |
| 901 | A is A-3, | Q³ is CH₂, | R¹ is CHF₂, | R² is H, | R³ is H, | R⁴ is CF₃ and |
| 902 | A is A-3, | Q³ is CH₂, | R¹ is CHF₂, | R² is H, | R³ is H, | R⁴ is CHF₂ and |
| 903 | A is A-3, | Q³ is CH₂, | R¹ is CHF₂, | R² is CH₃, | R³ is H, | R⁴ is CH₃ and |
| 904 | A is A-3, | Q³ is CH₂, | R¹ is CHF₂, | R² is CH₃, | R³ is H, | R⁴ is Et and |
| 905 | A is A-3, | Q³ is CH₂, | R¹ is CHF₂, | R² is CH₃, | R³ is H, | R⁴ is c-Pr and |
| 906 | A is A-3, | Q³ is CH₂, | R¹ is CHF₂, | R² is CH₃, | R³ is H, | R⁴ is Pr and |
| 907 | A is A-3, | Q³ is CH₂, | R¹ is CHF₂, | R² is CH₃, | R³ is H, | R⁴ is i-Pr and |
| 908 | A is A-3, | Q³ is CH₂, | R¹ is CHF₂, | R² is CH₃, | R³ is H, | R⁴ is Bu and |
| 909 | A is A-3, | Q³ is CH₂, | R¹ is CHF₂, | R² is CH₃, | R³ is H, | R⁴ is CF₃ and |
| 910 | A is A-3, | Q³ is CH₂, | R¹ is CHF₂, | R² is CH₃, | R³ is H, | R⁴ is CHF₂ and |
| 911 | A is A-3, | Q³ is CH₂, | R¹ is CHF₂, | R² is Cl, | R³ is H, | R⁴ is CH₃ and |
| 912 | A is A-3, | Q³ is CH₂, | R¹ is CHF₂, | R² is Cl, | R³ is H, | R⁴ is Et and |
| 913 | A is A-3, | Q³ is CH₂, | R¹ is CHF₂, | R² is Cl, | R³ is H, | R⁴ is c-Pr and |
| 914 | A is A-3, | Q³ is CH₂, | R¹ is CHF₂, | R² is Cl, | R³ is H, | R⁴ is Pr and |
| 915 | A is A-3, | Q³ is CH₂, | R¹ is CHF₂, | R² is Cl, | R³ is H, | R⁴ is i-Pr and |
| 916 | A is A-3, | Q³ is CH₂, | R¹ is CHF₂, | R² is Cl, | R³ is H, | R⁴ is Bu and |
| 917 | A is A-3, | Q³ is CH₂, | R¹ is CHF₂, | R² is Cl, | R³ is H, | R⁴ is CF₃ and |
| 918 | A is A-3, | Q³ is CH₂, | R¹ is CHF₂, | R² is Cl, | R³ is H, | R⁴ is CHF₂ and |

### Table 919

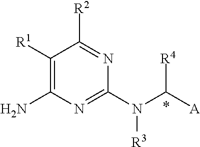

wherein A is

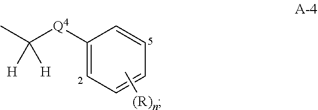

Q⁴ is O, R¹ is CF₃, R² is H, R³ is H, R⁴ is CH₃ and

(R)ₙ

H (i.e. n = 0)
2-F
3-F
4-F
2-Cl
3-Cl
4-Cl
2-Br
3-Br
4-Br
2-I
3-I
4-I
2-CH₃
3-CH₃
4-CH₃
2-CF₃
3-CF₃
4-CF₃
2-CN
3-CN
4-CN
2-OCH₃
3-OCH₃
4-OCH₃
2-OCF₃
3-OCF₃
4-OCF₃
2-C≡CH
3-C≡CH
4-C≡CH
2-Et
3-Et
4-Et
2-C≡CH
2,6-di-Me
3,6-di-Me
4,6-di-Me
2,4-di-Me
3,4-di-Me
2,3-di-Me
3,6-di-Cl
4,6-di-Cl
5,6-di-Cl
2,5-di-Cl
3,5-di-Cl
4,5-di-Cl
2,4-di-Cl
3,4-di-Cl
2,6-di-Br
3,6-di-Br
4,6-di-Br
5,6-di-Br
2,5-di-Br
3,5-di-Br
4,5-di-Br
2,4-di-Br
3,4-di-Br
3-F,4-Br
3-F,5-Br
3-F,6-Br
2-Br,3-F
3-F,4-CF₃
3-F,5-CF₃
3-F,6-CF₃
2-CF₃,3-F
3-CF₃,4-F
3-CF₃,5-F
3-CF₃,6-F
2-F,3-CF₃
3-CF₃,4-CH₃
3-CF₃,5-CH₃
3-CF₃,6-CF3
3-CF₃,4-Cl
3-CF₃,5-Cl
3-CF₃,6-Cl
2-Cl,3-CF₃
2-CF₃,3-Br
3-CF₃,4-Br
3-CF₃,5-Br
3-CF₃,6-Br
2-Br,3-CF₃
3-Cl,4-F
3-Cl,6-F
2-F,3-Cl
2,5-di-Me
3,5-di-Me
2,3-di-Br
2,6-di-F
3,6-di-F
4,6-di-F
5,6-di-F
2,5-di-F
3,5-di-F
4,5-di-F
2,4-di-F
3,4-di-F
2,3-di-F
2,6-di-Cl
2,3-di-Cl
2-F,3-Me
3-Me,4-Cl
3-Me,5-Cl
3-Me,6-Cl
2-Cl,3-Me
3-Me,4-OCF₃
3-Me,5-OCF₃
3-Me,6-OCF₃
2-OCF₃,3-Me
3-F,4-OCF₃
3-F,5-OCF₃
3-F,6-OCF₃
2-F,3-Me
3-Cl,4-OCF₃
3-Cl,5-OCF₃
3-Cl,6-OCF₃
2-OCF₃,3-Cl
3,5-di-CF₃
3,5-di-OCF₃
3-Me,4-Br
3-Me,5-Br
3-Me,6-Br
2-Br,3-Me
3-F,4-Cl
3-F,5-Cl
3-F,6-Cl
2-Cl,3-F
3-Br,4-F
3-Br,6-F
2-F,3-Br
3-Me,4-F
3-Me,5-F
3-Me,6-F

The present disclosure also includes Tables 920 through 1152, each of which is constructed the same as Table 919 above, except that the Header Row in Table 920 (i.e. Q⁴ is O, R¹ is CF₃, R² is H, R³ is H, and R⁴ is CH₃) is replaced with the respective Header Row shown below in Tables 920 through 1152. For example, the first entry in Table 920 is a compound of Formula 1 wherein Q⁴ is O, R¹ is CF₃, R² is H, R³ is H, R⁴ is Et and (R)ₙ is H (i.e. n=0). Tables 921 through 1152 are constructed similarly.

| Table | Header Row | | | | | |
|---|---|---|---|---|---|---|
| 920 | Q⁴ is O, | R¹ is CF₃, | R² is H, | R³ is H, | R⁴ is Et | and |
| 921 | Q⁴ is O, | R¹ is CF₃, | R² is H, | R³ is H, | R⁴ is c-Pr | and |
| 922 | Q⁴ is O, | R¹ is CF₃, | R² is H, | R³ is H, | R⁴ is Pr | and |
| 923 | Q⁴ is O, | R¹ is CF₃, | R² is H, | R³ is H, | R⁴ is i-Pr | and |
| 924 | Q⁴ is O, | R¹ is CF₃, | R² is H, | R³ is H, | R⁴ is Bu | and |
| 925 | Q⁴ is O, | R¹ is CF₃, | R² is H, | R³ is H, | R⁴ is CF₃ | and |
| 926 | Q⁴ is O, | R¹ is CF₃, | R² is H, | R³ is H, | R⁴ is CHF₂ | and |
| 927 | Q⁴ is O, | R¹ is CF₃, | R² is CH₃, | R³ is H, | R⁴ is CH₃ | and |
| 928 | Q⁴ is O, | R¹ is CF₃, | R² is CH₃, | R³ is H, | R⁴ is Et | and |
| 929 | Q⁴ is O, | R¹ is CF₃, | R² is CH₃, | R³ is H, | R⁴ is c-Pr | and |
| 930 | Q⁴ is O, | R¹ is CF₃, | R² is CH₃, | R³ is H, | R⁴ is Pr | and |
| 931 | Q⁴ is O, | R¹ is CF₃, | R² is CH₃, | R³ is H, | R⁴ is i-Pr | and |
| 932 | Q⁴ is O, | R¹ is CF₃, | R² is CH₃, | R³ is H, | R⁴ is Bu | and |
| 933 | Q⁴ is O, | R¹ is CF₃, | R² is CH₃, | R³ is H, | R⁴ is CF₃ | and |
| 934 | Q⁴ is O, | R¹ is CF₃, | R² is CH₃, | R³ is H, | R⁴ is CHF₂ | and |
| 935 | Q⁴ is O, | R¹ is CF₃, | R² is Cl, | R³ is H, | R⁴ is CH₃ | and |
| 936 | Q⁴ is O, | R¹ is CF₃, | R² is Cl, | R³ is H, | R⁴ is Et | and |
| 937 | Q⁴ is O, | R¹ is CF₃, | R² is Cl, | R³ is H, | R⁴ is c-Pr | and |
| 938 | Q⁴ is O, | R¹ is CF₃, | R² is Cl, | R³ is H, | R⁴ is Pr | and |
| 939 | Q⁴ is O, | R¹ is CF₃, | R² is Cl, | R³ is H, | R⁴ is i-Pr | and |
| 940 | Q⁴ is O, | R¹ is CF₃, | R² is Cl, | R³ is H, | R⁴ is Bu | and |
| 941 | Q⁴ is O, | R¹ is CF₃, | R² is Cl, | R³ is H, | R⁴ is CF₃ | and |
| 942 | Q⁴ is O, | R¹ is CF₃, | R² is Cl, | R³ is H, | R⁴ is CHF₂ | and |
| 943 | Q⁴ is O, | R¹ is CN, | R² is H, | R³ is H, | R⁴ is CH₃ | and |
| 944 | Q⁴ is O, | R¹ is CN, | R² is H, | R³ is H, | R⁴ is Et | and |
| 945 | Q⁴ is O, | R¹ is CN, | R² is H, | R³ is H, | R⁴ is c-Pr | and |
| 946 | Q⁴ is O, | R¹ is CN, | R² is H, | R³ is H, | R⁴ is Pr | and |
| 947 | Q⁴ is O, | R¹ is CN, | R² is H, | R³ is H, | R⁴ is i-Pr | and |
| 948 | Q⁴ is O, | R¹ is CN, | R² is H, | R³ is H, | R⁴ is Bu | and |
| 949 | Q⁴ is O, | R¹ is CN, | R² is H, | R³ is H, | R⁴ is CF₃ | and |
| 950 | Q⁴ is O, | R¹ is CN, | R² is H, | R³ is H, | R⁴ is CHF₂ | and |
| 951 | Q⁴ is O, | R¹ is CN, | R² is CH₃, | R³ is H, | R⁴ is CH₃ | and |
| 952 | Q⁴ is O, | R¹ is CN, | R² is CH₃, | R³ is H, | R⁴ is Et | and |
| 953 | Q⁴ is O, | R¹ is CN, | R² is CH₃, | R³ is H, | R⁴ is c-Pr | and |
| 954 | Q⁴ is O, | R¹ is CN, | R² is CH₃, | R³ is H, | R⁴ is Pr | and |
| 955 | Q⁴ is O, | R¹ is CN, | R² is CH₃, | R³ is H, | R⁴ is i-Pr | and |
| 956 | Q⁴ is O, | R¹ is CN, | R² is CH₃, | R³ is H, | R⁴ is Bu | and |
| 957 | Q⁴ is O, | R¹ is CN, | R² is CH₃, | R³ is H, | R⁴ is CF₃ | and |
| 958 | Q⁴ is O, | R¹ is CN, | R² is CH₃, | R³ is H, | R⁴ is CHF₂ | and |
| 959 | Q⁴ is O, | R¹ is CN, | R² is Cl, | R³ is H, | R⁴ is CH₃ | and |
| 960 | Q⁴ is O, | R¹ is CN, | R² is Cl, | R³ is H, | R⁴ is Et | and |
| 961 | Q⁴ is O, | R¹ is CN, | R² is Cl, | R³ is H, | R⁴ is c-Pr | and |
| 962 | Q⁴ is O, | R¹ is CN, | R² is Cl, | R³ is H, | R⁴ is Pr | and |
| 963 | Q⁴ is O, | R¹ is CN, | R² is Cl, | R³ is H, | R⁴ is i-Pr | and |
| 964 | Q⁴ is O, | R¹ is CN, | R² is Cl, | R³ is H, | R⁴ is Bu | and |
| 965 | Q¹ is O, | R¹ is CN, | R² is Cl, | R³ is H, | R⁴ is CF₃ | and |
| 966 | Q⁴ is O, | R¹ is CN, | R² is Cl, | R³ is H, | R⁴ is CHF₂ | and |
| 967 | Q⁴ is O, | R¹ is CHF₂, | R² is H, | R³ is H, | R⁴ is CH₃ | and |
| 968 | Q⁴ is O, | R¹ is CHF₂, | R² is H, | R³ is H, | R⁴ is Et | and |
| 969 | Q⁴ is O, | R¹ is CHF₂, | R² is H, | R³ is H, | R⁴ is c-Pr | and |
| 970 | Q⁴ is O, | R¹ is CHF₂, | R² is H, | R³ is H, | R⁴ is Pr | and |
| 971 | Q⁴ is O, | R¹ is CHF₂, | R² is H, | R³ is H, | R⁴ is i-Pr | and |
| 972 | Q⁴ is O, | R¹ is CHF₂, | R² is H, | R³ is H, | R⁴ is Bu | and |
| 973 | Q⁴ is O, | R¹ is CHF₂, | R² is H, | R³ is H, | R⁴ is CF₃ | and |
| 974 | Q⁴ is O, | R¹ is CHF₂, | R² is H, | R³ is H, | R⁴ is CHF₂ | and |
| 975 | Q⁴ is O, | R¹ is CHF₂, | R² is CH₃, | R³ is H, | R⁴ is CH₃ | and |
| 976 | Q⁴ is O, | R¹ is CHF₂, | R² is CH₃, | R³ is H, | R⁴ is Et | and |
| 977 | Q⁴ is O, | R¹ is CHF₂, | R² is CH₃, | R³ is H, | R⁴ is c-Pr | and |
| 978 | Q⁴ is O, | R¹ is CHF₂, | R² is CH₃, | R³ is H, | R⁴ is Pr | and |
| 979 | Q⁴ is O, | R¹ is CHF₂, | R² is CH₃, | R³ is H, | R⁴ is i-Pr | and |
| 980 | Q⁴ is O, | R¹ is CHF₂, | R² is CH₃, | R³ is H, | R⁴ is Bu | and |
| 981 | Q⁴ is O, | R¹ is CHF₂, | R² is CH₃, | R³ is H, | R⁴ is CF₃ | and |
| 982 | Q⁴ is O, | R¹ is CHF₂, | R² is CH₃, | R³ is H, | R⁴ is CHF₂ | and |
| 983 | Q⁴ is O, | R¹ is CHF₂, | R² is Cl, | R³ is H, | R⁴ is CH₃ | and |
| 984 | Q⁴ is O, | R¹ is CHF₂, | R² is Cl, | R³ is H, | R⁴ is Et | and |
| 985 | Q⁴ is O, | R¹ is CHF₂, | R² is Cl, | R³ is H, | R⁴ is c-Pr | and |
| 986 | Q⁴ is O, | R¹ is CHF₂, | R² is Cl, | R³ is H, | R⁴ is Pr | and |
| 987 | Q⁴ is O, | R¹ is CHF₂, | R² is Cl, | R³ is H, | R⁴ is i-Pr | and |
| 988 | Q⁴ is O, | R¹ is CHF₂, | R² is Cl, | R³ is H, | R⁴ is Bu | and |
| 989 | Q⁴ is O, | R¹ is CHF₂, | R² is Cl, | R³ is H, | R⁴ is CF₃ | and |
| 990 | Q⁴ is O, | R¹ is CHF₂, | R² is Cl, | R³ is H, | R⁴ is CHF₂ | and |
| 991 | Q⁴ is O, | R¹ is CF₃, | R² is H, | R³ is H, | R⁴ is CH₃ | and |
| 992 | Q⁴ is O, | R¹ is CF₃, | R² is H, | R³ is H, | R⁴ is Et | and |
| 993 | Q⁴ is O, | R¹ is CF₃, | R² is H, | R³ is H, | R⁴ is c-Pr | and |
| 994 | Q⁴ is O, | R¹ is CF₃, | R² is H, | R³ is H, | R⁴ is Pr | and |
| 995 | Q⁴ is O, | R¹ is CF₃, | R² is H, | R³ is H, | R⁴ is i-Pr | and |
| 996 | Q⁴ is O, | R¹ is CF₃, | R² is H, | R³ is H, | R⁴ is Bu | and |
| 997 | Q⁴ is O, | R¹ is CF₃, | R² is H, | R³ is H, | R⁴ is CF₃ | and |
| 998 | Q⁴ is O, | R¹ is CF₃, | R² is H, | R³ is H, | R⁴ is CHF₂ | and |
| 999 | Q⁴ is O, | R¹ is CF₃, | R² is CH₃, | R³ is H, | R⁴ is CH₃ | and |
| 1000 | Q⁴ is O, | R¹ is CF₃, | R² is CH₃, | R³ is H, | R⁴ is Et | and |
| 1001 | Q⁴ is O, | R¹ is CF₃, | R² is CH₃, | R³ is H, | R⁴ is c-Pr | and |
| 1002 | Q⁴ is O, | R¹ is CF₃, | R² is CH₃, | R³ is H, | R⁴ is Pr | and |
| 1003 | Q⁴ is O, | R¹ is CF₃, | R² is CH₃, | R³ is H, | R⁴ is i-Pr | and |
| 1004 | Q⁴ is O, | R¹ is CF₃, | R² is CH₃, | R³ is H, | R⁴ is Bu | and |
| 1005 | Q⁴ is O, | R¹ is CF₃, | R² is CH₃, | R³ is H, | R⁴ is CF₃ | and |
| 1006 | Q⁴ is O, | R¹ is CF₃, | R² is CH₃, | R³ is H, | R⁴ is CHF₂ | and |
| 1007 | Q⁴ is O, | R¹ is CF₃, | R² is Cl, | R³ is H, | R⁴ is CH₃ | and |
| 1008 | Q⁴ is O, | R¹ is CF₃, | R² is Cl, | R³ is H, | R⁴ is Et | and |
| 1009 | Q⁴ is O, | R¹ is CF₃, | R² is Cl, | R³ is H, | R⁴ is c-Pr | and |
| 1010 | Q⁴ is O, | R¹ is CF₃, | R² is Cl, | R³ is H, | R⁴ is Pr | and |
| 1011 | Q⁴ is O, | R¹ is CF₃, | R² is Cl, | R³ is H, | R⁴ is i-Pr | and |
| 1012 | Q⁴ is O, | R¹ is CF₃, | R² is Cl, | R³ is H, | R⁴ is Bu | and |
| 1013 | Q⁴ is O, | R¹ is CF₃, | R² is Cl, | R³ is H, | R⁴ is CF₃ | and |
| 1014 | Q⁴ is O, | R¹ is CF₃, | R² is Cl, | R³ is H, | R⁴ is CHF₂ | and |
| 1015 | Q⁴ is S, | R¹ is CF₃, | R² is H, | R³ is H, | R⁴ is CH₃ | and |
| 1016 | Q⁴ is S, | R¹ is CF₃, | R² is H, | R³ is H, | R⁴ is Et | and |
| 1017 | Q⁴ is S, | R¹ is CF₃, | R² is H, | R³ is H, | R⁴ is c-Pr | and |
| 1018 | Q⁴ is S, | R¹ is CF₃, | R² is H, | R³ is H, | R⁴ is Pr | and |
| 1019 | Q⁴ is S, | R¹ is CF₃, | R² is H, | R³ is H, | R⁴ is i-Pr | and |
| 1020 | Q⁴ is S, | R¹ is CF₃, | R² is H, | R³ is H, | R⁴ is Bu | and |
| 1021 | Q⁴ is S, | R¹ is CF₃, | R² is H, | R³ is H, | R⁴ is CF₃ | and |
| 1022 | Q⁴ is S, | R¹ is CF₃, | R² is H, | R³ is H, | R⁴ is CHF₂ | and |
| 1023 | Q⁴ is S, | R¹ is CF₃, | R² is CH₃, | R³ is H, | R⁴ is CH₃ | and |
| 1024 | Q⁴ is S, | R¹ is CF₃, | R² is CH₃, | R³ is H, | R⁴ is Et | and |
| 1025 | Q⁴ is S, | R¹ is CF₃, | R² is CH₃, | R³ is H, | R⁴ is c-Pr | and |
| 1026 | Q⁴ is S, | R¹ is CF₃, | R² is CH₃, | R³ is H, | R⁴ is Pr | and |
| 1027 | Q⁴ is S, | R¹ is CF₃, | R² is CH₃, | R³ is H, | R⁴ is i-Pr | and |
| 1028 | Q⁴ is S, | R¹ is CF₃, | R² is CH₃, | R³ is H, | R⁴ is Bu | and |
| 1029 | Q⁴ is S, | R¹ is CF₃, | R² is CH₃, | R³ is H, | R⁴ is CF₃ | and |
| 1030 | Q⁴ is S, | R¹ is CF₃, | R² is CH₃, | R³ is H, | R⁴ is CHF₂ | and |
| 1031 | Q⁴ is S, | R¹ is CF₃, | R² is Cl, | R³ is H, | R⁴ is CH₃ | and |
| 1032 | Q⁴ is S, | R¹ is CF₃, | R² is Cl, | R³ is H, | R⁴ is Et | and |
| 1033 | Q⁴ is S, | R¹ is CF₃, | R² is Cl, | R³ is H, | R⁴ is c-Pr | and |
| 1034 | Q⁴ is S, | R¹ is CF₃, | R² is Cl, | R³ is H, | R⁴ is Pr | and |
| 1035 | Q⁴ is S, | R¹ is CF₃, | R² is Cl, | R³ is H, | R⁴ is i-Pr | and |
| 1036 | Q⁴ is S, | R¹ is CF₃, | R² is Cl, | R³ is H, | R⁴ is Bu | and |
| 1037 | Q⁴ is S, | R¹ is CF₃, | R² is Cl, | R³ is H, | R⁴ is CF₃ | and |
| 1038 | Q⁴ is S, | R¹ is CF₃, | R² is Cl, | R³ is H, | R⁴ is CHF₂ | and |
| 1039 | Q⁴ is SO₂, | R¹ is CF₃, | R² is H, | R³ is H, | R⁴ is CH₃ | and |
| 1040 | Q⁴ is SO₂, | R¹ is CF₃, | R² is H, | R³ is H, | R⁴ is Et | and |
| 1041 | Q⁴ is SO₂, | R¹ is CF₃, | R² is H, | R³ is H, | R⁴ is c-Pr | and |
| 1042 | Q⁴ is SO₂, | R¹ is CF₃, | R² is H, | R³ is H, | R⁴ is Pr | and |
| 1043 | Q⁴ is SO₂, | R¹ is CF₃, | R² is H, | R³ is H, | R⁴ is i-Pr | and |
| 1044 | Q⁴ is SO₂, | R¹ is CF₃, | R² is H, | R³ is H, | R⁴ is Bu | and |
| 1045 | Q⁴ is SO₂, | R¹ is CF₃, | R² is H, | R³ is H, | R⁴ is CF₃ | and |
| 1046 | Q⁴ is SO₂, | R¹ is CF₃, | R² is H, | R³ is H, | R⁴ is CHF₂ | and |
| 1047 | Q⁴ is SO₂, | R¹ is CF₃, | R² is CH₃, | R³ is H, | R⁴ is CH₃ | and |
| 1048 | Q⁴ is SO₂, | R¹ is CF₃, | R² is CH₃, | R³ is H, | R⁴ is Et | and |
| 1049 | Q⁴ is SO₂, | R¹ is CF₃, | R² is CH₃, | R³ is H, | R⁴ is c-Pr | and |
| 1050 | Q⁴ is SO₂, | R¹ is CF₃, | R² is CH₃, | R³ is H, | R⁴ is Pr | and |
| 1051 | Q⁴ is SO₂, | R¹ is CF₃, | R² is CH₃, | R³ is H, | R⁴ is i-Pr | and |
| 1052 | Q⁴ is SO₂, | R¹ is CF₃, | R² is CH₃, | R³ is H, | R⁴ is Bu | and |
| 1053 | Q⁴ is SO₂, | R¹ is CF₃, | R² is CH₃, | R³ is H, | R⁴ is CF₃ | and |
| 1054 | Q⁴ is SO₂, | R¹ is CF₃, | R² is CH₃, | R³ is H, | R⁴ is CHF₂ | and |
| 1055 | Q⁴ is SO₂, | R¹ is CF₃, | R² is Cl, | R³ is H, | R⁴ is CH₃ | and |
| 1056 | Q⁴ is SO₂, | R¹ is CF₃, | R² is Cl, | R³ is H, | R⁴ is Et | and |
| 1057 | Q⁴ is SO₂, | R¹ is CF₃, | R² is Cl, | R³ is H, | R⁴ is c-Pr | and |
| 1058 | Q⁴ is SO₂, | R¹ is CF₃, | R² is Cl, | R³ is H, | R⁴ is Pr | and |
| 1059 | Q⁴ is SO₂, | R¹ is CF₃, | R² is Cl, | R³ is H, | R⁴ is i-Pr | and |
| 1060 | Q⁴ is SO₂, | R¹ is CF₃, | R² is Cl, | R³ is H, | R⁴ is Bu | and |
| 1061 | Q⁴ is SO₂, | R¹ is CF₃, | R² is Cl, | R³ is H, | R⁴ is CF₃ | and |
| 1062 | Q⁴ is SO₂, | R¹ is CF₃, | R² is Cl, | R³ is H, | R⁴ is CHF₂ | and |
| 1063 | Q⁴ is CH₂, | R¹ is CF₃, | R² is H, | R³ is H, | R⁴ is CH₃ | and |
| 1064 | Q⁴ is CH₂, | R¹ is CF₃, | R² is H, | R³ is H, | R⁴ is Et | and |
| 1065 | Q⁴ is CH₂, | R¹ is CF₃, | R² is H, | R³ is H, | R⁴ is c-Pr | and |
| 1066 | Q⁴ is CH₂, | R¹ is CF₃, | R² is H, | R³ is H, | R⁴ is Pr | and |
| 1067 | Q⁴ is CH₂, | R¹ is CF₃, | R² is H, | R³ is H, | R⁴ is i-Pr | and |
| 1068 | Q⁴ is CH₂, | R¹ is CF₃, | R² is H, | R³ is H, | R⁴ is Bu | and |
| 1069 | Q⁴ is CH₂, | R¹ is CF₃, | R² is H, | R³ is H, | R⁴ is CF₃ | and |
| 1070 | Q⁴ is CH₂, | R¹ is CF₃, | R² is H, | R³ is H, | R⁴ is CHF₂ | and |
| 1071 | Q⁴ is CH₂, | R¹ is CF₃, | R² is CH₃, | R³ is H, | R⁴ is CH₃ | and |
| 1072 | Q⁴ is CH₂, | R¹ is CF₃, | R² is CH₃, | R³ is H, | R⁴ is Et | and |
| 1073 | Q⁴ is CH₂, | R¹ is CF₃, | R² is CH₃, | R³ is H, | R⁴ is c-Pr | and |
| 1074 | Q⁴ is CH₂, | R¹ is CF₃, | R² is CH₃, | R³ is H, | R⁴ is Pr | and |
| 1075 | Q⁴ is CH₂, | R¹ is CF₃, | R² is CH₃, | R³ is H, | R⁴ is i-Pr | and |
| 1076 | Q⁴ is CH₂, | R¹ is CF₃, | R² is CH₃, | R³ is H, | R⁴ is Bu | and |
| 1077 | Q⁴ is CH₂, | R¹ is CF₃, | R² is CH₃, | R³ is H, | R⁴ is CF₃ | and |
| 1078 | Q⁴ is CH₂, | R¹ is CF₃, | R² is CH₃, | R³ is H, | R⁴ is CHF₂ | and |
| 1079 | Q⁴ is CH₂, | R¹ is CF₃, | R² is Cl, | R³ is H, | R⁴ is CH₃ | and |
| 1080 | Q⁴ is CH₂, | R¹ is CF₃, | R² is Cl, | R³ is H, | R⁴ is Et | and |
| 1081 | Q⁴ is CH₂, | R¹ is CF₃, | R² is Cl, | R³ is H, | R⁴ is c-Pr | and |
| 1082 | Q⁴ is CH₂, | R¹ is CF₃, | R² is Cl, | R³ is H, | R⁴ is Pr | and |
| 1083 | Q⁴ is CH₂, | R¹ is CF₃, | R² is Cl, | R³ is H, | R⁴ is i-Pr | and |
| 1084 | Q⁴ is CH₂, | R¹ is CF₃, | R² is Cl, | R³ is H, | R⁴ is Bu | and |
| 1085 | Q⁴ is CH₂, | R¹ is CF₃, | R² is Cl, | R³ is H, | R⁴ is CF₃ | and |
| 1086 | Q⁴ is CH₂, | R¹ is CF₃, | R² is Cl, | R³ is H, | R⁴ is CHF₂ | and |
| 1087 | Q⁴ is CH₂, | R¹ is CF₃, | R² is H, | R³ is H, | R⁴ is CH₃ | and |
| 1088 | Q⁴ is CH₂, | R¹ is CF₃, | R² is H, | R³ is H, | R⁴ is Et | and |
| 1089 | Q⁴ is CH₂, | R¹ is CF₃, | R² is H, | R³ is H, | R⁴ is c-Pr | and |
| 1090 | Q⁴ is CH₂, | R¹ is CF₃, | R² is H, | R³ is H, | R⁴ is Pr | and |
| 1091 | Q⁴ is CH₂, | R¹ is CF₃, | R² is H, | R³ is H, | R⁴ is i-Pr | and |
| 1092 | Q⁴ is CH₂, | R¹ is CF₃, | R² is H, | R³ is H, | R⁴ is Bu | and |
| 1093 | Q⁴ is CH₂, | R¹ is CF₃, | R² is H, | R³ is H, | R⁴ is CF₃ | and |
| 1094 | Q⁴ is CH₂, | R¹ is CF₃, | R² is H, | R³ is H, | R⁴ is CHF₂ | and |
| 1095 | Q⁴ is CH₂, | R¹ is CF₃, | R² is CH₃, | R³ is H, | R⁴ is CH₃ | and |
| 1096 | Q⁴ is CH₂, | R¹ is CF₃, | R² is CH₃, | R³ is H, | R⁴ is Et | and |
| 1097 | Q⁴ is CH₂, | R¹ is CF₃, | R² is CH₃, | R³ is H, | R⁴ is c-Pr | and |
| 1098 | Q⁴ is CH₂, | R¹ is CF₃, | R² is CH₃, | R³ is H, | R⁴ is Pr | and |
| 1099 | Q⁴ is CH₂, | R¹ is CF₃, | R² is CH₃, | R³ is H, | R⁴ is i-Pr | and |
| 1100 | Q⁴ is CH₂, | R¹ is CF₃, | R² is CH₃, | R³ is H, | R⁴ is Bu | and |
| 1101 | Q⁴ is CH₂, | R¹ is CF₃, | R² is CH₃, | R³ is H, | R⁴ is CF₃ | and |
| 1102 | Q⁴ is CH₂, | R¹ is CF₃, | R² is CH₃, | R³ is H, | R⁴ is CHF₂ | and |
| 1103 | Q⁴ is CH₂, | R¹ is CF₃, | R² is Cl, | R³ is H, | R⁴ is CH₃ | and |
| 1104 | Q⁴ is CH₂, | R¹ is CF₃, | R² is Cl, | R³ is H, | R⁴ is Et | and |
| 1105 | Q⁴ is CH₂, | R¹ is CF₃, | R² is Cl, | R³ is H, | R⁴ is c-Pr | and |
| 1106 | Q⁴ is CH₂, | R¹ is CF₃, | R² is Cl, | R³ is H, | R⁴ is Pr | and |
| 1107 | Q⁴ is CH₂, | R¹ is CF₃, | R² is Cl, | R³ is H, | R⁴ is i-Pr | and |
| 1108 | Q⁴ is CH₂, | R¹ is CF₃, | R² is Cl, | R³ is H, | R⁴ is Bu | and |
| 1109 | Q⁴ is CH₂, | R¹ is CF₃, | R² is Cl, | R³ is H, | R⁴ is CF₃ | and |
| 1110 | Q⁴ is CH₂, | R¹ is CF₃, | R² is Cl, | R³ is H, | R⁴ is CHF₂ | and |
| 1111 | Q⁴ is CO, | R¹ is CF₃, | R² is H, | R³ is H, | R⁴ is CH₃ | and |
| 1112 | Q⁴ is CO, | R¹ is CF₃, | R² is H, | R³ is H, | R⁴ is Et | and |
| 1113 | Q⁴ is CO, | R¹ is CF₃, | R² is H, | R³ is H, | R⁴ is c-Pr | and |
| 1114 | Q⁴ is CO, | R¹ is CF₃, | R² is H, | R³ is H, | R⁴ is Pr | and |
| 1115 | Q⁴ is CO, | R¹ is CF₃, | R² is H, | R³ is H, | R⁴ is i-Pr | and |
| 1116 | Q⁴ is CO, | R¹ is CF₃, | R² is H, | R³ is H, | R⁴ is Bu | and |
| 1117 | Q⁴ is CO, | R¹ is CF₃, | R² is H, | R³ is H, | R⁴ is CF₃ | and |
| 1118 | Q⁴ is CO, | R¹ is CF₃, | R² is H, | R³ is H, | R⁴ is CHF₂ | and |
| 1119 | Q⁴ is CO, | R¹ is CF₃, | R² is CH₃, | R³ is H, | R⁴ is CH₃ | and |
| 1120 | Q⁴ is CO, | R¹ is CF₃, | R² is CH₃, | R³ is H, | R⁴ is Et | and |
| 1121 | Q⁴ is CO, | R¹ is CF₃, | R² is CH₃, | R³ is H, | R⁴ is c-Pr | and |
| 1122 | Q⁴ is CO, | R¹ is CF₃, | R² is CH₃, | R³ is H, | R⁴ is Pr | and |
| 1123 | Q⁴ is CO, | R¹ is CF₃, | R² is CH₃, | R³ is H, | R⁴ is i-Pr | and |
| 1124 | Q⁴ is CO, | R¹ is CF₃, | R² is CH₃, | R³ is H, | R⁴ is Bu | and |
| 1125 | Q⁴ is CO, | R¹ is CF₃, | R² is CH₃, | R³ is H, | R⁴ is CF₃ | and |
| 1126 | Q⁴ is CO, | R¹ is CF₃, | R² is CH₃, | R³ is H, | R⁴ is CHF₂ | and |
| 1127 | Q⁴ is CO, | R¹ is CF₃, | R² is Cl, | R³ is H | R⁴ is CH₃ | and |
| 1128 | Q⁴ is CO, | R¹ is CF₃, | R² is Cl, | R³ is H, | R⁴ is Et | and |
| 1129 | Q⁴ is CO, | R¹ is CF₃, | R² is Cl, | R³ is H, | R⁴ is c-Pr | and |
| 1130 | Q⁴ is CO, | R¹ is CF₃, | R² is Cl, | R³ is H, | R⁴ is Pr | and |
| 1131 | Q⁴ is CO, | R¹ is CF₃, | R² is Cl, | R³ is H, | R⁴ is i-Pr | and |
| 1132 | Q⁴ is CO, | R¹ is CF₃, | R² is Cl, | R³ is H, | R⁴ is Bu | and |
| 1133 | Q⁴ is CO, | R¹ is CF₃, | R² is Cl, | R³ is H, | R⁴ is CF₃ | and |
| 1134 | Q⁴ is CO, | R¹ is CF₃, | R² is Cl, | R³ is H, | R⁴ is CHF₂ | and |
| 1135 | Q⁴ is O, | R¹ is CF₃, | R² is H, | R³ is CH₃, | R⁴ is CH₃ | and |
| 1136 | Q⁴ is O, | R¹ is CF₃, | R² is H, | R³ is COCH₃, | R⁴ is CH₃ | and |
| 1137 | Q⁴ is O, | R¹ is CF₃, | R² is H, | R³ is CO₂CH₃, | R⁴ is CH₃ | and |
| 1138 | Q⁴ is O, | R¹ is CF₃, | R² is H, | R³ is COCF₃, | R⁴ is CH₃ | and |
| 1139 | Q⁴ is O, | R¹ is CF₃, | R² is CHF₂, | R³ is H, | R⁴ is CH₃ | and |
| 1140 | Q⁴ is O, | R¹ is CF₃, | R² is F, | R³ is H, | R⁴ is CH₃ | and |
| 1141 | Q⁴ is O, | R¹ is CF₃, | R² is Et, | R³ is H, | R⁴ is CH₃ | and |
| 1142 | Q⁴ is O, | R¹ is CF₃, | R² is CF₃, | R³ is H, | R⁴ is CH₃ | and |
| 1143 | Q⁴ is O, | R¹ is CF₃, | R² is CCH, | R³ is H, | R⁴ is CH₃ | and |
| 1144 | Q⁴ is O, | R¹ is CF₃, | R² is H, | R³ is CH₃, | R⁴ is CH₃ | and |
| 1145 | Q⁴ is O, | R¹ is CF₃, | R² is H, | R³ is COCH₃, | R⁴ is CH₃ | and |
| 1146 | Q⁴ is O, | R¹ is CF₃, | R² is H, | R³ is CO₂CH₃, | R⁴ is CH₃ | and |
| 1147 | Q⁴ is O, | R¹ is CF₃, | R² is CHF₂ | R³ is COCF₃, | R⁴ is CH₃ | and |
| 1148 | Q⁴ is O, | R¹ is CF₃, | R² is F, | R³ is H, | R⁴ is CH₃ | and |
| 1149 | Q⁴ is O, | R¹ is CF₃, | R² is F, | R³ is H, | R⁴ is CH₃ | and |
| 1150 | Q⁴ is O, | R¹ is CF₃, | R² is Et, | R³ is H, | R⁴ is CH₃ | and |
| 1151 | Q⁴ is O, | R¹ is CF₃, | R² is CF₃, | R³ is H, | R⁴ is CH₃ | and |
| 1152 | Q⁴ is O, | R¹ is CF₃, | R² is CCH, | R³ is H, | R⁴ is CH₃ | and |

TABLE 1153

| 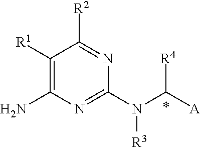 | |
|---|---|
| wherein A is selected from | |
| 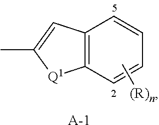 | 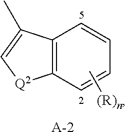 |
| 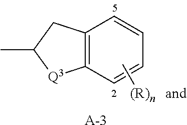 | 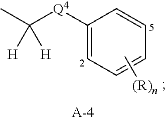 |

A is A-1, Q¹ is CH═CH, R³ is H, R⁴ is CH₃, (R)ₙ is H and

| R¹ | R² |
|---|---|
| CF₃ | Et |
| CF₃ | Pr |
| CF₃ | i-Pr |
| CF₃ | t-Bu |
| CF₃ | CF₃ |
| CF₃ | CHF₂ |
| CF₃ | CH₂F |
| CF₃ | CHFCH₃ |
| CF₃ | CF(CH₃)₂ |
| CF₃ | CF₂CH₃ |
| CF₃ | CF₂CF₃ |
| CF₃ | CN |
| CF₃ | CH═CH₂ |
| CF₃ | C≡CH |
| CF₃ | C≡CCH₃ |
| CF₃ | CONH₂ |
| CF₃ | CONHCH₃ |
| CF₃ | CON(CH₃)₂ |
| CF₃ | Ph |
| CF₃ | CH₂Ph |
| S(O)₂CH₃ | H |
| S(O)₂CH₃ | CH₃ |
| S(O)₂CH₃ | Et |
| S(O)₂CH₃ | Pr |
| S(O)₂CH₃ | i-Pr |
| S(O)₂CH₃ | t-Bu |
| S(O)₂CH₃ | CF₃ |
| S(O)₂CH₃ | CHF2 |
| S(O)₂CH₃ | c-Pr |
| S(O)₂CH₃ | OCH₃ |
| S(O)₂CH₃ | N(CH₃)₂ |
| S(O)₂CH₃ | COCH₃ |
| S(O)₂CH₃ | Cl |
| S(O)₂CH₃ | F |
| S(O)₂CH₃ | Br |
| S(O)₂CH₃ | I |
| S(O)₂CH₃ | CN |
| S(O)₂CH₃ | CH═CH₂ |
| S(O)₂CH₃ | C≡CH |
| S(O)₂CH₃ | C≡CCH₃ |
| S(O)₂CH₃ | Ph |
| C≡CH | H |
| C≡CH | CH₃ |
| C≡CH | i-Pr |
| C≡CH | t-Bu |
| C≡CH | CF₃ |
| C≡CH | CHF₂ |
| OCH₃ | H |
| CHF₂ | Ph |
| CHF₂ | CH₂Ph |
| CN | Et |
| CN | Pr |
| CN | i-Pr |
| CN | t-Bu |
| CN | CF₃ |
| CN | CHF₂ |
| CN | CH₂F |
| CN | CHFCH₃ |
| CN | CF(CH₃)₂ |
| CN | CF₂CH₃ |
| CN | CF₂CF₃ |
| CN | CF₂CHF₂ |
| CN | CF₂CH₂F |
| CN | CH₂CF₃ |
| CN | CH₂CF₂H |
| CN | CH₂CH₂F |
| CN | CF₂CF₂CF₃ |
| CN | CH(OH)CH₃ |
| CN | C(OH)(CH₃)₂ |
| CN | c-Pr |
| CN | c-Bu |
| CN | c-Pn |
| CN | c-Hx |
| CN | CH₂OCH₃ |
| CN | CH₂OCF₃ |
| CN | OCH₃ |
| CN | N(CH₃)₂ |
| CN | COCH₃ |
| CN | CO |
| CN | CO₂CH₃ |
| CN | F |
| CN | Br |
| CN | I |
| CN | CN |
| CN | CH═CH₂ |
| S(O)CH₃ | i-Pr |
| S(O)CH₃ | t-Bu |
| S(O)CH₃ | CF₃ |
| S(O)CH₃ | CHF₂ |
| S(O)CH₃ | c-Pr |
| S(O)CH₃ | OCH₃ |
| S(O)CH₃ | N(CH₃)₂ |
| S(O)CH₃ | COCH₃ |
| S(O)CH₃ | Cl |
| S(O)CH₃ | F |
| S(O)CH₃ | Br |
| S(O)CH₃ | I |
| S(O)CH₃ | CN |
| S(O)CH₃ | CH═CH₂ |
| S(O)CH₃ | C≡CH |
| S(O)CH₃ | C≡CCH₃ |
| S(O)CH₃ | Ph |
| F | H |
| CF₃ | CF₂CHF₂ |
| CF₃ | CF₂CH₂F |
| CF₃ | CH₂CF₃ |
| CF₃ | CH₂CF₂H |
| CF₃ | CH₂CH₂F |
| CF₃ | CF₂CF₂CF₃ |
| CF₃ | CH(OH)CH₃ |
| CF₃ | C(OH)(CH₃)₂ |
| CF₃ | c-Pr |
| CF₃ | 1-F-c-Pr |
| CF₃ | 2,2-di-F-c-Pr |
| CF₃ | c-Bu |
| OCH₃ | CH₃ |
| OCH₃ | i-Pr |
| OCH₃ | t-Bu |
| OCH₃ | CF₃ |
| OCH₃ | CHF₂ |
| CH₃ | H |
| CH₃ | CH₃ |
| CH₃ | i-Pr |
| CH₃ | t-Bu |
| CH₃ | CF₃ |
| CH₃ | CHF₂ |
| COCH₃ | H |
| COCH₃ | CH₃ |
| COCH₃ | Et |
| COCH₃ | Pr |
| COCH₃ | i-Pr |
| COCH₃ | t-Bu |
| COCH₃ | CF₃ |
| COCH₃ | CHF₂ |
| COCH₃ | c-Pr |
| COCH₃ | OCH₃ |
| COCH₃ | N(CH₃)₂ |
| COCH₃ | COCH₃ |
| COCH₃ | Cl |
| COCH₃ | F |
| COCH₃ | Br |
| COCH₃ | I |
| COCH₃ | CN |
| COCH₃ | CH═CH₂ |
| COCH₃ | C≡CH |
| COCH₃ | C≡CCH₃ |
| COCH₃ | Ph |
| CHF₂ | Et |
| CHF₂ | Pr |
| CHF₂ | i-Pr |
| CHF₂ | t-Bu |
| CHF₂ | CF₃ |
| CN | C≡CH |
| CN | C≡CCH₃ |
| CN | CONH₂ |
| CN | CONHCH₃ |
| CN | CON(CH₃)₂ |
| CN | Ph |
| CN | CH₂Ph |
| C≡CCH₃ | H |
| C≡CCH₃ | CH₃ |
| C≡CCH₃ | i-Pr |
| C≡CCH₃ | t-Bu |
| C≡CCH₃ | CF₃ |
| C≡CCH₃ | CHF₂ |
| NO₂ | H |
| NO₂ | CH₃ |
| NO₂ | i-Pr |
| NO₂ | t-Bu |
| NO₂ | CF₃ |
| NO₂ | CHF₂ |
| CHO | H |
| CHO | CH₃ |
| CHO | i-Pr |
| CHO | t-Bu |
| CHO | CF₃ |
| CHO | CHF₂ |
| CF₂CF₃ | H |
| CF₂CF₃ | CH₃ |
| CF₂CF₃ | Et |
| CF₂CF₃ | Pr |
| CF₂CF₃ | i-Pr |
| CF₂CF₃ | t-Bu |
| CF₂CF₃ | CF₃ |
| CF₂CF₃ | CHF₂ |
| CF₂CF₃ | CH₂F |
| CF₂CF₃ | CHFCH₃ |
| CF₂CF₃ | CF(CH₃)₂ |
| CF₂CF₃ | CF₂CH₃ |
| F | CH₃ |
| F | i-Pr |
| F | t-Bu |
| F | CF₃ |
| F | CHF₂ |
| Cl | H |
| Cl | CH₃ |
| Cl | i-Pr |
| Cl | t-Bu |
| Cl | CF₃ |
| Cl | CHF₂ |
| Br | H |
| Br | CH₃ |
| Br | i-Pr |
| Br | t-Bu |
| Br | CF₃ |
| Br | CHF₂ |
| I | H |
| CF₃ | c-Pn |
| CF₃ | c-Hx |
| CF₃ | CH₂OCH₃ |
| CF₃ | CH₂OCF₃ |
| CF₃ | OCH₃ |
| CF₃ | N(CH₃)₂ |
| CF₃ | COCH₃ |
| CF₃ | COCF₃ |
| CF₃ | CO₂CH₃ |
| CF₃ | F |
| CF₃ | Br |
| CF₃ | I |
| CHF₂ | CHF₂ |
| CHF₂ | CH₂F |
| CHF₂ | CHFCH₃ |
| CHF₂ | CF(CH₃)₂ |
| CHF₂ | CF₂CH₃ |
| CHF₂ | CF₂CF₃ |
| CHF₂ | CF₂CHF₂ |
| CHF₂ | CF₂CH₂F |
| CHF₂ | CH₂CF₃ |
| CHF₂ | CH₂CF₂H |
| CHF₂ | CH₂CH₂F |
| CHF₂ | CF₂CF₂CF₃ |
| CHF₂ | CH(OH)CH₃ |
| CHF₂ | C(OH)(CH₃)₂ |
| CHF₂ | c-Pr |
| CHF₂ | 1-F-c-Pr |
| CHF₂ | 2,2-di-F-c-Pr |
| CHF₂ | c-Bu |
| CHF₂ | c-Pn |
| CHF₂ | c-Hx |
| CHF₂ | CH₂OCH₃ |
| CHF₂ | CH₂OCF₃ |
| CHF₂ | OCH₃ |
| CHF₂ | N(CH₃)₂ |
| CHF₂ | COCH₃ |
| CHF₂ | COCF₃ |
| CHF₂ | CO₂CH₃ |
| CHF₂ | F |
| CHF₂ | Br |
| CHF₂ | I |
| CHF₂ | CN |
| CHF₂ | CH═CH₂ |
| CHF₂ | C≡CH |
| CHF₂ | C≡CCH₃ |
| CHF₂ | CONH₂ |
| CHF₂ | CONHCH₃ |
| CHF₂ | CON(CH₃)₂ |
| CF₂CF₃ | CF₂CF₃ |
| CF₂CF₃ | CF₂CHF₂ |
| CF₂CF₃ | CF₂CH₂F |
| CF₂CF₃ | CH₂CF₃ |
| CF₂CF₃ | CH₂CF₂H |
| CF₂CF₃ | CH₂CH₂F |
| CF₂CF₃ | CF₂CF₂CF₃ |
| CF₂CF₃ | CH(OH)CH₃ |
| CF₂CF₃ | C(OH)(CH₃)₂ |
| CF₂CF₃ | c-Pr |
| CF₂CF₃ | c-Bu |
| CF₂CF₃ | c-Pn |
| CF₂CF₃ | c-Hx |
| CF₂CF₃ | CH₂OCH₃ |
| CF₂CF₃ | CH₂OCF₃ |
| CF₂CF₃ | OCH₃ |
| CF₂CF₃ | N(CH₃)₂ |
| CF₂CF₃ | COCH₃ |
| CF₂CF₃ | COCF₃ |
| CF₂CF₃ | CO₂CH₃ |
| CF₂CF₃ | Cl |
| CF₂CF₃ | F |
| CF₂CF₃ | Br |
| CF₂CF₃ | I |
| CF₂CF₃ | CN |
| CF₂CF₃ | CH═CH₂ |
| CF₂CF₃ | C≡CH |
| CF₂CF₃ | C≡CCH₃ |
| CF₂CF₃ | CONH₂ |
| CF₂CF₃ | CONHCH₃ |
| CF₂CF₃ | CON(CH₃)₂ |
| CF₂CF₃ | Ph |
| CF₂CF₃ | CH₂Ph |
| S(O)CH₃ | H |
| S(O)CH₃ | CH₃ |
| S(O)CH₃ | Et |
| S(O)CH₃ | Pr |
| I | CH₃ |
| I | i-Pr |
| I | t-Bu |
| I | CF₃ |
| I | CHF₂ |
| H | H |
| H | CH₃ |
| H | CF₃ |
| H | CHF₂ |
| H | Et |
| CO₂CH₃ | H |
| CO₂CH₃ | CH₃ |
| CO₂CH₃ | i-Pr |
| CO₂CH₃ | t-Bu |
| CO₂CH₃ | CF₃ |
| CO₂CH₃ | CHF₂ |

The present disclosure also includes Tables 1154 through 1218, each of which is constructed the same as Table 1153 above, except that the Header Row in Table 1153 (i.e. A is A-1, QI is CH═CH, R³ is H, R⁴ is CH₃ and (R)ₙ is H ((i.e. n=0)) is replaced with the respective Header Row shown below in Tables 1154 through 1218. For example, the first entry in Table 1154 is a compound of Formula 1 wherein A is A-1, QI is CH═CH, R³ is H, R⁴ is CH₃, and (R)ₙ is 6-F. Tables 1155 through 1218 are constructed similarly.

| Table | Header Row | | | | | |
|---|---|---|---|---|---|---|
| 1145 | A is A-1 | Q¹ is CH═CH | R³ is H | R⁵ is CH₃ | (R)ₙ is 3-F | and |
| 1155 | A is A-1, | Q¹ is CH═CH, | R³ is H, | R⁴ is CH₃ | (R)ₙ is 4-F | and |
| 1156 | A is A-1, | Q¹ is CH═CH, | R³ is H, | R⁴ is CH₃ | (R)ₙ is 3-Me | and |
| 1157 | A is A-1, | Q¹ is CH═CH, | R³ is H, | R⁴ is CH₃ | (R)ₙ is 3-CF₃ | and |
| 1158 | A is A-1, | Q¹ is CH═CH, | R³ is H, | R⁴ is CH₃ | (R)ₙ is 3,4-di-CH₃ | and |
| 1159 | A is A-1, | Q¹ is CH═CH, | R³ is H, | R⁴ is Et | (R)ₙ is H (n = 0) | and |
| 1160 | A is A-1, | Q¹ is CH═CH, | R³ is H, | R⁴ is Et | (R)ₙ is 3-F | and |
| 1161 | A is A-1, | Q¹ is CH═CH, | R³ is H, | R⁴ is Et | (R)ₙ is 4-F | and |
| 1162 | A is A-1, | Q¹ is CH═CH, | R³ is H, | R⁴ is Et | (R)ₙ is 3-Me | and |
| 1163 | A is A-1, | Q¹ is CH═CH, | R³ is H, | R⁴ is Et | (R)ₙ is 3-CF₃ | and |
| 1164 | A is A-1, | Q¹ is CH═CH, | R³ is H, | R⁴ is Et | (R)ₙ is 3,4-di-CH₃ | and |
| 1165 | A is A-1, | Q¹ is O, | R³ is H, | R⁴ is CH₃ | (R)ₙ is H (n = 0) | and |
| 1166 | A is A-1, | Q¹ is O, | R³ is H, | R⁴ is CH₃ | (R)ₙ is 3-F | and |
| 1167 | A is A-1, | Q¹ is O, | R³ is H, | R⁴ is CH₃ | (R)ₙ is 4-F | and |
| 1168 | A is A-1, | Q¹ is O, | R³ is H, | R⁴ is CH₃ | (R)ₙ is 3-Me | and |
| 1169 | A is A-1, | Q¹ is O, | R³ is H, | R⁴ is CH₃ | (R)ₙ is 3-CF₃ | and |
| 1170 | A is A-1, | Q¹ is O, | R³ is H, | R⁴ is CH₃ | (R)ₙ is 3,4-di-CH₃ | and |
| 1171 | A is A-1, | Q¹ is O, | R³ is H, | R⁴ is Et | (R)ₙ is H (n = 0) | and |
| 1172 | A is A-1, | Q¹ is O, | R³ is H, | R⁴ is Et | (R)ₙ is 3-F | and |
| 1173 | A is A-1, | Q¹ is O, | R³ is H, | R⁴ is Et | (R)ₙ is 4-F | and |
| 1174 | A is A-1, | Q¹ is O, | R³ is H, | R⁴ is Et | (R)ₙ is 3-Me | and |
| 1175 | A is A-1, | Q¹ is O, | R³ is H, | R⁴ is Et | (R)ₙ is 3-CF₃ | and |
| 1176 | A is A-1, | Q¹ is O, | R³ is H, | R⁴ is Et | (R)ₙ is 3,4-di-CH₃ | and |
| 1177 | A is A-1, | Q¹ is S, | R³ is H, | R⁴ is CH₃ | (R)ₙ is H (n = 0) | and |
| 1178 | A is A-1, | Q¹ is S, | R³ is H, | R⁴ is CH₃ | (R)ₙ is 3-F | and |
| 1179 | A is A-1, | Q¹ is S, | R³ is H, | R⁴ is CH₃ | (R)ₙ is 4-F | and |
| 1180 | A is A-1, | Q¹ is S, | R³ is H, | R⁴ is CH₃ | (R)ₙ is 3-Me | and |
| 1181 | A is A-1, | Q¹ is S, | R³ is H, | R⁴ is CH₃ | (R)ₙ is 3-CF₃ | and |
| 1182 | A is A-1, | Q¹ is S, | R³ is H, | R⁴ is CH₃ | (R)ₙ is 3,4-di-CH₃ | and |
| 1183 | A is A-1, | Q¹ is S, | R³ is H, | R⁴ is Et | (R)ₙ is H (n = 0) | and |
| 1184 | A is A-1, | Q¹ is S, | R³ is H, | R⁴ is Et | (R)ₙ is 3-F | and |
| 1185 | A is A-1, | Q¹ is S, | R³ is H, | R⁴ is Et | (R)ₙ is 4-F | and |
| 1186 | A is A-1, | Q¹ is S, | R³ is H, | R⁴ is Et | (R)ₙ is 3-Me | and |
| 1187 | A is A-1, | Q¹ is S, | R³ is H, | R⁴ is Et | (R)ₙ is 3-CF₃ | and |
| 1188 | A is A-1, | Q¹ is S, | R³ is H, | R⁴ is Et | (R)ₙ is 34-di-CH₃ | and |
| 1189 | A is A-2, | Q² is CH═CH, | R³ is H, | R⁴ is CH₃ | (R)ₙ is H (n = 0) | and |
| 1190 | A is A-2, | Q² is CH═CH, | R³ is H, | R⁴ is CH₃ | (R)ₙ is 3-F | and |
| 1191 | A is A-2, | Q² is CH═CH, | R³ is H, | R⁴ is CH₃ | (R)ₙ is 4-F | and |
| 1192 | A is A-2, | Q² is CH═CH, | R³ is H, | R⁴ is CH₃ | (R)ₙ is 3-Me | and |
| 1193 | A is A-2, | Q² is CH═CH, | R³ is H, | R⁴ is CH₃ | (R)ₙ is 3-CF₃ | and |
| 1194 | A is A-2, | Q² is CH═CH, | R³ is H, | R⁴ is CH₃ | (R)ₙ is 34-di-CH₃ | and |
| 1195 | A is A-3, | Q³ is O, | R³ is H, | R⁴ is CH₃ | (R)ₙ is H (n = 0) | and |
| 1196 | A is A-3, | Q³ is O, | R³ is H, | R⁴ is CH₃ | (R)ₙ is 3-F | and |
| 1197 | A is A-3, | Q³ is O, | R³ is H, | R⁴ is CH₃ | (R)ₙ is 4-F | and |
| 1198 | A is A-3, | Q³ is O, | R³ is H, | R⁴ is CH₃ | (R)ₙ is 3-Me | and |
| 1199 | A is A-3, | Q³ is O, | R³ is H, | R⁴ is CH₃ | (R)ₙ is 3-CF₃ | and |
| 1200 | A is A-3, | Q³ is O, | R³ is H, | R⁴ is CH₃ | (R)ₙ is 3,4-di-CH₃ | and |
| 1201 | A is A-3, | Q³ is CH₂, | R³ is H, | R⁴ is CH₃ | (R)ₙ is H (n = 0) | and |
| 1202 | A is A-3, | Q³ is CH₂, | R³ is H, | R⁴ is CH₃ | (R)ₙ is 3-F | and |
| 1203 | A is A-3, | Q³ is CH₂, | R³ is H, | R⁴ is CH₃ | (R)ₙ is 4-F | and |
| 1204 | A is A-3, | Q³ is CH₂, | R³ is H, | R⁴ is CH₃ | (R)ₙ is 3-Me | and |
| 1205 | A is A-3, | Q³ is CH₂, | R³ is H, | R⁴ is CH₃ | (R)ₙ is 3-CF₃ | and |
| 1206 | A is A-3, | Q³ is CH₂, | R³ is H, | R⁴ is CH₃ | (R)ₙ is 3,4-di-CH₃ | and |
| 1207 | A is A-4, | Q⁴ is O, | R³ is H, | R⁴ is CH₃ | (R)ₙ is H (n = 0) | and |
| 1208 | A is A-4, | Q⁴ is O, | R³ is H, | R⁴ is CH₃ | (R)ₙ is 3-F | and |
| 1209 | A is A-4, | Q⁴ is O, | R³ is H, | R⁴ is CH₃ | (R)ₙ is 4-F | and |
| 1210 | A is A-4, | Q⁴ is O, | R³ is H, | R⁴ is CH₃ | (R)ₙ is 3-Me | and |
| 1211 | A is A-4, | Q⁴ is O, | R³ is H, | R⁴ is CH₃ | (R)ₙ is 3-CF₃ | and |
| 1212 | A is A-4, | Q⁴ is O, | R³ is H, | R⁴ is CH₃ | (R)ₙ is 3,4-di-CH₃ | and |
| 1213 | A is A-4, | Q⁴ is CH₂, | R³ is H, | R⁴ is CH₃ | (R)ₙ is H (n = 0) | and |
| 1214 | A is A-4, | Q⁴ is CH₂, | R³ is H, | R⁴ is CH₃ | (R)ₙ is 3-F | and |
| 1215 | A is A-4, | Q⁴ is CH₂, | R³ is H, | R⁴ is CH₃ | (R)ₙ is 4-F | and |
| 1216 | A is A-4, | Q⁴ is CH₂, | R³ is H, | R⁴ is CH₃ | (R)ₙ is 3-Me | and |
| 1217 | A is A-4, | Q⁴ is CH₂, | R³ is H, | R⁴ is CH₃ | (R)ₙ is 3-CF₃ | and |
| 1218 | A is A-4, | Q⁴ is CH₂, | R³ is H, | R⁴ is CH₃ | (R)ₙ is 3,4-di-CH₃ | and |

A compound of this invention will generally be used as a herbicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively, the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions and Solutions (including | | | |
| Emulsifiable Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Mixtures of nonionic and anionic surfactants or mixtures of nonionic and cationic surfactants are also useful for the present compositions. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight and all formulations are prepared in conventional ways. Compound numbers refer to compounds in Index Tables A and B. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 1 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 1 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 1 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 1 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 1 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Suspension Concentrate

| | |
|---|---|
| Compound 1 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example H

Emulsion in Water

| | |
|---|---|
| Compound 1 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example I

Oil Dispersion

| | |
|---|---|
| Compound 1 | 25% |
| polyoxyethylene sorbitol hexaoleate | 15% |
| organically modified bentonite clay | 2.5% |
| fatty acid methyl ester | 57.5% |

Additional Example Formulations include Examples A through I above wherein “Compound 1” is replaced in each of the Examples A through I with the respective compounds from Index Table A as shown below.

Compound No.

Compound 3
Compound 4
Compound 6
Compound 8
Compound 9
Compound 13
Compound 14
Compound 15
Compound 16
Compound 18
Compound 21
Compound 22
Compound 24
Compound 25
Compound 27

Test results indicate that the compounds of the present invention are highly active preemergent and/or postemergent herbicides and/or plant growth regulants. The compounds of the inention generally show highest activity for postemergence weed control (i.e. applied after weed seedlings emerge from the soil) and preemergence weed control (i.e. applied before weed seedlings emerge from the soil). Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, because of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but is not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation.

A herbicidally effective amount of a compound of this invention is determined by factors that include but are not limited to: formulation selected, method of application, amount and type of vegetation present, growing conditions, etc. In general, a herbicidally effective amount of a compound of this invention is about 0.001 to 20 kg/ha with a preferred range of about 0.004 to 1 kg/ha. One skilled in the art can easily determine the herbicidally effective amount necessary for the desired level of weed control.

In one common embodiment, a compound of the invention is applied, typically in a formulated composition, to a locus comprising desired vegetation (e.g., crops) and undesired vegetation (i.e. weeds), both of which may be seeds, seedlings and/or larger plants, in contact with a growth medium (e.g., soil). In this locus, a composition comprising a compound of the invention can be directly applied to a plant or a part thereof, particularly of the undesired vegetation, and/or to the growth medium in contact with the plant.

Plant varieties and cultivars of the desired vegetation in the locus treated with a compound of the invention can be obtained by conventional propagation and breeding methods or by genetic engineering methods. Genetically modified plants (transgenic plants) are those in which a heterologous gene (transgene) has been stably integrated into the plant's genome. A transgene that is defined by its location in the plant genome is called a transformation or transgenic event.

Genetically modified plant cultivars in the locus that can be treated according to the invention include those that are resistant to one or more biotic stresses (pests such as nematodes, insects, mites, fungi, etc.) or abiotic stresses (drought, cold temperature, soil salinity, etc.), or that contain other desirable characteristics. Plants can be genetically modified to exhibit traits of, for example, herbicide tolerance, insect-resistance, modified oil profiles or drought tolerance.

Although most typically, compounds of the invention are used to control undesired vegetation, contact of desired vegetation in the treated locus with compounds of the invention may result in super-additive or synergistic effects with genetic traits in the desired vegetation, including traits incorporated through genetic modification. For example, resistance to phytophagous insect pests or plant diseases, tolerance to biotic/abiotic stresses or storage stability may be greater than expected from the genetic traits in the desired vegetation.

Compounds of this invention can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the invention with other herbicides can broaden the spectrum of activity against additional weed species, and suppress the proliferation of any resistant biotypes. Thus, the present invention also pertains to a composition comprising a compound of Formula 1 (in a herbicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined before application (e.g., in a spray tank) or, alternatively, applied in succession.

A mixture of one or more of the following herbicides with a compound of this invention may be particularly useful for weed control: acetochlor, acifluorfen and its sodium salt, aclonifen, acrolein (2-propenal), alachlor, alloxydim, ametryn, amicarbazone, amidosulfuron, aminocyclopyrachlor and its esters (e.g., methyl, ethyl) and salts (e.g., sodium, potassium), aminopyralid, amitrole, ammonium sulfamate, anilofos, asulam, atrazine, azimsulfuron, beflubutamid, benazolin, benazolin-ethyl, bencarbazone, benfluralin, benfuresate, bensulfuron-methyl, bensulide, bentazone, benzobicyclon, benzofenap, bicyclopyrone, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clacyfos, clefoxydim, clethodim, clodinafop-propargyl, clomazone, clomeprop, clopyralid, clopyralid-olamine, cloransulam-methyl, cumyluron, cyanazine, cycloate, cyclopyrimorate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, EPTC, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fenoxasulfone, fenquinotrione, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, fluazolate, flucarbazone, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, flurochloridone, fluroxypyr, flurtamone, fluthiacet-methyl, fomesafen, foramsulfuron, fosamine-ammonium, glufosinate, glufosinate-ammonium, glufosinate-P, glyphosate and its salts such as ammonium, isopropylammonium, potassium, sodium (including sesquisodium) and trimesium (alternatively named sulfosate), halauxifen, halauxifen-methyl, halosulfuron-methyl, haloxyfop-etotyl, haloxyfop-methyl, hexazinone, hydantocidin, imazamethabenz-methyl, imazamox, imazapic, imazapyr, imazaquin, imazaquin-ammonium, imazethapyr, imazethapyr-ammonium, imazosulfuron, indanofan, indaziflam, iofensulfuron, iodosulfuron-methyl, ioxynil, ioxynil octanoate, ioxynil-sodium, ipfencarbazone, isoproturon, isouron, isoxaben, isoxaflutole, isoxachlortole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, metazosulfuron, methabenzthiazuron, methylarsonic acid and its calcium, monoammonium, monosodium and disodium salts, methyldymron, metobenzuron, metobromuron, metolachlor, S-metolachlor, metosulam, metoxuron, metribuzin, metsulfuron-methyl, molinate, monolinuron, naproanilide, napropamide, napropamide-M, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperophos, pretilachlor, primisulfuron-methyl, prodiamine, profoxydim, prometon, prometryn, propachlor, propanil, propaquizafop, propazine, propham, propisochlor, propoxycarbazone, propyrisulfuron, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimsulfuron, rinskor, saflufenacil, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, thenylchlor, thiazopyr, thiencarbazone, thifensulfuron-methyl, thiobencarb, tiafenacil, tiocarbazil, tolpyralate, topramezone, tralkoxydim, tri-allate, triafamone, triasulfuron, triaziflam, tribenuron-methyl, triclopyr, triclopyr-butotyl, triclopyr-triethylammonium, tridiphane, trietazine, trifloxysulfuron, trifludimoxazin, trifluralin, triflusulfuron-methyl, tritosulfuron, vemolate, 3-(2-chloro-3,6-difluorophenyl)-4-hydroxy-1-methyl-1,5-naphthyridin-2(1H)-one, 5-chloro-3-[(2-hydroxy-6-oxo-1-cyclohexen-1-yl)carbonyl]-1-(4-methoxyphenyl)-2(1H)-quinoxalinone, 2-chloro-N-(1-methyl-1H-tetrazol-5-yl)-6-(trifluoromethyl)-3-pyridinecarboxamide, 7-(3,5-dichloro-4-pyridinyl)-5-(2,2-difluoroethyl)-8-hydroxypyrido[2,3-b]pyrazin-6(5H)-one), 4-(2,6-diethyl-4-methylphenyl)-5-hydroxy-2,6-dimethyl-3(2H)-pyridazinone), 5-[[(2,6-difluorophenyl)methoxy]methyl]-4,5-dihydro-5-methyl-3-(3-methyl-2-thienyl)isoxazole (previously methioxolin), 4-(4-fluorophenyl)-6-[(2-hydroxy-6-oxo-1-cyclohexen-1-yl)carbonyl]-2-methyl-1,2,4-triazine-3,5(2H,4H)-dione, methyl 4-amino-3-chloro-6-(4-chloro-2-fluoro-3-methoxyphenyl)-5-fluoro-2-pyridinecarboxylate, 2-methyl-3-(methylsulfonyl)-N-(1-methyl-1H-tetrazol-5-yl)-4-(trifluoromethyl)benzamide and 2-methyl-N-(4-methyl-1,2,5-oxadiazol-3-yl)-3-(methylsulfinyl)-4-(trifluoromethyl)benzamide. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butl.) Butl. and Puccinia thlaspeos Schub.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

General references for agricultural protectants (i.e. herbicides, herbicide safeners, insecticides, fungicides, nematocides, acaricides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Farnham, Surrey, U. K., 2001.

For embodiments where one or more of these various mixing partners are used, the mixing partners are typically used in the amounts similar to amounts customary when the mixture partners are used alone. More particularly in mixtures, active ingredients are often applied at an application rate between one-half and the full application rate specified on product labels for use of active ingredient alone. These amounts are listed in references such as The Pesticide Manual and The BioPesticide Manual. The weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of weeds controlled beyond the spectrum controlled by the compound of Formula 1 alone.

In certain instances, combinations of a compound of this invention with other biologically active (particularly herbicidal) compounds or agents (i.e. active ingredients) can result in a greater-than-additive (i.e. synergistic) effect on weeds and/or a less-than-additive effect (i.e. safening) on crops or other desirable plants. Reducing the quantity of active ingredients released in the environment while ensuring effective pest control is always desirable. Ability to use greater amounts of active ingredients to provide more effective weed control without excessive crop injury is also desirable. When synergism of herbicidal active ingredients occurs on weeds at application rates giving agronomically satisfactory levels of weed control, such combinations can be advantageous for reducing crop production cost and decreasing environmental load. When safening of herbicidal active ingredients occurs on crops, such combinations can be advantageous for increasing crop protection by reducing weed competition.

Of note is a combination of a compound of the invention with at least one other herbicidal active ingredient. Of particular note is such a combination where the other herbicidal active ingredient has a site of action different from that of the compound of the invention. In certain instances, a combination with at least one other herbicidal active ingredient having a similar spectrum of control but a different site of action will be particularly advantageous for resistance management. Thus, a composition of the present invention can further comprise (in a herbicidally effective amount) at least one additional herbicidal active ingredient having a similar spectrum of control but a different site of action.

Compounds of this invention can also be used in combination with herbicide safeners such as allidochlor, benoxacor, cloquintocet-mexyl, cumyluron, cyometrinil, cyprosulfonamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone naphthalic anhydride (1,8-naphthalic anhydride), oxabetrinil, N-(aminocarbonyl)-2-methylbenzenesulfonamide, N-(aminocarbonyl)-2-fluorobenzenesulfonamide, 1-bromo-4-[(chloromethyl)sulfonyl]benzene (BCS), 4-(dichloroacetyl)-1-oxa-4-azospiro[4.5]decane (MON 4660), 2-(dichloromethyl)-2-methyl-1,3-dioxolane (MG 191), ethyl 1,6-dihydro-1-(2-methoxyphenyl)-6-oxo-2-phenyl-5-pyrimidinecarboxylate, 2-hydroxy-N,N-dimethyl-6-(trifluoromethyl)pyridine-3-carboxamide, and 3-oxo-1-cyclohexen-1-yl 1-(3,4-dimethylphenyl)-1,6-dihydro-6-oxo-2-phenyl-5-pyrimidinecarboxylate, 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide to increase safety to certain crops. Antidotally effective amounts of the herbicide safeners can be applied at the same time as the compounds of this invention, or applied as seed treatments. Therefore, an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore, a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

Compounds of the invention cans also be mixed with: (1) polynucleotides including but not limited to DNA, RNA, and/or chemically modified nucleotides influencing the amount of a particular target through down regulation, interference, suppression or silencing of the genetically derived transcript that render a herbicidal effect; or (2) polynucleotides including but not limited to DNA, RNA, and/or chemically modified nucleotides influencing the amount of a particular target through down regulation, interference, suppression or silencing of the genetically derived transcript that render a safening effect.

Of note is a composition comprising a compound of the invention (in a herbicidally effective amount), at least one additional active ingredient selected from the group consisting of other herbicides and herbicide safeners (in an effective amount), and at least one component selected from the group consisting of surfactants, solid diluents and liquid diluents.

Table A1 lists specific combinations of a Component (a) with Component (b) illustrative of the mixtures, compositions and methods of the present invention. Compound 1 in the Component (a) column is identified in Index Table A. The second column of Table A1 lists the specific Component (b) compound (e.g., “2,4-D” in the first line). The third, fourth and fifth columns of Table A1 lists ranges of weight ratios for rates at which the Component (a) compound is typically applied to a field-grown crop relative to Component (b) (i.e. (a):(b)). Thus, for example, the first line of Table A1 specifically discloses the combination of Component (a) (i.e. Compound 1 in Index Table A) with 2,4-D is typically applied in a weight ratio between 1:192-6:1. The remaining lines of Table A1 are to be construed similarly.

TABLE A1

| Component (a) | | Typical | More Typical | Most Typical |
|---|---|---|---|---|
| (Compound #) | Component (b) | Weight Ratio | Weight Ratio | Weight Ratio |
| 1 | 2,4-D | 1:192-6:1 | 1:64-2:1 | 1:24-1:3 |
| 1 | Acetochlor | 1:768-2:1 | 1:256-1:2 | 1:96-1:11 |
| 1 | Acifluorfen | 1:96-12:1 | 1:32-4:1 | 1:12-1:2 |
| 1 | Aclonifen | 1:857-2:1 | 1:285-1:3 | 1:107-1:12 |
| 1 | Alachlor | 1:768-2:1 | 1:256-1:2 | 1:96-1:11 |
| 1 | Ametryn | 1:384-3:1 | 1:128-1:1 | 1:48-1:6 |
| 1 | Amicarbazone | 1:192-6:1 | 1:64-2:1 | 1:24-1:3 |
| 1 | Amidosulfuron | 1:6-168:1 | 1:2-56:1 | 1:1-11:1 |
| 1 | Aminocyclopyrachlor | 1:48-24:1 | 1:16-8:1 | 1:6-2:1 |
| 1 | Aminopyralid | 1:20-56:1 | 1:6-19:1 | 1:2-4:1 |
| 1 | Amitrole | 1:768-2:1 | 1:256-1:2 | 1:96-1:11 |
| 1 | Anilofos | 1:96-12:1 | 1:32-4:1 | 1:12-1:2 |
| 1 | Asulam | 1:960-2:1 | 1:320-1:3 | 1:120-1:14 |
| 1 | Atrazine | 1:192-6:1 | 1:64-2:1 | 1:24-1:3 |
| 1 | Azimsulfuron | 1:6-168:1 | 1:2-56:1 | 1:1-11:1 |
| 1 | Beflubutamid | 1:342-4:1 | 1:114-2:1 | 1:42-1:5 |
| 1 | S-Beflubutamid | 1:171-2:1 | 1:57-1:1 | 1:21-1:2.5 |
| 1 | Benfuresate | 1:617-2:1 | 1:205-1:2 | 1:77-1:9 |
| 1 | Bensulfuron-methyl | 1:25-45:1 | 1:8-15:1 | 1:3-3:1 |
| 1 | Bentazone | 1:192-6:1 | 1:64-2:1 | 1:24-1:3 |
| 1 | Benzobicyclon | 1:85-14:1 | 1:28-5:1 | 1:10-1:2 |
| 1 | Benzofenap | 1:257-5:1 | 1:85-2:1 | 1:32-1:4 |
| 1 | Bicyclopyrone | 1:42-27:1 | 1:14-9:1 | 1:5-2:1 |
| 1 | Bifenox | 1:257-5:1 | 1:85-2:1 | 1:32-1:4 |
| 1 | Bispyribac-sodium | 1:10-112:1 | 1:3-38:1 | 1:1-7:1 |
| 1 | Bromacil | 1:384-3:1 | 1:128-1:1 | 1:48-1:6 |
| 1 | Bromobutide | 1:384-3:1 | 1:128-1:1 | 1:48-1:6 |
| 1 | Bromoxynil | 1:96-12:1 | 1:32-4:1 | 1:12-1:2 |
| 1 | Butachlor | 1:768-2:1 | 1:256-1:2 | 1:96-1:11 |
| 1 | Butafenacil | 1:42-27:1 | 1:14-9:1 | 1:5-2:1 |
| 1 | Butylate | 1:1542-1:2 | 1:514-1:5 | 1:192-1:22 |
| 1 | Carfenstrole | 1:192-6:1 | 1:64-2:1 | 1:24-1:3 |
| 1 | Carfentrazone-ethyl | 1:128-9:1 | 1:42-3:1 | 1:16-1:2 |
| 1 | Chlorimuron-ethyl | 1:8-135:1 | 1:2-45:1 | 1:1-9:1 |
| 1 | Chlorotoluron | 1:768-2:1 | 1:256-1:2 | 1:96-1:11 |
| 1 | Chlorsulfuron | 1:6-168:1 | 1:2-56:1 | 1:1-11:1 |
| 1 | Cincosulfuron | 1:17-68:1 | 1:5-23:1 | 1:2-5:1 |
| 1 | Cinidon-ethyl | 1:384-3:1 | 1:128-1:1 | 1:48-1:6 |
| 1 | Cinmethylin | 1:34-34:1 | 1:11-12:1 | 1:4-3:1 |
| 1 | Clacyfos | 1:34-34:1 | 1:11-12:1 | 1:4-3:1 |
| 1 | Clethodim | 1:48-24:1 | 1:16-8:1 | 1:6-2:1 |
| 1 | Clodinafop-propargyl | 1:20-56:1 | 1:6-19:1 | 1:2-4:1 |
| 1 | Clomazone | 1:384-3:1 | 1:128-1:1 | 1:48-1:6 |
| 1 | Clomeprop | 1:171-7:1 | 1:57-3:1 | 1:21-1:3 |
| 1 | Clopyralid | 1:192-6:1 | 1:64-2:1 | 1:24-1:3 |
| 1 | Cloransulam-methyl | 1:12-96:1 | 1:4-32:1 | 1:1-6:1 |
| 1 | Cumyluron | 1:384-3:1 | 1:128-1:1 | 1:48-1:6 |
| 1 | Cyanazine | 1:384-3:1 | 1:128-1:1 | 1:48-1:6 |
| 1 | Cyclopyrimorate | 1:17-68:1 | 1:5-23:1 | 1:2-5:1 |
| 1 | Cyclosulfamuron | 1:17-68:1 | 1:5-23:1 | 1:2-5:1 |
| 1 | Cycloxydim | 1:96-12:1 | 1:32-4:1 | 1:12-1:2 |
| 1 | Cyhalofop | 1:25-45:1 | 1:8-15:1 | 1:3-3:1 |
| 1 | Daimuron | 1:192-6:1 | 1:64-2:1 | 1:24-1:3 |
| 1 | Desmedipham | 1:322-4:1 | 1:107-2:1 | 1:40-1:5 |
| 1 | Dicamba | 1:192-6:1 | 1:64-2:1 | 1:24-1:3 |
| 1 | Dichlobenil | 1:1371-1:2 | 1:457-1:4 | 1:171-1:20 |
| 1 | Dichlorprop | 1:925-2:1 | 1:308-1:3 | 1:115-1:13 |
| 1 | Diclofop-methyl | 1:384-3:1 | 1:128-1:1 | 1:48-1:6 |
| 1 | Diclosulam | 1:10-112:1 | 1:3-38:1 | 1:1-7:1 |
| 1 | Difenzoquat | 1:288-4:1 | 1:96-2:1 | 1:36-1:4 |
| 1 | Diflufenican | 1:857-2:1 | 1:285-1:3 | 1:107-1:12 |
| 1 | Diflufenzopyr | 1:12-96:1 | 1:4-32:1 | 1:1-6:1 |
| 1 | Dimethachlor | 1:768-2:1 | 1:256-1:2 | 1:96-1:11 |
| 1 | Dimethametryn | 1:192-6:1 | 1:64-2:1 | 1:24-1:3 |
| 1 | Dimethenamid-P | 1:384-3:1 | 1:128-1:1 | 1:48-1:6 |
| 1 | Dithiopyr | 1:192-6:1 | 1:64-2:1 | 1:24-1:3 |
| 1 | Diuron | 1:384-3:1 | 1:128-1:1 | 1:48-1:6 |
| 1 | EPTC | 1:768-2:1 | 1:256-1:2 | 1:96-1:11 |
| 1 | Esprocarb | 1:1371-1:2 | 1:457-1:4 | 1:171-1:20 |
| 1 | Ethalfluralin | 1:384-3:1 | 1:128-1:1 | 1:48-1:6 |
| 1 | Ethametsulfuron-methyl | 1:17-68:1 | 1:5-23:1 | 1:2-5:1 |
| 1 | Ethoxyfen | 1:8-135:1 | 1:2-45:1 | 1:1-9:1 |
| 1 | Ethoxysulfuron | 1:20-56:1 | 1:6-19:1 | 1:2-4:1 |
| 1 | Etobenzanid | 1:257-5:1 | 1:85-2:1 | 1:32-1:4 |
| 1 | Fenoxaprop-ethyl | 1:120-10:1 | 1:40-4:1 | 1:15-1:2 |
| 1 | Fenoxasulfone | 1:85-14:1 | 1:28-5:1 | 1:10-1:2 |
| 1 | Fenquinotrione | 1:17-68:1 | 1:5-23:1 | 1:2-5:1 |
| 1 | Fentrazamide | 1:17-68:1 | 1:5-23:1 | 1:2-5:1 |
| 1 | Flazasulfuron | 1:17-68:1 | 1:5-23:1 | 1:2-5:1 |
| 1 | Florasulam | 1:2-420:1 | 1:1-140:1 | 2:1-27:1 |
| 1 | Fluazifop-butyl | 1:192-6:1 | 1:64-2:1 | 1:24-1:3 |
| 1 | Flucarbazone | 1:8-135:1 | 1:2-45:1 | 1:1-9:1 |
| 1 | Flucetosulfuron | 1:8-135:1 | 1:2-45:1 | 1:1-9:1 |
| 1 | Flufenacet | 1:257-5:1 | 1:85-2:1 | 1:32-1:4 |
| 1 | Flumetsulam | 1:24-48:1 | 1:8-16:1 | 1:3-3:1 |
| 1 | Flumiclorac-pentyl | 1:10-112:1 | 1:3-38:1 | 1:1-7:1 |
| 1 | Flumioxazin | 1:25-45:1 | 1:8-15:1 | 1:3-3:1 |
| 1 | Fluometuron | 1:384-3:1 | 1:128-1:1 | 1:48-1:6 |
| 1 | Flupyrsulfuron-methyl | 1:3-336:1 | 1:1-112:1 | 2:1-21:1 |
| 1 | Fluridone | 1:384-3:1 | 1:128-1:1 | 1:48-1:6 |
| 1 | Fluroxypyr | 1:96-12:1 | 1:32-4:1 | 1:12-1:2 |
| 1 | Flurtamone | 1:857-2:1 | 1:285-1:3 | 1:107-1:12 |
| 1 | Fluthiacet-methyl | 1:48-42:1 | 1:16-14:1 | 1:3-3:1 |
| 1 | Fomesafen | 1:96-12:1 | 1:32-4:1 | 1:12-1:2 |
| 1 | Foramsulfuron | 1:13-84:1 | 1:4-28:1 | 1:1-6:1 |
| 1 | Glufosinate | 1:288-4:1 | 1:96-2:1 | 1:36-1:4 |
| 1 | Glyphosate | 1:288-4:1 | 1:96-2:1 | 1:36-1:4 |
| 1 | Halosulfuron-methyl | 1:17-68:1 | 1:5-23:1 | 1:2-5:1 |
| 1 | Halauxifen | 1:20-56:1 | 1:6-19:1 | 1:2-4:1 |
| 1 | Halauxifen methyl | 1:20-56:1 | 1:6-19:1 | 1:2-4:1 |
| 1 | Haloxyfop-methyl | 1:34-34:1 | 1:11-12:1 | 1:4-3:1 |
| 1 | Hexazinone | 1:192-6:1 | 1:64-2:1 | 1:24-1:3 |
| 1 | Hydantocidin | 1:1100-16:1 | 1:385-8:1 | 1:144-4:1 |
| 1 | Imazamox | 1:13-84:1 | 1:4-28:1 | 1:1-6:1 |
| 1 | Imazapic | 1:20-56:1 | 1:6-19:1 | 1:2-4:1 |
| 1 | Imazapyr | 1:85-14:1 | 1:28-5:1 | 1:10-1:2 |
| 1 | Imazaquin | 1:34-34:1 | 1:11-12:1 | 1:4-3:1 |
| 1 | Imazethabenz-methyl | 1:171-7:1 | 1:57-3:1 | 1:21-1:3 |
| 1 | Imazethapyr | 1:24-48:1 | 1:8-16:1 | 1:3-3:1 |
| 1 | Imazosulfuron | 1:27-42:1 | 1:9-14:1 | 1:3-3:1 |
| 1 | Indanofan | 1:342-4:1 | 1:114-2:1 | 1:42-1:5 |
| 1 | Indaziflam | 1:25-45:1 | 1:8-15:1 | 1:3-3:1 |
| 1 | Iodosulfuron-methyl | 1:3-336:1 | 1:1-112:1 | 2:1-21:1 |
| 1 | Ioxynil | 1:192-6:1 | 1:64-2:1 | 1:24-1:3 |
| 1 | Ipfencarbazone | 1:85-14:1 | 1:28-5:1 | 1:10-1:2 |
| 1 | Isoproturon | 1:384-3:1 | 1:128-1:1 | 1:48-1:6 |
| 1 | Isoxaben | 1:288-4:1 | 1:96-2:1 | 1:36-1:4 |
| 1 | Isoxaflutole | 1:60-20:1 | 1:20-7:1 | 1:7-2:1 |
| 1 | Lactofen | 1:42-27:1 | 1:14-9:1 | 1:5-2:1 |
| 1 | Lenacil | 1:384-3:1 | 1:128-1:1 | 1:48-1:6 |
| 1 | Linuron | 1:384-3:1 | 1:128-1:1 | 1:48-1:6 |
| 1 | MCPA | 1:192-6:1 | 1:64-2:1 | 1:24-1:3 |
| 1 | MCPB | 1:288-4:1 | 1:96-2:1 | 1:36-1:4 |
| 1 | Mecoprop | 1:768-2:1 | 1:256-1:2 | 1:96-1:11 |
| 1 | Mefenacet | 1:384-3:1 | 1:128-1:1 | 1:48-1:6 |
| 1 | Mefluidide | 1:192-6:1 | 1:64-2:1 | 1:24-1:3 |
| 1 | Mesosulfuron-methyl | 1:5-224:1 | 1:1-75:1 | 1:1-14:1 |
| 1 | Mesotrione | 1:42-27:1 | 1:14-9:1 | 1:5-2:1 |
| 1 | Metamifop | 1:42-27:1 | 1:14-9:1 | 1:5-2:1 |
| 1 | Metazachlor | 1:384-3:1 | 1:128-1:1 | 1:48-1:6 |
| 1 | Metazosulfuron | 1:25-45:1 | 1:8-15:1 | 1:3-3:1 |
| 1 | Methabenzthiazuron | 1:768-2:1 | 1:256-1:2 | 1:96-1:11 |
| 1 | Metolachlor | 1:768-2:1 | 1:256-1:2 | 1:96-1:11 |
| 1 | Metosulam | 1:8-135:1 | 1:2-45:1 | 1:1-9:1 |
| 1 | Metribuzin | 1:192-6:1 | 1:64-2:1 | 1:24-1:3 |
| 1 | Metsulfuron-methyl | 1:2-560:1 | 1:1-187:1 | 3:1-35:1 |
| 1 | Molinate | 1:1028-2:1 | 1:342-1:3 | 1:128-1:15 |
| 1 | Napropamide | 1:384-3:1 | 1:128-1:1 | 1:48-1:6 |
| 1 | Napropamide-M | 1:192-6:1 | 1:64-2:1 | 1:24-1:3 |
| 1 | Naptalam | 1:192-6:1 | 1:64-2:1 | 1:24-1:3 |
| 1 | Nicosulfuron | 1:12-96:1 | 1:4-32:1 | 1:1-6:1 |
| 1 | Norflurazon | 1:1152-1:1 | 1:384-1:3 | 1:144-1:16 |
| 1 | Orbencarb | 1:1371-1:2 | 1:457-1:4 | 1:171-1:20 |
| 1 | Orthosulfamuron | 1:20-56:1 | 1:6-19:1 | 1:2-4:1 |
| 1 | Oryzalin | 1:514-3:1 | 1:171-1:2 | 1:64-1:8 |
| 1 | Oxadiargyl | 1:384-3:1 | 1:128-1:1 | 1:48-1:6 |
| 1 | Oxadiazon | 1:548-3:1 | 1:182-1:2 | 1:68-1:8 |
| 1 | Oxasulfuron | 1:27-42:1 | 1:9-14:1 | 1:3-3:1 |
| 1 | Oxaziclomefone | 1:42-27:1 | 1:14-9:1 | 1:5-2:1 |
| 1 | Oxyfluorfen | 1:384-3:1 | 1:128-1:1 | 1:48-1:6 |
| 1 | Paraquat | 1:192-6:1 | 1:64-2:1 | 1:24-1:3 |
| 1 | Pendimethalin | 1:384-3:1 | 1:128-1:1 | 1:48-1:6 |
| 1 | Penoxsulam | 1:10-112:1 | 1:3-38:1 | 1:1-7:1 |
| 1 | Penthoxamid | 1:384-3:1 | 1:128-1:1 | 1:48-1:6 |
| 1 | Pentoxazone | 1:102-12:1 | 1:34-4:1 | 1:12-1:2 |
| 1 | Phenmedipham | 1:102-12:1 | 1:34-4:1 | 1:12-1:2 |
| 1 | Picloram | 1:96-12:1 | 1:32-4:1 | 1:12-1:2 |
| 1 | Picolinafen | 1:34-34:1 | 1:11-12:1 | 1:4-3:1 |
| 1 | Pinoxaden | 1:25-45:1 | 1:8-15:1 | 1:3-3:1 |
| 1 | Pretilachlor | 1:192-6:1 | 1:64-2:1 | 1:24-1:3 |
| 1 | Primisulfuron-methyl | 1:8-135:1 | 1:2-45:1 | 1:1-9:1 |
| 1 | Prodiamine | 1:384-3:1 | 1:128-1:1 | 1:48-1:6 |
| 1 | Profoxydim | 1:42-27:1 | 1:14-9:1 | 1:5-2:1 |
| 1 | Prometryn | 1:384-3:1 | 1:128-1:1 | 1:48-1:6 |
| 1 | Propachlor | 1:1152-1:1 | 1:384-1:3 | 1:144-1:16 |
| 1 | Propanil | 1:384-3:1 | 1:128-1:1 | 1:48-1:6 |
| 1 | Propaquizafop | 1:48-24:1 | 1:16-8:1 | 1:6-2:1 |
| 1 | Propoxycarbazone | 1:17-68:1 | 1:5-23:1 | 1:2-5:1 |
| 1 | Propyrisulfuron | 1:17-68:1 | 1:5-23:1 | 1:2-5:1 |
| 1 | Propyzamide | 1:384-3:1 | 1:128-1:1 | 1:48-1:6 |
| 1 | Prosulfocarb | 1:1200-1:2 | 1:400-1:4 | 1:150-1:17 |
| 1 | Prosulfuron | 1:6-168:1 | 1:2-56:1 | 1:1-11:1 |
| 1 | Pyraclonil | 1:42-27:1 | 1:14-9:1 | 1:5-2:1 |
| 1 | Pyraflufen-ethyl | 1:5-224:1 | 1:1-75:1 | 1:1-14:1 |
| 1 | Pyrasulfotole | 1:13-84:1 | 1:4-28:1 | 1:1-6:1 |
| 1 | Pyrazolynate | 1:857-2:1 | 1:285-1:3 | 1:107-1:12 |
| 1 | Pyrazosulfuron-ethyl | 1:10-112:1 | 1:3-38:1 | 1:1-7:1 |
| 1 | Pyrazoxyfen | 1:5-224:1 | 1:1-75:1 | 1:1-14:1 |
| 1 | Pyribenzoxim | 1:10-112:1 | 1:3-38:1 | 1:1-7:1 |
| 1 | Pyributicarb | 1:384-3:1 | 1:128-1:1 | 1:48-1:6 |
| 1 | Pyridate | 1:288-4:1 | 1:96-2:1 | 1:36-1:4 |
| 1 | Pyriftalid | 1:10-112:1 | 1:3-38:1 | 1:1-7:1 |
| 1 | Pyriminobac-methyl | 1:20-56:1 | 1:6-19:1 | 1:2-4:1 |
| 1 | Pyrimisulfan | 1:17-68:1 | 1:5-23:1 | 1:2-5:1 |
| 1 | Pyrithiobac | 1:24-48:1 | 1:8-16:1 | 1:3-3:1 |
| 1 | Pyroxasulfone | 1:85-14:1 | 1:28-5:1 | 1:10-1:2 |
| 1 | Pyroxsulam | 1:5-224:1 | 1:1-75:1 | 1:1-14:1 |
| 1 | Quinclorac | 1:192-6:1 | 1:64-2:1 | 1:24-1:3 |
| 1 | Quizalofop-ethyl | 1:42-27:1 | 1:14-9:1 | 1:5-2:1 |
| 1 | Rimsulfuron | 1:13-84:1 | 1:4-28:1 | 1:1-6:1 |
| 1 | Rinskor | 1:20-56:1 | 1:6-19:1 | 1:2-4:1 |
| 1 | Saflufenacil | 1:25-45:1 | 1:8-15:1 | 1:3-3:1 |
| 1 | Sethoxydim | 1:96-12:1 | 1:32-4:1 | 1:12-1:2 |
| 1 | Simazine | 1:384-3:1 | 1:128-1:1 | 1:48-1:6 |
| 1 | Sulcotrione | 1:120-10:1 | 1:40-4:1 | 1:15-1:2 |
| 1 | Sulfentrazone | 1:147-8:1 | 1:49-3:1 | 1:18-1:3 |
| 1 | Sulfometuron-methyl | 1:34-34:1 | 1:11-12:1 | 1:4-3:1 |
| 1 | Sulfosulfuron | 1:8-135:1 | 1:2-45:1 | 1:1-9:1 |
| 1 | Tebuthiuron | 1:384-3:1 | 1:128-1:1 | 1:48-1:6 |
| 1 | Tefuryltrione | 1:42-27:1 | 1:14-9:1 | 1:5-2:1 |
| 1 | Tembotrione | 1:31-37:1 | 1:10-13:1 | 1:3-3:1 |
| 1 | Tepraloxydim | 1:25-45:1 | 1:8-15:1 | 1:3-3:1 |
| 1 | Terbacil | 1:288-4:1 | 1:96-2:1 | 1:36-1:4 |
| 1 | Terbuthylazine | 1:857-2:1 | 1:285-1:3 | 1:107-1:12 |
| 1 | Terbutryn | 1:192-6:1 | 1:64-2:1 | 1:24-1:3 |
| 1 | Thenylchlor | 1:85-14:1 | 1:28-5:1 | 1:10-1:2 |
| 1 | Thiazopyr | 1:384-3:1 | 1:128-1:1 | 1:48-1:6 |
| 1 | Thiencarbazone | 1:3-336:1 | 1:1-112:1 | 2:1-21:1 |
| 1 | Thifensulfuron-methyl | 1:5-224:1 | 1:1-75:1 | 1:1-14:1 |
| 1 | Tiafenacil | 1:17-68:1 | 1:5-23:1 | 1:2-5:1 |
| 1 | Thiobencarb | 1:768-2:1 | 1:256-1:2 | 1:96-1:11 |
| 1 | Tolpyralate | 1:31-37:1 | 1:10-13:1 | 1:3-3:1 |
| 1 | Topramzone | 1:6-168:1 | 1:2-56:1 | 1:1-11:1 |
| 1 | Tralkoxydim | 1:68-17:1 | 1:22-6:1 | 1:8-2:1 |
| 1 | Triafamone | 1:2-420:1 | 1:1-140:1 | 2:1-27:1 |
| 1 | Triallate | 1:768-2:1 | 1:256-1:2 | 1:96-1:11 |
| 1 | Triasulfuron | 1:5-224:1 | 1:1-75:1 | 1:1-14:1 |
| 1 | Triaziflam | 1:171-7:1 | 1:57-3:1 | 1:21-1:3 |
| 1 | Tribenuron-methyl | 1:3-336:1 | 1:1-112:1 | 2:1-21:1 |
| 1 | Triclopyr | 1:192-6:1 | 1:64-2:1 | 1:24-1:3 |
| 1 | Trifloxysulfuron | 1:2-420:1 | 1:1-140:1 | 2:1-27:1 |
| 1 | Trifludimoxazin | 1:25-45:1 | 1:8-15:1 | 1:3-3:1 |
| 1 | Trifluralin | 1:288-4:1 | 1:96-2:1 | 1:36-1:4 |
| 1 | Triflusulfuron-methyl | 1:17-68:1 | 1:5-23:1 | 1:2-5:1 |
| 1 | Tritosulfuron | 1:13-84:1 | 1:4-28:1 | 1:1-6:1 |

Table A2 is constructed the same as Table A1 above except that entries below the “Component (a)” column heading are replaced with the respective Component (a) Column Entry shown below. Compound 3 in the Component (a) column is identified in Index Table A. Thus, for example, in Table A2 the entries below the “Component (a)” column heading all recite “Compound 3” (i.e. Compound 3 identified in Index Table A), and the first line below the column headings in Table A2 specifically discloses a mixture of Compound 3 with 2,4-D. Tables A3 through A16 are constructed similarly.

| Table | Component (a) |
|---|---|
| Number | Column Entry |
| A2 | Compound 3 |
| A3 | Compound 4 |
| A4 | Compound 6 |
| A5 | Compound 8 |
| A6 | Compound 9 |
| A7 | Compound 13 |
| A8 | Compound 14 |
| A9 | Compound 15 |
| A10 | Compound 16 |
| A11 | Compound 18 |
| A12 | Compound 21 |
| A13 | Compound 22 |
| A14 | Compound 24 |
| A15 | Compound 25 |
| A16 | Compound 27 |

Preferred for better control of undesired vegetation (e.g., lower use rate such as from synergism, broader spectrum of weeds controlled, or enhanced crop safety) or for preventing the development of resistant weeds are mixtures of a compound of this invention with a herbicide selected from the group consisting of atrazine, azimsulfuron, beflubutamid, S-beflubutamid, benzisothiazolinone, carfentrazone-ethyl, chlorimuron-ethyl, chlorsulfuron-methyl, clomazone, clopyralid potassium, cloransulam-methyl, 2-[(2,4-dichlorophenyl)methyl]-4,4-dimethyl-3-isoxazolidinone (CA No. 81777-95-9) and 2-[(2,5-dichlorophenyl)methyl]-4,4-dimethyl-3-isoxazolidinone (CA No. 81778-66-7) ethametsulfuron-methyl, flumetsulam, 4-(4-fluorophenyl)-6-[(2-hydroxy-6-oxo-1-cyclohexen-1-yl)carbonyl]-2-methyl-1,2,4-triazine-3,5-(2H,4H)-dione, flupyrsulfuron-methyl, fluthiacet-methyl, fomesafen, imazethapyr, lenacil, mesotrione, metribuzin, metsulfuron-methyl, pethoxamid, picloram, pyroxasulfone, quinclorac, rimsulfuron, rinskor, S-metolachlor, sulfentrazone, thifensulfuron-methyl, triflusulfuron-methyl and tribenuron-methyl.

The following Tests demonstrate the control efficacy of the compounds of this invention against specific weeds. The weed control afforded by the compounds is not limited, however, to these species. See Index Tables A and B for compound descriptions. The following abbreviations are used in Index Table A which follows: c means cyclo, Me means methyl, Et means ethyl and c-Pr means cyclopropyl. (R) or (S) denotes the absolute chirality of the asymmetric carbon center. “Rac” denotes racemic and (ND) denotes “not determined”. The abbreviation “Cmpd. No.” stands for “Compound Number”. The abbreviation “Ex.” stands for “Example” and is followed by a number indicating in which example the compound is prepared. Mass spectra (M.S.) are reported with an estimated precision within ±0.5 Da as the molecular weight of the highest isotopic abundance parent ion (M+1) formed by addition of H⁺ (molecular weight of 1) to the molecule observed by using atmospheric pressure chemical ionization (AP+), or electrospray ionization (ESI) where indicated.

### Index Table A

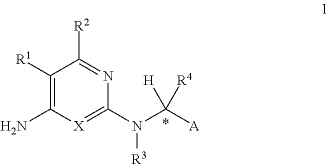

### A is

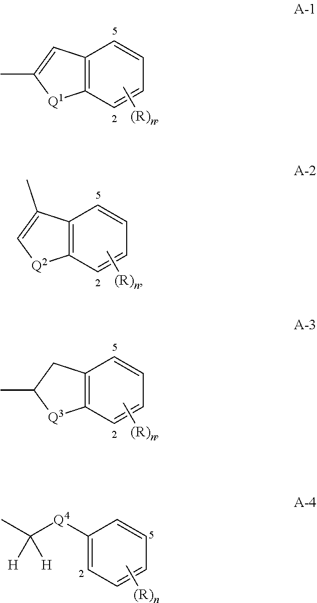

| Cmpd. | Chirality | | | | | | | | | | |
|---|---|---|---|---|---|---|---|---|---|---|---|
| No. | at* | X | A | Q* | R¹ | R² | R³ | R⁴ | (R)ₙ | M.S. | M.P. (° C.) |
| 1 | (R) | N | A-1 | CH═CH | CF₃ | H | H | Me | n = 0 | 333.1 | |
| 2 | (R) | N | A-1 | O | CF₃ | H | H | Me | 4-Cl | 357.1 | |
| 3 | (R) | N | A-2 | CH═CH | CF₃ | H | H | Me | n = 0 | 333.2 | |
| 4 (Ex. 3) | (R) | N | A-1 | S | CF₃ | H | H | Me | n = 0 | 339.2 (ESI) | |
| 5 | (S) | N | A-1 | S | CF₃ | H | H | Me | n = 0 | 339.2 (ESI) | |
| 6 | Rac | N | A-3 | CH₂ | CF₃ | H | H | Me | n = 0 | 323.41 (ESI) | |
| 7 | (R) | N | A-1 | O | CF₃ | H | H | Me | 4-CF₃ | 390.9 (ESI) | |
| 8 | (R) | N | A-1 | O | CF₃ | H | H | Me | 3-F | 341.15 | |
| 9 | (R) | N | A-1 | O | CF₃ | H | H | Me | 4-F | 341.11 | |
| 10 | (R) | N | A-1 | O | CF₃ | H | H | Me | 4-Me | 337.15 | |
| 11 | (R) | N | A-1 | O | CF₃ | H | H | Me | 3,4-di-Me | 351.11 | |
| 12 | (S) | N | A-3 | CH₂ | CF₃ | H | H | Me | n = 0 | 322.8 (ESI) | |
| 13 | (R) | N | A-3 | CH₂ | CF₃ | H | H | Me | n = 0 | 322.8 | |
| 14 | (R) | N | A-1 | O | CF₃ | H | H | Me | 3-Me | 337.17 | |
| 15 | (R) | N | A-1 | O | CF₃ | H | H | Me | 3-CF₃ | 391.09 | |
| 16 | Rac | N | A-1 | S | CF₃ | H | H | Me | n = 0 | 339.2 (ESI) | |
| 17 | (R) | N | A-1 | O | CF₃ | H | H | Me | 4-Et | 351.18 | |
| 18 | Rac | N | A-1 | O | CF₃ | H | H | c-Pr | n = 0 | 349.4 (ESI) | |
| 19 | Rac | N | A-1 | O | CN | H | H | Me | n = 0 | ** | |
| 20 | Rac | N | A-1 | O | CN | H | H | Et | n = 0 | 293.8 (ESI) | |
| 21 | (R) | N | A-1 | O | CF₃ | H | H | Et | n = 0 | 337.0 (ESI) | |
| 22 (Ex. 1) | (R) | N | A-1 | O | CF₃ | H | H | Me | n = 0 | 323.2 (ESI) | |
| 23 | (ND) | N | A-4 | CH₂ | CF₃ | H | H | Me | n = 0 | 311.3 (ESI) | |
| 24 | Rac | N | A-1 | O | CF₃ | H | H | Me | n = 0 | ** | |
| 25 (Ex. 2) | (R) | N | A-4 | O | CF₃ | H | H | Me | 3,5-di-Me | 341.5 (ESI) | |
| 26 | (R) | N | A-4 | O | CF₃ | H | H | Me | n = 0 | 313.2 (ESI) | |
| 27 | (R) | N | A-4 | O | CF₃ | H | H | Et | 3,5-di-Me | 355 (ESI) | |
| 28 | Rac | N | A-1 | O | CF₃ | H | H | Et | n = 0 | 337.4 (ESI) | |
| 29 | (R) | N | A-1 | O | CF₃ | H | H | Me | 3-Cl | 357.1 | |
| 30 | (R) | N | A-1 | O | CF₃ | H | H | Me | 3,4-di-F | 359.1 (ESI) | |
| 31 | (R) | N | A-1 | O | CF₃ | H | H | Me | 4-F,3-Me | 355.2 | |
| 32 | (R) | N | A-4 | O | CF₃ | H | H | Me | 3-Me | 327.2 | |
| 33 | (R) | N | A-4 | O | CF₃ | H | H | Me | 3,5-di-F | 349.2 (ESI) | |
| 34 | (R) | N | A-4 | O | CF₃ | H | H | Me | 3-CF₃ | 381.3 (ESI) | |
| 35 | (R) | N | A-4 | O | CF₃ | H | H | Me | 3,5-di-Cl | 382.2 (ESI) | |
| 36 | (R) | N | A-4 | O | CF₃ | H | H | Me | 3,4,5-tri-F | 367.2 (ESI) | |
| 37 | (R) | N | A-4 | O | CF₃ | H | H | Me | 3-F | 331.3 (ESI) | |
| 38 | (R) | N | A-4 | O | CF₃ | H | H | Me | 4-F,3-Me | 345.2 (ESI) | |
| 39 | (R) | N | A-4 | O | CF₃ | H | H | Me | 3-F,5-Me | 345.3 (ESI) | |
| 40 | (ND) | N | A-2 | S | CF₃ | H | H | Me | n = 0 | 339.2 (ESI) | |
| 41 | (R) | N | A-2 | S | CF₃ | H | H | Me | n = 0 | | 109-113 |
| 42 | (S) | N | A-2 | S | CF₃ | H | H | Me | n = 0 | | 106-110 |
| 43 | (R) | N | A-1 | S | CF₃ | H | H | Et | n = 0 | 353 | |
| 44 | (S) | N | A-1 | S | CF₃ | H | H | Et | n = 0 | 353 | |
| 45 | (R) | N | A-2 | S | CF₃ | H | H | Et | n = 0 | 357 | |
| 46 | (R) | N | A-1 | S | CF₃ | H | H | Me | 3-F | 357 | |
| 47 | (S) | N | A-1 | S | CF₃ | H | H | Me | 3-F | | 197.6-212.3 |
| 48 | (R) | N | A-1 | S | CF₃ | H | H | Et | 3-F | | 162.4-167.6 |
| 49 | (S) | N | A-1 | S | CF₃ | H | H | Et | 3-F | 371 | |
| 50 | (R) | N | A-1 | S | CF₃ | H | H | i-Pr | n = 0 | 367 | |
| 51 | Rac | N | A-2 | O | CF₃ | H | H | Me | 3-F | 341 | |
| 52 | (R) | N | A-2 | O | CF₃ | H | H | Me | n = 0 | 323 | |
| 53 | Rac | N | A-2 | O | CF₃ | H | H | Et | n = 0 | 337 | |
| 54 | Rac | N | A-1 | S | CF₃ | CF₃ | H | Me | n = 0 | 337 | |
| 55 | (R) | N | A-2 | O | CF₃ | H | H | Me | 3-Cl | 357 | |
| 56 | (S) | N | A-2 | O | CF₃ | H | H | Me | 4-F | 341 | |
| 57 | | N | A-1 | S | C(O)Me | H | H | Me | n = 0 | 313 | |
| 58 | Rac | N | A-1 | O | CF₃ | CF₃ | H | Me | n = 0 | 391 | |
| 59 | (R) | N | A-1 | S | CF₃ | CF₃ | H | Me | n = 0 | 407 | |
| 60 | (S) | N | A-1 | S | CF₃ | CF₃ | H | Me | n = 0 | 407 | |
| 61 | | N | A-1 | S | CF₃ | H | H | Me | 5-F | | 137-141 |
| 62 | | N | A-1 | S | CF₃ | H | H | Me | 3-Me | | 63-67 |
| 63 | (R) | N | A-1 | O | CF₃ | H | H | Et | 3-F | 355 | |
| 64 | (S) | N | A-1 | O | CF₃ | H | H | Et | 3-F | 355 | |
| 65 | (R) | N | A-1 | O | CF₃ | CF₃ | H | Me | n = 0 | 391 | |
| 66 | (S) | N | A-1 | O | CF₃ | CF₃ | H | Me | n = 0 | 391 | |
| 67 | (R) | N | A-3 | CH₂ | CF₃ | CF₃ | H | Me | n = 0 | 391 | |
| 68 | (S) | N | A-3 | CH₂ | CF₃ | CF₃ | H | Me | n = 0 | 391 | |
| 69 | Rac | N | A-3 | CH₂ | CF₃ | CF₃ | H | Me | n = 0 | 391 | |
| 70 | | N | A-1 | S | CF₃ | H | H | Me | 3-Cl | 373 | |
| 71 | (R) | N | A-1 | O | CF₃ | H | H | Me | 2-F | 341 | |
| 72 | (S) | N | A-1 | O | CF₃ | H | H | Me | 2-F | | 196.2-214 |
| 73 | (R) | N | A-1 | O | CF₃ | H | H | Me | 5-F | 341 | |
| 74 | (S) | N | A-1 | O | CF₃ | H | H | Me | 5-F | 341 | |
| 75 | | N | A-1 | S | CF₃ | H | H | Me | 2-F | 357 | |
| 76 | (S) | N | A-1 | NMe | CF₃ | H | H | Me | n = 0 | | |
| 77 | Rac | N | A-2 | O | CF₃ | H | H | Me | 3-Cl | 357 | |
| 78 | (S) | N | A-2 | O | CF₃ | H | H | c-Pr | n = 0 | 349 | |
| 79 | (R) | N | A-2 | O | CF₃ | H | H | c-Pr | n = 0 | 349 | |
| 80 | (R) | N | A-2 | O | CF₃ | H | H | Et | n = 0 | 337 | |
| 81 | (S) | N | A-2 | O | CF₃ | H | H | Et | n = 0 | 337 | |
| 82 | (R) | N | A-2 | O | CF₃ | H | H | Et | 3-F | 341 | |
| 83 | (S) | N | A-2 | O | CF₃ | H | H | Et | 3-F | 341 | |
| 84 | (R) | N | A-1 | S | CF₃ | H | H | c-Pr | n = 0 | 365 | |
| 85 | | N | A-2 | O | CF₃ | H | H | Me | 3-Me | 337 | |
| 86 | | N | A-1 | S | CF₃ | H | H | Me | 2,3-di-F | 375 | |
| 87 | | N | A-2 | O | CF₃ | CF₃ | H | Me | n = 0 | 391 | |
| 88 | (R) | N | A-1 | O | CF₃ | H | H | c-Pr | n = 0 | 349 | |
| 89 | (S) | N | A-1 | O | CF₃ | H | H | c-Pr | n = 0 | 349 | |
| 90 | (R) | N | A-2 | O | CF₃ | H | H | c-Pr | 3-Me | 363 | |
| 91 | (S) | N | A-2 | O | CF₃ | H | H | c-Pr | 3-Me | 363 | |
| 92 | | N | A-1 | S | CF₃ | H | H | Me | 4-F | | 46-50 |
| 93 | | N | A-1 | S | CF₃ | H | H | Me | 3,5-di-F | | 131-135 |
| 94 | Rac | N | A-1 | S | CF₃ | H | H | c-Pr | n = 0 | 365 | |
| 95 | Rac | N | A-2 | O | CF₃ | H | H | c-Pr | n = 0 | 349 | |
| 96 | | N | A-2 | O | CF₃ | H | H | Me | 5-F | 341 | |
| 97 | (R) | N | A-1 | S | H | CF₃ | H | Me | n = 0 | 339.1 | |
| 98 | (R) | N | A-1 | NMe | CF₃ | H | H | Me | n = 0 | 336.1 | |
| 99 | | N | A-1 | S | NO₂ | H | H | Me | n = 0 | 316 | |
| 100 | | N | A-1 | O | CF₃ | H | H | Me | 5-Cl | 357 | |
| 101 | | N | A-1 | O | CF₃ | H | H | Me | 5-Me | 337 | |
| 102 | | N | A-1 | O | CF₃ | H | H | Me | 5-Br | 402 | |
| 103 | | N | A-1 | S | CF₃ | H | Me | Me | n = 0 | | 97-101 |
| 104 | (R) | N | A-1 | S | CN | H | H | Me | n = 0 | 296.2 | |
| 105 | | N | A-1 | S | CF₃ | Me | H | Me | n = 0 | 353 | |
| 106 | | N | A-2 | O | CF₃ | Me | H | Me | n = 0 | 337 | |
| 107 | | N | A-1 | S | SO₂Me | H | H | Me | n = 0 | 349 | |
| 108 | (R) | N | A-1 | S | CF₃ | CFHCH₃ | H | Me | n = 0 | | 121-124 |
| 109 | (R) | N | A-1 | S | CF₃ | CF₂H | H | Me | n = 0 | 389 | |
| 110 | (R) | N | A-1 | S | SOMe | H | H | Me | n = 0 | | 78-81 |
| 111 | (R) | N | A-1 | O | CF₃ | CF₂H | H | Me | n = 0 | 373 | |
| 112 | (R) | N | A-1 | O | CF₃ | CFHCH₃ | H | Me | n = 0 | 369 | |
| 113 | (R) | N | A-1 | S═O | CF₃ | H | H | Me | n = 0 | | |
| 114 | (R) | N | A-1 | S | CF₃ | H | H | Me | 2,5-di-F | 375 | |
| 115 | (R) | N | A-1 | S | CF₃ | H | H | Me | 5-Me | 353 | |
| 116 | (R) | N | A-1 | S | CF₃ | H | H | Me | 5-Cl | 373 | |
| 117 | (R) | N | A-3 | O | CF₃ | H | H | Me | n = 0 | 325 | |
| 118 | (R) | N | A-1 | O | CF₃ | CH₃ | H | Me | 5-F | 355 | |
| 119 | (R) | N | A-1 | S | CF₃ | H | H | Me | 5-CF₃ | 407 | |
| 120 | Rac | N | A-3 | S | CF₃ | H | H | Me | n = 0 | | 192-209 |
| 121 | (R) | N | A-1 | O | CF₃ | H | H | Me | 2-Me | 337 | |
| 122 | (R) | N | A-1 | O | CF₃ | H | H | Me | 5-OMe | 353 | |
| 123 | (R) | N | A-1 | O | CF₃ | H | H | Me | 2-Cl | 357 | |
| 124 | (R) | N | A-1 | O | CF₃ | H | H | Me | 2-OMe | 353 | |
| 125 | Non | N | A-1 | S | CF₃ | H | H | c-Pr | 5-F | | 61-64 |
| 126 | (R) | N | A-1 | O | CF₃ | H | H | Et | 5-F | 355 | |
| 127 | (ND) | N | A-1 | O | CF₃ | H | H | c-Pr | 5-F | 367 | |
| 128 | (ND) | N | A-1 | O | CF₃ | H | H | c-Pr | 5-F | 367 | |
| 129 | (R) | N | A-1 | S | CF₃ | H | H | Et | 5-F | 371 | |
| 130 | (R) | N | A-1 | O | CF₃ | CF₂H | H | Me | 5-F | 391 | |
| 131 | (R) | N | A-1 | O | CF₃ | CF₂H | H | Me | 2-F | 391 | |

*The listed value for “Q” refers to the respective Q listed for each value of A; For A-1, Q is Q¹; for A-2, Q is Q²; for A-3, Q is Q³; for A-4, Q is Q⁴.
** See Index Table B for ¹H NMR data.

### Index Table B

INDEX TABLE B

| Cmpd. | ¹H NMR Data (CDCl₃ solution at |
|---|---|
| No. | 500 MHz unless indicated otherwise)^{a} |
| 19 | δ 8.3-7.98 (m, 1H), 7.55-7.36 (m, 2H), 7.28-7.14 (m, 1H), |
| | 6.95-6.70 (m, 1H), 6.54 (s, 1H), 5.69-5.20 (m, 3H), 1.60 |
| | (br d, 3H) |
| 24 | δ 8.20-8.04 (m, 1H), 7.52-7.47 (m, 1H), 7.44-7.37 (m, 1H), |
| | 7.29-7.14 (m, 3H), 6.61-6.52 (m, 1H), 5.51-5.38 (m, 1H), |
| | 5.23-5.10 (m, 2H), 1.62 (d, 3H) |

^{a1}H NMR data are in ppm downfield from tetramethylsilane. Couplings are designated by (s)—singlet, (d)—doublet, (m)—multiplet, (br d)—broad doublet.

### Biological Examples of the Invention

### Test A

Seeds of plant species selected from barnyardgrass (Echinochloa crus-galli), kochia (Kochia scoparia), ragweed (common ragweed, Ambrosia elatior), ryegrass, Italian (Lolium multiflorum), foxtail, giant (Setaria faberii), foxtail, green (Setaria viridis), and pigweed (Amaranthus retroflexus) were planted into a blend of loam soil and sand and treated preemergence with a directed soil spray using test chemicals formulated in a non-phytotoxic solvent mixture which included a surfactant.

At the same time, plants selected from these weed species and also wheat (Triticum aestivum), corn (Zea mays), blackgrass (Alopecurus myosuroides), and galium (catchweed bedstraw, Galium aparine) were planted in pots containing the same blend of loam soil and sand and treated with postemergence applications of test chemicals formulated in the same manner. Plants ranged in height from 2 to 10 cm and were in the one- to two-leaf stage for the postemergence treatment. Treated plants and untreated controls were maintained in a greenhouse for approximately 10 d, after which time all treated plants were compared to untreated controls and visually evaluated for injury. Plant response ratings, summarized in Table A, are based on a 0 to 100 scale where 0 is no effect and 100 is complete control. A dash (-) response means no test result.

TABLE A

| 1000 g ai/ha | Compounds | | |
|---|---|---|---|
| Postemergence | 21 | 22 | 26 |
| Barnyardgrass | 80 | 100 | 0 |
| Blackgrass | 80 | 90 | 0 |
| Corn | 100 | 100 | 10 |
| Foxtail, Giant | 70 | 90 | 0 |
| Galium | 80 | 90 | 60 |
| Kochia | 90 | 80 | 30 |
| Pigweed | 100 | 100 | 50 |
| Ragweed | 80 | 80 | 30 |
| Ryegrass, Italian | 50 | 100 | 10 |
| Wheat | 0 | 40 | 0 |

| 500 g ai/ha | Compounds | | | | | | | | | | | | | |
|---|---|---|---|---|---|---|---|---|---|---|---|---|---|---|
| Postemergence | 1 | 2 | 3 | 4 | 5 | 6 | 7 | 8 | 9 | 10 | 11 | 12 | 13 | 14 |
| Barnyardgrass | 0 | 10 | 80 | 60 | 0 | 10 | 0 | 90 | 70 | 20 | 30 | 0 | 60 | 40 |
| Blackgrass | 0 | 0 | 30 | 60 | 0 | 10 | 0 | 60 | 30 | 20 | 20 | 0 | 30 | 30 |
| Corn | 0 | 0 | 10 | 30 | 0 | 20 | 0 | 20 | 20 | 0 | 0 | 0 | 60 | 10 |
| Foxtail, Giant | 0 | 0 | 40 | 70 | 0 | 20 | 0 | 90 | 90 | 40 | 50 | 0 | 100 | 90 |
| Foxtail, Green | — | — | — | — | — | — | — | — | — | — | — | — | — | — |
| Galium | 70 | 30 | 90 | 80 | 40 | 70 | 20 | 90 | 80 | 70 | 70 | 0 | 80 | 80 |
| Kochia | 50 | 0 | 90 | 100 | 20 | 90 | 0 | 90 | 90 | 0 | 70 | 30 | 100 | 90 |
| Pigweed | 100 | 90 | 100 | 100 | 90 | 100 | 90 | 100 | 90 | 90 | 80 | 40 | 100 | 90 |
| Ragweed | 50 | 20 | 90 | 100 | 10 | 40 | 0 | 90 | 80 | 80 | 80 | 0 | 80 | 70 |
| Ryegrass, Italian | 20 | 0 | 0 | 20 | 0 | 20 | 0 | 0 | 30 | 0 | 0 | 0 | 20 | 0 |
| Wheat | 0 | 0 | 0 | 0 | 0 | 0 | 0 | 0 | 0 | 0 | 0 | 0 | 20 | 0 |

| 500 g ai/ha | Compounds | | | | | | | | | | | | | |
|---|---|---|---|---|---|---|---|---|---|---|---|---|---|---|
| Postemergence | 15 | 16 | 17 | 18 | 19 | 20 | 23 | 24 | 25 | 27 | 28 | 29 | 30 | 31 |
| Barnyardgrass | 30 | 60 | 0 | 80 | 0 | 40 | 0 | 60 | 90 | 20 | 30 | 80 | 30 | 80 |
| Blackgrass | 30 | 30 | 0 | 80 | 60 | 100 | 0 | 70 | 70 | 50 | 80 | 10 | 0 | 10 |
| Corn | 20 | 20 | 0 | 0 | 10 | 20 | 0 | 20 | 60 | 20 | 80 | 10 | 0 | 30 |
| Foxtail, Giant | 80 | 90 | 20 | 70 | 0 | 10 | — | — | 80 | 20 | 40 | 50 | 30 | 70 |
| Foxtail, Green | — | — | — | — | — | — | 0 | 90 | — | — | — | — | — | — |
| Galium | 80 | 80 | 0 | 90 | 70 | 80 | 0 | 90 | 90 | 90 | 90 | 100 | 60 | 100 |
| Kochia | 80 | 80 | 0 | 90 | 30 | 90 | 0 | 90 | 80 | 70 | 80 | 90 | 80 | 90 |
| Pigweed | 90 | 100 | 20 | 100 | 50 | 90 | 0 | 100 | 90 | 90 | 100 | 100 | 100 | 100 |
| Ragweed | 40 | 80 | 20 | 70 | 0 | 70 | 0 | 80 | 90 | 90 | 60 | 90 | 80 | 90 |
| Ryegrass, Italian | 30 | 40 | 0 | 30 | 20 | 0 | 0 | 50 | 20 | 0 | 0 | 10 | 0 | 10 |
| Wheat | 0 | 0 | 0 | 10 | 0 | 10 | 0 | 0 | 30 | 20 | 10 | 0 | 0 | 0 |

| 500 g ai/ha | Compounds | | | | | | | | |
|---|---|---|---|---|---|---|---|---|---|
| Postemergence | 32 | 33 | 34 | 35 | 36 | 37 | 38 | 39 | 40 |
| Barnyardgrass | 20 | 0 | 20 | 0 | 0 | 0 | 0 | 0 | 60 |
| Blackgrass | 0 | 0 | 30 | 0 | 0 | 0 | 0 | 0 | 10 |
| Corn | 0 | 0 | 10 | 0 | 0 | 0 | 0 | 20 | 10 |
| Foxtail, Giant | 40 | 0 | 30 | 0 | 0 | 0 | 0 | 10 | 20 |
| Foxtail, Green | — | — | — | — | — | — | — | — | — |
| Galium | 70 | 60 | 70 | 60 | 0 | 40 | 70 | 70 | 90 |
| Kochia | 60 | 30 | 70 | 20 | 0 | 0 | 30 | 70 | 90 |
| Pigweed | 60 | 30 | 60 | 50 | 0 | 40 | 80 | 60 | 100 |
| Ragweed | 40 | 0 | 0 | 0 | 0 | 0 | 0 | 40 | 60 |
| Ryegrass, Italian | 0 | 0 | 60 | 30 | 0 | 0 | 0 | 50 | 0 |
| Wheat | 0 | 0 | 0 | 0 | 0 | 0 | 0 | 0 | 0 |

| 125 g ai/ha | Compounds | | | | | | | | | | | | | |
|---|---|---|---|---|---|---|---|---|---|---|---|---|---|---|
| Postemergence | 1 | 2 | 3 | 4 | 5 | 6 | 7 | 8 | 9 | 10 | 11 | 12 | 13 | 14 |
| Barnyardgrass | 0 | 0 | 20 | 20 | 0 | 10 | 0 | 50 | 10 | 0 | 20 | 0 | 30 | 10 |
| Blackgrass | 0 | 0 | 0 | 0 | 0 | 0 | 0 | 20 | 0 | 0 | 0 | 0 | 10 | 30 |
| Corn | 0 | 0 | 0 | 0 | 10 | 0 | 0 | 0 | 0 | 0 | 40 | 0 | 10 | 10 |
| Foxtail, Giant | 0 | 0 | 0 | 30 | 0 | 0 | 0 | 40 | 0 | 0 | 0 | 0 | 80 | 10 |
| Foxtail, Green | — | — | — | — | — | — | — | — | — | — | — | — | — | — |
| Galium | 30 | 0 | 30 | 80 | 0 | 40 | 0 | 70 | 80 | 50 | 50 | 0 | 80 | 80 |
| Kochia | 0 | 0 | 30 | 90 | 0 | 70 | 0 | 80 | 80 | 0 | 30 | 0 | 90 | 90 |
| Pigweed | 80 | 40 | 90 | 100 | 20 | 90 | 30 | 100 | 90 | 80 | 80 | 30 | 100 | 90 |
| Ragweed | 0 | 0 | 30 | 80 | 0 | 20 | 0 | 90 | 60 | 20 | 40 | 0 | 30 | 0 |
| Ryegrass, Italian | 0 | 0 | 0 | 0 | 0 | 0 | 0 | 0 | 0 | 0 | 0 | 0 | 0 | 0 |
| Wheat | 0 | 0 | 20 | 0 | 0 | 0 | 0 | 0 | 0 | 0 | 0 | 0 | 0 | 0 |

| 125 g ai/ha | Compounds | | | | | | | | | | | | | |
|---|---|---|---|---|---|---|---|---|---|---|---|---|---|---|
| Postemergence | 15 | 16 | 17 | 18 | 19 | 20 | 23 | 24 | 25 | 27 | 28 | 29 | 30 | 31 |
| Barnyardgrass | 30 | 30 | 0 | 10 | 0 | 0 | 0 | 30 | 50 | 0 | 0 | 20 | 0 | 20 |
| Blackgrass | 0 | 10 | 0 | 20 | 20 | 30 | 0 | 30 | 0 | 10 | 10 | 0 | 0 | 20 |
| Corn | 0 | 0 | 0 | 0 | 0 | 0 | 0 | 0 | 20 | 0 | 0 | 0 | 10 | 0 |
| Foxtail, Giant | 30 | 20 | 20 | 0 | 0 | 0 | — | — | 10 | 0 | 10 | 0 | 0 | 10 |
| Foxtail, Green | — | — | — | — | — | — | 0 | 40 | — | — | — | — | — | — |
| Galium | 60 | 60 | 0 | 40 | 0 | 30 | 0 | 90 | 80 | 90 | 70 | 80 | 50 | 70 |
| Kochia | 40 | 70 | 0 | 50 | 0 | 50 | 0 | 70 | 60 | 0 | 30 | 50 | 20 | 60 |
| Pigweed | 90 | 90 | 0 | 90 | 0 | 60 | 0 | 90 | 70 | 60 | 90 | 100 | 100 | 90 |
| Ragweed | 20 | 0 | 0 | 20 | 0 | 20 | 0 | 40 | 60 | 20 | 20 | 20 | 20 | 60 |
| Ryegrass, Italian | 0 | 0 | 0 | 0 | 0 | 0 | 0 | 30 | 0 | 0 | 0 | 0 | 0 | 30 |
| Wheat | 0 | 0 | 0 | 20 | 0 | 0 | 0 | 0 | 0 | 0 | 0 | 0 | 0 | 0 |

| 125 g ai/ha | Compounds | | | | | | | | |
|---|---|---|---|---|---|---|---|---|---|
| Postemergence | 32 | 33 | 34 | 35 | 36 | 37 | 38 | 39 | 40 |
| Barnyardgrass | 0 | 0 | 0 | 20 | 0 | 0 | 0 | 0 | 0 |
| Blackgrass | 0 | 0 | 0 | 0 | 0 | 0 | 0 | 0 | 0 |
| Corn | 0 | 0 | 0 | 0 | 10 | 0 | 0 | 10 | 0 |
| Foxtail, Giant | 0 | 0 | 0 | 0 | 0 | 0 | 0 | 0 | 0 |
| Foxtail, Green | — | — | — | — | — | — | — | — | — |
| Galium | 60 | 20 | 30 | 0 | 0 | 20 | 30 | 70 | 60 |
| Kochia | 20 | 0 | 30 | 0 | 0 | 0 | 0 | 40 | 50 |
| Pigweed | 30 | 0 | 0 | 0 | 0 | 0 | 20 | 20 | 80 |
| Ragweed | 0 | 0 | 0 | 0 | 0 | 0 | 0 | 0 | 30 |
| Ryegrass, Italian | 0 | 0 | 0 | 40 | 0 | 0 | 0 | 0 | 30 |
| Wheat | 0 | 0 | 0 | 0 | 0 | 0 | 0 | 0 | 0 |

| 1000 g ai/ha | Compounds | | |
|---|---|---|---|
| Preemergence | 21 | 22 | 26 |
| Barnyardgrass | 100 | 100 | 10 |
| Foxtail, Giant | 100 | 100 | 50 |
| Kochia | 80 | 100 | 0 |
| Pigweed | 100 | 100 | 80 |
| Ragweed | 100 | 100 | 100 |
| Ryegrass, Italian | 80 | 100 | 20 |

| 500 g ai/ha | Compounds | | | | | | | | | | | | | |
|---|---|---|---|---|---|---|---|---|---|---|---|---|---|---|
| Preemergence | 1 | 2 | 3 | 4 | 5 | 6 | 7 | 8 | 9 | 10 | 11 | 12 | 13 | 14 |
| Barnyardgrass | 0 | 0 | 100 | 100 | 0 | 60 | 0 | 90 | 80 | 30 | 70 | 0 | 100 | 100 |
| Foxtail, Giant | 30 | 0 | 90 | 100 | 10 | 100 | 0 | 100 | 90 | 60 | 80 | 0 | 100 | 100 |
| Foxtail, Green | — | — | — | — | — | — | — | — | — | — | — | — | — | — |
| Kochia | 10 | 0 | 100 | 100 | 0 | 100 | 0 | 90 | 70 | 0 | 10 | 0 | 100 | 90 |
| Pigweed | 90 | 20 | 100 | 100 | 50 | 100 | 30 | 100 | 100 | 60 | 100 | 50 | 100 | 100 |
| Ragweed | 0 | 0 | 100 | 100 | 0 | 100 | 0 | 100 | 90 | 50 | 90 | — | 100 | 100 |
| Ryegrass, Italian | 0 | 0 | 90 | 90 | 20 | 70 | 0 | 0 | 50 | 20 | 30 | 0 | 100 | 60 |

| 500 g ai/ha | Compounds | | | | | | | | | | | | | |
|---|---|---|---|---|---|---|---|---|---|---|---|---|---|---|
| Preemergence | 15 | 16 | 17 | 18 | 19 | 20 | 23 | 24 | 25 | 27 | 28 | 29 | 30 | 31 |
| Barnyardgrass | 70 | 100 | 0 | 80 | 0 | 50 | 0 | 100 | 100 | 90 | 90 | 90 | 80 | 100 |
| Foxtail, Giant | 90 | 100 | 20 | 90 | 20 | 80 | — | — | 100 | 90 | 90 | 90 | 70 | 100 |
| Foxtail, Green | — | — | — | — | — | — | 0 | 100 | — | — | — | — | — | — |
| Kochia | 50 | 100 | 0 | 60 | 20 | 30 | 0 | 80 | 90 | 40 | 90 | 80 | 30 | 90 |
| Pigweed | 100 | 100 | 0 | 100 | 90 | 100 | 50 | 100 | 100 | 80 | 100 | 100 | 100 | 100 |
| Ragweed | 90 | — | 50 | 100 | 50 | 60 | 0 | 100 | 100 | 90 | 100 | 80 | — | — |
| Ryegrass, Italian | 70 | 10 | 20 | 50 | 60 | 90 | 0 | 70 | 70 | 80 | 60 | 70 | 0 | 80 |

| 500 g ai/ha | Compounds | | | | | | | | |
|---|---|---|---|---|---|---|---|---|---|
| Preemergence | 32 | 33 | 34 | 35 | 36 | 37 | 38 | 39 | 40 |
| Barnyardgrass | 90 | 0 | 100 | 0 | 0 | 0 | 90 | 90 | 80 |
| Foxtail, Giant | 80 | 30 | 90 | 0 | 0 | 0 | 90 | 90 | 90 |
| Foxtail, Green | — | — | — | — | — | — | — | — | — |
| Kochia | 30 | 30 | 80 | 40 | 30 | 0 | 60 | 80 | 60 |
| Pigweed | 60 | 50 | 90 | 0 | 0 | 70 | 90 | 80 | 100 |
| Ragweed | 100 | 0 | 80 | 0 | 0 | 100 | — | 100 | 100 |
| Ryegrass, Italian | 0 | 0 | 30 | 0 | 0 | 0 | — | 10 | 100 |

| 125 g ai/ha | Compounds | | | | | | | | | | | | | |
|---|---|---|---|---|---|---|---|---|---|---|---|---|---|---|
| Preemergence | 1 | 2 | 3 | 4 | 5 | 6 | 7 | 8 | 9 | 10 | 11 | 12 | 13 | 14 |
| Barnyardgrass | 0 | 0 | 10 | 90 | 0 | 0 | 0 | 0 | 80 | 20 | 20 | 0 | 90 | 90 |
| Foxtail, Giant | 0 | 0 | 10 | 90 | 0 | 90 | 0 | 50 | 40 | 0 | 40 | 0 | 100 | 90 |
| Foxtail, Green | — | — | — | — | — | — | — | — | — | — | — | — | — | — |
| Kochia | 0 | 0 | 30 | 100 | 0 | 80 | 0 | 60 | 60 | 0 | 0 | 0 | 100 | 60 |
| Pigweed | 10 | 10 | 100 | 100 | 0 | 100 | 0 | 80 | 100 | 0 | 20 | 0 | 100 | 90 |
| Ragweed | 0 | 0 | 100 | 100 | 0 | 30 | 0 | 90 | 0 | 0 | 0 | — | 100 | 100 |
| Ryegrass, Italian | 0 | 0 | 20 | 80 | 20 | 30 | 0 | 0 | 0 | 0 | 20 | 0 | 60 | 0 |

| 125 g ai/ha | Compounds | | | | | | | | | | | | | |
|---|---|---|---|---|---|---|---|---|---|---|---|---|---|---|
| Preemergence | 15 | 16 | 17 | 18 | 19 | 20 | 23 | 24 | 25 | 27 | 28 | 29 | 30 | 31 |
| Barnyardgrass | 20 | 70 | 0 | 50 | 0 | 40 | 0 | 80 | 90 | 10 | 50 | 0 | 0 | 70 |
| Foxtail, Giant | 20 | 50 | 0 | 90 | 0 | 0 | — | — | 50 | 20 | 60 | 60 | 0 | 90 |
| Foxtail, Green | — | — | — | — | — | — | 0 | 90 | — | — | — | — | — | — |
| Kochia | 40 | 70 | 0 | 0 | 0 | 0 | 0 | 20 | 20 | 0 | 10 | 0 | 0 | 40 |
| Pigweed | 80 | 90 | 0 | 90 | 30 | 50 | 0 | 100 | 60 | 10 | 90 | 90 | 40 | 90 |
| Ragweed | 30 | — | 0 | 80 | 0 | 40 | 0 | 50 | 80 | 70 | 20 | 20 | — | — |
| Ryegrass, Italian | 0 | 0 | 0 | 50 | 0 | 30 | 0 | 60 | 20 | 30 | 20 | 20 | 0 | 20 |

| | |
|---|---|
| 125 g ai/ha | Compounds |

| Preemergence | 32 | 33 | 34 | 35 | 36 | 37 | 38 | 39 | 40 |
|---|---|---|---|---|---|---|---|---|---|
| Barnyardgrass | 0 | 0 | 80 | 0 | 0 | 0 | 30 | 40 | 30 |
| Foxtail, Giant | 20 | 0 | 50 | 0 | 0 | 0 | 20 | — | 10 |
| Foxtail, Green | — | — | — | — | — | — | — | — | — |
| Kochia | 0 | 0 | 0 | 0 | 70 | 0 | 0 | 0 | 60 |
| Pigweed | 10 | 20 | 10 | 0 | 0 | 50 | 50 | 70 | 100 |
| Ragweed | 0 | 0 | 20 | 0 | 0 | 0 | — | 70 | 90 |
| Ryegrass, Italian | 0 | 0 | 0 | 0 | 0 | 0 | — | 0 | 20 |

| 1000 g ai/ha | Compound |
|---|---|
| Preemergence | 97 |
| Barnyardgrass | 0 |
| Foxtail, Giant | 0 |
| Kochia | 0 |
| Pigweed | 0 |
| Ragweed | 0 |
| Ryegrass, Italian | 0 |

| 500 g ai/ha | Compounds | | | | | | | | | | | | | |
|---|---|---|---|---|---|---|---|---|---|---|---|---|---|---|
| Preemergence | 41 | 42 | 43 | 44 | 45 | 46 | 47 | 48 | 49 | 50 | 51 | 52 | 53 | 54 |
| Barnyardgrass | 90 | 0 | 70 | 40 | 30 | 90 | 0 | 40 | 0 | 10 | 100 | 100 | 80 | 0 |
| Foxtail, Giant | 90 | 0 | 90 | 20 | 80 | 100 | 0 | 90 | 0 | 10 | 90 | 100 | 90 | 10 |
| Kochia | 90 | 0 | 100 | 0 | 0 | 100 | 0 | 80 | 0 | 60 | 100 | 100 | 90 | 80 |
| Pigweed | 100 | 0 | 100 | 20 | 60 | 100 | 30 | 100 | 0 | 40 | 100 | 100 | 90 | 50 |
| Ragweed | 100 | 0 | 100 | 50 | 50 | 100 | 0 | 100 | 0 | 0 | 100 | 100 | 90 | 30 |
| Ryegrass, Italian | 80 | 0 | 50 | 0 | 20 | 80 | 0 | 60 | 0 | 0 | 80 | 90 | 20 | 0 |

| 500 g ai/ha | Compounds | | | | | | | | | | | | | |
|---|---|---|---|---|---|---|---|---|---|---|---|---|---|---|
| Preemergence | 55 | 56 | 57 | 58 | 59 | 60 | 61 | 62 | 63 | 64 | 65 | 66 | 67 | 68 |
| Barnyardgrass | 80 | 70 | 70 | 0 | 0 | 50 | 90 | 80 | 100 | 0 | 0 | 0 | 0 | 0 |
| Foxtail, Giant | 90 | 0 | 90 | 0 | 30 | 0 | 100 | 90 | 100 | 0 | 0 | 0 | 0 | 0 |
| Kochia | 90 | 0 | 60 | 0 | 80 | 0 | 90 | 90 | 100 | 0 | 0 | 0 | 90 | 0 |
| Pigweed | 100 | 30 | 100 | 0 | 80 | 0 | 100 | 90 | 100 | 0 | 0 | 0 | 90 | 0 |
| Ragweed | 100 | 80 | 100 | 0 | 30 | 0 | 100 | 100 | 100 | 0 | 40 | 0 | 0 | 0 |
| Ryegrass, Italian | 70 | 0 | 50 | 0 | 0 | 20 | 100 | 80 | 90 | 0 | 0 | 0 | 0 | 0 |

| 500 g ai/ha | Compounds | | | | | | | | | | | | | |
|---|---|---|---|---|---|---|---|---|---|---|---|---|---|---|
| Preemergence | 69 | 70 | 71 | 72 | 73 | 74 | 75 | 76 | 77 | 78 | 79 | 80 | 81 | 82 |
| Barnyardgrass | 0 | 30 | 100 | 0 | 100 | 0 | 90 | 0 | 0 | 0 | 100 | 100 | 0 | 100 |
| Foxtail, Giant | 0 | 50 | 100 | 0 | 100 | 0 | 100 | 0 | 40 | 0 | 100 | 100 | 20 | 90 |
| Kochia | 70 | 40 | 100 | 0 | 100 | 0 | 100 | 0 | 60 | 90 | 0 | 100 | 20 | 90 |
| Pigweed | 70 | 90 | 100 | 80 | 100 | 10 | 100 | 0 | 90 | 20 | 100 | 100 | 10 | 100 |
| Ragweed | 70 | 90 | 100 | 0 | 100 | 40 | 100 | 0 | 90 | 20 | 100 | 100 | 70 | 100 |
| Ryegrass, Italian | 0 | 0 | 80 | 0 | 100 | 0 | 100 | 30 | 30 | 50 | 100 | 100 | 60 | 100 |

| 500 g ai/ha | Compounds | | | | | | | | | | | | | |
|---|---|---|---|---|---|---|---|---|---|---|---|---|---|---|
| Preemergence | 83 | 84 | 85 | 86 | 87 | 88 | 89 | 90 | 91 | 92 | 93 | 94 | 95 | 96 |
| Barnyardgrass | 0 | 90 | 100 | 90 | 0 | 90 | 0 | 80 | 0 | 80 | 0 | 90 | 100 | 90 |
| Foxtail, Giant | 0 | 100 | 100 | 100 | 0 | 100 | 0 | 100 | 0 | 100 | 90 | 100 | 100 | 80 |
| Kochia | 0 | 90 | 90 | 100 | 0 | 100 | 0 | 90 | 0 | 90 | 90 | 90 | 90 | 0 |
| Pigweed | 10 | 100 | 100 | 100 | 10 | 100 | 0 | 100 | 0 | 100 | 100 | 100 | 100 | 90 |
| Ragweed | 40 | 100 | 100 | 100 | 0 | 100 | 20 | 100 | 0 | 100 | 100 | 90 | 100 | 70 |
| Ryegrass, Italian | 0 | 90 | 50 | 70 | 0 | 100 | 0 | 50 | 30 | 40 | 50 | 90 | 60 | 50 |

| 500 g ai/ha | Compounds | | | | | | | | | |
|---|---|---|---|---|---|---|---|---|---|---|
| Preemergence | 98 | 99 | 100 | 101 | 102 | 103 | 104 | 105 | 106 | 107 |
| Barnyardgrass | 50 | 90 | 90 | 90 | 80 | 0 | 0 | 60 | 0 | 100 |
| Foxtail, Giant | 40 | 100 | 100 | 100 | 100 | 0 | 60 | 100 | 50 | 100 |
| Kochia | 0 | 90 | 90 | 90 | 90 | 40 | 90 | 100 | 90 | 100 |
| Pigweed | 100 | 100 | 100 | 100 | 100 | 70 | 80 | 100 | 100 | 100 |
| Ragweed | 80 | 100 | 100 | 100 | 80 | 30 | 100 | 100 | 100 | 100 |
| Ryegrass, Italian | 40 | 80 | 90 | 90 | 60 | 30 | 60 | 90 | 30 | 90 |

| 125 g ai/ha | Compounds | | | | | | | | | | | | | |
|---|---|---|---|---|---|---|---|---|---|---|---|---|---|---|
| Preemergence | 41 | 42 | 43 | 44 | 45 | 46 | 47 | 48 | 49 | 50 | 51 | 52 | 53 | 54 |
| Barnyardgrass | 40 | 0 | 70 | 0 | 40 | 20 | 0 | 0 | 0 | 0 | 40 | 70 | 0 | 0 |
| Foxtail, Giant | 30 | 0 | 80 | 0 | 0 | 70 | 0 | 60 | 0 | 0 | 10 | 90 | 90 | 0 |
| Kochia | 70 | 0 | 90 | 0 | 0 | 90 | 0 | 0 | 0 | 40 | 90 | 90 | 60 | 40 |
| Pigweed | 80 | 0 | 90 | 20 | 0 | 100 | 0 | 60 | 0 | 0 | 90 | 100 | 80 | 50 |
| Ragweed | 90 | 0 | 100 | 20 | 0 | 80 | 0 | 30 | 0 | 0 | 100 | 100 | 100 | 0 |
| Ryegrass, Italian | 20 | 0 | 20 | 0 | 0 | 20 | 0 | 0 | 0 | 0 | 60 | 80 | 20 | 0 |

| 125 g ai/ha | Compounds | | | | | | | | | | | | | |
|---|---|---|---|---|---|---|---|---|---|---|---|---|---|---|
| Preemergence | 55 | 56 | 57 | 58 | 59 | 60 | 61 | 62 | 63 | 64 | 65 | 66 | 67 | 68 |
| Barnyardgrass | 80 | 70 | 0 | 0 | 0 | 0 | 80 | 10 | 70 | 0 | 0 | 0 | 0 | 0 |
| Foxtail, Giant | 50 | 0 | 30 | 0 | 0 | 0 | 100 | 20 | 60 | 0 | 0 | 0 | 0 | 0 |
| Kochia | 40 | 0 | 0 | 0 | 70 | 0 | 90 | 70 | 80 | 0 | 0 | 0 | 0 | 0 |
| Pigweed | 80 | 0 | 30 | 0 | 30 | 0 | 100 | 20 | 90 | 0 | 0 | 0 | 30 | 0 |
| Ragweed | 100 | 20 | 60 | 0 | 0 | 0 | 90 | 50 | 50 | 0 | 0 | 0 | 0 | 0 |
| Ryegrass, Italian | 20 | 0 | 20 | 0 | 0 | 0 | 40 | 20 | 40 | 0 | 0 | 0 | 0 | 0 |

| 125 g ai/ha | Compounds | | | | | | | | | | | | | |
|---|---|---|---|---|---|---|---|---|---|---|---|---|---|---|
| Preemergence | 69 | 70 | 71 | 72 | 73 | 74 | 75 | 76 | 77 | 78 | 79 | 80 | 81 | 82 |
| Barnyardgrass | 0 | 0 | 70 | 0 | 90 | 0 | 90 | 0 | 0 | 0 | 90 | 90 | 0 | 90 |
| Foxtail, Giant | 0 | 0 | 90 | 0 | 100 | 0 | 90 | 0 | 20 | 0 | 100 | 90 | 0 | 80 |
| Kochia | 0 | 0 | 90 | 0 | 100 | 0 | 90 | 0 | 50 | 80 | 0 | 90 | 0 | 90 |
| Pigweed | 0 | 30 | 100 | 0 | 100 | 0 | 100 | 0 | 40 | 0 | 90 | 90 | 0 | 100 |
| Ragweed | 0 | 20 | 100 | 0 | 100 | 0 | 100 | 0 | 80 | 0 | 100 | 100 | 0 | 100 |
| Ryegrass, Italian | 0 | 0 | 20 | 0 | 100 | 0 | 80 | 0 | 20 | 20 | 100 | 70 | 0 | 90 |

| 125 g ai/ha | Compounds | | | | | | | | | | | | | |
|---|---|---|---|---|---|---|---|---|---|---|---|---|---|---|
| Preemergence | 83 | 84 | 85 | 86 | 87 | 88 | 89 | 90 | 91 | 92 | 93 | 94 | 95 | 96 |
| Barnyardgrass | 0 | 90 | 70 | 70 | 0 | 70 | 0 | 0 | 0 | 0 | 0 | 0 | 80 | 0 |
| Foxtail, Giant | 0 | 90 | 70 | 70 | 0 | 90 | 0 | 70 | 0 | 30 | 90 | 70 | 90 | 20 |
| Kochia | 0 | 90 | 30 | 80 | 0 | 70 | 0 | 0 | 0 | 70 | 90 | 60 | 80 | 0 |
| Pigweed | 0 | 100 | 100 | 100 | 0 | 100 | 0 | 70 | 0 | 100 | 90 | 60 | 90 | 70 |
| Ragweed | 0 | 100 | 90 | 100 | 0 | 80 | 0 | 50 | 0 | 100 | 90 | 50 | 50 | 50 |
| Ryegrass, Italian | 0 | 70 | 40 | 20 | 0 | 50 | 0 | 30 | 0 | 20 | 20 | 50 | 40 | 0 |

| | |
|---|---|
| 125 g ai/ha | Compounds |

| Preemergence | 98 | 99 | 100 | 101 | 102 | 103 | 104 | 105 | 106 | 107 |
|---|---|---|---|---|---|---|---|---|---|---|
| Barnyardgrass | 20 | 40 | 60 | 90 | 30 | 0 | 0 | 40 | 0 | 20 |
| Foxtail, Giant | 0 | 70 | 90 | 100 | 80 | 0 | 20 | 40 | 0 | 30 |
| Kochia | 0 | 0 | 90 | 90 | 70 | 0 | 30 | 90 | 80 | 80 |
| Pigweed | 0 | 40 | 100 | 70 | 100 | 20 | 50 | 100 | 30 | 60 |
| Ragweed | 20 | 90 | 50 | 70 | 50 | 0 | 60 | 100 | 60 | 100 |
| Ryegrass, Italian | 20 | 20 | 0 | 30 | 30 | 0 | 30 | 20 | 0 | 50 |

| 125 g ai/ha | Compounds | | | | | | | | | |
|---|---|---|---|---|---|---|---|---|---|---|
| Preemergence | 108 | 109 | 110 | 111 | 112 | 113 | 114 | 115 | 116 | 118 |
| Barnyardgrass | 30 | 0 | 0 | 0 | 0 | 0 | 40 | 60 | 30 | 80 |
| Foxtail, Giant | 30 | 30 | 0 | 10 | 0 | 10 | 70 | 90 | 60 | 90 |
| Kochia | 90 | 90 | 0 | 70 | 70 | 80 | 60 | 80 | 40 | 90 |
| Pigweed | 90 | 90 | 30 | 70 | 80 | — | 100 | 100 | 80 | 100 |
| Ragweed | 70 | 90 | 80 | 90 | 30 | 20 | 60 | 80 | 70 | 100 |
| Ryegrass, Italian | 30 | 40 | 0 | 0 | 0 | 30 | 50 | 40 | 40 | 40 |
| 31 g ai/ha | Compounds | | | | | | | | | |
| Preemergence | 108 | 109 | 110 | 111 | 112 | 113 | 114 | 115 | 116 | 118 |
| Barnyardgrass | 40 | 0 | 0 | 0 | 0 | 0 | 0 | 20 | 0 | 20 |
| Foxtail, Giant | 30 | 0 | 0 | 0 | 0 | 0 | 10 | 30 | 20 | 50 |
| Kochia | 90 | 80 | 0 | 40 | 0 | 20 | 30 | 40 | 30 | 70 |
| Pigweed | 70 | 70 | 0 | 10 | 0 | — | 50 | 90 | 40 | 70 |
| Ragweed | 50 | 30 | 40 | 30 | 0 | 0 | 40 | 60 | 60 | 50 |
| Ryegrass, Italian | 0 | 20 | 0 | 0 | 0 | 0 | 30 | 20 | 20 | 0 |

| 1000 g ai/ha | Compound |
|---|---|
| Postemergence | 97 |
| Barnyardgrass | 0 |
| Blackgrass | 0 |
| Corn | 0 |
| Foxtail, Giant | 0 |
| Galium | 20 |
| Kochia | 20 |
| Pigweed | 0 |
| Ragweed | 0 |
| Ryegrass, Italian | 0 |
| Wheat | 0 |

| 500 g ai/ha | Compounds | | | | | | | | | | | | | |
|---|---|---|---|---|---|---|---|---|---|---|---|---|---|---|
| Postemergence | 41 | 42 | 43 | 44 | 45 | 46 | 47 | 48 | 49 | 50 | 51 | 52 | 53 | 54 |
| Barnyardgrass | 50 | 0 | 100 | 0 | 10 | 60 | 0 | 30 | 0 | 0 | 50 | 90 | 30 | 20 |
| Blackgrass | 0 | 0 | 20 | 20 | 0 | 30 | 30 | 30 | 0 | 0 | 10 | 90 | 0 | 20 |
| Corn | 20 | 0 | 20 | 0 | 0 | 60 | 0 | 30 | 0 | 10 | 10 | — | 0 | 10 |
| Foxtail, Giant | 100 | 0 | 100 | 10 | 40 | 60 | 0 | 30 | 10 | 40 | 50 | 90 | 30 | 10 |
| Galium | 70 | 20 | 90 | 0 | 30 | 90 | 0 | 90 | 0 | 20 | 90 | 90 | 80 | 40 |
| Kochia | 90 | 0 | 90 | 50 | 50 | 90 | 60 | 90 | 0 | 60 | 90 | 90 | 90 | 100 |
| Pigweed | 100 | 0 | 100 | 60 | 80 | 100 | 80 | 100 | 50 | 90 | 100 | 100 | 100 | 100 |
| Ragweed | 90 | 0 | 90 | 0 | 20 | 80 | 0 | 70 | 0 | 30 | 100 | 100 | 80 | 60 |
| Ryegrass, Italian | 70 | 0 | 40 | 0 | 40 | 40 | 0 | 40 | 0 | 0 | 50 | 100 | 60 | 0 |
| Wheat | 0 | 0 | 0 | 0 | 0 | 0 | 0 | 0 | 0 | 0 | 30 | 30 | 0 | 0 |

| 500 g ai/ha | Compounds | | | | | | | | | | | | | |
|---|---|---|---|---|---|---|---|---|---|---|---|---|---|---|
| Postemergence | 55 | 56 | 57 | 58 | 59 | 60 | 61 | 62 | 63 | 64 | 65 | 66 | 67 | 68 |
| Barnyardgrass | 70 | 30 | 70 | 50 | 0 | 50 | 100 | 60 | 60 | 20 | 0 | 30 | 0 | 0 |
| Blackgrass | 20 | 0 | 10 | 0 | 0 | 20 | 70 | 10 | 20 | 0 | 0 | 30 | 0 | 0 |
| Corn | 0 | 10 | 20 | 0 | 0 | 0 | 60 | 10 | 10 | 0 | 0 | 20 | 0 | 0 |
| Foxtail, Giant | 90 | 20 | 50 | 10 | 0 | 50 | 90 | 90 | 90 | 0 | 0 | 0 | 0 | 0 |
| Galium | 90 | 0 | 80 | 20 | 20 | 40 | 100 | 70 | 90 | 30 | 0 | 40 | 40 | 0 |
| Kochia | 90 | 40 | 60 | 80 | 70 | 100 | 90 | 90 | 90 | 30 | 60 | 90 | 70 | 40 |
| Pigweed | 100 | 60 | 80 | 60 | 80 | 90 | 100 | 100 | 100 | 60 | 30 | 100 | 60 | 0 |
| Ragweed | 80 | 30 | 70 | 20 | 0 | 60 | 100 | 70 | 60 | 20 | 0 | 20 | 0 | 50 |
| Ryegrass, Italian | 30 | 0 | 60 | 0 | 0 | 0 | 90 | 0 | 0 | 0 | 0 | 0 | 0 | 0 |
| Wheat | 0 | 0 | 20 | 0 | 0 | 0 | 30 | 0 | 0 | 0 | 30 | 0 | 0 | 0 |

| 500 g ai/ha | Compounds | | | | | | | | | | | | | |
|---|---|---|---|---|---|---|---|---|---|---|---|---|---|---|
| Postemergence | 69 | 70 | 71 | 72 | 73 | 74 | 75 | 76 | 77 | 78 | 79 | 80 | 81 | 82 |
| Barnyardgrass | 0 | 70 | 100 | 0 | 100 | 0 | 100 | 0 | 0 | 0 | 100 | 100 | 0 | 80 |
| Blackgrass | 30 | 30 | 50 | 0 | 80 | 0 | 30 | 0 | 0 | 0 | 90 | 70 | 0 | 70 |
| Corn | 0 | 0 | 40 | 0 | 40 | 0 | 10 | 0 | 20 | 0 | 70 | 50 | 0 | 40 |
| Foxtail, Giant | 0 | 60 | 90 | 0 | 100 | 0 | 90 | 0 | 20 | 0 | 90 | 0 | 10 | 90 |
| Galium | 0 | 80 | 90 | 40 | 90 | 50 | 80 | 30 | 80 | 10 | 90 | 90 | 30 | 90 |
| Kochia | 80 | 80 | 90 | 30 | 100 | 60 | 90 | 0 | 90 | 20 | 90 | 90 | 40 | 90 |
| Pigweed | 80 | 100 | 100 | 50 | 100 | 90 | 100 | 0 | 100 | 70 | 100 | 100 | 80 | 100 |
| Ragweed | 0 | 50 | 90 | 30 | 100 | 30 | 90 | 30 | 90 | 0 | 90 | 90 | 20 | 90 |
| Ryegrass, Italian | 0 | 0 | 70 | 0 | 100 | 0 | 50 | 0 | 40 | 0 | 80 | 90 | 0 | 90 |
| Wheat | 0 | 0 | 0 | 0 | 0 | 0 | 0 | 0 | 30 | 0 | 20 | 30 | 0 | 0 |

| 500 g ai/ha | Compounds | | | | | | | | | | | | | |
|---|---|---|---|---|---|---|---|---|---|---|---|---|---|---|
| Postemergence | 83 | 84 | 85 | 86 | 87 | 88 | 89 | 90 | 91 | 92 | 93 | 94 | 95 | 96 |
| Barnyardgrass | 30 | 100 | 80 | 50 | 0 | 90 | 0 | — | — | — | — | 90 | 70 | 40 |
| Blackgrass | 0 | 0 | 50 | 10 | 0 | 50 | 0 | 60 | 0 | 0 | 0 | 0 | 70 | 30 |
| Corn | 0 | 0 | 40 | 0 | 0 | 30 | 0 | 0 | 0 | 0 | 0 | 20 | 30 | 60 |
| Foxtail, Giant | 10 | 90 | 90 | 60 | 0 | 90 | 0 | 50 | 0 | 60 | 50 | 90 | 90 | 60 |
| Galium | 0 | 80 | 90 | 70 | 0 | 90 | 0 | 70 | 0 | 100 | 70 | 80 | 80 | 80 |
| Kochia | 30 | 90 | 90 | 90 | 30 | 90 | 0 | 90 | 0 | 20 | 90 | 90 | 90 | 90 |
| Pigweed | 80 | 100 | 10 | 100 | 60 | 100 | 0 | 100 | 0 | 100 | 100 | 100 | 100 | 90 |
| Ragweed | 0 | 90 | 90 | 80 | 20 | 90 | 0 | 70 | 0 | 50 | 50 | 70 | 80 | 80 |
| Ryegrass, Italian | 0 | 70 | 60 | 50 | 0 | 60 | 0 | 40 | 0 | 50 | 40 | 90 | 100 | 0 |
| Wheat | 0 | 0 | 30 | 0 | 0 | 0 | 0 | 30 | 0 | 30 | 30 | 30 | 30 | 0 |

| | |
|---|---|
| 500 g ai/ha | Compounds |

| Postemergence | 98 | 99 | 100 | 101 | 102 | 103 | 104 | 105 | 106 | 107 |
|---|---|---|---|---|---|---|---|---|---|---|
| Barnyardgrass | 0 | 70 | 90 | 100 | 70 | 0 | 20 | 60 | 30 | 100 |
| Blackgrass | 0 | 0 | 70 | 70 | 40 | 20 | 20 | 0 | 0 | 80 |
| Corn | 0 | 30 | 20 | 40 | 20 | 0 | 0 | 0 | 0 | 30 |
| Foxtail, Giant | 0 | 60 | 50 | 100 | 20 | 0 | 20 | 60 | 0 | 60 |
| Galium | 70 | 70 | 100 | 90 | 100 | 0 | 80 | 90 | 80 | 80 |
| Kochia | 40 | 80 | 100 | 100 | 90 | 40 | 60 | 100 | 90 | 90 |
| Pigweed | 90 | 70 | 100 | 100 | 100 | 100 | 100 | 60 | 100 | 100 |
| Ragweed | 60 | 80 | 100 | 100 | 100 | 0 | 80 | 100 | 100 | 100 |
| Ryegrass, Italian | 0 | 40 | 70 | 70 | 30 | 0 | 70 | 100 | 0 | 40 |
| Wheat | 0 | 0 | 40 | 40 | 40 | 30 | 0 | 0 | 0 | 30 |

| 125 g ai/ha | Compounds | | | | | | | | | | | | | |
|---|---|---|---|---|---|---|---|---|---|---|---|---|---|---|
| Postemergence | 41 | 42 | 43 | 44 | 45 | 46 | 47 | 48 | 49 | 50 | 51 | 52 | 53 | 54 |
| Barnyardgrass | 0 | 30 | 20 | 0 | 0 | 20 | 0 | 0 | 0 | 0 | 0 | 30 | 0 | 30 |
| Blackgrass | 0 | 20 | 0 | 0 | 0 | 20 | 0 | 30 | 0 | 0 | 0 | 50 | 0 | 0 |
| Corn | 10 | 0 | 0 | 0 | 0 | 0 | 0 | 0 | 0 | 0 | 0 | — | 0 | 0 |
| Foxtail, Giant | 50 | 0 | 30 | 0 | 0 | 10 | 0 | 10 | 0 | 0 | 20 | 40 | 10 | 0 |
| Galium | 50 | 20 | 70 | 0 | 0 | 60 | 0 | 60 | 0 | 10 | 70 | 80 | 30 | 20 |
| Kochia | 70 | 0 | 90 | 0 | 0 | 90 | 0 | 30 | 0 | 20 | 80 | 90 | 50 | 50 |
| Pigweed | 90 | 0 | 100 | 0 | 20 | 100 | 20 | 90 | 0 | 60 | 100 | 100 | 60 | 70 |
| Ragweed | 60 | 30 | 60 | 0 | 0 | 50 | 0 | 20 | 0 | 0 | 90 | 80 | 20 | 20 |
| Ryegrass, Italian | 20 | 0 | 40 | 0 | 0 | 0 | 0 | 0 | 0 | 30 | 30 | 60 | 20 | 0 |
| Wheat | 0 | 0 | 0 | 0 | 0 | 0 | 0 | 0 | 0 | 0 | 0 | 0 | 0 | 0 |

| 125 g ai/ha | Compounds | | | | | | | | | | | | | |
|---|---|---|---|---|---|---|---|---|---|---|---|---|---|---|
| Postemergence | 55 | 56 | 57 | 58 | 59 | 60 | 61 | 62 | 63 | 64 | 65 | 66 | 67 | 68 |
| Barnyardgrass | 30 | 50 | 0 | 0 | 0 | 0 | 80 | 20 | 30 | 0 | 0 | 0 | 0 | 0 |
| Blackgrass | 0 | 0 | 0 | 0 | 0 | 0 | 80 | 20 | 0 | 0 | 0 | 0 | 0 | 0 |
| Corn | 10 | 0 | 0 | 0 | 0 | 0 | 10 | 0 | 0 | 0 | 0 | 0 | 0 | 20 |
| Foxtail, Giant | 20 | 10 | 0 | 0 | 0 | 20 | 90 | 20 | 20 | 0 | 0 | 0 | 0 | 0 |
| Galium | 70 | 0 | 60 | 0 | 20 | 30 | 70 | 50 | 50 | 0 | 0 | 30 | 20 | 0 |
| Kochia | 70 | 0 | 0 | 30 | 50 | 30 | 80 | 70 | 80 | 0 | 0 | 20 | 50 | 20 |
| Pigweed | 100 | 20 | 60 | 20 | 60 | 50 | 100 | 90 | 100 | 20 | 0 | 30 | 20 | 0 |
| Ragweed | 50 | 0 | 40 | 0 | 0 | 20 | 60 | 30 | 30 | 0 | 0 | 0 | 0 | 40 |
| Ryegrass, Italian | 30 | 0 | 0 | 0 | 0 | 0 | 50 | 0 | 0 | 0 | 0 | 0 | 0 | 0 |
| Wheat | 0 | 0 | 0 | 0 | 0 | 0 | 20 | 0 | 0 | 0 | 30 | 0 | 0 | 0 |

| 125 g ai/ha | Compounds | | | | | | | | | | | | | |
|---|---|---|---|---|---|---|---|---|---|---|---|---|---|---|
| Postemergence | 69 | 70 | 71 | 72 | 73 | 74 | 75 | 76 | 77 | 78 | 79 | 80 | 81 | 82 |
| Barnyardgrass | 0 | 20 | 30 | 0 | 60 | 0 | 30 | 0 | 0 | 0 | 70 | 40 | 0 | 30 |
| Blackgrass | 0 | 0 | 0 | 0 | 50 | 0 | 0 | 0 | 0 | 0 | 60 | 0 | 0 | 20 |
| Corn | 20 | 0 | 0 | 0 | 30 | 0 | 0 | 0 | 0 | 0 | 0 | 20 | 0 | 0 |
| Foxtail, Giant | 0 | 0 | 60 | 0 | 70 | 0 | 60 | 0 | 0 | 0 | 60 | 0 | 0 | 20 |
| Galium | 0 | 0 | 80 | 0 | 90 | 0 | 80 | 30 | 50 | 0 | 70 | 70 | 0 | 80 |
| Kochia | 30 | 50 | 90 | 0 | 90 | 20 | 80 | 0 | 30 | 0 | 90 | 80 | 20 | 80 |
| Pigweed | 20 | 100 | 100 | 40 | 100 | 60 | 100 | 0 | 70 | 60 | 100 | 100 | 0 | 90 |
| Ragweed | 0 | 0 | 80 | 0 | 90 | 20 | 70 | 0 | 20 | 0 | 80 | 90 | 0 | 80 |
| Ryegrass, Italian | 0 | 0 | 30 | 0 | 90 | 0 | 0 | 0 | 0 | 0 | 60 | 0 | 0 | 0 |
| Wheat | 0 | 0 | 0 | 0 | 0 | 0 | 0 | 0 | 20 | 0 | 0 | 30 | 0 | 0 |

| 125 g ai/ha | Compounds | | | | | | | | | | | | | |
|---|---|---|---|---|---|---|---|---|---|---|---|---|---|---|
| Postemergence | 83 | 84 | 85 | 86 | 87 | 88 | 89 | 90 | 91 | 92 | 93 | 94 | 95 | 96 |
| Barnyardgrass | 0 | 70 | 50 | 40 | 0 | 10 | 0 | — | — | — | — | 40 | 0 | 30 |
| Blackgrass | 0 | 0 | 40 | 0 | 0 | 50 | 0 | 0 | 0 | 0 | 40 | 0 | 0 | 0 |
| Corn | 0 | 0 | 10 | 0 | 0 | 20 | 0 | 0 | 0 | 0 | 10 | 0 | 10 | 40 |
| Foxtail, Giant | 0 | 50 | 40 | 60 | 0 | 30 | 0 | 0 | 0 | 20 | 30 | 60 | 50 | 0 |
| Galium | 10 | 60 | 80 | 70 | 0 | 90 | 0 | 40 | 0 | 50 | 50 | 40 | 80 | 60 |
| Kochia | 10 | 80 | 70 | 70 | 0 | 90 | 0 | 20 | 0 | 70 | 70 | 70 | 80 | 20 |
| Pigweed | 30 | 100 | 90 | 90 | 0 | 100 | 0 | 60 | 0 | 80 | 80 | 90 | 100 | 60 |
| Ragweed | 0 | 40 | 50 | 20 | 0 | 30 | 0 | 30 | 0 | 20 | 30 | 50 | 60 | 20 |
| Ryegrass, Italian | 0 | 0 | 50 | 0 | 0 | 50 | 0 | 30 | 0 | 50 | 50 | 50 | 50 | 0 |
| Wheat | 0 | 0 | 30 | 0 | 0 | 0 | 0 | 0 | 0 | 0 | 0 | 0 | 0 | 0 |

| | |
|---|---|
| 125 g ai/ha | Compounds |

| Postemergence | 98 | 99 | 100 | 101 | 102 | 103 | 104 | 105 | 106 | 107 |
|---|---|---|---|---|---|---|---|---|---|---|
| Barnyardgrass | 0 | 30 | 30 | 30 | 10 | 0 | 0 | 30 | 0 | 20 |
| Blackgrass | 0 | 0 | 30 | 20 | 0 | 0 | 0 | 0 | 0 | 0 |
| Corn | 0 | 0 | 0 | 0 | 0 | 10 | 0 | 0 | 0 | 0 |
| Foxtail, Giant | 0 | 10 | 20 | 10 | 0 | 0 | 0 | 0 | 0 | 10 |
| Galium | 50 | 20 | 100 | 80 | 100 | 20 | 40 | 90 | 70 | 70 |
| Kochia | 20 | 50 | 60 | 70 | 70 | 10 | 10 | 80 | 80 | 50 |
| Pigweed | 60 | 50 | 100 | 100 | 100 | 30 | 80 | 0 | 70 | 80 |
| Ragweed | 30 | 20 | 60 | 30 | 30 | 0 | 20 | 70 | 20 | 40 |
| Ryegrass, Italian | 0 | 0 | 40 | 60 | 0 | 0 | 0 | 100 | 0 | 0 |
| Wheat | 0 | 0 | 50 | 0 | 0 | 0 | 0 | 0 | 0 | 0 |

| 125 g ai/ha | Compounds | | | | | | | | | |
|---|---|---|---|---|---|---|---|---|---|---|
| Postemergence | 108 | 109 | 110 | 111 | 112 | 113 | 114 | 115 | 116 | 118 |
| Barnyardgrass | 0 | 0 | 10 | 0 | 0 | 0 | 30 | 100 | 50 | 90 |
| Blackgrass | 0 | 10 | 0 | 0 | 0 | 0 | 30 | 30 | 0 | 10 |
| Corn | 10 | 0 | 10 | 20 | 0 | 0 | 30 | 100 | 30 | 50 |
| Foxtail, Giant | 20 | 20 | 0 | 0 | 0 | 0 | 100 | 100 | 90 | 40 |
| Galium | 70 | 70 | 70 | 50 | 60 | 70 | 90 | 90 | 80 | 90 |
| Kochia | 70 | 70 | 10 | 60 | 60 | 70 | 80 | 90 | 80 | 90 |
| Pigweed | 90 | 90 | 80 | 80 | 60 | 100 | 100 | 100 | 100 | 100 |
| Ragweed | 50 | 60 | 30 | 50 | 0 | 40 | 80 | 80 | 60 | 90 |
| Ryegrass, Italian | 50 | 0 | 0 | 0 | 0 | 0 | 40 | 50 | 30 | 50 |
| Wheat | 0 | 0 | 0 | 0 | 0 | 0 | 30 | 20 | 0 | 0 |
| 31 g ai/ha | Compounds | | | | | | | | | |
| Postemergence | 108 | 109 | 110 | 111 | 112 | 113 | 114 | 115 | 116 | 118 |
| Barnyardgrass | 20 | 0 | 0 | 10 | 0 | 0 | 10 | 10 | 10 | 20 |
| Blackgrass | 0 | 10 | 0 | 0 | 0 | 0 | 0 | 0 | 0 | 0 |
| Corn | 0 | 0 | 0 | 0 | 30 | 20 | 10 | 10 | 20 | 30 |
| Foxtail, Giant | 0 | 0 | 0 | 0 | 0 | 0 | 10 | 60 | 10 | 20 |
| Galium | 50 | 40 | 30 | 20 | 20 | 40 | 60 | 50 | 60 | 60 |
| Kochia | 40 | 50 | 0 | 0 | 10 | 30 | 50 | 70 | 30 | 80 |
| Pigweed | 70 | 80 | 60 | 50 | 30 | 40 | 100 | 100 | 90 | 90 |
| Ragweed | 30 | 50 | 0 | 40 | 0 | 0 | 40 | 30 | 40 | 50 |
| Ryegrass, Italian | 0 | 0 | 0 | 0 | 0 | 0 | 0 | 30 | 30 | 0 |
| Wheat | 0 | 0 | 0 | 0 | 0 | 0 | 0 | 0 | 0 | 0 |

### Test B

Plant species in the flooded paddy test selected from rice (Oryza sativa), sedge, umbrella (small-flower umbrella sedge, Cyperus difformis), ducksalad (Heteranthera limosa), and barnyardgrass (Echinochloa crus-galli) were grown to the 2-leaf stage for testing. At the time of treatment, test pots were flooded to 3 cm above the soil surface, treated by application of test compounds directly to the paddy water, and then maintained at that water depth for the duration of the test. Treated plants and controls were maintained in a greenhouse for 13 to 15 d, after which time all species were compared to controls and visually evaluated. Plant response ratings, summarized in Table B, are based on a scale of 0 to 100 where 0 is no effect and 100 is complete control. A dash (-) response means no test result.

TABLE B

| 250 g ai/ha | Compounds | | | | | | | | | | | | | |
|---|---|---|---|---|---|---|---|---|---|---|---|---|---|---|
| Flood | 1 | 2 | 3 | 4 | 5 | 6 | 7 | 8 | 9 | 10 | 11 | 12 | 13 | 14 |
| Barnyardgrass | 0 | 0 | 0 | 95 | 0 | 0 | 0 | 0 | 0 | 0 | 0 | 0 | 90 | 0 |
| Ducksalad | 0 | 0 | 30 | 45 | 0 | 0 | 0 | 98 | 0 | 0 | 0 | 0 | 75 | 0 |
| Rice | 0 | 0 | 0 | 0 | 0 | 0 | 0 | 40 | 0 | 0 | 0 | 0 | 10 | 0 |
| Sedge, Umbrella | 0 | 0 | 65 | 100 | 0 | 0 | 0 | 100 | 0 | 0 | 0 | 0 | 95 | 0 |

| 250 g ai/ha | Compounds | | | | | | | | | | | | | |
|---|---|---|---|---|---|---|---|---|---|---|---|---|---|---|
| Flood | 15 | 16 | 17 | 18 | 19 | 20 | 21 | 22 | 23 | 24 | 25 | 26 | 27 | 28 |
| Barnyardgrass | 0 | 50 | 0 | 0 | 0 | 0 | 0 | 40 | 0 | 0 | 0 | 0 | 0 | 0 |
| Ducksalad | 0 | 15 | 0 | 85 | 0 | 0 | 60 | 100 | 0 | 85 | 0 | 0 | 0 | 75 |
| Rice | 0 | 0 | 0 | 0 | 0 | 0 | 0 | 0 | 0 | 0 | 0 | 0 | 0 | 20 |
| Sedge, Umbrella | 0 | 95 | 0 | 98 | 0 | 0 | 65 | 95 | 0 | 100 | 100 | 0 | 50 | 100 |

| 250 g ai/ha | Compounds | | | | | | | | | | | |
|---|---|---|---|---|---|---|---|---|---|---|---|---|
| Flood | 29 | 30 | 31 | 32 | 33 | 34 | 35 | 36 | 37 | 38 | 39 | 40 |
| Barnyardgrass | 0 | 0 | 0 | 0 | 0 | 0 | 0 | 0 | 0 | 0 | 0 | 20 |
| Ducksalad | 0 | 0 | 0 | 0 | 70 | 0 | 0 | 0 | 0 | 0 | 0 | 20 |
| Rice | 0 | 0 | 0 | 0 | 0 | 0 | 0 | 0 | 0 | 0 | 0 | 0 |
| Sedge, Umbrella | 0 | 80 | 0 | 0 | 25 | 0 | 0 | 0 | 0 | 0 | 0 | 40 |

| 250 g ai/ha | Compounds | | | | | | | | | | | | | |
|---|---|---|---|---|---|---|---|---|---|---|---|---|---|---|
| Flood | 41 | 42 | 43 | 44 | 45 | 46 | 47 | 48 | 49 | 50 | 51 | 52 | 53 | 54 |
| Barnyardgrass | 15 | 0 | 0 | 0 | 0 | 40 | 0 | 0 | 0 | 0 | 0 | 90 | 0 | 0 |
| Ducksalad | 35 | 0 | 0 | 0 | 0 | 30 | 0 | 0 | 0 | 0 | 0 | 100 | 0 | 0 |
| Rice | 0 | 0 | 0 | 0 | 0 | 10 | 0 | 0 | 0 | 0 | 0 | 10 | 0 | 0 |
| Sedge, Umbrella | 65 | 0 | 0 | 0 | 0 | 95 | 0 | 0 | 0 | 0 | 0 | 95 | 0 | 0 |

| 250 g ai/ha | Compounds | | | | | | | | | | | | | |
|---|---|---|---|---|---|---|---|---|---|---|---|---|---|---|
| Flood | 55 | 56 | 57 | 58 | 59 | 60 | 61 | 62 | 63 | 64 | 65 | 66 | 67 | 68 |
| Barnyardgrass | 30 | 0 | 0 | 0 | 0 | 0 | 40 | 0 | 45 | 0 | 0 | 0 | 0 | 0 |
| Ducksalad | 70 | 0 | 30 | 0 | 0 | 0 | 50 | 15 | 75 | 0 | 0 | 0 | 0 | 0 |
| Rice | 0 | 0 | 0 | 0 | 0 | 0 | 0 | 0 | 0 | 0 | 0 | 0 | 0 | 0 |
| Sedge, Umbrella | 98 | 0 | 80 | 0 | 0 | 0 | 90 | 95 | 80 | 0 | 0 | 0 | 0 | 0 |
| 250 g ai/ha | Compounds | | | | | | | | | | | | | |
| Flood | 69 | 70 | 71 | 72 | 73 | 74 | 75 | 76 | 77 | 78 | 79 | 80 | 81 | 82 |
| Barnyardgrass | 0 | 0 | 0 | 0 | 65 | 0 | 40 | 0 | 0 | 0 | 55 | 90 | 0 | 40 |
| Ducksalad | 0 | 0 | 0 | 0 | 98 | 0 | 95 | 0 | 0 | 0 | 98 | 85 | 0 | 15 |
| Rice | 0 | 0 | 0 | 0 | 10 | 0 | 0 | 0 | 0 | 0 | 10 | 10 | 0 | 0 |
| Sedge, Umbrella | 0 | 0 | 0 | 0 | 90 | 0 | 98 | 0 | 0 | 0 | 95 | 95 | 0 | 90 |
| 250 g ai/ha | Compounds | | | | | | | | | | | | | |
| Flood | 83 | 84 | 85 | 86 | 87 | 88 | 89 | 90 | 91 | 92 | 93 | 94 | 95 | 96 |
| Barnyardgrass | 0 | 85 | 0 | 60 | 0 | 80 | 30 | 40 | 0 | 0 | 0 | 35 | 35 | 0 |
| Ducksalad | 0 | 65 | 30 | 75 | 0 | 65 | 30 | 25 | 0 | 0 | 0 | 40 | 55 | 0 |
| Rice | 0 | 20 | 0 | 0 | 0 | 0 | 0 | 0 | 0 | 0 | 0 | 10 | 0 | 0 |
| Sedge, Umbrella | 0 | 90 | 85 | 98 | 0 | 95 | 95 | 20 | 0 | 0 | 0 | 45 | 70 | 0 |

| 250 g ai/ha | Compounds | | | | | | |
|---|---|---|---|---|---|---|---|
| Flood | 98 | 99 | 100 | 104 | 105 | 106 | 107 |
| Barnyardgrass | 0 | 0 | 45 | 0 | 0 | 0 | 45 |
| Ducksalad | 0 | 0 | 20 | 0 | 0 | 0 | 70 |
| Rice | 0 | 0 | 0 | 0 | 0 | 0 | 0 |
| Sedge, Umbrella | 0 | 0 | 35 | 0 | 0 | 0 | 75 |

| 250 g ai/ha | Compounds | | | | | | | | |
|---|---|---|---|---|---|---|---|---|---|
| Flood | 108 | 109 | 110 | 111 | 112 | 113 | 114 | 115 | 116 |
| Barnyardgrass | 35 | 35 | 20 | 0 | 0 | 0 | 65 | 90 | 35 |
| Ducksalad | 30 | 65 | 60 | 0 | 0 | 0 | 65 | 65 | 60 |
| Rice | 0 | 20 | 15 | 0 | 0 | 0 | 20 | 0 | 0 |
| Sedge, Umbrella | 75 | 75 | 90 | 0 | 0 | 0 | 80 | 95 | 65 |

What is claimed is:

1. A compound selected from Formula 1, N-oxides and salts thereof, 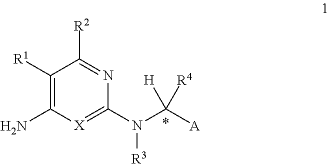
wherein A is selected from 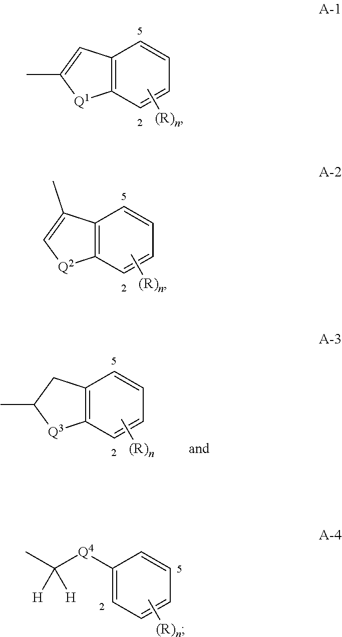
X is N or CR⁵;
R¹ and R² are independently H, halogen, hydroxy, cyano, nitro, amino, SF₅, C(O)OH, C(O)NH₂, C(S)NH₂, C₁-C₆ alkyl, C₁-C₆ haloalkyl, C₂-C₆ alkylcarbonyl, C₂-C₆ haloalkylcarbonyl, C₂-C₆ alkylcarbonyloxy, C₂-C₆ haloalkylcarbonyloxy, C₁-C₆ alkoxy, C₁-C₆ haloalkoxy, C₄-C₁₄ cycloalkylalkyl, C₃-C₈ cycloalkoxy, C₃-C₈ cyclohaloalkoxy, C₄-C₁₂ cycloalkylalkoxy, C₂-C₆ alkoxycarbonyl, C₂-C₆ haloalkoxycarbonyl, C₂-C₆ alkoxycarbonyl-C₁-C₆ haloalkyl, C₂-C₆ alkenyl, C₂-C₆ haloalkenyl, C₃-C₆ alkenylcarbonyl, C₃-C₆ haloalkenylcarbonyl, C₂-C₆ alkenyloxy, C₂-C₆ haloalkenyloxy, C₃-C₆ alkenyloxycarbonyl, C₃-C₆ haloalkenyloxycarbonyl, C₂-C₄ cyanoalkyl, C₂-C₄ cyanoalkoxy, C₁-C₄ nitroalkyl, C₁-C₄ nitroalkoxy, C₂-C₆ alkynyl, C₂-C₆ haloalkynyl, C₃-C₆ alkynylcarbonyl, C₃-C₆ haloalkynylcarbonyl, C₂-C₆ alkynyloxy, C₂-C₆ haloalkynyloxy, C₃-C₆ alkynyloxycarbonyl, C₃-C₆ haloalkynyloxycarbonyl, C₁-C₄ alkylthio, C₁-C₄ haloalkylthio, C₂-C₄ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylsulfonyloxy, C₁-C₄ haloalkylsulfonyloxy, C₁-C₆ hydroxyalkyl, C₁-C₆ hydroxyalkoxy, C₂-C₁₂ alkoxyalkyl, C₂-C₁₂ alkylthioalkyl, C₂-C₁₂ haloalkoxyalkyl, C₂-C₁₀ haloalkylthioalkoxy, C₂-C₁₂ alkoxyalkoxy, C₂-C₁₀ alkylthioalkoxy, C₂-C₁₂ haloalkoxyalkoxy, C₂-C₁₀ haloalkylthio, C₁-C₄ aminoalkyl, C₂-C₈ alkylaminoalkyl, C₃-C₁₂ dialkylaminoalkyl, C₁-C₄ aminoalkoxy, C₂-C₈ alkylaminoalkoxy or C₃-C₁₂ dialkylamino; or
R¹ and R² are independently C₃-C₈ cycloalkyl, each cycloalkyl optionally substituted with halogen, hydroxy, cyano, nitro, amino, C(O)OH, C(O)NH₂, C₁-C₆ alkyl, C₁-C₆ haloalkyl, C₁-C₆ haloalkoxy, C₃-C₈ cycloalkoxy, C₃-C₈ cyclohaloalkoxy, C₂-C₆ alkylcarbonyl, C₂-C₆ alkoxycarbonyl, C₂-C₆ alkoxycarbonyloxy, C₂-C₆ haloalkylcarbonyloxy, C₄-C₈ cycloalkylcarbonyl, C₄-C₈ cycloalkoxycarbonyl, C₂-C₆ haloalkoxycarbonyl, C₄-C₁₀ cycloalkylcarbonyloxy, C₃-C₈ cycloalkoxycarbonyloxy, C₂-C₆ haloalkoxycarbonyloxy;
R³ is H, C₁-C₄ alkyl, C₁-C₆ alkylcarbonyl, C₁-C₆ haloalkylcarbonyl, C₂-C₆ alkoxycarbonyl or C₂-C₆ haloalkoxycarbonyl;
R⁴ is C₁-C₆ alkyl, C₁-C₆ haloalkyl, C₂-C₆ alkenyl, C₂-C₆ alkynyl, C₃-C₇ cycloalkyl or C₃-C₇ cyclohaloalkyl;
R⁵ is H, halogen, cyano, C₁-C₆ alkyl or C₁-C₆ haloalkyl;
each R is independently halogen, hydroxy, cyano, amino, nitro, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₁-C₄ hydroxyalkyl, C₃-C₇ cycloalkyl, C₃-C₇ cyclohaloalkyl, C₄-C₈ cycloalkylalkyl, C₁-C₄ alkoxy, C₁-C₄ haloalkoxy, C₃-C₇ cycloalkoxy, C₃-C₇ cyclohaloalkoxy, C₄-C₈ cycloalkylalkoxy, C₂-C₄ alkenyloxy, C₂-C₄ alkynyloxy, C₂-C₄ alkoxyalkyl, C₂-C₄ alkoxyhaloalkyl, C₂-C₆ alkylcarbonyloxy, C₁-C₄ alkylthio, C₁-C₄ haloalkylthio, C₁-C₄ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylsulfonyloxy, C₂-C₄ cyanoalkyl, C₂-C₄ cyanoalkoxy, C₁-C₄ nitroalkyl, C₁-C₄ alkylamino, C₂-C₈ dialkylamino, C₃-C₆ cycloalkylamino, C₂-C₄ alkylcarbonyl, C₂-C₆ alkoxycarbonyl, C₂-C₆ alkylaminocarbonyl, and C₃-C₈ dialkylaminocarbonyl, CONH₂ or CO₂H; or
each R is independently phenyl, phenylW¹, a 5- or 6-membered heterocyclic ring, a 5- or 6-membered heterocyclic ringW², naphthalenyl, or naphthalenylW², each optionally substituted with up to five substituents independently selected from the group consisting of H, halogen, hydroxy, cyano, amino, nitro, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₁-C₄ hydroxyalkyl, C₃-C₇ cycloalkyl, C₃-C₇ cyclohaloalkyl, C₄-C₈ cycloalkylalkyl, C₁-C₄ alkoxy, C₁-C₄ haloalkoxy, C₃-C₇ cycloalkoxy, C₃-C₇ cyclohaloalkoxy, C₄-C₈ cycloalkylalkoxy, C₂-C₄ alkenyloxy, C₂-C₄ alkynyloxy, C₂-C₄ alkoxyalkyl, C₂-C₄ alkoxyhaloalkyl, C₂-C₆ alkylcarbonyloxy, C₁-C₄ alkylthio, C₁-C₄ haloalkylthio, C₂-C₄ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylsulfonyloxy, C₂-C₄ cyanoalkyl, C₂-C₄ cyanoalkoxy, C₁-C₄ nitroalkyl, C₁-C₄ alkylamino, C₂-C₈ dialkylamino, C₃-C₆ cycloalkylamino, C₂-C₄ alkylcarbonyl, C₂-C₆ alkoxycarbonyl, C₂-C₆ alkylaminocarbonyl, C₃-C₈ dialkylaminocarbonyl, C(O)OH, C(O)NH₂ and C(S)NH₂;
each W¹ is independently C₁-C₆ alkanediyl or C₂-C₆ alkenediyl;
each W² is independently C₁-C₆ alkanediyl;
n is 0, 1, 2, 3 or 4;
Q¹ is O, S, carbonyl, sulfonyl, sulfinyl, CR^{6a}R^{6b}, —C(R⁶)═C(R⁷)—, —C(R^{6a})(R^{6b})—C(R^{7a})C(R^{7b})— or NR⁸;
Q² is O, S, carbonyl, sulfonyl, sulfinyl, CR^{6a}R^{6b}, —C(R⁶)═C(R⁷)—, —C(R^{6a})(R^{6b})—C(R^{7a})C(R^{7b})— or NR⁸;
Q³ is O, S, carbonyl, sulfonyl, sulfinyl, CR^{6a}R^{6b}, —C(R⁶)═C(R⁷)—, —C(R^{6a})(R^{6b})—C(R^{7a})C(R^{7b})— or NR⁸;
Q⁴ is O, S, carbonyl, sulfonyl, sulfinyl, CR^{6a}R^{6b}, —C(R⁶)═C(R⁷)—, —C(R^{6a})(R^{6b})—C(R^{7a})C(R^{7b})— or NR⁸;
wherein the bond projecting to the right of the —C(R⁶)═C(R⁷)— or —C(R^{6a})(R^{6b})—C(R^{7a})C(R^{7b})— moieties of Q¹, Q², Q³ or Q⁴ is attached to the benzene moiety of A-1, A-2, A-3 or A-4, respectively; and
each R⁶, R^{6a}, R^{6b}, R⁷, R^{7a}, R^{7b} and R⁸ is independently H, C₁-C₆ alkyl or C₁-C₆ haloalkyl.

2. The compound of claim 1 wherein
X is N;
R¹ is H, halogen, cyano, nitro, C₁-C₆ alkyl, C₁-C₆ haloalkyl, C₁-C₆ haloalkoxy, C₂-C₆ alkoxycarbonyl or C₂-C₆ haloalkoxycarbonyl;
R² is H, halogen, C₁-C₆ alkyl, C₁-C₆ haloalkyl, C₂-C₆ alkylcarbonyl, C₂-C₆ haloalkylcarbonyl, C₁-C₆ alkoxy, C₁-C₆ haloalkoxy, C₂-C₆ alkoxycarbonyl or C₂-C₆ haloalkoxycarbonyl;
R³ is H, C₁-C₄ alkyl or C₂-C₆ alkylcarbonyl; and
R⁴ is C₁-C₆ alkyl or C₃-C₇ cycloalkyl.

3. The compound of claim 2 wherein
R¹ is H, halogen, cyano, nitro, C₁-C₆ haloalkyl or C₁-C₆ haloalkoxy;
R² is H, halogen, C₁-C₆ alkyl or C₁-C₆ haloalkyl;
R³ is H or C₁-C₄ alkyl; and
R⁴ is C₁-C₆ alkyl.

4. The compound of claim 3 wherein
R¹ is C₁-C₂ haloalkyl;
R² is H or C₁-C₆ alkyl;
R³ is H or CH₃; and
R⁴ is CH₃ or CH₂CH₃.

5. The compound of claim 4 wherein
R¹ is CF₃;
R² is H;
R³ is H; and
R⁴ is CH₃.

6. The compound of any of claims 1 to 5 wherein A is A-1; and Q¹ is O.

7. The compound of any of claims 1 to 5 wherein A is A-4; and Q⁴ is O.

8. The compound of any of claims 1 to 5 wherein A is A-4; and Q⁴ is CH₂.

9. The compound of any of claims 1 to 8 wherein each R is independently halogen, C₁-C₄ alkyl or C₁-C₄ haloalkyl; and n is 0, 1, 2 or 3.

10. The compound of any of claims 1 to 9 wherein the stereocenter indicated by the * is predominantly in the R-configuration.

11. The compound of claim 1 selected from the group consisting of
N2-[(1R)-1-(6-fluoro-2-benzofuranyl)ethyl]-5-(trifluoromethyl)-2,4-pyrimidinediamine;
N2-[(1R)-1-(4-fluoro-2-benzofuranyl)ethyl]-5-(trifluoromethyl)-2,4-pyrimidinediamine;
N2-[(1R)-1-(7-fluoro-2-benzofuranyl)ethyl]-5-(trifluoromethyl)-2,4-pyrimidinediamine;
N2-[(1R)-1-benzo[b]thien-2-ylethyl]-5-(trifluoromethyl)-2,4-pyrimidinediamine;
N2-[(1R)-1-(4-fluorobenzo[b]thien-2-yl)ethyl]-5-(trifluoromethyl)-2,4-pyrimidinediamine;
N2-[(1R)-1-(7-fluorobenzo[b]thien-2-yl)ethyl]-5-(trifluoromethyl)-2,4-pyrimidinediamine;
N2-[(1R)-1-(3-benzofuranyl)ethyl]-5-(trifluoromethyl)-2,4-pyrimidinediamine;
N2-[(R)-3-benzofuranylcyclopropylmethyl]-5-(trifluoromethyl)-2,4-pyrimidinediamine; and
N2-[(1R)-1-(2,3-dihydro-1H-inden-2-yl)ethyl]-5-(trifluoromethyl)-2,4-pyrimidinediamine.

12. A compound of claim 1 selected from the group consisting of
N2-[(1R)-1-(2-benzofuranyl)ethyl]-5-(trifluoromethyl)-2,4-pyrimidinediamine; and
N2-[(1R)-2-(3,5-dimethylphenoxy)-1-methylethyl]-5-(trifluoromethyl)-2,4-pyrimidinediamine.

13. A compound of claim 1 selected from the group consisting of
a compound of Formula 1 wherein X is N; R¹ is CF₃; R² is H; R³ is H; R⁴ is Me; A is A-1, Q¹ is S; and n is 0; and
a compound of Formula 1 wherein X is N; R¹ is CF₃; R² is H; R³ is H; R⁴ is Me; A is A-1, Q¹ is 0; and (R)ₙ is 3-F.

14. A herbicidal composition comprising a compound of claim 1 and at least one component selected from the group consisting of surfactants, solid diluents and liquid diluents.

15. The herbicidal composition of claim 14, further comprising at least one additional active ingredient selected from the group consisting of other herbicides and herbicide safeners.

16. A herbicidal mixture comprising (a) a compound of claim 1, and (b) at least one additional active ingredient selected from (b1) through (b16) and salts of compounds of (b1) through (b16).

17. A method for controlling the growth of undesired vegetation comprising contacting the vegetation or its environment with a herbicidally effective amount of a compound of claim 1.

18. The method of claim 17 further comprising contacting the vegetation or its environment with a herbicidally effective amount of at least one additional active ingredient selected from (b1) through (b16) and salts of compounds of (b1) through (b16).
